SEQUENCE LISTING (516 sequences)

SEQ ID NO: 1 - DNA, 114 bp, Homo sapiens
  misc_feature 70: n = A,T,C or G
catcctcggacgccagcaaggtgacctctaagggggcagggctctcaaaggcctttgtgggccagaaggnaaggttccttcctggtggactgcagcaaagctggctccaacatg

SEQ ID NO: 2 - DNA, 430 bp, Homo sapiens
gggactcgccacctcctcttgcacccctgccaggcccagcagccaccacagcgcctgcttcctcggccctgaaatcatgcccctaggtctcctgtggctgggcctagccctgttgggggctctgcatgcccaggcccaggactccacctcagacctgatcccagccccacctctgaacaaggtccctctgcagcagaacttgcaggacaaccaattccaggggaagtggtatgtggtacgcctggcagggaatgcaattctcagagaagacaaagacccgcaaaagatgtatgccaccatctatgagctgaaagaagacaagagctacaatgtcacctccgtcctgtttaggaaaaagaagtgtgactactggatcacgacttttgttccaggttgccagcccggcgagttcacgctgggcaacattaag

SEQ ID NO: 3 - DNA, 527 bp, Homo sapiens
ctgctaatacagccctggctgtggaatccttcaccgtctcagctggtatcagccccagcctgccttgtgccatatctcagcttggatctctgctagagtccccccaaccatatatcatagagttgaatcacaatgagaccgttggctttgaatttgagtcgttggttcccatggtgagatgcttgttaagactttatacttgggtcaatctctcactttattttgtagaaccatttgaaatcctaggatgtgcttgttctggaaggatgacatgggcccagactgaacaagtcagcttgatgatcttaaatgatggaagtataggacgttgcttattttaaaacaagggaaggacacaaaatggaatgactgcttagtcctttctcagatactcttaaaacaattttttattgttaaatttgtggtaatacatggtcacaaccgtggatcaaacaaggtcagtctaaagtggcaggtcctaggtgtgacctgataccaccaccctttgtggcagcaccgggctggac

SEQ ID NO: 4 - DNA, 262 bp, Homo sapiens
  misc_feature 186, 188: n = A,T,C or G
ccggcctcgtggaccagcctgggctctcgctggaggaagtggcttgcaaggaggcttgggaggagtgtggctaccacttggccccctctgatctgcgccgggtcgccacatactggtctggagtgggactgactggctccagacagaccatgttctacacagaggtgacagatgcccagcgtacgntnccaggtgggggcctggtggaggagggtgagctcattgaggtggtgcacctgcccctggaaggcgcccaggcctt

SEQ ID NO: 5 - DNA, 201 bp, Homo sapiens
gccactgaaaatccttgttaaaaaccagatcacaaatctggggctcttggtcccattggagaaggaaggaagagcctcaaaataagtgtgcacccatgcacatattcaggaacagcttgtttagtctttacactttgcctgaaagttgcttctcctcgtccctttgtgtgcctgggtggcctcggccctgtgcgttggcaa

SEQ ID NO: 6 - DNA, 621 bp, Homo sapiens
tgagggtccccgctcagctcctggggctcctgctactctggctccgaggtgccagatgtgacatccagatgacccagtctccatcctccctgtctgcatctgttggagacagagtcaccatcgcttgccgggcaagtcagagcattggcatctatttaaattggtatcaacaaaaaccagggaaagcccctaaactcctgatctatgattcatccagattgcaaagtggggtcccatcaaggttcagtggcagtggaggtgggacacacttcactctcaccatcagcagtctgcaacctgaagatttagcaacttactactgtcaacaagggtacagtacacctggcaccttcggccaagggacacgactggaaattaaacgaactgtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggaggggtgtcacagagcaggacagcaaggacagcacctacagcctcagcagcaccctgacgctgagcaaagcagactacgag

SEQ ID NO: 7 - DNA, 548 bp, Homo sapiens
gacagcatggacatgagggtccccgctcagctcctggggctcctgctactctggctccgaggtgccagatgtgacatccagatgacccagtctccatcctccctgtctgcatctgttggagacagagtcaccatcgcttgccgggcaagtcagagcattggcatctatttaaattggtatcaacaaaaaccagggaaagcccctaaactcctgatctatgattcatccagattgcaaagtggggtcccatcaaggttcagtggcagtggaggtgggacacacttcactctcaccatcagcagtctgcaacctgaagatttagcaacttactactgtcaacaagggtacagtacacctggcaccttcggccaagggacacgactggaaattaaacgaactgtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactc

SEQ ID NO: 8 - DNA, 430 bp, Homo sapiens
tatacacaacatttatttcaaactattgggagggatgagagtggcttaaaaacttccatccctacttttcaagagtgcagttgattctgaatctgaaagcccgcctctgtcctaaaatacaaacaagcacagacattaaacctggatactatatgataaagagggatgtaactattgaattggatacaaggatcagaatggaaagaaactcacgatgaaattgaacctggtttttgtatatttatcaaacttgtgctgagaatagtgtctgattatacgacttttaagcaaagttgggtgtaattaggtgaaaacagcccaggtcctcccgggagcacagaggggctaggggctggtccttctcgtttgctctagtcttgctttgctgtctggtgtagctcctctgctgctcccatctgcactaattgac

SEQ ID NO: 9 - DNA, 493 bp, Homo sapiens
ctcactatttggaatttggccctcgaggccaagaattcggcacgaggcggcacgaggtgtaactattgaattggatacaaggatcagaatggaaagaaactcacgatgaaattgaacctggtttttgtatatttatcaaacttgtgctgagaatagtgtctgattatacgacttttaagcaaagttgggtgtaattaggtgaaaacagcccaggtcctcccgggagcacagaggggctaggggctggtccttctcgtttgctctagtcttgctttgctgtctggtgtagctcctctgctgctcccatctgcactaattgacccaaaacgtgggtatttcctgctacacaaaagccaaaaggtttcatgtagattttagttcactaaagggtgcccacaaaatagagattaattttaacttaaattttaagcttgaagattaggtactatctgtgaagttacactttttttttttttttaaaaggaaaaaatgt

SEQ ID NO: 10 - DNA, 472 bp, Homo sapiens
cggcacgaggtgtaactattgaattggatacaaggatcagaatggaaagaaactcacgatgaaattgaacctggtttttgtatatttatcaaacttgtgctgagaatagtgtctgattatacgacttttaagcaaagttgggtgtaattaggtgaaaacagcccaggtcctcccgggagcacagaggggctaggggctggtccttctcgtttgctctagtcttgctttgctgtctggtgtagctcctctgctgctcccatctgcactaattgacccaaaacgtgggtatttcctgctacacaaaagccaaaaggtttcatgtagattttagttcactaaagggtgcccacaaaatagagattaattttaacttaaattttaagcttgaagattaggtactatctgtgaagttacacttttttatttttttttaaaggtagagatgtgtgtgtgtgtaggtattaaagatgtg

SEQ ID NO: 11 - DNA, 271 bp, Homo sapiens
gtttttcttttttttatacacaacatttatttcaaactattgggagggatgagagtggcttaaaaacttccatccctacttttcaagagtgcagttgattctgggggggaaagcccgcctctgtcctaaaatacaaacaagcacagacattaaacctggatactatatgataaagagggatgtaactattgaattggatacaaggatcagaatggaaagaaactcacgatgaaattgaacctggtttttgtatatttatcaaacttgtgct

SEQ ID NO: 12 - DNA, 343 bp, Homo sapiens
gtttttcttttttttatacacaacatttatttcaaactattgggagggatgagagtggcttaaaaacttccatccctacttttcacgagtgcagctgattctgaatctgaaagcccgcctctgtcctaaaatacaaacacgcacagacattagacctggatactatatgatacagagggatgtaactattgaattggatacacggatcacaatggaaagaaactcacgatgaaattgaacctggcttttgtatatttatcaaacttgtgctgagaatagcgcctgattatacgacttttaagcaaagctgggtgtaattaggtgaaaacagcccacgtcctcc

SEQ ID NO: 13 - DNA, 345 bp, Homo sapiens
agtggcgagcaggttcccacttgccaaagatcccttttaaccaacactagcccttgtttttaacacacgctccagcccttcatcagcctgggcagtcttaccaaaatgtttaaagtgatctcagaggggcccatggattaacgccctcatcccaaggtccgtcccatgacataacactccacacccgccccagccaacttcatgggtcactttttctggaaaataatgatctgtacagacaggacagaatgaaactcctgcgggtctttggcctgaaagttgggaatggttgggggagagaagggcagcagcttattggtggtcttttcaccattggcagaaacg

SEQ ID NO: 14 - DNA, 401 bp, Homo sapiens
ttttccaagtccgtttcagtcccttccttggtctgaagaaattctgcagtggcgagcagtttcccacttgccaaagatcccttttaaccaacactagcccttgtttttaacacacgctccagcccttcatcagcctgggcagtcttaccaaaatgtttaaagtgatctcagaggggcccatggattaacgccctcatcccaaggtccgtcccatgacataacactccacacccgccccagccaacttcatgggtcactttttctggaaaataatgatctgtacagacaggacagaatgaaactcctgcggctctttggcctgaaagttgggaatggttgggggagagaagggcagcagcttattggtggtcttttcaccattggcagaaacagtgagagct

SEQ ID NO: 15 - DNA, 442 bp, Homo sapiens
ggcagccggcccacatgtctctcaagtacctgtcccctcgctctggtgattatttcttgcagaatcaccacacgagaccatcccggcagtcatggttttgctttagttttccaagtccgtttcagtcccttccttggtctgaagaaattctgcagtggcgagcagtttcccacttgccaaagatcccttttaaccaacactagcccttgtttttaacacacgctccagcccttcatcagcctgggcagtcttaccaaaatgtttaaagtgatctcagaggggcccatggattaacgccctcatcccaaggtccgtcccatgacataacactccacacccgccccagccaacttcatgggtcactttttctggaaaataatgatctgtacagacaggacagaatgaaactcctgcggctctttggcctgaaagtgggaatggt

SEQ ID NO: 16 - DNA, 256 bp, Homo sapiens
  misc_feature 96: n = A,T,C or G
gaatatgtagatttgcttcttaatcctgagcgctacactggttacaagggaccagatgcttggaaaatatggaatgtcatctacgaagaaaactgntttaagccacagaccattaaaagaccttaaatcctttggcttctggtcaagggacaagtgaagagaacactttttacagttggctagaaggtctctgtgtagaaaaaagagcttctacagacttatatctggcctacatgcaagccattaatgtgcattt

SEQ ID NO: 17 - DNA, 405 bp, Homo sapiens
attctgtgatttatttgaaactgtgaaaccatgtgccataatagaattttgagaattttgcttttacctaaattcaagaaaatgaaattacacttttaagttagtggtgcttaagcataatttttcctatattaaccagtattaaaatctcaagtaagattttccagtgccagaacatgttaggtggaattttaaaagtgcctcggcatcctgtattacatgtcatagaattgtaaagtcaacatcaattactagtaatcattctgcactcactgggtgcatagcatggttagaggggctagagatggacagtcatcaactggcggatatagcggtacatatgatccttagccaccagggcacaagcttaccagtagacaatacagacagagcttttgttgagct

SEQ ID NO: 18 - DNA, 447 bp, Homo sapiens
tgtgatttcatttgaaactgtgaaaccatgtgccataatagaattttgagaattttgcttttacctaaattcaagaaaatgaaattacacttttaagttagtggtgcttaagcataatttttcctatattaaccagtattaaaatctcaagtaagattttccagtgccagaacatgttaggtggaattttaaaagtgcctcggcatcctgtattacatgtcatagaattgtaaagtcaacatcaattactagtaatcattctgcactcactgggtgcatagcatggttagaggggctagagatggacagtcatcaactggcggatatagcggtacatatgatccttagccaccagggcacaagcttaccagtagacaatacagacagagcttttgttgagctgtaactgagctatggaatagcttctttgatgtacctctttgcctt

SEQ ID NO: 19 - DNA, 294 bp, Homo sapiens
tgtgatttcatttgaaactgtgaaaccatgtgccataatagaattttgagaattttgcttttacctaaattcaagaaaatgaaattacacttttaagttagtggtgcttaagcataatttttcctatattaaccagtattaaaatctcaagtaagattttccagtgccagaacatgttaggtggaattttaaaagtgcctcggcatcctgtattacatgtcatagaattgtaaagtcaacatcaattactagtaatcattctgcactcactgggtgcatagcatggttagaggg

SEQ ID NO: 20 - DNA, 562 bp, Homo sapiens
aggagcaggttggactggccatccgaagcaagattgcagatggcagtgtgaagagagaagacatattctacacttcaaagctttggagcaattcccatcgaccagagttggtccgaccagccttggaaaggtcactgaaaaatcttcaattggactatgttgacctctatcttattcattttccagtgtctgtaaagccaggtgaggaagtgatcccaaaagatgaaaatggaaaaatactatttgacacagtggatctctgtgccacatgggaggccatggagaagtgtaaagatgcaggattggccaagtccatcggggtgtccaacttcaaccacaggctgctggagatgatcctcaacaagccagggctcaagtacaagcctgtctgcaaccaggtggaatgtcatccttacttcaaccagagaaaactgctggatttctgcaagtcaaaagacattgttctggttgcctatagtgctctgggatcccatcgagaagaaccatgggtggacccgaactccccggtgctcttggaggacccagtcctttgtgccttggc

SEQ ID NO: 21 - DNA, 721 bp, Homo sapiens
  misc_feature 626, 685, 696: n = A,T,C or G
ggcacgagatgaggagcaggttggactggccatccgaagcaagattgcagatggcagtgtgaagagagaagacatattctacacttcaaagctttggagcaattcccatcgaccagagttggtcccgaccagccttggaaaggtcactgaaaaatcttcaattggactatgttgacctctatcttattcattttccagtgtctgtaaagccaggtgaggaagtgatcccaaaagatgaaaatggaaaaatactatttgacacagtggatctctgtgccacatgggaggccatggagaagtgtaaagatgcaggattggccaagtccatcggggtgtccaacttcaaccacaggctgctggagatgatcctcaacaagccagggctcaagtacaagcctgtctgcaaccaggtggaatgtcatccttacttcaaccagagaaaactgctggatttctgcaagtcaaaagacattgttctggttgcctatagtgctctgggatcccatcgagaagaaccatgggtggacccgaactccccggtgctcttggaggacccagtcctttgtgccttggcaaaaaagcacaagcgaaccccaccctgattgccctgcgctaccagcttgcancgtggggttgtggtcctggccaagagcttcaatgagcacgcatcagacagaacgtgcangtgtttgaatncagttgacttcagaaggagatgaaa

SEQ ID NO: 22 - DNA, 496 bp, Homo sapiens
agatgataaccagaagtctgcatttgaagttcacaaaagtaatcaagctcaaacagttagtgagaggcagaagaacagacctaaatcttgtaaaaaaggaaaaaatattagggaagatgatcctgtaagaatgttgcaaactgttgcaaagaaattcgacttcagtaatttgagtagtaggttagatggagtcagatttgaaaatgaaaaaaattaatgttattgccaagaacactggtaataaactgaagctaagtcagaaaaaatggttgtttgctagatcccaatggagaaaagtgtgtaactgctcctcgtcaggtctctgctcttcaccataaagacattgctctgtctttggttgctgcaagtgatggagctacagtctgtgttaccacaaggggagatatttacttacttgcagactatcagtgcaagaagatggcttctaaacagttgaacttgaaaaaagttcttgtgtctgggggtcatatggaat

SEQ ID NO: 23 - DNA, 549 bp, Homo sapiens
ctgcatttgaagttcacaaaagtaatcaagctcaaacagttagtgagaggcagaagagcagacctaaatcttgtaaaaaaggaaaaaatattagggaagatgatcctgtaagaatgttgcaaactgttgcaaagaaattcgacttcagtaatttgagtagtaggttagatggagtcagatttgaaaatgaaaaaaattaatgttattgccaagaacactggtaataaactgaagctaagtcagaaaaaatggttgtttgctagatcccaatggagaaaagtgtgtaactgctcctcgtcaggtctctgctcttcaccataaagacattgctctgtctttggttgctgcaagtgatggagctacagtctgtgttaccacaaggggagatatttacttacttgcagactatcagtgcaagaagatggcttctaaacagttgaacttgaaaaaagttcttgtgtctgggggtcatatggaatacaaggttgatcctgaacatttgaaagaaaatgggggtcaaaaaatttgcattcttgcaatggatggagc

SEQ ID NO: 24 - DNA, 55 bp, Homo sapiens
gtgtctgccttcacaaatgtcattgtctactcctagaagaaccaaatacctcaat

SEQ ID NO: 25 - DNA, 498 bp, Homo sapiens
tccttatttatttaacttcacccgagttcctctgggtttctaagcagttatggtgatgacttagcgtcaagacatttgctgaactcagcacattcgggaccaatatatagtgggtacatcaagttcatctgacaaaatggggcagaagagaaaggactcagtgtgtgatccggtttctttttgctcgcccctgttttttgtagaatctcttcatgcttgacatacctaccagtattattcccgacgacacatatacatatgagaatataccttatttatttttgtgtaggtgtctgccttcacaaatgtcattgtctactcctagaagaaccaaatacctcaatttttgtttttgagtactgtactatcctgtaaatatatcttaagcaggtttgttttcagcactgatggaaaataccagtgttgggtttttttttagttgccacagttgtatgtttgctgattatttatgacccgaaaaatatatttcttctccta

SEQ ID NO: 26 - DNA, 325 bp, Homo sapiens
gtcgctgcctctgggggcgctgtacaccgcggccgtcgcggctttagtgctgtacaagtgtgtgggggggggagatgaaactgcggttctccaccaggaggcaagcaagcagcagccactgcagtcagagcaacagctggcccagttgacacaacagctggcccagacagagcagcacctgaacaacctgatggcccagctggaccccctttttgagccgtgtgactactctggctggagcccagcaggagcttctgaacatgaagctatggaccatccacgagctgctgcaagatagcaagccggacaaggatatggaggcttc

SEQ ID NO: 27 - DNA, 166 bp, Homo sapiens
gaatccagcatcttaaagttgcatatgtgtagcactaatgtttctttttaaatagttgggggaaaatgacctagaaaaccaaattgcagtttggtagccaaaattaactcttggtttatttgtcctttgtgtgtgaaaagtcctactattccgtgcgtcagacttc

SEQ ID NO: 28 - DNA, 501 bp, Homo sapiens
tttttttttttttttttttttttttcgcagctgaattacatttactgtacaaagaacggttcggagagaaccaggaatggcggagtgtctaacagcagcgcgggtagtgttgatgccgtgaatgcaggaccatccaggtcctcaaagtctgcgaggtttgttcataatcccaaacaagggccctgctggcagcaacaggacaggtggggccaggacagggaagctggagcaggaggccagtgtctttgggggctgtggcagggccgcctgcctggggttcccttactcatctggtagttcatgcaggccacggccctcatctcccaggaacgggccatggggcgagtccactggtgcccagtaacaccctccgtgggaccaccttgggaagcatgtgccgcggagtccaccacggggggtcctgggtcccgggagggctccttctgcgtgctggccatgtcgtgccgcacggcctgaggacaggaggtagaggtgagcacc

SEQ ID NO: 29 - DNA, 149 bp, Homo sapiens
cgtcccggaggtgcggtgtggggcaccgggcggggccgcgggaaccggcgccccacggagctgctgctgtcagaccaaccccgggcccccatcatcactgcgccgcgctttcaggcgccgagaactaccgttcccggcatgccatgaaa

SEQ ID NO: 30 - DNA, 475 bp, Homo sapiens
agcagtaaacagggctgctatgcctgctctgtagtggtggacggcgaagtaaagcattgtgtcataaacaaaacagcaactggctatggctttgccgagccctataacttgtacagctctctgaaagaactggtgctacattaccaacacacctcccttgtgcagcacaacgactccctcaatgtcacactagcctacccagtatatgcacagcagaggcgatgaagcgcttactctttgatccttctcctgaagttcagccaccctgaggcctctggaaagcaaagggctcctctccagtctgatctgtgaattgagctgcagaaacgaagccatctttctttggatgggactagagctttctttcacaaaaaagaagtaggggaagacatgcagcctaaggctgtatgatgaccacacgttcctaagctggagtgcttatcccttctttttctttttttctttggtttaattt

SEQ ID NO: 31 - DNA, 570 bp, Homo sapiens
cttttttttttttttttttttactggcatcctgtacatttacttttaaaaaaggataacaaaaatgaatattaacaaaaatccgggacaacaatattttcaagcaacaaaaactggggtggggaagcttattctgaaggtacatttaaaactgaaataacaacttaatgaaaattaagaattgcatagcgctgtgaatttagccttcagcaaaacaaaacagaagctatttggtattgatacaaatccatctatttgatagttagtcatccaatattatgtacatattttatatactgaatgtcattttaagtcctgttttccaaactccatttttctgttgctgggtttttgttttttgacaagttaaacactttctggcactttctatgacagaatttcttctgaacatacatgaactgacattctcccaaagcgtcccttgtgagtggacgcgcctttctgctacatatcgttcatttgttacaaaatgaaataatccacagtgcgatgtgtctgggtccaccgtgcacagcaacatccaggctaaaccaggctggaccaaaccttc

SEQ ID NO: 32 - DNA, 645 bp, Homo sapiens
tccgagcgtcgggagcctgtggaagagaagagcgcgcgggcgacagttaaacaggcccgaggcagagaaaccgccctagcagctctcgcgcgcccggtgcaggcggcggttgctgcggaggtccgtgcacagactgctttgcctgttgttgctcttcggaggcggcgatccccgaaggcgagctgaaatacggctgcaggctacaatttgcagccgacgattaaggaagacgacgagcgggagaggtggcccaccctcatggagcgcttgtgctcggatggcttcgcatttccccattactacattaaaccgtatcatctgaagaggatccacagagctgtcttacgtggtaatctggagaaactgaagtaccttctgctcacgtattatgacgccaataagagagacaggaaggaaaggactgccctacatttggcctgtgccactggccaaccggaaatggtacatctcctggtgtccagaagatgtgagcttaacctctgcgaccgtgaagacaggacacctctgatcaaggctgtacaactgaggcaggaggcttgtgcaactcttctgctgcaaatggcgccgatccaaatattacggatgtctttggaaggactgctctgcactacgctgtgtataatg

SEQ ID NO: 33 - DNA, 572 bp, Homo sapiens
ctaactgagtaacattcatgaaatgaggctttctgtggcggcgtagtgtttggaattagaaggtaattcagtagagtgtaacttagagaatattgcaagtgacacattgaatcctgcccgtcagggcaccttttcctcagagcaatccggccacacgaatagaaggctgtcgtgaatcacatcagatgtaaaatcattccttctgtttactcttttaattttcatcctttgcaggtagtgcaaattcaacttcaaatatggtgtaggttttgctagattccatatttttttcttggatttttgctaattatttttagcaaaaaatttttgctcagtggcaccctccctagtgtccatgggttagggccatgctggggaaaacgggccggtatttacacacgcgcaaaacacccagagacggcacaaggaggttgaactcatgtttcagttcgcgaacattgactccttacgaaagtcacttcattctaactagatgcgcccacttctggtcattatttcgtttgcatgatgtattgcttcttcacgttttgtttttattgagcacggagtag

SEQ ID NO: 34 - DNA, 701 bp, Homo sapiens
  misc_feature 34, 41, 43, 52, 58, 72, 180, 204, 205, 211, 214, 228,243, 253, 269, 271, 295, 315, 343, 429, 439, 457, 483, 517, 529,546, 554, 555, 557, 560, 561, 565, 627, 632, 637, 644, 655,659, 662, 672, 680, 689, 690, 698: n = A,T,C or G
ggcacgaggctaactgtgtaacatttatgaaatntgctctntntggcggcgnaggggncggaatgagaaggnaattcagtagagtgtaacttagagaatattgcaagggacacattgaatcctgcccgtcagggcaccttttcctcagagcaatccggccacacgaatagaaggctgcgngaatcacatcagatgtaaaatcannccttcnggngactcttttaattntcatcctttgcaggnagggcaaatncaacttcaaatatggngnaggttttgctagattccatatttntttcttggatttttgctaantatttttagcaaaaaatttttgctcagnggcaccctccctagtgtccatgggttagggccatgctggggaaaacgggccggtatttacacacgcgcaaaacacccagagacggnacaaggaggntgaactcatgtttcagtncgcgaacattgactccttacgaaagncacttcattctaactagatgcgcccacttctggncattattacgantgcatgaaggattgctncttcacgnntnggnnttantgagcacgggagtagaaattccagggctggcttgacatcttccctgcatgctccctcccagnggacngtccntcccttncacatgagganctgncgnccatggtggntttctccnttgggcctnntgggactngga

SEQ ID NO: 35 - DNA, 300 bp, Homo sapiens
gctaactgagtaacattcatgaaatgaggctttctgtggcggcgtagtgtttggaattagaaggtaattcagtagagtgtaacttagagaatattgcaagtgacacattgaatcctgcccgtcagggcaccttttcctcagagcaatccggccacacgaatagaaggctgtcgtgaatcacatcagatgtaaaatcattccttctgtttactcttttaattttcatcctttgcaggtagtgcaaattcaacttcaaatatggtgtaggttttgctagattccatatttttttcttggatt

SEQ ID NO: 36 - DNA, 374 bp, Homo sapiens
tggtacgcctgcaggtaccggtccggaattcccgggtcgacccacgcgtccggaggggtcctggagaatgggttaccccagttgtcttatttaaatggttacccatcagattttaattttatcttctctttgagagcttggtaataagaagcacttaaatcactccaaagaagactttaaaaagggagcagtgaaaaggtcttaataatttattgattgaattaagaaatactagctaattaagaatctgagtctaaacagcacagattttttctttctgcttttaaattgtgttttaaaaaaagagacagggggctgggcgtggtggctcacgcctgtaatcctagcactttgggaggccgaggcgggtggat

SEQ ID NO: 37 - DNA, 290 bp, Homo sapiens
gaggggtcctggagaaatgggttaccccagttgtcttatttaaatggttacccatcagattttaattttatcttctctttgagagcttggtaataagaagcacttaaatcactccaaagaagactttaaaaagggagcagtgaaaaggtcttaataatttattgattgaattaagaaatactagctaattaagaatctgagtctaaacagcacagattttttctttctgcttttaaattgtgttttaaaaaaagagacagggggctgggcgtggtggctcacgcctgtaa

SEQ ID NO: 38 - DNA, 405 bp, Homo sapiens
gccctttcgagcggccgcccgggcaggtacctgggattacaggcacccaccaccacgcctggctaatttttttttgtatctttagtagggttttgccatgttggccaggctggtctttaactcctacctcgtgatccacccgcctcggccccccaaagtgctaggaccacaggcgtgagccaccacgcccagccccctgtctctttttttaaaacacaatttaaaagcagaaagaaaaaatctgtgctgtttagactcagattcttaattagctagtatttcttaattcaatcaataaattattaagaccttttcactgctccctttttaaagtcttctttggagtgatttaagtgcttcttattaccaagctctcaaagagaagataaaattaaaatctgatgg

SEQ ID NO: 39 - DNA, 736 bp, Homo sapiens
  misc_feature 2, 3, 4, 5, 6, 7, 8, 9, 14, 15, 16, 17, 18, 19, 20, 21,22, 23, 24, 25, 26, 27, 28, 29, 30, 31, 32, 33, 34, 35, 36,37, 38, 39, 40, 41, 42, 43, 44, 45, 46, 47, 48, 49, 50,51, 52, 53, 54, 55, 56, 57, 58, 59, 60, 61, 62, 63, 64,65: n = A,T,C or G
  misc_feature 66, 67, 68, 69, 70, 71, 72, 73, 74, 75, 76, 77, 78, 79,80, 81, 82, 83, 84, 85, 86, 87, 88, 89, 90, 91, 92, 93, 94,95, 96, 97, 98, 99, 100, 101, 102, 103, 104, 105, 106, 107,108, 109, 110, 111, 112, 113, 114, 115, 116, 117, 118: n = A,T,C or G
  misc_feature 119, 626, 627, 628, 629, 630, 631, 632, 633, 634, 635,636, 637, 638, 639, 640, 641, 642, 643, 644, 645, 646, 647, 648,649, 650, 651, 652, 653, 654, 655, 656, 657, 658, 659, 660,661, 662, 663, 664, 665, 666, 667, 668, 669, 670, 671: n = A,T,C or G
  misc_feature 672, 673, 674, 675, 676, 677, 678, 679, 680, 681, 682,683, 684, 685, 686, 687, 688, 689, 690, 691, 692, 693, 694, 695,696, 697, 698, 699, 700, 701, 702, 703, 704, 705, 706, 707,708, 709, 710, 711, 712, 713, 714, 729, 736: n = A,T,C or G
gnnnnnnnnagacnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnacctgggattacaggcacccaccaccacgcctggctaatttttttttgtatctttagtagggttttgccatgttggccaggctggtctttaactcctacctcgtgatccacccgcctcggccccccaaagtgctaggaccacaggcgtgagccaccacgcccagccccctgtctctttttttaaaacacaatttaaaagcagaaagaaaaaatctgtgctgtttagactcagattcttaattagctagtatttcttaattcaatcaataaattattaagaccttttcactgctccctttttaaagtcttctttggagtgatttaagtgcttcttattaccaagctctcaaagagaagataaaattaaaatctgatgggtaaccatttaaataagacaactggggtaacccatttctccaggacccctctctgcaacagagagctattctctttctttggcctagtaaacctctgctcttaacctttaaaaaaaaaaaaaaagtaccnnnnnnnnnnnnnnnnnnnnnnnnnnnnnnnnnnnnnnnnnnnnnnnnnnnnnnnnnnnnnnnnnnnnnnnnnnnnnnnnnnnnnnnnncatagtggttcctgngtgaaan

SEQ ID NO: 40 - DNA, 725 bp, Homo sapiens
  misc_feature 2, 3, 4, 5, 6, 7, 8, 9, 10, 12, 13, 15, 16, 17, 18, 19,20, 21, 22, 23, 24, 25, 26, 27, 28, 29, 30, 31, 32, 33, 34,35, 36, 37, 38, 39, 40, 41, 42, 43, 44, 45, 46, 47, 48,49, 50, 51, 52, 53, 54, 55, 56, 57, 58, 59, 60, 61, 62,63: n = A,T,C or G
  misc_feature 64, 65, 66, 67, 68, 69, 70, 71, 72, 73, 74, 75, 76, 77,78, 79, 80, 81, 82, 83, 84, 85, 86, 87, 88, 89, 90, 91, 92,93, 94, 95, 96, 97, 98, 605, 606, 607, 608, 609, 610, 611,612, 613, 614, 615, 616, 617, 618, 619, 620, 621, 622, 623: n = A,T,C or G
  misc_feature 624, 625, 626, 627, 628, 629, 630, 631, 632, 633, 634,635, 636, 637, 638, 639, 640, 641, 642, 643, 644, 645, 646, 647,648, 649, 650, 651, 652, 653, 654, 655, 656, 657, 658, 659,660, 661, 662, 663, 664, 665, 666, 667, 668, 669, 670: n = A,T,C or G
  misc_feature 671, 672, 673, 674, 675, 676, 677, 678, 679, 680, 681,682, 683, 684, 685, 686, 687, 688, 689, 690, 691, 692, 693, 694,695, 696, 697, 698, 699, 700, 701, 702, 703, 704, 705, 706,707, 708, 709, 710, 711, 712, 713, 714, 715, 716, 717: n = A,T,C or G
gnnnnnnnnnanngnnnnnnnnnnnnnnnnnnnnnnnnnnnnnnnnnnnnnnnnnnnnnnnnnnnnnnnnnnnnnnnnnnnnnnnnnnnnnnnnnnnnacctgggattacaggcacccaccaccacgcctggctaatttttttttgtatctttagtagggttttgccatgttggccaggctggtctttaactcctacctcgtgatccacccgcctcggccccccaaagtgctaggaccacaggcgtgagccaccacgcccagccccctgtctctttttttaaaacacaatttaaaagcagaaagaaaaaatctgtgctgtttagactcagattcttaattagctagtatttcttaattcaatcaataaattattaagaccttttcactgctccctttttaaagtcttctttggagtgatttaagtgcttcttattaccaagctctcaaagagaagataaaattaaaatctgatgggtaaccatttaaataagacaactggggtaacccatttctccaggacccctctctgcaacagagagctattctctttctttggcctagtaaacctctgctcttaacctttaaaaaaaaaaaaaaagtaccnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnggtatccg

SEQ ID NO: 41 - DNA, 474 bp, Homo sapiens
  misc_feature 243, 267: n = A,T,C or G
ccggaaaaaaagaaccatttggatacataggtatggtctgagctatgatatcaattggcttcctagggtttatcgtgtgagcacaccatatatttacagtaggaatagacgtagacacacgagcatatttcacctccgctaccataatcatcgctatccccaccggcgtcaaagtatttagctgactcgccacactccacggaagcaatatgaaatgatctgctgcagtgctctgagccctangattcatctttcttttcaccgtangtggcctgactggcattgtattagcaaactcatcactagacatcgtactacacgacacgtactacgttgtagcccacttccactatgtcctatcaataggagctgtatttgccatcataggaggcttcattcactgatttcccctattctcaggctacaccctagaccaaacctacgccaaaatccatttcactatcatattcatcg

SEQ ID NO: 42 - DNA, 540 bp, Homo sapiens
cataggtatggtctgagctatgatatcaattggcttcctagggtttatcgtgtgagcacaccatatatttacagtaggaatagacgtagacacacgagcatatttcacctccgctaccataatcatcgctatccccaccggcgtcaaagtatttagctgactcgccacactccacggaagcaatatgaaatgatctgctgcagtgctctgagccctaggattcatctttcttttcaccgtaggtggcctgactggcattgtattagcaaactcatcactagacatcgtactacacgacacgtactacgttgtagcccacttccactatgtcctatcaataggagctgtatttgccatcataggaggcttcattcactgatttcccctattctcaggctacaccctagaccaaacctacgccaaaatccatttcactatcatattcatcggcgtaaatctaactttcttcccacaacactttctcggcctatccggaatgccccgacgttactcggactaccccgatgcatacaccacatg

SEQ ID NO: 43 - DNA, 587 bp, Homo sapiens
gaccatgagtcatttagaatagtgataaatagaatacacagaatagtgatgaaattcaatttaaaaaatcacgttagcctccaaaccatttaattcaaatgaacccatcaactggatgccaactctggcgaatgtaggacctctgagtggctgtataattgttaattcaaatgaaattcatttaaacagttgacaaactgtcattcaacaattagctccagtaaataacagttatttcatcataaaacagtcccttcaaacacacaattgttctgctgaagagttgtcatcaacaatccaatgctcacctattcagttgctctgtggtcagtgtggctgcatagcagtggattccatgaaaggagtcattttagtgatgagctgccagtccattcccaggccaggctgtcgctggccatccattcagtcgattcagtcataggcgaatctgttctgcccgaggcttgtggtcaagcaaaaattcagccctgaaatcaggcacatctgttcgttggactaaacccacaggttagttcagtcaaagcaggcaacccccttgtgggcactgaccctgccactggggtcat

SEQ ID NO: 44 - DNA, 622 bp, Homo sapiens
  misc_feature 491, 541, 556, 561, 568, 578, 585: n = A,T,C or G
accatgagtcatttagaatagtgataaatagaatacacagaatagtgatgaaattcaatttaaaaaatcacgttagcctccaaaccatttaattcaaatgaacccatcaactggatgccaactctggcgaatgtaggacctctgagtggctgtataattgttaattcaaatgaaattcatttaaacagttgacaaactgtcattcaacaattagctccagtaaataacagttatttcatcataaaacagtcccttcaaacacacaattgttctgctgaagagttgtcatcaacaatccaatgctcacctattcagttgctctgtggtcagtgtggctgcatagcgtgggattccatgaaaggagtcattttagtggtggagctgccagtccttcccgggccgggtgtcgctgggccatccttcagtcgtttcgtcataggcgatctgttctgcccgagggttgtggtcaggcaaaattcagccctgaattngggcactctgttcgttgggctaaacccccggttagttcagtcaaggcggnaacccccttgtgggnactgncctggcnttggggtctnggcggtntggccgttggggaggtttggccccacggcctctgtgg

SEQ ID NO: 45 - DNA, 340 bp, Homo sapiens
aaggcaggaatgtcaggcctctgagcccaagccaagccatcgcatcccctgtgacttgcacgtatacacccagatggcctgaagtaactgaagaatcacaaaagaagtgaaaaggccctgccccgcctcaactgatgacattccaccatggtgatttgttcctgccccaccttaactgagtgattaaccctgtgaatttccttctcctggctcagaagctcccccactgagcaccttgtgacccccgcccctgcccaccagagaacaaccccctttgactgtaatttcccatcaccttcccaaatcctataaaacggccccacccctatctccctttgct

SEQ ID NO: 46 - DNA, 394 bp, Homo sapiens
aaggcaggaatgtcaggcctctgagcccaagccaagccatcgcatcccctgtgacttgcacgtatacacccagatggcctgaagtaactgaagaatcacaaaagaagtgaaaaggccctgccccgcctcaactgatgacattccaccatggtgatttgttcctgccccaccttaactgagtgattaaccctgtgaatttccttctcctggctcagaagctcccccactgagcaccttgtgacccccgcccctgcccaccagagaacaaccccctttgactgtaatttcccatcaccttcccaaatcctataaaacggccccacccctatctccctttgctgactctctttttggactcagcccgcctgcacccaggtgaaataaacagccatgt

SEQ ID NO: 47 - DNA, 246 bp, Homo sapiens
tagccctgataggcgctattttcctcctggttttgtatttgaaccgcaaggggataaaaaagtggatgcataacatcagagatgcctgcagggatcacatggaagggtatcattacagatatgaaatcaatgcggacccggggattaacaaacctcagttctaactcggatgtctgagaaatattagaggacagaccaaggacaactctgcatgagatgtagacttaagctttatccctactaggc

SEQ ID NO: 48 - DNA, 336 bp, Homo sapiens
acatatttccttttcctccattggccacaatgggctccaaacaaccacatgcagattttacaaaaagaaagttccaaaactgctcaatcaaaagaaagattcaactctgtgagatgaatacacacatcacaacgaagtttctcagaatgcttctgtgttgtttttatgtgaagatatttccttttccatcataggcctctaagtgcatcaactatccacttgcagattctacaaaaagagtgtttcaaaactgctcaatcaaaagaaagtatcaactctgtgaggaaatgcacacatcacaaagaagtttctcagaatgattctgtgtagttttta

SEQ ID NO: 49 - DNA, 518 bp, Homo sapiens
cagaagggtctgcaagatgctgttcttggccactttctttcccacctgggaaggcggcatctatgacttcattggggagttcatgaaggccagcgtggatgtgccagacctgataggtctaaaccttgtcatgtcccggaatgccggcaagggagagtacaagatcatggttgctgccctgggctgggccactgctgagcttattatgtcccgctgcattcccctatgggtcggagcccggggcattgagtttgactggaagtacatccagatgagcatagactccaacatcagtctggtccattacatcgtcgcgtctgctcaggtctggatgataacacgctatgatctgtaccacaccttccggccagctgtcctcctgctgatgttcctcagtgtctacaaggcctttgttatggagaccttcgtccacctctgctcgctgggcagttgggcagctctactggcccgagcagtggtaacggggctgctggccctcaacactttggccctgtatg

SEQ ID NO: 50 - DNA, 326 bp, Homo sapiens
tctgcaagatgctgttcttggccactttctttcccacctgggaaggcggcatctatgacttcattggggagttcatgaaggccagcgtggatgtgccagacctgataggtctaaaccttgtcatgtcccggaatgccggcaagggagagtacaagatcatggttgctgccctgggctgggccactgctgagcttattatgtcccgctgcattcccctatgggtcggagcccggggcattgagtttgactggaagtacatccagatgagcatagactccaacatcagtctggtccattacatcgtcgcgtctgctcaggtctggatg

SEQ ID NO: 51 - DNA, 331 bp, Homo sapiens
acattgaaaaaagtctagacaaactgaaaggcaataaatcctatgtgaacatggacctctctccggtggtagagtgcatggaccacgctctaacaagtctcttccctaagactcattatgccgctggaaaagatgccaaaattttctggatacctctgtctcacatgccagcagctttgcaagactttttattgttgaaacagaaagcagagctggctaatcccaaggcagtgtgactcagctaaccacaaatgtctcctccaggctatgaaattggccgatttcaagaacacatctccttttcaaccccattccttatctgctccaaccg

SEQ ID NO: 52 - DNA, 253 bp, Homo sapiens
acagaagggatcgaagacaaattgaagggagagatgatcgatctccaacatggcagccttttccttagaacaccaaagattgtctctggcaaagactctaatgtaactgcaaactccaagctggtcattatcacggctggggcacgtcagcaagagggagaaagccgtcttaatttggtccagcgtaacgtgaacatatttaaattcatcattcctaatgttgtaaaatacagcccgaactgcaagttgctta

SEQ ID NO: 53 - DNA, 356 bp, Homo sapiens
atcgaagacaaattgaagggagagatgatggatctccaacatggcagccttttccttacaacaccaaagattgtctctggcaaagactataatgtaactgcaaactccaagctggtcattatcacggctggggcacgtcagcaagagggagaaagccgtcttaatttggtccagcgtaacgtgaacatatttaaattcatcattcctaaagttgtaaaatacagcccgaactgcaagttgcttattgtttcaaatccagtggatatcttgacctacgtggcttggaagataagtggttttcccaaaaaccgtgttattggaagaggttgcaatctggattcaacccgattccgcta

SEQ ID NO: 54 - DNA, 570 bp, Homo sapiens
ccgctgccgccgattccggatctcattgccacgcgcccccgacgaccgcccgacgtgcattcccgattccttttggttccaagtccaatatggcaactctaaaggatcagctgatttataatcttctaaaggaagaacagaccccccagaataagattacagttgttggggttggtgctgttggcatggcctgtgccatcagtatcttaatgaaggacttggcagatgaacttgctcttgttgatgtcatcgaagacaaattgaagggagagatgatggatctccaacatggcagccttttccttagaacaccaaagattgtctctggcaaagactataatgtaactgcaaactccaagctggtcattatcacggctggggcacgtcagcaagagggagaaagccgtcttaatttggtccagcgtaacgtgaacatatttaaattcatcattcctaatgttgtaaaatacagcccgaactgcaagttgcttattgtttcaaatccagtggatatcttgacctacgtggcttggaagataagtggttttcccaaaaaccgtgttattggaa

SEQ ID NO: 55 - DNA, 223 bp, Homo sapiens
gccgctgccgccgattccggatctcattgccacgcgcccccgacgaccgcccgacgtgcattcccgattccttttggttccaagtccaatatggcaactctaaaggatcagctgatttataatcttctaaaggaagaacagaccccccagaataagattacagttgttggggttggtgctgttggcatggcctgtgccatcagtatcttaatgaaggacttgg

SEQ ID NO: 56 - DNA, 337 bp, Homo sapiens
gatgcccataagatatgggaagctatgttatcaagccatattagatatcaagcattaatatggaaataaaccagcctgtttggtgggctcttcacatggacgcgcatgaaatttggtgccgtgactaggatcgggggacctcccttgggagatcaatcccctgtcctcctgctctttgctccgtgagaaacatgcacctatggcctcatgttctcaaaccgaccaaaccaagaaacatctcaccaattttaaatccgcctggcttgtgaggccttttgaccccaattcaagtcttttgataccctgtgaattgcacccatactgcccagatggctag

SEQ ID NO: 57 - DNA, 473 bp, Homo sapiens
aaagatcaaagtgctgggctccggtgcgttcggcacggtgtataagggactctggatcccagaaggtgagaaagttaaaattcccgtcgctatcaaggaattaagagaagcaacatctccgaaagccaacaaggaaatcctcgatgaagcctacgtgatggccagcgtggacaacccccacgtgtgccgcctgctgggcatctgcctcacctccaccgtgcaactcatcacgcagctcatgcccttcggctgcctcctggactatgtccgggaacacaaagacaatattggctcccagtacctgctcaactggtgtgtgcagatcgcaaagggcatgaactacttggaggaccgtcgcttggtgcaccgcgacctggcagccaggaacgtactggtgaaaacaccgcagcatgtcaagatcacagattttgggctggccaaactgctgggtgcggaagagaaagaataccatg

SEQ ID NO: 58 - DNA, 487 bp, Homo sapiens
  misc_feature 7: n = A,T,C or G
actatcncccaggacatgggaccatgctcagctggttgctatcaagaccttgaaagactataacaacccccagcaatggatggaatttcaacaagaagcctccctaatggcagaactgcaccaccccaatattgtctgccttctaggtgccgtcactcaggaacaacctgtgtgcatgctttttgagtatattaatcagggggatctccatgagttcctcatcatgagatccccacactctgatgttggctgcagcagtgatgaagatgggactgtgaaatccagcctggaccacggagattttctgcacattgcaattcagattgcagctggcatggaatacctgtctagtcacttctttgtccacaaggaccttggcagctcgcaatattttaatcggagaggcaactttcatgttaaaggttttcaggacttgggggctttccagaggaaattttactccgctgatttactacagggtacccaa

SEQ ID NO: 59 - DNA, 532 bp, Homo sapiens
atagaagtctgggaaaaaaataaaaacagaatttgagaaccttggaccactcctgtccctgtagctcagtcatcaaagcagaagtctggctttgctctattaagattggaaatgtacactaccaaacactcagtccactgttgagccccagtgctggaagggaggaaggcctttcttctgtgttaattgcgtaaaggctacaggggttagcctggactaaaggcatccttgtcttttgagctattcacctcagtagaaaaggatctaagggaagatcactgtagtttagttctgttgacctgtgcacctaccccttggaaatgtctgctggtatttctaattccacaggtcatcagatgcctgcttgataatatataaacaataaaaacaaccttcacttcttcctattgtaatcgtgtgccatggatctgatctgtaccatgaccctacataaggctggatggcaccccaggctgagggccccaatgtatgtgtggctgtgggtgtgggtgggagtgtgtctgctgagtaa

SEQ ID NO: 60 - DNA, 608 bp, Homo sapiens
tacggccgggatagagtctggaaaaaataaaaacagaatttgagaaccttggaccactcctgtccctgtagctcagtcatcaaagcagaagtctggctttgctctattaagattggaaatgtacactaccaaacactcagtccactgttgagccccagtgctggaagggaggaaggcctttcttctgtgttaattgcgtagaggctacaggggttagcctggactaaaggcatccttgtcttttgagctattcacctcagtagaaaaggatctaagggaagatcactgtagtttagttctgttgacctgtgcacctaccccttggaaatgtctgctggtatttctaattccacaggtcatcagatgcctgcttgataatatataaacaataaaaacaaccttcacttcttcctattgtaatcgtgtgccatggatctgatctgtaccatgaccctacataaggctggatggcaccccaggctgagggccccaatgtatgtgtggctgtgggtgtgggtgggagtgtgtctgctgagtaaggaacacgattttcaagattctaaagctcaattcaagtgacacattaatgataaactcagatctgatca

SEQ ID NO: 61 - DNA, 480 bp, Homo sapiens
tagatgacactgatgattctcaccagtcttatgagtctcaccattctgatgaatctgatgaactggtcactgattttcccacggacctgccagcaaccgaagttttcactccagttgtccccacagtagacacatatgatggccgaggtgatagtgtggtttatggactgaggtcaaaatctaagaagtttcgcagacctgacatccagtaccctgatgctacagacgaggacatcacctcacacatggaaagcgaggagttgaatggtgcatacaaggccatccccgttgcccaggacctgaacgcgccttctgattgggacagccgtgggaaggacagttatgaaacgagtcagctggatgaccagagtgctgaaacccacagccacaagcagtccagattatataagcggaaagccaatgatgagagcaatgagcattccgatgtgattgatagtcaggaactttccaaagtcagcc

SEQ ID NO: 62 - DNA, 440 bp, Homo sapiens
aggagatccggcagatgggcactgagtgccattacttcatctgtgatgtgggcaaccgggaggaggtgtaccagacggccaaggccgtccgggagaaggtgggtgacatcaccatcctggtgaacaatgccgccgtggtccatgggaagggcctaatggacagtgatgatgatgccctcctcaagtcccaacacatcaacaccctgggccagttctggaccaccaaggccttcctgccgcgtatgctggagctgcagaatggccacatcgtgtgcctcaactccgtgctggcactgtctgccatccccggtgccatcgactaccgcacatccaaagcgtcagccttcgccttcatggagagcctgaccctggggctgctggactgtccgggagtcagcgccaccacagtgctgcccttccacaccagcaccgagatgttc

SEQ ID NO: 63 - DNA, 589 bp, Homo sapiens
ggcactgagtgccattacttcatctgtgatgtgggcaaccgggaggaggtgtaccagacggccaaggccgtccgggagaaggtgggtgacatcaccatcctggtgaacaatgccgccgtggtccatgggaagggcctaatggacagtgatgatgatgccctcctcaagtcccaacacatcaacaccctgggccagttctggaccaccaaggccttcctgccgcgtatgctggagctgcagaatggccacatcgtgtgcctcaactccgtgctggcactgtctgccatccccggtgccatcgactaccgcacatccaaagcgtcagccttcgccttcatggagagcctgaccctggggctgctggactgtccgggagtcagcgccaccacagtgctgcccttccacaccagcaccgagatgttccagggcatgagagtcaggtttcccaacctctttcccccactgaagccggagacggtggcccggaggacagtggaagctgtgcagctcaaccaggccctcctcctcctcccatggacaatgcatgccctcgttatcttgaaaagcatacttccacaggctgcactcg

SEQ ID NO: 64 - DNA, 313 bp, Homo sapiens
gcatattgtgctcggggaagggttcttgtcattgtgggaagtgcatttgttctgctgaagagtggtatatttctggggagttctgtgactgtgatgacagagactgcgacaaacatgatggtctcatttgtacagggaatggaatatgtagctgtggaaactgtgaatgctgggatggatggaatggaaatgcatgtgaaatctggcttggctcagaatatccttaacaattacatgagagaggtctggattcttattttttctgggccattagaacatataaatgcgaaggaaaccatgtatattcaccact

SEQ ID NO: 65 - DNA, 223 bp, Homo sapiens
tgtgaatcagcagatggcatattgtgctcggggaagggttcttgtcattgtgggaagtgcatttgttctgctgaagagtggtatatttctggggagttctgtgactgtgatgacagagactgcgacaaacatgatggtctcatttgtacagggaatggaatatgtagctgtggaaactgtgaatgctgggatggatggaatggaaatgcatgtgaaatctggc

SEQ ID NO: 66 - DNA, 424 bp, Homo sapiens
ggtacagatttagagcctgtaatcccagctacttgggagtctaaggcaagagaatcccttgaacctgggaggtggagattgcagtgagctgagatcacaccattgccctacagcctgggtgacagtgagactgccccaagaaaaaacaaaagagacagccctagtgatcttgtaagttgcctttggtgggtcagtctttccttttcttaaagaatagtacacattgacagccaggtagctctatgatcctgttctatagaattcaaaaagtcgacaaccttcctttgttcctttctgttttctctgcctacgttagtttaaattggcagtgtctctgctggaataatcccatctctcttcctggcttctgctgagatggctgattaaatccttgggtcacacccattatctctttatcaaatgg

SEQ ID NO: 67 - DNA, 487 bp, Homo sapiens
ctgtaatcccagctacttgggagtctaaggcaagagaatcccttgaacctgggaggtggagattgcagtgagctgagatcacaccattgccctacagcctgggtgacagtgagactgccccaagaaaaaacaaaagagacagccctagtgatcttgtaagttgcctttggtgggtcagtctttccttttcttaaagaatagtacacattgacagccaggtagctctatgatcctgttctatagaattcaaaaagtcgacaaccttcctttgttcctttctgttttctctgcctacgttagtttaaattggcagtgtctctgctggaataatcccatctctcttcctggcttctgctgagatggctgattaaatccttgggtcacacccattatctctttatcaaatggttgttcaggctaggctcagtgtttcacgcctgtaatcccaacactttgggagactgaggagggcagatcacttgagctc

SEQ ID NO: 68 - DNA, 492 bp, Homo sapiens
agtcgcgcaccgacgctcaaacgcgcgctccaacccgcagcctcctcctgcctcaccgcccgaagatggcggctctcaaactcctctcctccgggcttcggctctgcgcctctgcccgcggatctggggcaacctggtacaagggatgtgtttgttccttttccaccagtgctcatcgccataccaagttttatacagatccagtagaagctgtaaaagacatccctgatggtgccacggttttggttggtggttttgggctatgtggaattccagagaatcttatagatgctttactgaaaactggagtaaaaggactaactgcagtcagcaacaatgcaggggttgacaattttggtttggggcttttgcttcggtccaagcagataaaacgcatggtctcttcatatgtgggagaaaatgcagaatttgaacgacagtacttatctggtgaattagaagtggagctgacaccacagggcacacttgctg

SEQ ID NO: 69 - DNA, 494 bp, Homo sapiens
tttttttttttttttttttctccctttataaggcgatgtacataaatctgaggaatatggatgtcttctggagcaaatgctccaatatccacaatttcttcaacctctaccactgtggtttctgcagctttgcacattggcaagttgaaattccttgcacttttcctgaaaatcacgtttcctgctcggtccgccttccaggctttcaccaaagcaaaatcccctgtaattgcttcctccaaaataaagtgctgaccattgaactccctcacctctcttggcttattggcaatggcaacactgccatctttgttgtatttgatgggcgatcctccttcttgtaccagggtcccataccctgttggggtgtaaaatgcaggaactccagccccgcctgcacggatcctctcagcaagtgtgccctgtggtgtcagctccacttctaattcaccagataagtactgtcgttcaaattctgcattttctcccacata

SEQ ID NO: 70 - DNA, 462 bp, Homo sapiens
catgatgtattacaaggaggccttctggaagaagaaggattactgtggctgcatgatcattgaagatgaagatgctccaatttcaataaccttggatgacaccaagccagatgggtcactgcctgccatcatgggctttattcttgcccggaaagctggtcgacttgctaagctacataaggaaataaggaagaagaaaatctgtgagctctatgccaaagtgctgggatcccaagaagctttacatccagtgcattatgaagagaagaactggtgtgaggagcagtactctgggggctgctacacggcctacttccctcctgggatcatgactcaatatggaagggtgattcgtcaacccgtgggcaggattttctttgcgggcacagagactgccacaaagtggagcggctacatggaaggggcagttgaggctggagaacgagcagctagggaggtctt

SEQ ID NO: 71 - DNA, 626 bp, Homo sapiens
catgatgtattacaaggaggccttctggaagaagaaggattactgtggctgctgatcattgaaaatgaagatgctcaatttcaataaccttggatgacaccaagccagatgggtcactgcctgccatcatgggcttcattcttgcccggaaagctggtcgacttgctaagctacataaggaaataaggaagaagaaaatctgtgagctctatgccaaagtgctgggatcccaagaagctttacatccagtgcattatgaagagaagaactggtgtgaggagcagtactctgggggctgctacacggcctacttccctcctgggatcatgactcaatatggaagggtgattcgtcaacccgtgggcaggattttctttgcgggcacagagactgccacaaagtggagcggctacatggaaggggcagttgaggctggagaacgagcagctagggaggtcttaaatggtctcgggaaggtgaccgagaaagacatctgggtacaagaacctgaatcaaaggacgttccagcggtagaaatcacccacaccttctgggaaaggaacctgccctctgtttctggcctgctgaagatcattggatttccacatcagtaactgccctggggc

SEQ ID NO: 72 - DNA, 348 bp, Homo sapiens
tggtgaactggtcatccatgaaaaagggttttactacatctattcccaaacatactttcgatttcaggaggaaataaaagaaaacacaaagaacgacaaacaaatggtccaatatatttacaaatacacaagttatcctgaccctatattgttgatgaaaagtgctagaaatagttgttggtctaaagatgcagaatatggactctattccatctatcaagggggaatatttgagcttaaggaaaatgacagaatttttgtttctgtaacaaatgagcacttgatagacatggaccatgaagccagttttttcggggcctttttagttggctaactgacctggaaaga

SEQ ID NO: 73 - DNA, 207 bp, Homo sapiens
  misc_feature 122, 123: n = A,T,C or G
tcaactcagtggaacacggttctcccaaacagattttgtaattccgaaaaccacgcatgcgcaaacatacgcatacactcccatgttcctggacagtttatagctaccataacctggcattnnccaaaacataccatggtagactcttggatacacaaggtaattttagagccacattaggatgaaccttctgaaaaagttatgcat

SEQ ID NO: 74 - DNA, 497 bp, Homo sapiens
gagcttaaggaaaatgacagaatttttgtttctgtaacaaatgagcacttgatagacatggaccatgaagccagttttttcggggcctttttagttggctaactgacctggaaagaaaaagcaataacctcaaagtgactattcagttttcaggatgatacactatgaagatgtttcaaaaaatctgaccaaaacaaacaaacagaaaacagaaaacaaaaaaacctctatgcaatctgagtagagcagccacaaccaaaaaattctacaacacacactgttctgaaagtgactcacttatcccaagaaaatgaaattgctgaaagatctttcaggactctacctcatatcagtttgctagcagaaatctagaagactgtcagcttccaaacattaatgcaatggttaacatcttctgtctttataatctactccttgtaaagactgtagaagaaagcgcaacaatccatctctcaagtagtgtatcacagtagtag

SEQ ID NO: 75 - DNA, 275 bp, Homo sapiens
  misc_feature 96: n = A,T,C or G
tgagcttaaggaaaatgacagaatttttgtttctgtaacaaatgagcacttgatagacatggaccatgaagccagttttttcggggcctttttagntggctaactgaccctggaaagaaaaagcaataacctcaaagtgactattcagttttcaggatgatacactatgaagatgtttcaaaaaatctgaccaaaacaaacaaacagaaaacagaaaacaaaaaaacctctatgcaatctgagtagagcagccacaaccaaaaaattctacaaca

SEQ ID NO: 76 - DNA, 530 bp, Homo sapiens
gacagaaggggcctctccgccccgcgtccagctcgcccagctcgcccagcgtccgccgcgcctcggccaaggcttcaacggaccacaccaaaatgccatctcaaatggaacacgccatggaaaccatgatgtttacatttcacaaattcgctggggataaaggctacttaacaaaggaggacctgagagtactcatggaaaaggagttccctggatttttggaaaatcaaaaagaccctctggctgtggacaaaataatgaaggacctggaccagtgtagagatggcaaagtgggcttccagagcttcttttccctaattgcgggcctcaccattgcatgtaatgactattttgtagtacacatgaagcagaagggaaagaagtaggcagaaatgagcagttcgctcctccttgataagagttgtcccaaagggtcgcttaaggaatctgccccacagcttcccccatagaaggatttcatgagcagatcaggacacttagcaaatgtaaaaataaaatctaactctcat

SEQ ID NO: 77 - DNA, 341 bp, Homo sapiens
gcctctccgccccgcgtccagctcgcccagctcgcccagcgtccgccgcgcctcggccaaggcttcaacggaccacaccaaaatgccatctcaaatggaacacgccatggaaaccatgatgtttacatttcacaaattcgctggggataaaggctacttaacaaaggaggacctgagagtactcatggaaaaggagttccctggatttttggaaaatcaaaaagaccctctggctgtggacaaaataatgaaggacctggaccagtgtagagatggcaaagtgggcttccagagcttcttttccctaattgcgggcctcaccattgcatgtaatgactatt

SEQ ID NO: 78 - DNA, 350 bp, Homo sapiens
ggcctctccgccccgcgtgcagctcgcccagctcgcccagcgtccgccgcgcctcggccaaggcttcaacggaccacaccaaaatgccatctcaaatggaacacgccatggaaaccatgatgtttacatttcacaaattcgctggggataaaggctacttaacaaaggaggacctgagagtactcatggaaaaggagttccctggatttttggaaaatcaaaaagaccctctggctgtggacaaaataatgaaggacctggaccagtgtagagatggcaaagtgggcttccagagcttcttttccctaattgcgggcctcaccattgcatgcaatgactattttgtagta

SEQ ID NO: 79 - DNA, 171 bp, Homo sapiens
acagaagggacaaagagatctggacagaatcgccggacaggtggcagctgccaacaagaagcattagaacaaaccatgctgggttaataaattgcctcattcgtaaacaaaaaaaaaaaaaaaaaaaaaaagttttttttttttccccccattttttattttttttccccc

SEQ ID NO: 80 - DNA, 389 bp, Homo sapiens
tggcgctgtgttctatggaggaaaacaaagcaggagaggggagagtgactgctgggtaaggtcttcctccacctcctttgcatctttgctcacatgccagcttctcctgggcttcacagaccaccaatttataatttccatttaaaacttccattttatttttttaatttttatttatttatttatttattacgagatggggtttcgctcttgttgcccaagattgcaccactgcactgcagcctgggtgacagagcgagactttgtcaaaaagaaagaaagaaagaaggaaaggaaggaaggaaggaaggaaggaaggaaaagaaaagaaagggaaagaaaaaagaaaaagaaagaaagaaagaaaaaaaaaaaaaaggggggccccc

SEQ ID NO: 81 - DNA, 430 bp, Homo sapiens
tgcagatacagtggtggagtggaagtttgcgttggtagagaatgggggagttacccgctgggaagaatgcagcaatagattcctagaaactggccatgaggataaagtggttcacgcatggtgggggattcactgattcagtttgcatagtaatggagaagctgtagaacaatgtggaagaagctgaggttgtggaacacactgaataaaataaaggcagtgtgactccaaattcagccatctgaattgtttaaatttgctagtggattttgtctactgtgcagaaatatatatgtctaatgtgcagaaatatatatgtgtgtatgtgtgtatatatatgcacacacacacagataatgcttccagtgaatgtgaacttcttttccctgtggcactgattgacagacttgtgctgatccattattacttt

SEQ ID NO: 82 - DNA, 556 bp, Homo sapiens
ttttttttttttttttttttttttttttaagatattaaaattcaggttttattatttgttcagttataataatttaagttaatatttgctgtattctcagagcaaagatgtatttctgtaccactgtcctgtataaatttgttacccaagatagtgactggtatgaaaggagagggaagagggtgacagatggaaacgattgctgtaggacagtccatctggccagatgcggtgggggaggggagaagaagtgggagagagatggtcctacagatgctcccatgggtaaatgatgggtgcatccctccctgcagtcgggctgtgcctgaacttcacagtcctctaagaggtgtcattcaggccacctcactcagcctatgcccaaccccactcactttccctttccttatgggctgcccccgcaactgacttccatggtgattggttctcattaggccctttgtttctacaccagccttagatcattaagacaaagacgtacttgctaccctcatagcacataacaacgcctggcagatgaaaatcaaacaaaaag

SEQ ID NO: 83 - DNA, 543 bp, Homo sapiens
tgcagtggacatgtcgggcgggacggtcacagtccttgaaaagtccctgtatcaaaaggccaactgaagcaatacttctacgagaccaagtgcaatcccatgggttacacaaaagaaggctgcaggggcatagacaaaaggcattggaactcccagtgccgaactacccagtcgtacgtgcgggcccttaccatggatagcaaaaagagaattggctggcgattcataaggatagacacttcttgtgtatgtacattgaccattaaaaggggaagatagtggatttatgttgtatagattagattatattgagacaaaaattatctatttgtatatatacataacagggtaaattattcagttaagaaaaaaataattttatgaactgcatgtataaatgaagtttatacagtacagtggttctacaatctatttattggacatgtccatgaccagaagggaaacagtcatttgcgcacaacttaaaaagtctgcattacattccttgataatgttgtggtttgttgccgttgccaagaactg

SEQ ID NO: 84 - DNA, 242 bp, Homo sapiens
cggcggcagacaaaaagactgcagtggacatgtcgggcgggacggtcacagtccttgaaaaggtccctgtatcaaaaggccaactgaagcaatacttctacgagaccaagtgcaatcccatgggttacacaaaagaaggctgcaggggcatagacaaaaggcattggaactcccagtgccgaactacccagtcgtacgtgcgggcccttaccatggatagcaaaaagagaattggctggcga

SEQ ID NO: 85 - DNA, 350 bp, Homo sapiens
tttttttttttttttttttttctttttttttttttttttttttattattaattatcttctttattaatactcacatgtaacctttgctttttacacaaaagtctgctttagaagaatgcctcctcggcttatcatgcccaatggggctttttgtttctggaccacttcccctttctccacccccacccccacatccaaattactcttaacatgttcacagataccacgaatattttgtaaacaagatttgggttactggaacttgatttcattaacatcccacttcaaaatggaaggcaggtggaggacagggtaagaaataggagaaagaggacaagagaaggcaaaga

SEQ ID NO: 86 - DNA, 448 bp, Homo sapiens
acagtttaagaagtggtgacattttgcatgatgaatgacctgacttttagccaccaggtactctttaaacagttttccttatcagaggccctcctgtgctggtgacccagcatctgagttaggttccagcatgtaaagagctgggagggcggagaattcttagcatacattcagacgttttttctgcacaataataagtccatctgtcacttgcattccactttttgttacatagaaagagtctgaccctttaatccaaaaggtctttttacattgtgaatgctgtgggaaggcaatttctctgcacacaagaggctacgttttggaagtgatgtatgttatttgatgactgaaaatgaactgtaaatgctcctagagtatattcctctgctgaacaaaattaaacttcaaaaaaatctaacagtaacacacccctgcttgggaccct

SEQ ID NO: 87 - DNA, 586 bp, Homo sapiens
aatttacagaacagtttaagaagtggtgacattttgcatgatgaatgacctgacttttagccaccaggtactctttaaacagttttccttatcagaggccctcctgtgctggtgacccagcatctgagttaggttccagcatgtaaagagctgggagggcggagaattcttagcatacattcagacgttttttctgcacaataataagtccatctgtcacttgcattccactttttgttacatagaaagagtctgaccctttaatccaaaaggtctttttacattgtgaatgctgtgggaaggcaatttctctgcacacaagaggctacgttttggaagtgatgtatgttatttgatgactgaaaatgaactgtaaatgctcctagagtatattcctctgctgaacaaaattaaacttcaaaaaaatctaacagtaacacacccctgcttgggaccctagctatatgcattttatgtgaccttgccatgcttcagtgaacatactaattctatgtctagcacatgttgatttcctatgtattctgggtattctattaaaggaaactttgaactatgtcaaaaaaaa

SEQ ID NO: 88 - DNA, 203 bp, Homo sapiens
aatgaatttacagaacagtttaagaagtggtgacattttgcatgatgaatgacctgacttttagccaccaggtactctttaaacagttttccttatcagaggccctcctgtgctggtgacccagcatctgagttaggttccagcatgtaaagagctgggagggcggagaattcttagcatacattcagacgttttttctgcac

SEQ ID NO: 89 - DNA, 548 bp, Homo sapiens
tgctggaaggcattcgcatctgccggcgagggcttccgcaaccggatcgtcttccaggagttccgccaacgctacgagatcctggcggcgaatgccatccccaaaggcttcatggacgggaagcaggcctgcattctcatgatcaaagccctggaacttgaccccaacttatacaggatagggcagagcaaaatcttcttccgaactggcgtcctggcccacctagaggaggagcgagatttgaagatcaccgatgtcatcatggccttccaggcgatgtgtcgtggctacttggccagaaaggcttttgccaagaggcagcagcagctgaccgccatgaaggtgattcagaggaactgcgctgcctacctcaagctgcggaactggcagtggtggaggcttttcaccaaagtgaagccactgctgcaggtgacacggcaggaggaggagatgcaagccaaggaggatgaactgcagaagaccaaggagcggcagcagaaggcagagaatgagcttaaggagctggaacagaagcactcgcagctgac

SEQ ID NO: 90 - DNA, 595 bp, Homo sapiens
tgcaatggggtgctggaaggcattcgcatctgccggcagggcttccccaaccggatcgtcttccaggagttccgccaacgctacgagatcctggcggcgaatgccatccccaaaggcttcatggacgggaagcaggcctgcattctcatgatcaaagccctggaacttgaccccaacttatacaggatagggcagagcaaaatcttcttccgaactggcgtcctggcccacctagaggaggagcgagatttgaagatcaccgatgtcatcatggccttccaggcgatgtgtcgtggctacttggccagaaaggcttttgccaagaggcagcagcagctgaccgccatgaaggtgattcagaggaactgcgctgcctacctcaagctgcggaactggcagtggtggaggcttttcaccaaagtgaagccactgctgcaggtgacacggcaggaggaggagatgcaggccaaggaggatgaactgcagaagaccaaggagcggcagcagaaggcagagaatgagcttaaggagctggaacagaagcactcgcagctgaccgaggagaagaacctgctacaggaacagctgcaggcag

SEQ ID NO: 91 - DNA, 498 bp, Homo sapiens
tgacagagcaagacttggtttcaaaaaagagaaacacagttggccctccatatctgagtttcacagacgaaaaatattcagaagaaaaaaaaatcaatggctgtatttgtactaaacatgcccaggctttttttcttattgttatcccctaaacaatacaacaactatttttatagcatttacattgtattagatgttataactactctaaagaggatttaaagtatatggaatgatgtgcataggttatatgcaaatactatactatttatatcagggacttgagcatccttggattttggtatgtgtgggaggtcctgaaaccaatgtcctgtggatactgaaggataactgtactaatttggagatttctctctactatgatcaagattttcaaacattacattgctgattacattacatcgttacattgtgattctttccaagacttgagataaagtttgggaagaagtaccacttgtttcagtttatgaaata

SEQ ID NO: 92 - DNA, 510 bp, Homo sapiens
aaacacagttggccctccatatctgagtttcacagacgaaaaatattcagaagaaaaaaaaatcaatggctgtatttgtactaaacatgcccaggctttttttcttattgttatcccctaaacaatacaacaactatttttatagcatttacattgtattagatgttataactactctaaagaggatttaaagtatatggaatgatgtgcataggttatatgcaaatactatactatttatatcagggacttgagcatccttggattttggtatgtgtgggaggtcctgaaaccaatgtcctgtggatactgaaggataactgtactaatttggagatttctctctactatgatcaagattttcaaacattacattgctgattacattacatcgttacattgtgattctttccaagacttgagataaagtttgggaagaagttaccacttgtttcagtttatgaaatagaaaaaaaaaaaaggggtaaagcatgaaataaaaacctaaac

SEQ ID NO: 93 - DNA, 299 bp, Homo sapiens
tggatcccccgggctgcaggaattcggcacgagcagaagtgcctgagacgcggagacatggctggtgttaaatggagctattcaatagcagtgacgcgctctcctcagccaccaaatgtccctgacaccctccccagcccccacagataacatcagctgaggtttttttcagtatgaacctgtcctaaatcaattcctcaaagtgtgcacaaaactaaagaatataaataaacaaaagaaaggtgaaaaaaaaaaaaaaaaaaaaaactcgggggggggcccgggccccaattccccct

SEQ ID NO: 94 - DNA, 234 bp, Homo sapiens
  misc_feature 163, 189, 219, 222, 225, 226, 228, 233: n = A,T,C or G
cagaagtgcctgagacgcggagacatggctggtgttaaatggagctattcaatagcagtgacgcgctctcctcagccaccaaatgtccctgacaccctccccagcccccacagataacatcagctgaggtttttttcagtatgaacctgtcctaaatcaattnctcaaagtgtgcacaaaactaaagantataaataaacaaaagaaaggtgaaaaaanaanaannanaaaana

SEQ ID NO: 95 - DNA, 534 bp, Homo sapiens
tgaagcagaagtacctggactatgccagagtccccaatagcaatccccctgaatatgagttcttctggggcctgcgctcttactatgagaccagcaagatgaaagtcctcaagtttgcctgcaaggtacaaaagaaggatcccaaggaatgggcagctcagtaccgagaggcgatggaagcagatttgagggctgcagctgaggctgcagctgaagccaaggctagggccgagattagagctcgaatgggcattgggctcggctcggagaatgctgccgggccctgcaactgggacgaagctgatatcggaccctgggccaaagcccggatccaggcgggagcagaagctaaagccaaagcccaagagagtggcagtgccagcactggtgccagtaccagtaccaataacagtgccagtgccagtgccagcaccagtggtggcttcagtgctggtgccagcctgaccgccactctcacatttgggctcttcgctggccttggtggagctggtgccagcaccagtggcagctctg

SEQ ID NO: 96 - DNA, 351 bp, Homo sapiens
tttttttttttttttttttttttctgaaatggcaaatagatttaatgcagagtgtcaacttcaattgattgatagtggctgcctagagtgctgtgttgagtaggtttctgaggatgcaccctggcttgaagagaaagactggcaggattaacaatatctaaaatctcacttgtaggagaaaccacaggcaccagagctgccactggtgctggcaccagctccaccaaggccagcgaagagcccaaatgtgagagtggcggtcaggctggcaccagcactgaagccaccactggtgctggcactggcactggcactgttattggtactggtactggcaccagtgctggcact

SEQ ID NO: 97 - DNA, 610 bp, Homo sapiens
tttatgaatgataaagatgtttccggaaagatgaacaggtcacaatttgaagaactctgtgctgaacttctgcaaaagatagaagtacccctttattcactgttggaacaaactcatctcaaagtagaagatgtgagtgcagttgagattgttggaggcactacacgaattccagctgtgaaggaaagaattgccaaattctttggaaaagatattagcacaacactcaatgcagatgaagcagtagccagaggatgtgcattacagtgtgcaatactttccccggcatttaaagttagagaattttccgtcacagatgcagttccttttccaatatctctgatctggaaccatgattcagaagatactgaaggtgttcatgaagtctttagtcgaaaccatgctgctcctttctccaaagttctcacctttctgagaagggggccttttgagctagaagctttctattctgatccccaaggagttccatatccagaagcaaaaataggccgctttgtagttcagaatgtttctgcacagaaagatggagaaaaatctagagtaaaagtcaaagtgcgagtcaacacccatggcattttcaccatctcta

SEQ ID NO: 98 - DNA, 551 bp, Homo sapiens
tttttttttttttttttttttagcattatcatcttaccctctgtctcaatatacatgttaagaaggtctttccctaactgccagaccaagttggcttcaataggcagctcaacattcaccacctttattttgggctttttagcttctggaggctggtcaacttttttttcatttgctttgtcagcatctgggattttgttttcttctgaggtaagttcaggtgaagggggagactgtgaggtttgttgagcatcagtttgtacctggggctgtgttccagcttcactgttgtcttgctggacatttttatcagtgtctgggttttctggtggtctctgattcagacactccatgtcagcttcagaagacatttcattctcctcagttgggactttctccaccatagatgccgtagagatggtgaaaatgccatgggtgttgactcgcactttgacttttactctagatttttctccatctttctgtgcagaaacattctgaactacaaagcggcctatttttgcttctggatatggaactccttggggatc

SEQ ID NO: 99 - DNA, 550 bp, Homo sapiens
tgtggggctctatttttgctttggctttctggtgagagagtgaggaagcattctttccttcactaagtttgtctttcttgtcttctggatagattgattttaagagactaagggaatttacaaactaaagattttagtcatctggtggaaaaggagactttaagattgtttagggctgggcggggtgactcacatctgtaatcccagcactttgggaggccaaggcaggcagaacacttgaaggagttcgagaccagcgtggccaacgtggtgaaaccctgtctctactaaaaatacaaaaattgtttagctctgtttttcataatagaaatagaaaaggtaaaattgcttttcttctgaaaagaacaagtattgttcatccaagaagggtttttgtgactgaatcagcagtgcctgccctagtcatagctgtgcttcaaaaacctcagcatgattagtgttggagcaaaacaaggaagcaaagcaaatactgtttttgaaattctatctgttgcttgaactattttgtaataattaaactttgatgttg

SEQ ID NO: 100 - DNA, 300 bp, Homo sapiens
ctaagctttaagatttaaaaaatgttcaatgttgaaatttctgtggggctctatttttgctttggctttctggtgagagagtgaggaagcattctttccttcactaagtttgtctttcttgtcttctggatagattgattttaagagactaagggaatttacaaactaaagattttagtcatctggtggaaaaggagactttaagattgtttagggctgggcggggtgactcacatctgtaatcccagcactttgggaggccaaggcaggcagaacacttgaaggagttcaagaccagcg

SEQ ID NO: 101 - DNA, 583 bp, Homo sapiens
gtttgagtcatgagcatgctgttgtctagagtgggcggggatgacgtggttggagtgggtgcgctgctctgtacttgatttttttgagtctgaaattagctttccaggctggggcagggaggggagcacaggtggatcagtactgcccccaagcggtggagctttggtggtggatcaaatactgctgccgcctgtctgcacaaacatatttctctcttccagcccttcagaagtgtattggaatatgtcgataacaataatgatggtggtgaagatgatgatgatgtgggtaattctggctaccttattgggtccaagctccccacaattcgttgcacaaagcactctacatacattctctttagtcctgatcaaaccacctttcagagtaggatttagtgtcctattttaaagatgaaggagctcgggctcagagagagatcgtttagacacacacacaactttggaatgaaacatttacagccgggcgcggtggcgcgtgcctgtagtcccagctacttgggaggctgaggctggaggatcgcttgagtccaggagttctgggctgtagtgcgctatgccg

SEQ ID NO: 102 - DNA, 517 bp, Homo sapiens
cccggaaggcgacgggaaggagccgagcttgggtcatggcggcgccgggcgcgctgctggtgatgggcgtgagcggctcggggaaatccaccgtgggcgccctgctggcatctgagctgggatggaaattctatgatgctgatgattatcacccggaggaaaatcgaaggaagatgggaaaaggcataccgctcaatgaccaggaccggattccatggctctgtaacttgcatgacattttactaagagatgtagcctcgggacagcgtgtggttctagcctgttcagccctgaagaaaacgtacagagacatattaacacaaggaaaagatggtgtagctctgaagtgtgaggagtcgggaaaggaagcaaagcaggctgagatgcagctcctggtggtccatctgagcgggtcgtttgaggtcatctctggacgcttactcaaaagagagggacattttatgccccctgaattattgcagtcccagtttgagactctggagcccccagcagctcc

SEQ ID NO: 103 - DNA, 590 bp, Homo sapiens
ttttttttttttttttttttttttttactagcgaagtttcatttatttgtgcaaatacaggcatgagcaagaatgttctaaacaatgtaacgatttccagcattgattacagaatttcctctgatcatttgatttggttatagatgaatttaaacttcaatttaagcttgacttttaaaactccccctctgcttcctgatgaaccagcataattcctaaaattacacctaaacaagtctgtcttgacacattggggtttgcctttagaaacatttagaatctattatgggcaaggcggctggaacgaggtttgggatggcacaatgatttatgcttagttctgtttggaccactgatacaaaatcattgtcatttcatttttagggtttccataattgtagcaattatctctgaaacatttttgtccacacttatttggataaagttttctggagctgctgggggctccagagtctcaaactgggactgcaataattcagggggcataaaatgtccctctcttttgagtaagcgtccagagatgacctcaaacgacccgctcagatggaccaccaggagctgcatctcag

SEQ ID NO: 104 - DNA, 116 bp, Homo sapiens
gacacttacaaattgctgcttgtccaaatcaggatccactgcaaggaacaacaggccttattccactgctggggattggtgtgtgggagcacgcttactaccttcagtataaaaat

SEQ ID NO: 105 - DNA, 574 bp, Homo sapiens
ttcttttttttttttttttttttgcacaaagcatttactattttcaatcacttgcccaataacaaaatgtttagtaagaaattattcagaacattaagttgtttatgaaataagtgactaagcaacatcaagaaatgctacaatagagcagcttactgtattctgcagtactctataccactacaaaaacagtcataaagagcttaacatactcagcataacgatcgtggtctactttttgcaagccatgtatctttcagttacattctcccagttgattacattccaaatagcttttagataatcaggcctgacatttttatactgaaggtagtaagcgtgctcccacacaccaatccccagcagtggaataaggcctgttgttccttgcagtggatcctgatttggacaagcagcaatttgtaagtgtccccgttccttattgaaaccaagccaaccccaacctgagccttggacaccaacagatgcagccgtcagcttctccttaaacttgtcaaaggaaccaaagtcacgtttgatggcttccagcaactcccctttgggttctccacca

SEQ ID NO: 106 - DNA, 474 bp, Homo sapiens
ttttttttttttttttttttttgggggggtgacagattcttttattaacagtcaaaaacttcacacaattggaaaataaatgtttcttcaatgaataatcaaacaaaaattatccaggaccttatagggttttcagtatgtaccaggcttgatgcacatcttagaagacaggacattatcttgctgggatcattagggtatgatcagcataacgatcgtggtttactttttgcaagccatgtatctttcagttacattctcccagttgattacattccaaatagcttttagataatcaggcctgacatttttatactgaaggtagtaagcgtgctcccacacatcaatccccagcagtggaataaggcctgttgttccttgcagtggatcctgatttggacaagcagcaatttgtaagtgtccccgttccttattgaaaccaagccaaccccaacctgagccttggacaccaac

SEQ ID NO: 107 - DNA, 526 bp, Homo sapiens
gggaacccggggcgcggcgcactgcgcaggcggccggactccgctcagtttccggtgcggcgaacaccaaagtccgggaacttaagcattttcggtttctagggttgttacgaagctgcaggagcgagatggaggtggacgcaccgggtgttgatggtcgagatggtctccgggagcggcgaggctttagcgagggagggaggcagaacttcgatgtgaggcctcagtctggggcaaatgggcttcccaaacactcctactggttggacctctggcttttcatccttttcgatgtggtggtgtttctctttgtgtattttttgccatgacttgttcgctgatatctaaattaagaagttggttcttgagtgaattctgaaaatggctacaaacttcttgaataaagaagacaggactctcaatagaagaatttcacatctccaagggacccttcctttcattttacactttgttactaatttgcagaactctattaattgggtaggatttcacccattcctagcta

SEQ ID NO: 108 - DNA, 344 bp, Homo sapiens
gaacccggggcgcggcgcactgcgcatgcggccggactccgctcagtttccggtgcggcgaacaccaaagtccgggaacttaagcattttcggtttctagggttgttacgaagctgcaggagcgagatggaggtggacgcaccgggtgttgatggtcgagatggtctccgggagcggcgaggctttagcgagggagggaggcagaacttcgatgtgaggcctcagtctggggcaaatgggcttcccaaacactcctactggttggacctctggcttttcatccttttcgatgggggggagcttctctctgtgtatttctcgccatgacctgttcagtgacaccc

SEQ ID NO: 109 - DNA, 332 bp, Homo sapiens
gaacccggggcgcggcgcactgcgcaggcggccggactccgctcagtttccggtgcggcgaacaccaaagtccgggaacttaagcattttcggtttctagggttgttacgaagctgcaggagcgagatggaggtggacgcaccgggtgttgatggtcgagatggtctccgggagcggcgaggctttagcgagggagggaggcagaacttcgatgtgaggcctcagtctggggcaaatgggcttcccaaacactcctactggttggacctctggcttttcatccttttcgatgtggaggagattctctttgtgtattttttgccatgacttgt

SEQ ID NO: 110 - DNA, 545 bp, Homo sapiens
cggctgcgagaagacgacagaaggggagtttccggtgcggcgaacaccaaagtccgggaacttaagcattttcggtttctagggttgttacgaagctgcaggagcgagatggaggtggacgcaccgggtgttgatggtcgagatggtctccgggagcggcgaggctttagcgagggagggaggcagaacttcgatgtgaggcctcagtctggggcaaatgggcttcccaaacactcctactggttggacctctggcttttcatccttttcgatgtggtggtgtttctctttgtgtattttttgccatgacttgttcgctgatatctaaattaagaagttggttcttgagtgaattctgaaaatggctacaaacttcttgaataaagaagacaggactctcaatagaagaatttcacatctccaagggacccttcctttcattttacactttgttactaatttgcagaactctattaattgggtaggatttcacccattcctagctaagttcttaaaattaaaccctttggttcgtgtttaaaaac

SEQ ID NO: 111 - DNA, 329 bp, Homo sapiens
gagtttccggtgcggcgaacaccaaagtccgggaacttaagcattttcggtttctagggttgttacgaagctgcaggagcgagatggaggtggacgcaccgggtgttgatggtcgagatggtctccgggagcggcgaggctttagcgagggagggaggcagaacttcgatgtgaggcctcagtctggggcaaatgggcttcccaaacactcctactggttggacctctggcttttcatccttttcgatgaggaggtgtttctctttgtgtattttttgccatgacttgttcgctgatatctaaatttacaagttggatcttgagtgaaa

SEQ ID NO: 112 - DNA, 284 bp, Homo sapiens
gcgcggcgcctcgcctcggccggcgcctatcagccgacttagaactggtgcggaccaggggaatccgactgtttaattaaaacaaagcatcgcgaaggcccgcggcgggtgttgacgcgatgtgatttctgcccagtgctctgaatgccatattaaaaataaactttaaaatttaaaagggggccgtttttctctgattcccaccccgttaaaaacccttttggggggggggccccccccccctcatggggcggggaaaaaaggccttttttgggaaatttggg

SEQ ID NO: 113 - DNA, 522 bp, Homo sapiens
gttgcaggtcactgtagcgggacttcttttggttttctttctctttggggcacctctggactcactccccagcatgaaggcgctgagcccggtgcgcggctgctacgaggcggtgtgctgcctgtcggaacgcagtctggccatcgcccggggccgagggaagggcccggcagctgaggagccgctgagcttgctggacgacatgaaccactgctactcccgcctgcgggaactggtacccggagtcccgagaggcactcagcttagccaggtggaaatcctacagcgcgtcatcgactacattctcgacctgcaggtagtcctggccgagccagcccctggaccccctgatggcccccaccttcccatccagacagccgagcccgctccggaacttgtcatctccaacgacaaaaggagcttttgccactgactccggccgtgtcctgacacctccagaacgcaggtgctggcgcccgttctgcctgggaccccgggaacctctcctgccggaagccggac

SEQ ID NO: 114 - DNA, 510 bp, Homo sapiens
gttgcaggtcactgtagcgggacttcttttggttttctttctctttggggcacctctggactcactccccagcatgaaggcgctgagcccggtgcgcggctgctacgaggcggtgtgctgcctgtcggaacgcagtctggccatcgcccggggccgagggaagggcccggcagctgaggagccgctgagcttgctggacgacatgaaccactgctactcccgcctgcgggaactggtacccggagtcccgagaggcactcagcttagccaggtggaaatcctacagcgcgtcatcgactacattctcgacctgcaggtagtcctggccgagccagcccctggaccccctgatggcccccaccttcccatccagacagccgagcccgctccggaacttgtcatctccaacgacaaaaggagcttttgccactgactcggccgtgtcctgacacctccagaacgcaggtgctggcgcccgttctgcctgggaccccgggaacctctcctgcc

SEQ ID NO: 115 - DNA, 385 bp, Homo sapiens
aatagtctgtgtccaagaaaataagaatcacgtcatctagctgtggacactgagcaaaaaggagcagcatgctattaagatggttgagacacacgagtgaacaaagatgggacaaactgtgcttcgttcaagaagtttcatcaagacccctaccgccccccgtccttcagctctgtacagtaactttagctttacatagagctgagataaaaataaagctttcttacaaattacatttttttccagtgaattacttttgcagtaaaaatagctgctacataaatccctcctgatctctgaaaaggagttgcatatttccaaaaataatattcttattttaatcacacagaagaacgtggagcacaggaaggaaatggctggctgg

SEQ ID NO: 116 - DNA, 645 bp, Homo sapiens
tacggccgggtcttttaaagaggccgggaatacacatgactcaggtgctcttttgaaacgactacaaaagtctccattttgatcaaaacgttttctccgaatgaatggctccgatgctttctctttcccatcttaagtccccgctctgtgcctcagaatagtctgtgtccaagaaaataagaatcacgtcatctagctgtggacactgagcaaaaaggagcagcatgctattaagatggttgagacacacgagtgaacaaagatgggacaaactgtgcttcgttcaagaggtttcatcaagacccctaccgccccccgtccttcagctctgtacagtaactttagctttacatagagctgagataaaaataaagctttcttacaaattacatttttttccagtgaattacttttgcagtaaaaatagctgctacataaatccctcctgatctctgaaaaggagttgcatatttccaaaaataatattcttattttaatcacacagaagaacgtggagcacaggaaggaaatggctggctggtcagggagaggtgagctgtcggagaaacacagtaaaactaaaaaataaaatccattttgtgtataaactgacttaaacgcatgcaaagaagtggaaaacatatg

SEQ ID NO: 117 - DNA, 500 bp, Homo sapiens
atgtcgagggaatgcagaaagagttaaggaaggcaggttgtccttctattcaggccactcttcgttttccatgtactgcatgctgtttgtggcactttatcttcaagccaggatgaagggagactgggcaagactcttacgccccacactgcaatttggtcttgttgccgtatccatttatgtgggcctttctcgagtttctgattataaacaccactggagcgatgtgttgactggactcattcagggagctctggttgcaatattagttgctgtatatgtatcggatttcttcaaagaaagaacttcttttaaagaaagaaaagaggaggactctcatacaactctgcatgaaacaccaacaactgggaatcactatccgagcaatcaccagccttgaaaggcagcagggtgcccaggtgaggctggcctgttttctaaaggaagatgattgccacaaggcaagaagatgcatctttcttcctggtgtacaagccttt

SEQ ID NO: 118 - DNA, 592 bp, Homo sapiens
taaggaaggcaggttgtccttctattcaggccactcttcgttttccatgtactgcatgctgtttgtggcactttatcttcaagccaggatgaagggagactgggcaagactcttacgccccacactgcaatttggtcttgttgccgtatccatttatgtgggcctttctcgagtttctgattataaacaccactggagcgatgtgttgactggactcattcagggagctctggttgcaatattagttgctgtatatgtatcggatttcttcaaagaaagaacttcttttaaagaaagaaaagaggaggactctcatacaactctgcatgaaacaccaacaactgggaatcactatccgagcaatcaccagccttgaaaggcagcagggtgcccaggtgaggctggcctgttttctaaaggaagatgattgccacaaggcaagaggatgcatctttcttcctggtgtacaagcctttaaagacttctgctgctgctatgcctcttggatgcacactttgtgtgtacatagttacctttaactcagtggttatctaatagctctaaactcattaaaaaaactccaagccttcca

SEQ ID NO: 119 - DNA, 197 bp, Homo sapiens
ggccgcccttttttttttttttttttttttttttttttggggaaaagggggtcttttttgggtcccccccccccttttaaaaaacccccctaaaaaatgcccccaaaaaaaaaaatttttttttttggggggggggaaaaaaagggggaaaaaaccccccccccccggggggggaaaaaaacccccccaaaaccccc

SEQ ID NO: 120 - DNA, 493 bp, Homo sapiens
ttttttttttttaatggtaaaaactttatttactatttataaatacattgcaagacaaacttctcaaaaatacttttccccccaaaaagttaaaaaaataaagaaaagctaataggtaggcagaatgtcttgagacccctctgttttcaaggagagctctatgcagcgtgtgtccacaccgaggtctgcagcagggcagagtctccctgagcctgactttgccagaccttcttgggtttggcctccgggagagcagcccagtctctgggtcgacgtcctttcctcagtcatggccacagttgtatcatatagcatctctaacatttcatctaggattatctagtatagatcttactatatttggggctatgttgtatacaatgttaacaagaacatatcttctctgcatatatgtgtgaattataaagaaaagcatgagaatgactctaagttcaacaaacatgggtgaatctctatgtgctcccagtgtcct

SEQ ID NO: 121 - DNA, 265 bp, Homo sapiens
tggtacgcctgcagtaccggtccggaattcccgggtcgacccacgcgtccgcttcctgttttctgttgtcaaatgatgataatgtgccatgatgttttatatatatcattcagaaaaagttttattttttaataacattctattaacattattttgcttgccgctggcatgcctgaggaatgtatttggctttgattacacactaagtttttgtaataaatttgactcattaaaaacctttttttttaaaaaaaaaaaaaaaaaa

SEQ ID NO: 122 - DNA, 186 bp, Homo sapiens
tttctgttttctgttgtcaaatgatgataatgtgccatgatgttttatatatatcattcagaaaaagttttattttttaataacattctattaacattattttgcttgccgctggcatgcctgaggaatgtatttggctttgattacacactaagtttttgtaataaatttgactcattaaaaacc

SEQ ID NO: 123 - DNA, 475 bp, Homo sapiens
cagcccgtccgcggcctctccagccccgggttcgcgctctcgactcccccgacccagtccgcggtgccccggcgggtgatgccaaatacagccatgaagaaaaaggtgctgctgatggggaagagcgggtcggggaagaccagcatgaggtcgataatcttcgccaattacattgctcgcgacacccggcgcctgggggccaccattgacgtggaacactcccacgtccgattcctagggaacctggtgctgaacctgtgggactgtggcggtcaggacaccttcatggaaaattacttcaccagccagcgagacaatatcttccgtaacgtggaagttttgatttacgtgtttgacgtggagagccgcgaactggaaaaggacatgcattattaccagtcgtgtctggaggccatcctccagaactctcctgacgccaaaatcttctgcctggtgcacaaaatggatctggttc

SEQ ID NO: 124 - DNA, 122 bp, Homo sapiens
agaagggttgctggagcctaggacgtcgaggctgcagtgagatatgatcacaccactgcactccagcatgactgagtgagaccctgtctcaaaaaaaaaaaaaaaaaagttttttttttttc

SEQ ID NO: 125 - DNA, 147 bp, Homo sapiens
ggaggggaaggttggtaggtaagctgtaacagattgctccagttgccttaaactacgcacatagctaagtgaccaaacttcttgttttgatttgaaaaagtgcattgttttcttgtccctccctttgatgaaacgttaccctttgac

SEQ ID NO: 126 - DNA, 607 bp, Homo sapiens
cagtgaagacttgcatgttgttttcactactgtacacttgacctgcacatgcgagaaaaaggtggaatgtttaaaacaccataatcagctcagggtatttgccaatctgaaataaaagtgggatgggagagtgtgtccttcagatcaagggtactaaagtccctttcgctgcagtgagtgagaggtatgttgtgtgtgaatgtacggatgtgtgtttgcgtgcatgtttgtgcatgtgtgactgtgcatgttatgtttctccatgtgggcaaagatttgaaatgtaagcttttatttattattttagaatgtgacataatgagcagccacactcgggggaggggaaggttggtaggtaagctgtaacagattgctccagttgccttaaactacgcacatagctaagtgaccaaacttcttgttttgatttgaaaaaagtgcattgttttcttgtccctccctttgatgaaacgttaccctttgacgggccttttgatgtgaacagatgttttctaggacaaactataaggactaattttaaacttcaaacattccacttttgtaatttgttttaaattgttttatgtatagtaagcacaactgtaat

SEQ ID NO: 127 - DNA, 463 bp, Homo sapiens
attccaattagccaggaatggaaggatgagaagcgggatttgctgactgaaggacaaagttttagcagccttgatgaagaagccctgggatcccgacacaggccagacctggtccctagcactccatcactgtttgaagctgcttccttggcaaccacaatttcatcttcttccttatacgtcaatgagcactatccacacgacaggcctacactctattcaaacagcaaagggttaccttccagttcaacatttaccttggaagaggggaccatctacttgaccgctgagcccaacactctggaagtgcaggatgacaatgcttctgtgcttgacgtctatttataagtgaaaatggtgatcacctaagcacatggatgagacgtgagcacagttatggcagagaagtttctccgcaccagaattatccacagcaacttggctgagccccactacacacaga

SEQ ID NO: 128 - DNA, 592 bp, Homo sapiens
ccaattagccaggaatggaaggatgagaagcgggatttgctgactgaaggacaaagttttagcagccttgatgaagaagccctgggatcccgacacaggccagacctggtccctagcactccatcactgtttgaagctgcttccttggcaaccacaatttcatcttcttccttatacgtcaatgagcactatccacacgacaggcctacactctattcaaacagcaaagggttaccttccagttcaacatttaccttggaagaggggaccatctacttgaccgctgagcccaacactctggaagtgcaggatgacaatgcttctgtgcttgacgtctatttataagtgaaaatggtgatcacctaagcacatggatgagacgtgagcacagttatggcagagaagtttctccgcaccagaattatccacagcaacttggctgagccccactacacacagagaaatcatcaacctgacttaagagttttcaagatgtcaacttcaggctgatcagcagatgggatgtgaaaaatactaccctattctatcatttgctgttgcttgctgaactgtgaagaactgcatgaactat

SEQ ID NO: 129 - DNA, 251 bp, Homo sapiens
caattagccaggaatggaaggatgagaagcgggatttgctgactgaaggacaaagttttagcagccttgatgaagaagccctgggatcccgacacaggccagacctggtccctagcactccatcactgtttgaagctgcttccttggcaaccacaatttcatcttcttccttatacgtcaatgagcactatccacacgacaggcctacactctattcaaacagcaaagggttaccttccagttcaacattt

SEQ ID NO: 130 - DNA, 229 bp, Homo sapiens
gtagcagaagcctcattccagaacccatctggccagagaagcagcagcatcctgggggatggccgtgcatggggtgtacactcgctataggcataggcccggcatggctgtcgctggacgccagctgtgcacacccagccacacctgctgcacgccgcgttagtgtgcggctccgggcctgagcattcgcaaagctcgcttctccagggagcctcctcttggctttgga

SEQ ID NO: 131 - DNA, 316 bp, Homo sapiens
cgccataacctggtcagaagtgtgcctgtcggcggggagagaggcaatatcaaggttttaaatctcggagaaatggctttcgtttgcttggctatcggatgcttatatacctttctgataagcacaacatttggctgtacttcatcttcagacaccgagataaaagttaaccctcctcaggattttgagatagtggatcccggatacttaggttatctctatttgcaatggcaacccccactgtctctggatcattttaaggaatgcacagtggaatatgaactaaaataccgaaacattggtagtgaaacatgga

SEQ ID NO: 132 - DNA, 270 bp, Homo sapiens
  misc_feature 37: n = A,T,C or G
agtcgccataacctggtcagaagtgtgcctgtcggcngggagagaggcaatatcaaggttttaaatctcggagaaatggctttcgtttgcttggctatcggatgcttatatacctttctgataagcacaacatttggctgtacttcatcttcagacaccgagataaaagttaaccctcctcaggattttgagatagtggatcccggatacttaggttatctctatttgcaatggcaacccccactgtctctggatcattttaaggaatgc

SEQ ID NO: 133 - DNA, 341 bp, Homo sapiens
ttacatacgtttttattactcgggggggacctgtacgtcaccaatgcccagcttcacgggggcatgtagtgtgactcacggctgaacacaaaatcactgtgaagcctgtgctacagaaggatgtccagtcgctgaggccaggagagaggtgggcaggcctgggtctggcagtggagacggtcctccagggagccgttgggcaggaagccgtacaccaggcagtagaagccgttctgagcacagtagccagcaaagtccacaatgtttgggtgacgaaacctggacagctgctccacctcggtcaggaagctctgcttcactgcagtccactccaggtcagc

SEQ ID NO: 134 - DNA, 466 bp, Homo sapiens
attatgtgattaatgatttgacagccgttccaatctccacgtctccagaagagattccacatgggagtttctcagactgattcttgacctctcaatgaaagtgttgaaacaggatgggaaatattttacacaggggaactgtgtcaatctgacagaagcactgtcgctctatgaagaacagctggggcgcctgtattgtcctgtggaattttcaaaggagatcgtctgtgtcccttcatacttggaattgtgggtattttacactgtttggaagaaagctaaaccctgaagatcagtagcccctaatcacatgtgctgcaaatagccttcctgacctccatatgctgtacatgacatcaaaatgagtcaggcaattgattgtgaattccttaaagttttcctttttttaataattatttttaatttaaaaaagcaaatggaaaatgtatattttgatgagcttagg

SEQ ID NO: 135 - DNA, 70 bp, Homo sapiens
agttttcctttttttaataattatttttaatttaaaaaagcaaatggaaaatgtatattttgatgagctt

SEQ ID NO: 136 - DNA, 442 bp, Homo sapiens
tttttttttttttttttcggctcagtataaagcttccttttcttagggaccatgcaaagattctttgattctagaagtgccatttcattatttctgtgactcctgtctgaatcatctgccaggtaactatcttgattttgtcttagcaatcgacttagcagaccattcttggagaaagaaaaatcctgaggtgaaacaggctccgatttaaagtcttcggacactggtaaggcaggtgcgcttctctgcacagcaggagccatacccaagaatggggcactcttagcatcatggctcaagtgcacatttgtgttaggaatttgtaagtcatcacaaggctcagattttattttcaccatcagtatttgttcacttaaagctctctctgagtgttcctgagtactttcatctcttaagggagttttctcttttttttcactct

SEQ ID NO: 137 - DNA, 275 bp, Homo sapiens
  misc_feature 244: n = A,T,C or G
agaaaaatacaaaaaatctgcattaaaaatattaatcctgcatgctggacatgtatggtaataatttctattttgtaccattttctgtttaactttagcatgttgttgatcatggatcatactcccctgtttcttgggtgagaagggatcgccagtttggaaactccggcggctgcgtgcggggtttcagtcccactgtaggcttgtaaataccgccccgccaaaccgcatagagacgtggcancactgagggctttgttgggttatatacgtat

SEQ ID NO: 138 - DNA, 353 bp, Homo sapiens
taagctcggaattcgggtcgaggaaaaatacaaaaaatctgcattaaaaatattaatcctgcatgctggacatgtatggtaataatttctattttgtaccattttcttgtttaactttagcatgttgttgatcatggatcatactccccttgtttctttgggtgagaagggatcgcagtttggaaactccggcggctgcgtgcggggtttcagtcccagctgtaggcttgtaaatacccgccccgccaaaccgcatagagaacgtggcagcaagctgagggtctttgtttgggtttattattacggtatttttgtttgtaagttaaaaaaaaaaaaaaaagggggggccccca

SEQ ID NO: 139 - DNA, 559 bp, Homo sapiens
gaatttggccctcgaggccaagaattcggcactagggcgcagaaggaccagcagaaagatgccgaggcggaagggctgagcggcacgaccctgctgccgaagctgattccctccggtgcaggccgggagtggctggagcggcgccgcgcgaccatccggccctggagcaccttcgtggaccagcagcgcttctcacggccccgcaacctgggagagctgtgccagcgcctcgtacgcaacgtggagtactaccagagcaactatgtgttcgtgttcctgggcctcatcctgtactgtgtggtgacgtcccctatgttgctggtggctctggctgtctttttcggcgcctgttacattctctatctgcgcaccttggagtccaagcttgtgctctttggccgagaggtgagcccagcgcatcagtatgctctggctggaggcatctccttccccttcttctggctggctggtgcgggctcggccgtcttctgggtgctgggagccaccctggtggtcatcggctcccacgctgccttccaccagattgaggctgtggacg

SEQ ID NO: 140 - DNA, 711 bp, Homo sapiens
  misc_feature 444: n = A,T,C or G
tttttttttttttttttttgacacccataacagctttattttcaaaggcgggatccctccccgggcttgtgatgggacggcgctgtgggcccgagcagcaaagccgtgcaggacaggcatgggcaggggtggggcagctggcccgggaggccggcaggtcccaaaagacacctcacacgggttccatctgcagctcctccccgtccacagcctcaatctggtggaaggcagcgtgggagccgatgaccaccagggtggctcccagcacccagaagacggccgagcccgcaccagccagccagaagaaggggaaggagatgcctccagccagagcatactgatgcgctgggctcacctctcggccaaagagcacaagcttggactccaaggtgcgcagatagagaatgtaacaggcgccgaaaaagaccagccagagccaccagncacataggggacgtcaccacacagtacaggatgaggcccaggaacacgaacacatagttgctctggtagtactccacgttgcgtacgaggcgctggcacagctctcccaggttgcggggccgtgagaagcgctgctggtccacgaaggtgctccaggggccggatggtcgcgcggcgccgctccagccactcccggcctgcacccggaggaatcagcttcggcagcaaggtcgtgccggtcagcccttccgcctcggcattctttct

SEQ ID NO: 141 - DNA, 468 bp, Homo sapiens
actcgcagtccttcttctctggcctctttggaggctcatccaaaatagaggaagcatgcgaaatctacgccagagcagcaaacatgttcaaaatggccaaaaactggagtgctgctggaaacgcgttctgccaggctgcacagctgcacctgcagctccagagcaagcacgacgcagccacctgctttgtggacgctggcaacgcattcaagaaagccgacccccaagaggccattaactgtttgatgcgagcaatcgagatctacacagacatgggccgattcacgattgcggccaagcaccacatctccattgctgagatctatgagacagagttggtggacatcgagaaggccattgcccactacgagcagtctgcagactactacaaaggcgaggagtccaacagctcagccaacaagtgtctgctgaaggtggctggttacgctgcgctgctggagcagtatc

SEQ ID NO: 142 - DNA, 203 bp, Homo sapiens
cgcaaagtgaagaactcgcagtccttcttctctggcctctttggaggctcatccaaaatagaggaagcatgcgaaatctacgccagagcagcaaacatgttcaaaatggccaaaaactggagtgctgctggaaacgcgttctgccaggctgcacagctgcacctgcagctccagagcaagcacgacgcagccacctgctttgt

SEQ ID NO: 143 - DNA, 212 bp, Homo sapiens
tctgcggggaacagaacatgatcggcatgacgcccacggtcatcgctgagcattacctggctgaaacggagcagcgggagaagttcgggctaaagaagcgggagggggcctgggagctcatgaagaaggggtacacccagcaactggccttcatacaacccagctctgcctttgcggccttcgtgaaacgggcacccagcacctggctgacc

SEQ ID NO: 144 - DNA, 226 bp, Homo sapiens
  misc_feature 109, 128, 153, 161, 167, 174, 175, 178, 196, 202, 206,211, 213: n = A,T,C or G
gaagcacctcattgtgaccccctcgggctgcggggaacagaacatgatcggcatgacgcccacggtcatcgctgtgcattacctggatgaaacggagcagtgggagaanttcggcctagagaagcggnagggggccttggagctcatcaaganggggtacncccagnagctggnnttnagacaacccagctctgcntttgcnggcnttcgnanaaagggcccccac

SEQ ID NO: 145 - DNA, 97 bp, Homo sapiens
ctgggctgcggctgatgcgcatccgttttcctgccctgggcatgtgtctctgaaaccgtatggcgggcggtgggcaacgggcactgctaagggaggc

SEQ ID NO: 146 - DNA, 120 bp, Homo sapiens
ggcacgagctcatctgtttgcggatcagaacccgagctgtgcttgtggctgcggctgctaactggctgcgcacagggagctgtcaccatgcctcactcgtacccagccctttctgctgag

SEQ ID NO: 147 - DNA, 273 bp, Homo sapiens
ggccgcccttttttttttttttttttttccccccttttttttggtgggggggtttttccaaggggttgaatgggggttttttttccccccttttaccccagaaaaagggggaggaaaaaaggaacccccggggaaaattttcctttttttggaaaatttgggggacccgaaaaagggggggggaacccccccccttttttttttcttttaaaaaatttttttgcccccaaaaaaaggggggccccctttcccccccttcttgggccccggggg

SEQ ID NO: 148 - DNA, 90 bp, Homo sapiens
cacttcatgcaaggcacatgtgctgtcctgcaggtctgcagggaaccgacccagagagcccagcggcaggccctggaacacccgcctctg

SEQ ID NO: 149 - DNA, 463 bp, Homo sapiens
gacttgtccgggaatccggtgcttcggatctactacacctcgaggcctgctctgttcaccttgtgtgctgggaatgagctcttctactgcctcctctacctgttccatttctctgagggacctttagttggctctgtgggactgttccggatgggcctctgggtcactgcccccatcgccttgctgaagtcgctcatcagcgtcatccacctgatcacggccgcccgcaacatggctgccctggacgcagcagaccgcgccaagaagaagtgacgctggagccccgggtcctggctgcccacctgccctgggagtcttgctgtgccacacagctccccaccccctgctaggaggtcccagtctcacgccttcctcatgtgttgttctacctgctgggatgggggtcagcctctctttggtgacgtcacgttctctgggatcctgaggacccgggcctcaaatc

SEQ ID NO: 150 - DNA, 693 bp, Homo sapiens
  misc_feature 285, 455, 597, 606, 636, 667, 686: n = A,T,C or G
ggcacgaggagagagagagtcacaagatgatcgacttgttcgggaatccggtgcttcggatctactacacctcgaggcctgctctgttcaccttgtgtgctgggaatgagctcttctactgcctcctctacctgttccatttctctgagggacctttagttggctctgtgggactgttccggatgggcctctgggtcactgcccccatcgccttgctgaagtcgctcatcagcgtcatccacctgatcacggccgcccgcaacatggctgccctggacgcagcanaccgcgccaagaagaagtgacgctggagccccgggtcctggctgccacctgccctgggagtcttgctgtgccacacagctccccaccccctgctaggaggtcccagtctcacgccttcctcatgtgttgttctacctgctgggatgggggtcagcctctctttggtgacntcacgttcttctgggatcctgaggaccgggcctcaaatcagggaggatacccgggagggccccttcatccaagcggtgcttctggggtgccgggaccgggcagtgtcacaccctgcctgctagtcctggggtccagatctanggaccttantgaaggagtggtgtgaggcagttctgaagnggataactcgcccacaacaagttgggacatncagaggaaactcaactctnacgtctt

SEQ ID NO: 151 - DNA, 300 bp, Homo sapiens
gagagagagagtcacaagatgatcgacttgtccgggaatccggtgcttcggatctactacacctcgaggcctgctctgttcaccttgtgtgctgggaatgagctcttctactgcctcctctacctgttccatttctctgagggacctttagttggctctgtgggactgttccggatgggcctctgggtcactgcccccatcgccttgctgaagtcgctcatcagcgtcatccacctgatcacggccgcccgcaacatggctgccctggacgcagcagaccgcgccaagaagaagtgacgc

SEQ ID NO: 152 - DNA, 300 bp, Homo sapiens
  misc_feature 37, 41: n = A,T,C or G
gacttgtccgggaatccggtgcttcggatctactacncctngaggcctgctctgttcaccttgtgtgctgggaatgagctcttctactgcctcctctacctgttccatttctctgagggacctttagttggctctgtgggactgttccggatgggcctctgggtcactgcccccatcgccttgctgaagtcgctcatcagcgtcatccacctgatcacggccgcccgcaacatggctgccctggacgcagcagaccgcgccaagaagaagtgacgctggagccccgggtcctggctgccc

SEQ ID NO: 153 - DNA, 239 bp, Homo sapiens
  misc_feature 168, 190, 203, 229: n = A,T,C or G
gttgccctgcctctggctccagaacagaaagggagcctcacgctggctcacacaaaacagctgacactgactaaggaactgcagcatttgcacaggggaggggggtgcctccttcctagaggccctgggggccaggctgattggggggcagattgacataggccccantcatcagatgtctgaaattcancacgggggtaacntggggggttagggactatttttaaantaggggtggc

SEQ ID NO: 154 - DNA, 113 bp, Homo sapiens
gacacatttgttacttcgtgagcaagcccggaggctcggagccccctgccgtgttcacaggtgacaccttgtttgtggctggctgcgggaagttctatgaagggactgcggat

SEQ ID NO: 155 - DNA, 294 bp, Homo sapiens
ttttttttttttttttttttttttggcgggaataaatacttgttaaacttctcttataaatatgcattaaaacgtccgataacacaagccaagggctgtaaaattaaggttaaatcaagactgaatttcccgcacggaccagcaggaaagccagttacctaaaagagcctaatccccaaatccgctgaaggtgcagggcggcctcagtcccggggcatcttgaactggtccttctccctgcgcacggcccgcatggtggtcaccgggtccgtctcacctgcgtgctgctgcacc

SEQ ID NO: 156 - DNA, 419 bp, Homo sapiens
tagccatggcaggacagctcctggaccaggtctcataatgcatgtggcacttaggtccaagctctccagagggtgaaagctggagtctgtcaatgtcctactgagacagcacagccaacctagctagcaacatttgttttagtctgaacaatatatacttatagaattcagtcaaagatacacaatctgaaacagcttcatggggtggactctaacagtagttgcaatgttttagaatgagacttacttctctgctatctagatctgaactccttggcttctttacttagttcaagccccagcctaggaaagccagttacataaaagttggctcaggagtcttagagctttacctaaatagagcccagaaaacggaggatgggggtggggcgccttcctggaggtgacacttgatgggg

SEQ ID NO: 157 - DNA, 357 bp, Homo sapiens
cgtattgctgtcaagccgtgagctagccatggcaggacagctcctggaccaggtctcataatgcatgtggcacttaggtccaagctctccagagggtgaaagctggagtctgtcaatgtcctactgagacagcacagccaacctagctagcaacatttgttttagtctgaacaatatatacttatagaattcagtcaaagatacacaatctgaaacagcttcatggggtggactctaacagtagttgcaatgttttagaatgagacttacttctctgctatctagatctgaactccttggcttctttacttagttcaagccccagcctaggaaagccagttacataaaagttggctc

SEQ ID NO: 158 - DNA, 408 bp, Homo sapiens
actttgtatcactgcagcgcttcacaccttcatcctgaagatatctggaacattcgtagtatctgcagcaccaccaatatccaatgcaagaacggcaagatgaactgccatgagggtgtagtgaaggtcacagattgcagggacacaggaagttccagggcacccaactgcagatatcgggccatagcgagcactagacgtgttgtcattgcctgtgagggtaacccacaggtgcctgtgcactttgacggttagatgccaccatgtagggattatcgcgagtggttgaccttacacttactccttaaatagcagtgagtaatgcatttgagctgccccaggctctgtctcctcagctcatttcttactctttttctctatataactcattctattaaatacattgca

SEQ ID NO: 159 - DNA, 550 bp, Homo sapiens
acaaggacgccaaccccacctagatgcaaagcaggattcaaaagaacatctttgcgttttctaccggctccccatcatcgtactagggaggaagaagcgggtgagaaacaaaacttctttccattgtcctgcccttttctgcggacttgttctgaggccgaggcacctctaagatactgatggctctgcagaggacccattcattgcttctgcttttgctgctgaccctgctggggctggggctggtccagccctcctatggccaggatggcatgtaccagcgattcctgcggcaacacgtgcaccctgaggagacaggtggcagtgatcgctactgcaacttgatgatgcaaagacggaagatgactttgtatcactgcaagcgcttcaacaccttcatccatgaagatatctggaacattcgtagtatctgcagcaccaccaatatccaatgcaagaacggcaagatgaactgccatgagggtgtagtgaaggtcacagattgcagggacacaggaagttccagggcacccaactgcagatatcgggc

SEQ ID NO: 160 - DNA, 554 bp, Homo sapiens
ccaaccccacctagatgcaaagcaggattcaaaagaacatctttgcgttttctaccggctccccatcatcgtactagggaggaagaagcgggtgagaaacaaaacttctttccattgtcctgcccgtttctgcggacttgttctgaggccgaggcacctctaagatactgatggctctgcagaggacccattcattgcttctgcttttgctgctgaccctgctggggctggggctggtccagccctcctatggccaggatggcatgtaccagcgattcctgcggcaacacgtgcaccctgaggagacaggtggcagtgatcgctactgcaacttgatgatgcaaagacggaagatgactttgtatcactgcaagcgcttcaacaccttcatccatgaagatatctggaacattcgtagtatctgcagcaccaccaatatccaatgcaagaacggcaagatgaactgccatgagggtgtagtgaaggtcacagattgcagggacacaggaagttccagggcacccaactgcagatatcgggccatagcgagcact

SEQ ID NO: 161 - DNA, 313 bp, Homo sapiens
aattacatcttctttaaagccaaatgggagatgccctttgacccccaagatactcatcagtcaaggggcgtacttgagcaggaaaaagtgggtaatggtgcccatgatgagtttgcatcacctgactataccttacttccgggacgaggagctgtcctgcaccgtggtggagctgaagtacacaggcaatgccagcgcactcttcatcctccctgatcaagacaagatggaggaagtggaagccatgctgctcccagagaccctgaagcggtggagagactctctggagttcagagagataggtgagctctac

SEQ ID NO: 162 - DNA, 519 bp, Homo sapiens
cggccgcccttttttttttttttggccccccggggcccccttattttaaaaaccccccccccccctggggggggggccccgaccttttaagttttttttttttcccccgggggaaaaaaaggggggaaaaaaaaaaaaaattcccccccctttttcccccccccaaaaaagggggggaccccccgggggggggggggtttccccgggggggaaaaaaaaaacccccgggggcccccccccaattttttcccccccccccttggggggggggggggggggggggggggggggggggcccccccccccccccccccccccattttggggggttgggttgggggaaatttttttttaaaaaaaaaaaaaaaaaatttgggggtccccccccccctttttttccccccccttttttccaaaaggggaccccccccccccccccccaaaaaaaccccccccccccccccaaaaaaaacccccccccggggggggaaaaaaaagggggggggggggggccccccc

SEQ ID NO: 163 - DNA, 422 bp, Homo sapiens
aactaaaaactacagtggaagaaaggaagtcttcagaagcctcccccactgcgcaaagaagtaaagatcacagtaaggaatgcataaacgctgccccagattctccgtccaaacagcttccagaccagatttcattcttcagtggaaatccatcagttgaaatagttcatggtattatgcacctatataagacaaataagatgacctccttaaaagaagatgtgcggcgcagtgccatgctgtgtattctcacagtccctgctgcaatgaccagtcatgaccttatgaagtttgttgccccatttaacgaagtaattgaacaaatgaaaattatcagagactctactcccaaccaatatatggtgctgataaagtttcgtgcacaggctgatgcggatagtttttatatgacatgcaatggc

SEQ ID NO: 164 - DNA, 626 bp, Homo sapiens
tacggccgggtgcgagctctgcgggaagcggttcctggatagtttgcggctgagaatgcacttactggctcattcagcgggtgccaaagcctttgtctgtgatcagtgcggtgcacagttttcgaaggaggatgccctggagacacacaggcagacccatactggcactgacatggccgtcttctgtctgctgtgtgggaagcgcatccaggcgcagagcgcactgcagcagcacatggaggtccacgcgggcgtgcgcagctacatctgcagtgagtgcaaccgcaccttccccagccacacggctctcaaacgccacctgcgctcacatacaggcgaccacccctacgagtgtgagttctgtggcagctgcttccgggatgagagcacactcaagagccacaaacgcatccacacgggtgagaaaccctacgagtgcaatggctgtggcaagaagttcagcctcaagcatcagctggagacgcactatagggtgcacacaggtgagaagccctttgagtgtaggctctgccaccagcgctcccgggactactcggccatgatcaagcacctgagaacgcacaacggcgcctcgccctaccagtgcaccatctgcacagagtact

SEQ ID NO: 165 - DNA, 515 bp, Homo sapiens
gatagtttgcggctgagaatgcacttactggctcattcagcgggtgccaaagcctttgtctgtgatcagtgcggtgcacagttttcgaaggaggatgccctggagacacacaggcagacccatactggcactgacatggccgtcttctgtctgctgtgtgggaagcgcatccaggcgcagagcgcactgcagcagcacatggaggtccacgcgggcgtgcgcagctacatctgcagtgagtgcaaccgcaccttccccagccacacggctctcaaacgccacctgcgctcacatacaggcgaccacccctacgagtgtgagttctgtggcagctgcttccgggatgagagcacactcaagagccacaaacgcatccacacgggtgagaaaccctacgagtgcaatggctgtggcaagaagttcagcctcaagcatcagctggagacgcactatagggtgcacacaggtgagaagccctttgagtgtaggctctgccaccagcgctcccgggacta

SEQ ID NO: 166 - DNA, 615 bp, Homo sapiens
actgttcaaggtttattgggggttttagttggtataacacttggatagttggttgcattgtttgtatgtagatctttttacattatatggtaatgtacactactgatatagttcacaaaataagatcctttggaagaattatgcacaagacatgatattggatttatacactggatcccaggatgtgactcactgggaaaaaatgttggactaggcatgttcagtgaaggagccaggaagttatataacacacggtaaacatccacctggctcaaggggcaaatgcagtacgtacagcattggcagtggtgcgtcagaggtggcagaactatttcacactaaccagttgaagactacacaagattaataccatccagcatcaggatatagctgtggattttacaaaccattcttatttctaacttcaggagttgatgtttttcccagtccatcttaaaatattactgctttaatcacagatcagataaaaaggacaacatgcacaacctccacctagaatcctgttgtagcctagacagtgaaatgatatgacatcagaagactttaaaattgcagctccttttggatcccccaaagtgtatctgcactcttctt

SEQ ID NO: 167 - DNA, 99 bp, Homo sapiens
ttttttttttccactgttcaaggtttattgggggttttagttggtataacacttggatagtgggttgcattgtttgtatgtaaatctttttacattata

SEQ ID NO: 168 - DNA, 612 bp, Homo sapiens
tacggccgggacatgaaggagctaggagtgggaatagctttgcgaaaaatgggcgcaatggccaagccagattgtatcatcacttgtgatggtaaaaacctcaccataaaaactgagagcactttgaaaacaacacagttttcttgtaccctgggagagaagtttgaagaaaccacagctgatggcagaaaaactcagactgtctgcaactttacagatggtgcattggttcagcatcaggagtgggatgggaaggaaagcacaataacaagaaaattgaaagatgggaaattagtggtggagtgtgtcatgaacaatgtcacctgtactcggatctatgaaaaagtagaataaaaattccatcatcactttggacaggagttaattaagagaatgtccaagctcagttcaatgagcaaatctccatactgtttctttctttttttttcattactgtgttcaattatctttatcataaacattttacatgcagctatttcaaagtgtgctggattaattaggatcatccctttggttaataaataaatgggtttgtgctaatatatcttgtatgcattctttaaaccttacaggaaattagtgatgagtttt

SEQ ID NO: 169 - DNA, 410 bp, Homo sapiens
gaaaacaacacagttttcttgtaccctgggagagaagtttgaagaaaccacagctgatggcagaaaaactcagactgtctgcaactttacagatggtgcattggttcagcatcaggagtgggatgggaaggaaagcacaataacaagaaaattgaaagatgggaaattagtggtggagtgtgtcatgaacaatgtcacctgtactcggatctatgaaaaagtagaataaaaattccatcatcactttggacaggagttaattaagagaatgtccaagctcagttcaatgagcaaatctccatactgtttctttctttttttttcattactgtgttcaattatctttatcataaacattttacatgcagctatttcaaagtgtgctggattaattaggatcatccctttgg

SEQ ID NO: 170 - DNA, 310 bp, Homo sapiens
gctcgggaattcgctcgagtgctgctccccacccatggacaggagatcctgggttgggcctccctctgatgaccccagccagatgagcgagtggggctcagcgtggcccatggtgcctgtcactcagcattcccatgcctgatgtttaccaagtgctgtgttggacactggctttctccaaacaggatttgcctcctccacgctccctacacacctgagatgtaaactggcagtcagtgttcactcaggacctaggattagaaaatggcagagttggtgctggatccaccttgcacttctatcaagccct

SEQ ID NO: 171 - DNA, 257 bp, Homo sapiens
tgctgctccccagcccatggacaggagatcctgggttgggcctccctctgatgaccccagccagatgagcgagtggggctcagcgtggcccatggtgcctgtcactcagcattcccatgcctgatgtttaccaagtgctgtgttggacactgactttctccaaacaggatttgcctcctccacgctccctacacacctgagatgtaaactggcagtcagtgttcactcaggacctaggattagaaaatggcagagtt

SEQ ID NO: 172 - DNA, 593 bp, Homo sapiens
tgaagaacggtgccacttacgaagccaaaatcaaggatgtggatgagaaagcagacatcgcactcatcaaaattgaccaccagggcaagctgcctgtcctgctgcttggccgctcctcagagctgcggccgggagagttcgtggtcgccatcggaagcccgttttcccttcaaaacacagtcaccaccgggatcgtgagcaccacccagcgaggcggcaaagagctggggctccgcaactcagacatggactacatccagaccgacgccatcatcaactatggaaactcgggaggcccgttagtaaacctggacggtgaagtgattggaattaacactttgaaagtgacagctggaatctcctttgcaatcccatctgataagattaaaaagttcctcacggagtcccatgaccgacaggccaaaggaaaagccatcaccaagaagaagtatattggtatccgaatgatgtcactcacgtccagcaaagccaaagagctgaaggaccggcaccgggacttcccagacgtgatctcaggagcgtatataattgaagtaattcctgataccccagcagaagctggtggtctcaag

SEQ ID NO: 173 - DNA, 304 bp, Homo sapiens
  misc_feature 106, 113, 125, 137: n = A,T,C or G
gggtcaaagttgagctgaagaacggtgccacttacgaagccaaaatcaaggatgtggatgagaaagcagacatcgcactcatcaaaattgaccaccagggcaagcngcctgtnctgctgcttggncgctcctcaganctgcggccgggagagttcgtggtcgccatcggaagcccgttttcccttcaaaacacagtcaccaccgggatcgtgagcaccacccagcgaggcggcaaagagctggggctccgcaactcagacatggactacatccagaccgacgccatcatcaactatggaaactc

SEQ ID NO: 174 - DNA, 258 bp, Homo sapiens
ggtcagaagagttgtgcacgcagattagcaggccaaggtctgagccacagcagcatttttatttcagattttgataactgtttatatgtgttgaaaaccaaaatgacatctttttaaagcttatccataaaaaaaaatagatgtcttttatagtggaaaaacacatggggaaaaaaatcatctattttgatgcagcatttgataatgataaaacacctcacacctcactctttatagtgcacaaaatgaatgaggtct

SEQ ID NO: 175 - DNA, 442 bp, Homo sapiens
aagtagccgctccgagtggaggcgactgggggctgaagagcgcgccgccctctcgtcccactttccaggtgtgtgatcctgtaaaattaaatcttccaagatgatctggtatatattaattataggaattctgcttccccagtctttggctcatccaggcttttttacttcaattggtcagatgactgatttgatccatactgagaaagatctggtgacttctctgaaagattatattaaggcagaagaggacaagttagaacaaataaaaaaatgggcagagaagttagatcggctaactagtacagcgacaaaagatccagaaggatttgttgggcatccagtaaatgcattcaaattaatgaaacgtctgaatactgagtggagtgagttggagaatctggtccttaagggtatgtcagatggctttatctctaaccta

SEQ ID NO: 176 - DNA, 611 bp, Homo sapiens
gggctgaggtaggaagtagccgctccgagtggaggcgactgggggctgaagagcgcgccgccctctcgtcccactttccaggtgtgtgatcctgtaaaattaaatcttccaagatgatctggtatatattaattataggaattctgcttccccagtctttggctcatccaggcttttttacttcaattggtcagatgactgatttgatccatactgagaaagatctggtgacttctctgaaagattatattaaggcagaagaggacaagttagaacaaataaaaaaatgggcagagaagttagatcggctaactagtacagcgacaaaagatccagaaggatttgttgggcatccagtaaatgcattcaaattaatgaaacgtctgaatactgagtggagtgagttggagaatctggtccttaagggtatgtcagatggctttatctctaacctaaccattcagagacagtactttcctaatgatgaagatcaggttggggcagccaaagctctgttacgtctccaggatacctacaatttggatacagataccatctcaaagggtaatcttccaggagtgaaacacaaatcttttctacggctgaggact

SEQ ID NO: 177 - DNA, 416 bp, Homo sapiens
ttacaaactcctgaaccataatattctcgtctccacagacacatactccataatttaaaaccaaatgcttgtgagaaagcttgctcatcatacttgctgcttcaaagaaagactctgaatagtttctgtgtgctttatccagaacttttaaaagaacttctgtttcatgcagttgaccgtagtctcctacttctcttcgtacgcctttaaaaatctttgtaaaagtgccttggccaaggctttcattaaatatcaaatcttcatttctgattttgtgaaacaccatttggttcatatgagtaggcctctgtaatgttggtgaggttggtacatcagaaacaccattcgttctgaagactagaaggtttgatttatcttttcggctttgggggacagcatttagtacacgggaaaat

SEQ ID NO: 178 - DNA, 163 bp, Homo sapiens
gggctttttttttttgcaaagttccaaatttatgggtcgggaaataaatccaaatttctcattaaaaaactcctttggaaaaacttgggcccaaaagtttcccatccgaactcagccttttttgccccgatccccgacttttttactcaaggcccgggaaggc

SEQ ID NO: 179 - DNA, 285 bp, Homo sapiens
aaagttacaaatttattggtctggaaataaatacaaatatctcattaagaaactcctctggaaagacttgtgcacaatagtttcccatccgtactcagcctctcttgccccgatccccgacttttctactcaaggccagggaaaggcctccaaggtgatgggcggcaggtaacgagtcattgcctctcacgccacctggaaggctggactacttcctcctcccaactgcggggtcccagaaatcctcgggtcccagtggctgacttacaatattcaattcactct

SEQ ID NO: 180 - DNA, 458 bp, Homo sapiens
tcgagccgccgccgcccctgtacaacaacaacaacaactgcgaggaaaatgagcagtctctgcccccgccggccggcctcaacagttcctgggtggagctacccatgaacagcagcaatggcaatgataatggcaatgggaaaaatggggggctggaacacgtaccatcctcatcctccatccacaatggagacatggagaagattcctttggatgcacaacatgaatcaggacagagtagttccagaggcagttctcactgtgacagcccttcgccacaagaagatgggcagatcatgtttgatgtggaaatgcacaccagcagggaccatagctctcagtcagaagaagaagttgtagaaggagagaaggaagtcgaggctttgaagaaaagtgcggactgggtatcagactggtccagtagacccgaaaacattccacccaaggagttccacttc

SEQ ID NO: 181 - DNA, 329 bp, Homo sapiens
tttttttttttttttttttttttctttttaataactatcaactcaaacttagggaaacttgcctttgtcttgggggaaaaaaacaactagacaataaagcttcttttacatcatttgctaacctgatctcgttttaagagagagatggtagttatgttgcaagagtaaaatttataccatgaatgatacaggtctagtctggtggcactaattagagataatagcattgctgacaaaattataatctgctggtggcatttgcggaaaagaggcccttgcaaatttctaaacaacagtaaactctgttaggaaattctaaaatgtcttca

SEQ ID NO: 182 - DNA, 527 bp, Homo sapiens
atacatgtaacttcattattttaaaaatatttttagaactccaatactcaccctgttatgtcttgctagtttaaattttgctaattaactgaaacatgcttaccagattcacactgttccagtgtctataaaagaaacactttgaagtctataaaaaataaaataattataaatgtcattgtacatagcatgtttatatctgcaaaaaacctaatagctaattaatctggaatatgcaacattgtccttaattgatgcaaataacacaaatgctgcaaagaaatctactatatcccttaatgaaatacatcattcttcatatatttctccttcagtccattcccttaggcaatttttaatttttaaaaattattatcaggggagaaaaattggcaacgctattatatgtaagggaaatatatacaaaaagaaaattaatcatagtcacctgactaagaaattctgactgctagttgccataaataactcaatggaaatattcctatgggataatgtattttaagtga

SEQ ID NO: 183 - DNA, 530 bp, Homo sapiens
atacatacatgtaacttcattattttaaaaatatttttagaactccaatactcaccctgttatgtcttgctaatttaaattttgctaattaactgaaacatgcttaccagattcacactgttccagtgtctataaaagaaacactttgaagtctataaaaaataaaataattataaatatcattgtacatagcatgtttatatctgcaaaaaacctaatagctaattaatctggaatatgcaacattgtccttaattgatgcaaataacacaaatgctcaaagaaatctactatatcccttaatgaaatacatcattcttcatatatttctccttcagtccattcccttaggcaatttttaatttttaaaaattattatcaggggagaaaaattggcaaaactattatatgtaagggaaatatatacaaaaagaaaattaatcatagtcacctgactaagaaattctgactgctagttgccataaataactcaatggaaatattcctatgggataatgtattttaagtga

SEQ ID NO: 184 - DNA, 253 bp, Homo sapiens
  misc_feature 98, 141, 162, 213: n = A,T,C or G
tatacatacatgtaacttcattattttaaaaatatttttagaactccaatactcaccctgttatgtcttgctaatttaaattttgctaattaactganacatgcttaccagattcacactgttccagtgtctataaaaganacactttgaagtctataaaanataaaataattataaatatcattgtacatagcatgtttatatctgcaaaanacctaatagctaattaatctggaatatgcaacattgtcct

SEQ ID NO: 185 - DNA, 421 bp, Homo sapiens
ccgttgctgtcgatcccagctccttgggaggctgaggcgggagaattgcgggaaggcggggacggaggttgcagtgagccgagatcgcactgctgtacccagcctgggccacagtgcaagactccatctcaaaaaaaaaagaaaagaaaaagcctgtttaatgcacaggtgtgagtggattgcttatggctatgagataggttgatctcgcccttaccccggggtctggtgtatgctgtgctttcctcagcagtatggctctgacatctcttaaatgtcccaacttcagctgttgggagatggtgatattttcaaccctacttcctaaacatctgtctggggttcctttagtcttgaatgtcttatgctcaattatttggtgttgagcctctcttccacaagagctcctccatgtttggat

SEQ ID NO: 186 - DNA, 377 bp, Homo sapiens
cagctccttgggaggctgaggcgggagaattgcttgaacccggggacggaggttgcagtgagccgagatcgcactgctgtacccagcctgggccacagtgcaagactccatctcaaaaaaaaaagaaaagaaaaagcctgtttaatgcacaggtgtgagtggattgcttatggctatgagataggttgatctcgcccttaccccggggtctggtgtatgctgtgctttcctcagcagtatggctctgacatctcttagatgtcccaacttcagctgttgggagatggtgatattttcaaccctacttcctaaacatctgtctggggttcctttagtcttgaatgtcttatgctcaattatttggtgttgagcctctc

SEQ ID NO: 187 - DNA, 243 bp, Homo sapiens
  misc_feature 228: n = A,T,C or G
gaggtattccacctcctaccggaatataattaaagggagaaatacactgtatgaagtatatgttgatactatgacatgttgccaacaccttgagaagcattatttgtttctaataaaagtaatggctttgtcaatatattggtgggtttaaaactttgctgcttttttacataaagcctgtgcctttcctagaaagttaagatgtaaatgtattctcacatgtaaatntgaaagttcaggggt

SEQ ID NO: 188 - DNA, 544 bp, Homo sapiens
tattccacctcctaccggaatataattaaagggagaaatacactgtatgaagtatatgttgatactatgacatgttgccaacaccttgagaagcattatttgtttctaataaaagtaatggctttgtcaatatattggtgggtttaaaactttgctgcttttttacataaagcctgtgcctttcctagaaagttaagatgtaaatgtattctcacatgtaaatttgaaagttcaggggtctattatgaaatgatacacatttttaaatgaaccataatttttttcactaagctgtttgccttccaaagtgtttacaccttaagccttaacatgtatcttcattcagaaaacagttatattgtcataccatagtaggaagaaaaacctttatttggaatatacactactgtaagtttgtacagatcatatacctaccacctgtctttgcttaaagagccttgattacataaatatgtaggaaaaaacatattgagttcaaaatttatatctaacattgtttatgttatgatttttttttaattgc

SEQ ID NO: 189 - DNA, 244 bp, Homo sapiens
  misc_feature 210: n = A,T,C or G
cacaaaaggtatgatcagcaacttgcttgggaaaggagccgtggaccagctgacacggctggtgctggtgaatgccctctacttcaacggccagtggaagactcccttccccgactccagcacccaccgccgcctcttccacaaatcagacggcagcactgtctctgtgcccatgatggctcagaccaacaagttcaactatactgagtncaccacgcccgatggccattatacgacatcctgg

SEQ ID NO: 190 - DNA, 209 bp, Homo sapiens
  misc_feature 140: n = A,T,C or G
gaacactgttgctcttggtggacgggcccagaggaattcagagttaaaccttgagtgcctgcgtccgtgagaattcagcatggaatgtctctactatttcctgggatttctgctcctggctgcaagattgccacttgatnccgccaaacgatttcatgatgtgctgggcaatgaaagaccttctgcttacatgagggagcacaatcaat

SEQ ID NO: 191 - DNA, 254 bp, Homo sapiens
  misc_feature 85, 100, 143, 155, 182, 203, 229, 245, 254: n = A,T,C or G
ctcccaaccaagctctcttgaggatcttgaaggaaactgaattcaaaaagatcaaagtcctgggctccggtgcgttcggcacggngtataagggactctngatcccagaaggtgagaaagttaaaattcccgtcgctatcaangaattaagagangcaacatctccgaaagccaacaagganatcctcgatgaagcctacgtnatggccagcgtggacaacccccacgngtgccgcctgctgggnatctgtctn

SEQ ID NO: 192 - DNA, 484 bp, Homo sapiens
tttttttttttttttttttcaaatatacctctttgaaagataaatttctgctcaaagggacaatattcttgctggatgcgttcctgtaaatgcttcacagtttgaagacaaaggaatgcaacttcccaaaatgtgcccgaggtggaagtacttcctggctagtcggtgtaaacgttgcaaaaccagtctgtgggtctaagagctaatgcgggcatggctgttgggatggaggacctgctgtggcttggtcctgggtatcgaaagagtctggatttttagggctcatactatcctccgtggtcatactccaataaattcactgctttgtggcgcgacccttaggtattctgcattttcagctgtggagcccttaaagatgccatttggcttggcttccttgggaaagaagtcctgctggtagtcagggttgtccaggctaatttggtggctgcctttctgggcccagtgggcagggctgtcgaat

SEQ ID NO: 193 - DNA, 660 bp, Homo sapiens
tttaatcatatccaggagtttgcaagaaacaggtgcttaacactaattcacctcctgaacaagaaaaatgggctgtgaccggaactgtgggctcatcgctggggctgtcattggtgctgtcctggctgtgtttggaggtattctaatgccagttggagacctgcttatccagaagacaattaaaaagcaagttgtcctcgaagaaggtacaattgcttttaaaaattgggttaaaacaggcacagaagtttacagacagttttggatctttgatgtgcaaaatccacaggaagtgatgatgaacagcagcaacattcaagttaagcaaagaggtccttatacgtacagagttcgttttctagccaaggaaaatgtaacccaggacgctgaggacaacacagtctctttcctgcagcccaatggtgccatcttcgaaccttcactatcagttggaacagaggctgacaacttcacagttctcaatctggctgtggcagctgcatcccatatctatcaaaatcaatttgttcaaatgatcctcaattcacttattaacaagtcaaaatcttctatgttccaagtcagaactttgagagaactgttatggggctatagggatccatttttgagtttggttccgtaccctgttactaccacagt

SEQ ID NO: 194 - DNA, 277 bp, Homo sapiens
ctttaatcatatccaggagtttgcaagaaacaggtgcttaacactaattcacctcctgaacaagaaaaatgggctgtgaccggaactgtgggctcatcgctggggctgtcattggtgctgtcctggctgtgtttggaggtattctaatgccagttggagacctgcttatccagaagacaattaaaaagcaagttgtcctcgaagaaggtacaattgcttttaaaaattgggttaaaacaggcacagaagtttacagacagttttggatctttgatgt

SEQ ID NO: 195 - DNA, 457 bp, Homo sapiens
gactgggtttgggtgcagacgttgttgcttgggcgcttctccgctgcgtgtaggtgaagggggcttcctgaccgagacatggatttaggtgctattacaaaatactcagcattacacgccaagcccaatggactgatccttcaatacgggactgctggatttcgaacgaaggcagaacatcttgatcatgtcatgtttcgcatgggattattagctgtcctgaggtcaaaacagacaaaatccactataggagtcatggtaacagcgtcccacaatcctgaggaagacaatggtgtaaaattggttgatcctttgggtgaaatgttggcaccatcctgggaggaacatgccacctgtttagcaaatgctgaggaacaagatatgcagagagtgcttattgacatcagcgagaaagaagctgtgaatctgcaacaagatgcctttgtagttattggta

SEQ ID NO: 196 - DNA, 361 bp, Homo sapiens
tttttttttttttttttttttttgggcaggagaccatgttactttattcatttgtttaactttaaccatgttcaataaacttttcacctgtttggtgagttccacaaaagccttagagagtttctggtagtaaccttctatagttgcctttccatatcggccacccgtgtttcgacaatacaccatgtagtgcagctggggtgttgttaacaagccataatcatggaattgacctcctagaacagtcacaccatctattacagattgtgaaagtttctcactgctgggcctggtatctctaccaataactacaaaggcatcttgttgcagattcacagcttctttctcgctgatgtcaata

SEQ ID NO: 197 - DNA, 551 bp, Homo sapiens
gagccgagctgatttgatcgaggagcgcggttaccggacgggctgggtctatggtcgctccgcgggccgctccgccggctggtgcttttttatcagggcaagctgtgttccatggcagggaacttttggcagatctcccactatttgcaatggattttggataaacaagatctgttgaaggagcgccaaaaggatttaaagtttctctcagaggaagaatattggaagttacaaatattttttacaaatgttatccaagcattaggtgaacatcttaaattaagacaacaagttattgccactgctacggtatatttcaagagattctatgccaggtattctctgaaaagtatagatcctgtattaatggctcctacatgtgtgtttttggcatccaaagtagaggaatttggagtagtttcaaatacaagattgattgctgctgctacttctgtattaaaaactagattttcatatgcctttccaaaggaatttccttataggatgaatcatatattagaatgtgaattctatctgttagaactaatgga

SEQ ID NO: 198 - DNA, 637 bp, Homo sapiens
tacggccgggagtcgagccgagctgatttgatcgaggagcgcggttaccggacgggctgggtctatggtcgctccgcgggccgctccgccggctggtgcttttttatcagggcaagctgtgttccatggcagggaacttttggcagagctcccactatttgcaatggattttggataaacaagatctgttgaaggagcgccaaaaggatttaaagtttctctcagaggaagaatattggaagttacaaatattttttacaaatgttatccaagcattaggtgaacatcttaaattaagacaacaagttattgccactgctacggtatatttcaagagattctatgccaggtattctctgaaaagtatagatcctgtattaatggctcctacatgtgtgtttttggcatccaaagtagaggaatttggagtagtttcaaatacaagattgattgctgctgctacttctgtattaaaaactagattttcatatgcctttccaaaggaatttccttataggatgaatcatatattagaatgtgaattctatctgttagaactaatggattgttgcttgatagtgtatcatccttatagacctttgctccagtatgtgcaggacatgggccaagaagacat

SEQ ID NO: 199 - DNA, 130 bp, Homo sapiens
tagaaagcctccacctggagtacaatgccctcaaggtccttcacaatggcaccctggctgagttgcaaggtctaccccacattagggttttcctggacaacaatccctgggtctgcgactgccacatggc

SEQ ID NO: 200 - DNA, 372 bp, Homo sapiens
  misc_feature 29, 100, 297, 298, 353, 357: n = A,T,C or G
gtgctgtttgaccaatggtcatgtggccnagattggggacttcgggctggctagggacatcatgaatgactccaactacattgtcaagggcaatgccgcnctgcctgtgaagtggatggccccagagagcatctttgactgtgtctacacggttcagagcgacgtctggtcctatggcatcctcctctgggagatcttctcacttgggctgaatccctaccctggcatcctggtgaacagcaagttctataaactggtgaaggatggataccaaatggcccagcctgcatttgcccnnaagaatatatacagcatcatgcaggcctgctgggcttgggagcccacccacaganccancttccagcagatctg

SEQ ID NO: 201 - DNA, 478 bp, Homo sapiens
  misc_feature 3, 10, 11, 78, 112, 130, 150, 231, 457: n = A,T,C or G
gancacctgnnacaaggaggatggacggcccctggagctccgggacctgcttcacttctccagccaagtagcccaggnatggccttcctcgcttccaagaattgcatccacngggacgtggcagcgcgtnacgtgctgttgaccaatggncatgtggccaagattggggacttcgggctggctagggacatcatgaatgactccaactacattgtcaagggcaatgccgcnctgcctgtgaagtggatggccccagagagcatctttgactgtgtctacacggttcagagcgacgtctggtcctatggcatcctcctctgggagatcttctcacttgggctgaatccctaccctggcatcctggtgaacagcaagttctataaactgggtgaaaggatggataccaaatggcccagcctgcattttgcccccaaagaatatatacaagcatccatgnaggcccttctgggccttggag

SEQ ID NO: 202 - DNA, 218 bp, Homo sapiens
gcgagcaaggggatatcgcccagcccttgctgcagcccaacaactatcagttctgctgaggagttgacgacagggagtaccactctcccctcccacaaacttcaactcctccatggatggggcgacacggggagaacatacaaactctgccttcggtcatttcactcaacagctcggcccagctctgaaacttgggaaggtgagggattcaggggagg

SEQ ID NO: 203 - DNA, 556 bp, Homo sapiens
taagctcggaattcggctcgaggcgagcaaggggatatcgcccagcccttgctgcagcccaacaactatcagttctgctgaggagttgacgacagggagtaccactctcccctcccacaaacttcaactcctccatggatggggcgacacggggagaacatacaaactctgccttcggtcatttcactcaacagctcggcccagctctgaaacttgggaaggtgagggattcaggggaggtcagaggatcccacttcctgagcatgggccatcactgccagtcaggggctgggggctgagccctcacccccccctcccctactgttctcatggtgttggcctcgtgtttgctatgccaactagtagaaccttctttcctaatccccttatcttcatggaaatggactgactttatgcctatgaagtccccaggagctacactgatactgagaaaaccaggctctttggggctagacagactggcagagagtgagatctccctctctgagaggagcagcagatgctcacagaccacactcagctcaggccccttgga

SEQ ID NO: 204 - DNA, 319 bp, Homo sapiens
tccttatttatttaacttcacccgagttcctctgggtttctaagcagttatggtgatgacttagcgtcaagacatttgctgaactcagcacattcgggaccaatatatagtgggtacatcaagtccatctgacaaaatggggcagaagagaaaggactcagtgtgtgatccggtttctttttgctcgcccctgttttttgtagaatctcttcatgcttgacatacctaccagtattattcccgacgacacatatacatatgagaatataccttatttatttttgtgtaggtgtctgccttcacaaatgtcatgtctact

SEQ ID NO: 205 - DNA, 456 bp, Homo sapiens
attccgttgctgtcgagggtcactaccagtacaagagcatccctgtggaggacaaccacaaggcagacatcagctcctggttcaacgaggccattgacttcatagactccatcaagaatgctggaggaagggtgtttgtccactgccaggcaggcatttcccggtcagccaccatctgcctggcttaccttatgaggactaatcgagtcaagctggacgaggcctttgagtttgtgaagcagaggcgaagcatcatctctcccaacttcagcttcatgggccagctgctgcagtttgagtcccaggtgctggctccgcactgttcggcagaggctgggagccccgccatggctgtgctcgaccgaggcacctccaccaccaccgtgttcaacttccccgtctccatccctgtccactccacgaacagtgcgctgagctaccttcagagccccatta

SEQ ID NO: 206 - DNA, 533 bp, Homo sapiens
agtttttaaataatgaatattatttaataccacaacagaattatccccaatttccaataagtcctatcattgaaaattcaaatataagtgaagaaaaaattagtagatcaacaatctaaacaaatccctcggttctaagatacaatggattccccatactggaaggactctgaggctttattcccccactatgcatatcttatcattttattattatacacacatccatcctaaactatactaaagcccttttcccatgcatggatggaaatggaagatttttttttaacttgttctaaaagtcttaatatgggctgttgccatgaaggcttgcagaattgagtccattttctagctgcctttattcacatagtggacggggtacctaaaagtactggggttgactcagagagtcgctgtcattctgtcattgctgctactctaacactgagcaacactctcccagtggcagatcccctgtatcattccaagaggagcattcatccctttgctctaatgatcaggaatgatgc

SEQ ID NO: 207 - DNA, 246 bp, Homo sapiens
aatgcactaactcaataccaagatgagtttttaaataatgaatattatttaataccacaacagaattatccccaatttccaataagtcctatcattgaaaattcaaatataagtgaagaaaaaattagtagatcaacaatctaaacaaatccctcggttctaagatacaatggattccccatactggaaggactctgaggctttattcccccactatgcatatcttatcattttattattatacac

SEQ ID NO: 208 - DNA, 407 bp, Homo sapiens
ggccgcccttttttttttttttttttttttttttttttgggcaaaaaggggcttttttttttttccccccccctttttttaacccttcccctaatatttcccccaaaaaaaaaaattttttttttttgggggggggaaaaaaaagggaaaaaaaacccccccccccggggggggaaaaaaaccccccaaaaaccccccttttgggggggtccccccccatgggggttccccccccaatttttttcccccccccaaaaaaatttttaacccccccccaagggggtggaaaaccttaaaaaaaaccccccggaaaaaccaaaaccccttttttaaaaaaaaaaaaaaaatttttggggggcaaaaccccccccccccaaaaaacccccccccccccccttaaaaaaaaa

SEQ ID NO: 209 - DNA, 359 bp, Homo sapiens
  misc_feature 1, 53, 121, 123, 128, 133, 142, 150, 174, 179, 183, 186,196, 200, 201, 204, 207, 212, 215, 218, 224, 229, 230, 231,243, 244, 249, 260, 261, 267, 268, 270, 273, 279, 289, 291,295, 301, 303, 305, 312, 315, 337, 345, 357: n = A,T,C or G
ncggggactgcgcggcggtgcagagccgggcgtgggcgagaacgaacgggctncctgcggctgagagcgtcgagtgtcaccatgggtatcacgcttggagcttcctaaaggacttcctggncngggcntcgcnctgcccgtntccaagancccggtcggccccaatcgagaggncaaanctgntgntgaaggtgcnagannccnagnaacangtnaantcttangaagnnntacaaaggggtnnattanttttttggtannattccnnangancaaggntttcctttcntnttgnagggtnancntggcaangtnattccttaatttcccaaccaangttttaantttggctttaangg

SEQ ID NO: 210 - DNA, 394 bp, Homo sapiens
ttttttttttgcattaagtggtctttattgatgtttcacattcagttattatcaattcttcagttaattgtacaagtatgataaattattttctatttgctgtgggaatttaaatgtaaaataaatacaaaatacatgtgtggtttaatgaacactcaatgaagcatctcttctgaggtattcctttcagtctggttttatcccaggatctttttacttcccctaggaatagtctattaaaccacacaatggatctgtgaacttgtagatcaagttcactgtaaatctgtgaacttgtgttttaattacattagacatattttttgatctcatcatacaacaccaatacaaaaggcaccgcccatgcctctcaggcacattgggaccgggcacc

SEQ ID NO: 211 - DNA, 292 bp, Homo sapiens
gggagcccaccagcaagaatgagttggagcaatcttttcatgtgacctccttaacagatatttactgaaggaatctaggttgtattttcagtggacaatgggaataaagcatttctaaagcaccgactggagaggaaggcaacagagacaaggagagaagccgagagacatgtctgcgtgctgccacgcatttgagcgattgctctgtgaagagttgtacactgaacactttcaggggaggctgtttacccaggcaatgtcctcaaacaagcctgtgccggggtgtcctgga

SEQ ID NO: 212 - DNA, 495 bp, Homo sapiens
aattccgttgctgtcgctgcgcccaggtaatttgagcaaaggccacagtgaactccggcgtggctgaggaaggaggaggcacccacaggctgctgggaggagagcataaggctcaaaatggaaaatcataaatccaataataaggaaaacataacaattgttgatatatccagaaaaattaaccagcttccagaagcagaaaggaatctacttgaaaatggatcggtttatgttggattaaatgctgctctttgtggcctcatagcaaacagtctttttcgacgcatcttgaatgtgacaaaggctcgcatagctgctggcttaccaatggcagggataccttttcttacaacagacttaacttacagatgttttgtaagttttcctttgaatacaggtgatttggattgtgaaacctgtaccataacacggagtggactgactggtcttgttattggtggtctataccctgttttcttggctatacctgtaaat

SEQ ID NO: 213 - DNA, 358 bp, Homo sapiens
tgcgaccgcgatctcctgcagctggtgcaccacctcggcgatggacagccgctcctccgggttcacctgcagcatggcgaggatgaggctgtggaagaccgtgtactgcgtgtcgtgcggggggatcgagtacttcccattgactattcgaagtttcgctccatcctcaaaagggtgctgccggaagcacagcaggtacaagatgcagcccagggcccagatatcctgcttctcgccgatcgggaagttggaatacaagtctatgatttctggtgttctatacattggtgttgtattcctcgtgatctgaaaaaatacaaacatttcaaaggaaaagttgcatcccacaaacagtatt

SEQ ID NO: 214 - DNA, 406 bp, Homo sapiens
tggtacgcctgcaggtaccggtccggaattcccgggtcgacccacgcgtccgaggacatctggaatgtcactggtgcccaggtgtacttgagctgtgaggtcatcggaatcccgacacctgtcctcatctggaacaaggtaaaaaggggtcactatggagttcaaaggacagaactcctgcctggtgaccgggacaacctggccattcagacccggggtggcccagaaaagcatgaagtaactggctgggtgctggtatctcctctaagtaaggaagatgctggagaatatgagtgccatgcatccaattcccaaggacaggcttcagcatcagcaaaaattacagtggttgatgccttacatgaaataccagtgaaaaaaggtgaaggtgccgagctataaacct

SEQ ID NO: 215 - DNA, 300 bp, Homo sapiens
  misc_feature 66, 71, 259: n = A,T,C or G
aggacatctggaatgtcactggtgcccaggtgtacttgagctgtgaggtcatcggaatcccgacanctgtnctcatctggaacaaggtaaaaaggggtcactatggagttcaaaggacagaacttctgcctggtgaccgggacaacctggccattcagacccggggtggcccagaaaagcatgaagtaactggctgggtgctggtatctcctctaagtaaggaagatgctggagaatatgagtgccatgcatccaattnccaaggacaggcttcagcatcagcaaaaattacagtggttg

SEQ ID NO: 216 - DNA, 232 bp, Homo sapiens
ttcaaaagcttagagagaataagcttcttggtggtgaaatacaactctcacgtgtgctccagttctaaaattaacctgtgcctggtctctgaagccctttcttgctctgtgcctttcagccacatccttaggtgctaacggccatgagctccgactctccaaagtgagctccactttgggtctgaggagcccctggcagagtccacgctgcctcaggtatcatgggcgtaat

SEQ ID NO: 217 - DNA, 453 bp, Homo sapiens
ataagcttcttggtggtgaaactacaactctcacgtgtgctccagttctaaaattaacctgtgcctggtctctgaagccctttcttgctctgtgcctttcagccacatccttaggtgctaacggccatgagctccgactctccaaagtgagctccactttgggtctgaggagcccctggcagagtccacgctgcctcaggtatcatgggcgtaatgatcacccaggctccgggagatctcatggatgattactgtatgagacagaggggacttcagtctttccagggccttggtggaatttttggctctggtgttttcgccagacaataaacttacactggaagctttgattcaccctccacagtactccagaaaggactgtcctataagttgtacactttaaaaggtcatgtagaggttgtagtagaatggcttttcaccctggtgactttg

SEQ ID NO: 218 - DNA, 520 bp, Homo sapiens
agatgtgtgagaagtgccccacctgcccggatgcatgcagcaccaagagagattgcgtcgagtgcctgctgctccactctgggaaacctgacaaccagacctgccacagcctatgcagggatgaggtgatcacatgggtggacaccatcgtgaaagatgaccaggaggctgtgctatgtttctacaaaaccgccaaggactgcgtcatgatgttcacctatgtggagctccccagtgggaagtccaacctgaccgtcctcagggagccagagtgtggaaacacccccaacgccatgaccatcctcctggctgtggtcggtagcctcctccttgttgggcttgcactcctggctatctggaagctgcttgtcaccatccacgaccggagggagtttgcaaagtttcagagcgagcgatccagggcccgctatgaaatggcttcaaatctattatacagaaagcctatctccacgcacactgtggacttcaccttcaacaagttcaacaaatcctacaatgg

SEQ ID NO: 219 - DNA, 404 bp, Homo sapiens
agatgtgtgagaagtgccccacctgcccggatgcatgcagcaccaagagagattgcgtcgagtgcctgctgctccactctgggaaacctgacaaccagacctgccacagcctatgcagggatgaggtgatcacatgggtggacaccatcgtgaaagatgaccaggaggctgtgctatgtttctacaaaaccgccaaggactgcgtcatgatgttcacctatgtggagctccccagtgggaagtccaacctgaccgtcctcagggagccagagtgtggaaacacccccaacgccatgaccatcctcctggctgtggtcggtagcctcctccttgttgggcttgcactcctggctatctggaagctgcttgtcaccatccacgaccggagggagtttgcaaagttt

SEQ ID NO: 220 - DNA, 80 bp, Homo sapiens
atggcttcaaatccattatacagaaagcctatctccacgcacactgtggacttcaccttcaacaagttcaacaaatccta

SEQ ID NO: 221 - DNA, 607 bp, Homo sapiens
tgccccacctgcccggatgcatgcagcaccaagagagattgcgtcgagtgcctgctgctccactctgggaaacctgacaaccagacctgccacagcctatgcagggatgaggtgatcacatgggtggacaccatcgtgaaagatgaccaggaggctgtgctatgtttctacaaaaccgccaaggactgcgtcatgatgttcacctatgtggagctccccagtgggaagtccaacctgaccgtcctcagggagccagagtgtggaaacacccccaacgccatgaccatcctcctggctgtggtcggtagcatcctccttgttgggcttgcactcctggctatctggaagctgcttgtcaccatccacgaccggagggagtttgcaaagtttcagagcgagcgatccagggcccgctatgaaatggcttcaaatccattatacagaaagcctatctccacgcacactgtggacttcaccttcaacaagttcaacaaatcctacaatggcactgtggactgatgtttccttctccgaggggctggagcggggatctgatgaaaaggtcagactgaaacgccttgcacggctgctcggcttgatcacaact

SEQ ID NO: 222 - DNA, 583 bp, Homo sapiens
ggtatgtgccatcacaagcagatgtggcagtatttgaagccgtgtccagcccaccgcctgccgacttgtgtcatgccctacgttggtataatcacatcaagtcttacgaaaaggaaaaggccagcctgccaggagtgaagaaagctttgggcaaatatggtcctgccgatgtggaagacactacaggaagtggagctacagatagtaaagatgatgatgacattgacctctttggatctgatgatgaggaggaaagtgaagaagcaaagaggctaagggaagaacgtcttgcacaatatgaatcaaagaaagccaaaaaacctgcacttgttgccaagtcttccatcttactagatgtgaaaccttgggatgatgagacagatatggcgaaattagaggagtgcgtcagaagcattcaagcagacggcttagtctggggctcatctaaactagttccagtgggatacggaattaagaaacttcaaatacagtgtgtagttgaagatgataaagttggaacagatatgctggaggagcagatcactgcttttgaggactatgtgcagtccatggatgtggctgc

SEQ ID NO: 223 - DNA, 296 bp, Homo sapiens
tacatcgaggggtatgtgccatcacaagcagatgtggcagtatttgaagccgtgtccagcccaccgcctgccgacttgtgtcatgccctacgttggtataatcacatcaagtcttacgaaaaggaaaaggccagcctgccaggagtgaagaaagctttgggcaaatatggtcctgccgatgtggaagacactacaggaagtggagctacagatagtaaagatgatgatgacattgacctctttggatctgatgatgaggaggaaagtgaaaaagcaaagaggctaagggaagaacg

SEQ ID NO: 224 - DNA, 208 bp, Homo sapiens
  misc_feature 97: n = A,T,C or G
gactacatcttggacctgcagatcgccctggactcgcatcccactattgtcagcctgcatcaccagagacccgggcagaaccaggcgtccaggacgncgctgaccaccctcaacacggatatcagcatcctgtccttgcaggcttctgaattcccttctgagttaatgtcaaatgacagcaaagcactgtgtggctgaataagcggtg

SEQ ID NO: 225 - DNA, 274 bp, Homo sapiens
  misc_feature 133: n = A,T,C or G
gcagcggctggagcggcagatcagccaggatgtcaagctggagccagacatcctgcttcgggccaagcaagatttcctgaagacggacagtgactcggacctacagctctacaaggaacagggtgaggggcanggtgaccggagcctgcgggagcgtgatgtgctggaacgggagtttcagcgggtcaccatctctggggaggagaagtgtggggtgccgttcacagacctgctggatgcagccaagatgtggtgcgggcgtcttcatccggga

SEQ ID NO: 226 - DNA, 330 bp, Homo sapiens
ggccgccctttttttttttttttttttttgggcccagggggggcccccttgggaaaaacacccgggaaacttcccaaaggggccttgggggaattttttttaaaaaaaaacctttttttaaaaaaaactttgggatttaaattttttttccggccccttttttgggccgggtaccccaatttaaaaaaggggggctttttaaaggttgggaaaaaaaaaaaattgggggggcccaaaaaattggggggcccccaaaaaaaaagcggggtttggaaaaattttgggggggtttggaaatttgggccccaaaacgggggacccctttccccc

SEQ ID NO: 227 - DNA, 525 bp, Homo sapiens
gaatttggccctcgaggccaagaattcggcacgagggttcacatagcaatttaatcaagtaatggttaattagttaccccctatatataaatatatgtaatcaatttcttcaaatagcttgcttacatgataatcaattagccaaccatgagtcatttagaatagtgataaatagaatacacagaatagtgatgaaattcaatttaaaaaatcacgttagcctccaaaccatttaattcaaatgaacccatcaactggatgccaactctggcgaatgtaggacctctgagtggctgtataattgttaattcaaatgaaattcatttaaacagttgacaaactgtcattcaacaattagctccaggaaataacagttatttcatcataaaacagtcccttcaaacacacaattgttctgctgaagagttgtcatcaacaatccaatgctcacctattcagttgctctgtggtcagtgtggctgcataacagtggattccatgaaaggagtcattttagtgatgagc

SEQ ID NO: 228 - DNA, 788 bp, Homo sapiens
  misc_feature 42, 44, 48, 49, 51, 52, 53, 54, 55, 57, 59, 61, 62, 63,64, 68, 69, 70, 71, 73, 74, 75, 76, 77, 79, 80, 83, 87, 89,92, 93, 94, 95, 97, 98, 107, 112, 113, 117, 122, 125, 127,130, 131, 133, 671, 677, 685, 706, 713, 718, 725, 757, 771: n = A,T,C or G
  misc_feature 783: n = A,T,C or G
gttcacatagcaatttaatcaagtaatcattaattagggggngngggnngnnnnngngntnnnngtgnnnngnnnnngnnggngtgngngtnnnngnngggaggtgnggaanngttnttttntgngngannantagaatacacagaatagtgatgaaattcaatttaaaaaatcacgttagcctccaaaccatttaattcaaatgaacccatcaactggatgccaactctggcgaatgtaggacctctgagtggctgtataattgttaattcaaatgaaattcatttaaacagttgacaaactgtcattcaacaattagctccaggaaataacagttatttcatcataaaacagtcccttcaaacacacaattgttctgctgaagagttgtcatcaacaatccaatgctcacctattcagttgctctgtggtcagtgtggctgcataacagtggattccatgaaaggagtcattttagtgatgagctgccagtccattcccaggccaggctgtcgctggccatccattcagtcgattcagtcataggcgaatctgttctgcccgaagcttgtggtcaagcaaaaattcagccctgaaaatcagcacatctgttcggtggactaaaccacagttagttcgtcaagcagcaacccctgtggcatgaccgccantgggtncatgcgtntgcactgggagttggccaaanctccggnggtcncggggtnttttttgtgggttttttttttttagtcttgtntttgggtaagtggnttttttttttcnttggg

SEQ ID NO: 229 - DNA, 156 bp, Homo sapiens
gccgagggaagggcccggcagctgaggagccgctgagcttgctggacgacatgaaccactgctactcccgcctgcgggaactggtacccggagtcccgagaggcactcagcttagccaggtggaaatcctacagcgcgtcatcgactacattctcg

SEQ ID NO: 230 - DNA, 538 bp, Homo sapiens
tacgactcctatagggaatttggccctcgaggccaagaattcggcacgagggtgactttggctttgctcgcatcatcggcgagaagtcgttccgccgctcagtggtgggcacgccggcctacctggcacccgaggtgctgctcaaccagggctacaaccgctcgctggacatgtggtcagtgggcgtgatcatgtacgtcagcctcagcggcaccttccctttcaacgaggatgaggacatcaatgaccagatccagaacgccgccttcatgtacccggccagcccctggagccacatctcagctggagccattgacctcatcaacaacctgctgcaggtgaagatgcgcaaacgctacagcgtggacaaatctctcagccacccctggttacaggagtaccagacgtggctggacctccgagagctggaggggaagatgggagagcgatacatcacgcatgagagtgacgacgcgcgctgggagcagtttgcagcagagcatccgctgcctgggtctgggctgcccacggacaggga

SEQ ID NO: 231 - DNA, 232 bp, Homo sapiens
  misc_feature 18, 56, 94, 103, 117, 128, 145, 184, 204, 219: n = A,T,C or G
tggctttgctcgcatcancggcgagaagtcgtcccgccgctcagtggtgggcacgncggcctacctggcacccgaggtcttgctcaaccagggntacaaccgntcgctcgacatgtngtcagtgggcntgatcatgtacgtcagnctcagcggcaccttccctttcaacgaggatgaggacatnaatgaccagatccagaacgncgacttcatgtaccnggccagaccctgg

SEQ ID NO: 232 - DNA, 420 bp, Homo sapiens
taccggtccggaattcccgggtcgacccacgcgtccggcgtctctgctccaccaaggtgccctggacatgctgaccaaggtgatggccctagagctcgggccccacaagatccgagtgaatgcagtaaaccccacagtggtgatgacgtccatgggccaggccacctggagtgacccccacaaggccaagactatgctgaaccgaatcccacttggcaagtttgctgaggtagagcacgtggtgaacgccatcctctttctgctgagtgaccgaagtggcatgaccacgggttccactttgccggtggaagggggcttctgggcctgctgagctccctccacacacctcaagccccatgccgtgctcatcctacccccaatccctccaataaacctgattctgctgcccaaaaaaaaaaa

SEQ ID NO: 233 - DNA, 294 bp, Homo sapiens
  misc_feature 2, 170: n = A,T,C or G
gngtctactgctccaccaagggtgccctggacatgctgaccaaggtgatggccctagagctcgggccccacaagatccgagtgaatgcagtaaaccccacagtggtgatgacgtccatgggccaggccacctggagtgacccccacaaggccaagactatgctgaaccgnatcccacttggcaagtttgctgaggtagagcacgtggtgaacgccatcctctttctgctgagtgaccgaagtggcatgaccacgggttccactttgccggtggaagggggttctggggctgctg

SEQ ID NO: 234 - DNA, 55 bp, Homo sapiens
  misc_feature 42: n = A,T,C or G
gtctcggtccatgactctggagatccgagaaggaagaggctntggcctgagaaag

SEQ ID NO: 235 - DNA, 394 bp, Homo sapiens
  misc_feature 22, 335, 365, 377, 383, 391: n = A,T,C or G
ttttttgttcatttatattttntttaagagctgtgcccagttttatcatctcacaagaatgaagcaagggacaaaggtaagtgccacgctccctggccactgggttcctggcaagctcccagccactaggtgccaatctcccttcaatgtactccttcttccccagagtgcagaagcgtatgaagacagttatgacatggacacatgcatgagctattatacataattacaaaagctgattctgtcatcaccacatcttgtctcatcagtaggagcgaatggctggcgggacggtggcacagtcagccttgttcaaagttttgtcgatcacgggncctatattccagagtgacctttcccagtgnccaacgttccanataggncagggtcntgc

SEQ ID NO: 236 - DNA, 468 bp, Homo sapiens
agctcgggattcggctcgaggacctggaaattccaggtggtgagctgcatcgaaggggagcctgggcccgtcaggagcgtcctcttcaacccagacggctgctgcctgtacagcggctgccaggactcactgcgtgtctacggctgggaacctgagcggtgctttgatgtggtcctcgtcaactggggcaaggtggccgacctggccatctgcaatgaccagttgataggtgtggccttctcccagagcaacgtctcctcctacgtggtggatctgacgcgtgtcaccaggactggcacggtggcccgggaccctgtgcaggaccaccggcccctggcacagccactgcccaaccccagcgcccccctccggcgcatctatgagcggcccagcacaacctgcagcaagcctcagagggtgaagcagaactcagagagcgagcgccgcacccccagcagcgaggatgac

SEQ ID NO: 237 - DNA, 254 bp, Homo sapiens
  misc_feature 48, 85, 97: n = A,T,C or G
gacctggagaagttccaggtggtgagctgcatcgaaggggagcctggncccgtcaggagcgtcctcttcaacccagacggctgcngcctgtacagcngctgccaggactcactgcgtgtctacggctgggaacctgagcggtgctttgatgtggtcctcgtcaactggggcaaggtggccgacctggccatctgcaatgaccagttgataggtgtggccttctcccagagcaacgtctcctcctacgtggtgga

SEQ ID NO: 238 - DNA, 419 bp, Homo sapiens
gacccacgcgtccgtcttcaacttctttagtcctcctgagattcctatgattgggaagctggaaccacgagaagatgctatcctggatgaggactttgaaattgggcagattttacatgataatgtcatcctgaaatcaatctattactatactggagaagtcaatggtacctactatcaatttggcaaacattatggaaacaagaaatacagaaaataagtcaatctgaaagatttttcaagaatcttaaaatctcaagaagtgaagcagattcatacagccttgaaaaaagtaaaaccctgacctgtaacctgaacactattattccttatagtcaagtttttgtggtttcttggtagtctatattttaaaaatagtcctaaaaagtgtctaagtgccagtttattctatctaggct

SEQ ID NO: 239 - DNA, 228 bp, Homo sapiens
  misc_feature 190: n = A,T,C or G
gaaccccgcccgcggccacagcgtctgctccacctccagcttgtacctgcaggatctgagcgccgccgcctcagagtgcatcgacccctcggtggtcttcccctaccctctcaacgacagcagctcgcccaagtcctgcgcctcgcaagactccagcgccttctctccgtcctcggattctctgcactcntcgacggagtcctccccgcagggcagccccgagcccct

SEQ ID NO: 240 - DNA, 525 bp, Homo sapiens
aaccccgcccgcggccacagcgtctgctccacctccagcttgtacctgcaggatctgagcgccgccgcctcagagtgcatcgacccctcggtggtcttcccctaccctctcaacgacagcagctcgcccaagtcctgcgcctcgcaagactccagcgccttctctccgtcctcggattctctgctctcctcgacggagtcctccccgcagggcagccccgagcccctggtgctccatgaggagacaccgcccaccaccagcagcgactctgaggaggaacaagaagatgaggaagaaatcgatgttgtttctgtggaaaagaggcaggctcctggcaaaaggtcagagtctggatcaccttctgctggaggccacagcaaacctcctcacagcccactggtcctcaagaggtgccacgtctccacacatcagcacaactacgcagcgcctccctccactcggaaggactatcctgctgccaagagggtcaagttggacagtgtcagagtcctgagacagatcagc

SEQ ID NO: 241 - DNA, 552 bp, Homo sapiens
tggaaggaactggtctgctcacacttgctggcttgcgcatcaggactggctttatctcctgactcacggtgcaaaggtgcactctgcgaacgttaagtccgtcccagcgcttggaatcctacggcccccacagccggatcccctcagccttccaggtcctcaactcccgcggacgctgaacaatggcctccatggggctacaggtaatgggcatcgcgctggccgtcctgggctggctggccgtcatgctgtgctgcgcgctgcccatgtggcgcgtgacggccttcatcggcagcaacattgtcacctcgcagaccatctgggagggcctatggatgaactgcgtggtgcagagcaccggccagatgcagtgcaaggtgtacgacttgctgctggcactgccgcaggacctgcaggcggcccgcgccctcgtcatcatcagcatcatcgtggctgctctgggcgtgctgctgtccgtggtggggggcaagtgtaccaactgcctggaggatgaaagcgccaaggccaagaccatgatcgtggcgggcgtgg

SEQ ID NO: 242 - DNA, 519 bp, Homo sapiens
tggaaggaactggtctgctcacacttgctggcttgcgcatcaggactggctttatctcctgactcacggtgcaaaggtgcactctgcgaacgttaagtccgtcccagcgcttggaatcctacggcccccacagccggatcccctcagccttccaggtcctcaactcccgcggacgctgaacaatggcctccatggggctacaggtaatgggcatcgcgctggccgtcctgggctggctggccgtcatgctgtgctgcgcgctgcccatgtggcgcgtgacggccttcatcggcagcaacattgtcacctcgcagaccatctgggagggcctatggatgaactgcgtggtgcagagcaccggccagatgcagtgcaaggtgtacgacttgctgctggcactgccgcaggacctgcaggcggcccgcgccctcgtcatcatcagcatcatcgtggctgctctgggcgtgctgctgtccgtggtggggggcaagtgtaccaactgcctggaggatgaaagcg

SEQ ID NO: 243 - DNA, 296 bp, Homo sapiens
  misc_feature 64, 187, 195: n = A,T,C or G
aggttcctcatctgctcgcgaggatgccttttctcttctgccttgcgaaataacagcagcctanctgttgcccgtgaccagtgagaaaggcagcgtcacgggctgattaggtttcacccaaagggtgccggcgccgaattggtttctaacgagaacttttaaaatgatccgttccaaaaaagggtangagccgcnagaccctccaactgcccagagaaaacaagtctcgtctggcaaaattctcggcccacgcggtccgcggccaaggggcaaaggtcctcgccccacgttgccga

SEQ ID NO: 244 - DNA, 273 bp, Homo sapiens
cttgcccatggcgaattgtggatgactgtggtggggcctttacgatgggtaccattggtggtggtatctttcaagcaatcaaaggttttcgcaattctccagtgggagtaaaccacagactacgagggagtttgacagctattaaaaccagggctccacagttaggaggtagctttgcagtttggggagggctgttttccatgattgactgtagtatggttcaagtcagaggaaaggaagatccctggaactccatcacaagtggtgccttaa

SEQ ID NO: 245 - DNA, 386 bp, Homo sapiens
ttcgaattcggcacgaggctcgatgtacgtcccggaggacctccttcccgtctacaaagaaaaagtggtgccgcttgcagacattatcacgcccaaccagtttgaggccgagttactgagtggccggaagatccacagccaggaggaagccttgcgggtgatggacatgctgcactctatgggccccgacaccgtggtcatcaccagctccgacctgccctccccgcagggcagcaactacctgattgtgctggggagtcagaggaggaggaatcccgctggctccgtggtgatggaacgcatccggatggacattcgcaaagtggacgccgtctttgtgggcactggggacctgtttgctggcatgctcctggcgtggacacaca

SEQ ID NO: 246 - DNA, 239 bp, Homo sapiens
ttttttttttcaaaaaagtcatggaggccatggggttggcttgaaaccagctttggggggttcgattccttccttttttgcctaaattttatgtatacgggttcttcaaatgtgtggtagggtggggggcatccatatagtcactccaggtttatggagggttcttctactattaggacttttcgcttcaaagcgaaggcttctcaaatcatgaaaattattaatattactgctgttaa

SEQ ID NO: 247 - DNA, 623 bp, Homo sapiens
aaaaagtcatggaggccatggggttggcttgaaaccagctttggggggttcgattccttccttttttgtctagattttatgtatacgggttcttcgaatgtgtggtagggtggggggcatccatatagtcactccaggtttatggagggttcttctactattaggacttttcgcttcgaagcgaaggcttctcaaatcatgaaaattattaatattactgctgttagagaaatgaatgagcctacagatgataggatgtttcatgtggtgtatgcatcggggtagtccgagtaacgtcggggcattccggataggccgagaaagtgttgtgggaagaaagttagatttacgccgatgaatatgatagtgaaatggattttggcgtaggtttggtctagggtgtagcctgagaataggggaaatcagtgaatgaagcctcctatgatggcaaatacagctcctattgataggacatagtggaagtgagctacaacgtagtacgtgtcgtgtagtacgatgtctagtgatgagtttgctaatacaatgccagtcaggccacctacggtgaaaagaaagatgaatcctagggctcagagcactgcagcagatcatttcatattgct

SEQ ID NO: 248 - DNA, 265 bp, Homo sapiens
ggcttagcggataacaatttcacacaggagttgcaccataatcatcgctatccccaccggcgtcaaagtatttagctgactcgccacactccacggaagcaatatgaaatgatctgctgcagtgctctgagccctaggattcatctttcttttcaccgtaggtggcctgactggcattgtattagcaaactcatcactagacatcgtactacacgacacgtactacgttgtagctcacttccactatgtcctatcaataggagct

SEQ ID NO: 249 - DNA, 625 bp, Homo sapiens
aatcatcgctatccccaccggcgtcaaagtatttagctgactcgccacactccacggaagcaatatgaaatgatctgctgcagtgctctgagccctaggattcatctttcttttcaccgtaggtggcctgactggcattgtattagcaaactcatcactagacatcgtactacacgacacgtactacgttgtagctcacttccactatgtcctatcaataggagctgtatttgccatcataggaggcttcattcactgatttcccctattctcaggctacaccctagaccaaacctacgccaaaatccatttcactatcatattcatcggcgtaaatctaactttcttcccacaacactttctcggcctatccggaatgccccgacgttactcggactaccccgatgcatacaccacatgaaacatcctatcatctgtaggctcattcatttctctaacagcagtaatattaataattttcatgatttgagaagccttcgcttcgaagcgaaaagtcctaatagtagaagaaccctccataaacctggagtgactatatggatgccccccaccctaccacacattcgaagaacccgtatacataaaatctagacaaaaaaggaag

SEQ ID NO: 250 - DNA, 253 bp, Homo sapiens
ggcttgtaatacgactcactatagggctttttttttttcaaaaaagtcatggaggccatggggttggcttgaaaccagctttggggggttcgattccttccttttttgtctaaattttatgtatacgggttcttcaaatgtgtggtagggtggggggcatccatatagtcactccaggtttatggagggttcttctactattaggacttttcgcttcaaagcgaaggcttctcaaatcatgaaaattattaat

SEQ ID NO: 251 - DNA, 290 bp, Homo sapiens
caaactcatcactagacatcgtactacacgacacgtactacgttgtagctcacttccactatgtcctatcaataggagctgtatttgccatcataggaggcgtcattcactgatttcccctattctcaggctacaccctagaccaaacctacgccaaaatccatttcactatcatattcatcggcgtaaatctaactttcttcccacaacactttctcggcctatccggaatgccccgacgttattcggactaccccgatgcatacaccacatgaaacatcctatcatct

SEQ ID NO: 252 - DNA, 638 bp, Homo sapiens
  misc_feature 522, 634, 636: n = A,T,C or G
atatttacagtaggaatagacgtagacacacgagcatatttcacctccgctaccataatcatcgctatccccaccggcgtcaaagtatttagctgactcgccacactccacggaagcaatatgaaatgatctgctgcagtgctctgagccctaggattcatctttcttttcaccgtaggtggcctgactggcattgtattagcaaactcatcactagacatcgtactacacgacacgtactacgttgtagctcacttccactatgtcctatcaataggagctgtatttgccatcataggaggcttcattcactgatttcccctattctcaggctacaccctagaccaaacctacgccaaaatccatttcactatcatattcatcggcgtaaatctaactttcttcccacaacactttctcggcctatccggaatgccccgacgttattcggactaccccgatgcatacaccacatgaaacatcctatcatctgtaggctcattcatttctctaacagcagtnatattaataattttcatgatttgagaagccttcgcttcgaagcgaaaagtcctaatagtagaagaacccttcataaacctggagtgactatatggatgccccccaccctacnanca

SEQ ID NO: 253 - DNA, 531 bp, Homo sapiens
ggcttagcggataacaatttcacacaggagttgcaccatatatttacagtaggaatagacgtagacacacgagcatatttcacctccgctaccataatcatcgctatccccaccggcgtcaaagtatttagctgactcgccacactccacggaagcaatatgaaatgatctgctgcagtgctctgagccctaggattcatctttcttttcaccgtaggtggcctgactggcattgtattagcaaactcatcactagacatcgtactacacgacacgtactacgttgtagctcacttccactatgtcctatcaataggagctgtatttgccatcataggaggcttcattcactgatttcccctattctcaggctacaccctagaccaaacctacgccaaaatccatttcactatcatattcatcggcgtaaatctaactttcttcccacaacactttctcggcctatccggaatgccccgacgttactcggactaccccgatgcatacaccacatgaaacatcctatcatct

SEQ ID NO: 254 - DNA, 625 bp, Homo sapiens
atatttacagtaggaatagacgtagacacacgagcatatttcacctccgctaccataatcatcgctatccccaccggcgtcaaagtatttagctgactcgccacactccacggaagcaatatgaaatgatctgctgcagtgctctgagccctaggattcatctttcttttcaccgtaggtggcctgactggcattgtattagcaaactcatcactagacatcgtactacacgacacgtactacgttgtagctcacttccactatgtcctatcaataggagctgtatttgccatcataggaggcttcattcactgatttcccctattctcaggctacaccctagaccaaacctacgccaaaatccatttcactatcatattcatcggcgtaaatctaactttcttcccacaacactttctcggcctatccggaatgccccgacgttactcggactaccccgatgcatacaccacatgaaacatcctatcatctgtaggctcattcatttctctaacagcagtaatattaataattttcatgatttgagaagtcttcgcttcgaagcgaaaagtcctaatagtagaagaacccttcataaacctggagtgactatatggatgccccc

SEQ ID NO: 255 - DNA, 217 bp, Homo sapiens
tttttttttttaaaaagtcatggaggccatggggttggcttgaaaccacctttggggggttcaatcccttccttctttgtctaaattttatgtatacgggttcttcaaatgtgtggtaggggggggggcatccatatagcccctccaggtttatggagggttcttctactattagaacttttcccttcaaagcaaaggcttctcaaatcatgaaaat

SEQ ID NO: 256 - DNA, 636 bp, Homo sapiens
  misc_feature 496, 562, 564, 605, 635: n = A,T,C or G
aaagtcatggaggccatggggttggcttgaaaccagctttggggggttcgattccttccttctttgtctagattttatgtatacgggttcttcgaatgtgtggtagggtggggggcatccatatagtcactccaggtttatggagggttcttctactattaggacttttcgcttcgaagcgaaggcttctcaaatcatgaaaattattaatattactgctgttagagaaatgaatgagcctacagatgataggatgtttcatgtggtgtatgcatcggggtagtccgagtaacgtcggggcattccggataggccgagaaagtgttgtgggaagaaagttagatttacgccgatgaatatgatagtgaaatggattttggcgtaggtttggtctagggtgtagcctgagaataggggaaatcagtgaatgaagcctcctatgatggcaaatacagctcctattgataggacatagtggaagtgagctacaacgtantacgtgtcgtgtagtacgatgtctagtgatgagtttgctaatacaatgccagtcaggccacctacngngaaaaagaaagatgaatcctagggctcaaaacacctgcacnagatcatttcatattggcttccgtggagtnc

SEQ ID NO: 257 - DNA, 279 bp, Homo sapiens
ggcttagcggataacaatttcacacaggagttgcaccataatcatcgctatccccaccggcgtcaaagtatttagctgactcgccacactccacggaagcaatatgaaatgatctgctgcagtgctctgagccctaggattcatctttcttttcaccgtaggtggcctgactggcattgtattagcaaactcatcactagacatcgtactacacgacacgtactacgttgtagctcacttccactatgtcctatcaataggagctgtatttgccatcat

SEQ ID NO: 258 - DNA, 623 bp, Homo sapiens
  misc_feature 537: n = A,T,C or G
aatcatcgctatccccaccggcgtcaaagtatttagctgactcgccacactccacggaagcaatatgaaatgatctgctgcagtgctctgagccctaggattcatctttcttttcaccgtaggtggcctgactggcattgtattagcaaactcatcactagacatcgtactacacgacacgtactacgttgtagctcacttccactatgtcctatcaataggagctgtatttgccatcataggaggcttcattcactgatttcccctattctcaggctacaccctagaccaaacctacgccaaaatccatttcactatcatattcatcggcgtaaatctaactttcttcccacaacactttctcggcctatccggaatgccccgacgttactcggactaccccgatgcatacaccacatgaaacatcctatcatctgtaggctcattcatttctctaacagcagtaatattaataattttcatgatttgagaagccttcgcttcgaagcgaaaagtcctaatagtagaagaaccctncataaacctggagtgactatatggatgccccccaccctaccacacattcgaagaacccgtatacataaaatctagacaaaaaagga

SEQ ID NO: 259 - DNA, 189 bp, Homo sapiens
  misc_feature 170, 173: n = A,T,C or G
tggcctttcccccttcatgggagacaacgataacgaaaccttggccaacgttacctcagccacctgggacttcgacgacgaggcattcgatgagatctccgacgatgccaaggatttcatcagcaatctgctgaagaaagatatgaaaaaccgcctggactgcacgcagnctntcagcatccatggcta

SEQ ID NO: 260 - DNA, 507 bp, Homo sapiens
cctttcccccttcatgggagacaacgataacgaaaccttggccaacgttacctcagccacctgggacttcgacgacgaggcattcgatgagatctccgacgatgccaaggatttcatcagcaatctgctgaagaaagatatgaaaaaccgcctggactgcacgcagtgccttcagcatccatggctaatgaaagataccaagaacatggaggccaagaaactctccaaggaccggatgaagaagtacatggcaagaaggaaatggcagaaaacgggcaatgctgtgagagccattggaagactgtcctctatggcaatgatctcagggctcagtggcaggaaatcctcaacagggtcaccaaccagcccgctcaatgcagaaaaactagaatctgaagaagatgtgtcccaagctttccttgaggctgttgctgaggaaaagcctcatgtaaaaccctatttctctaagaccattcgcgatttagaagttgtggagggaagtgctgc

SEQ ID NO: 261 - DNA, 193 bp, Homo sapiens
ttttttttttttttttttttttttttggccgagactccaagactattatttttatttccggacaaaaacatctgcttcacacagtgcacggcatcaaatgaagaggaaagaacttgtatcccaaagcctggctttctgtatcatccacaaattaagacagcatctgctgagcccatgctgagcctgtcacagt

SEQ ID NO: 262 - DNA, 235 bp, Homo sapiens
  misc_feature 183, 184, 185, 193: n = A,T,C or G
cccacttccccaggagcaggccacagacccccttgtggacagcctgggcagtggcattgtctactcagcccttacctgccacctgtgcggccacctgaaacagtgtcatggccaggaggatggtggccagacccctgtcatggccagtccttgctgtggctgctgctgtggagacaggtcctnnncccctacnacccccctgagggccccagacccctctccaggtggggttcca

SEQ ID NO: 263 - DNA, 493 bp, Homo sapiens
agaatttcagcagttctctgatttttatattttattcctcttcctatccaatccctgccttttgagtccaggtggtaagtacattttctttaacgtttttcctgcttttcttcccaaatgtgtctttttctttgggctactgtaccctgcttccagtgctgtccccggcataggtccatctctgcagaagccatttcaggagtacctggaggctcaacggcagaagcttcaccacaaaagcgaaatgggcacaccacagggagaaaactgcttgtcctggatgtttgaaaagtcggtcgatgtcatggtgtgttacttcatcctatctatcattaactccatggcacaaagttatgccaaacgaatccagcagcggttgaactcagaggagaaaactaaataagtagagaaagttttaaactgcagaaattggagtggatgggttctgccttatattgggaggactccaagccgggaaggaaaattccctttt

SEQ ID NO: 264 - DNA, 345 bp, Homo sapiens
agaatttcagcagttctctgatttttatattttattcctcttcctatccaatccctgccttttgagtccaggtggtaagtacattttctttaacgtttttcctgcttttcttcccaaatgtgtctttttctttgggctactgtaccctgcttccagtgctgtccccggcataggtccatctctgcagaagccatttcaggagtacctggaggctcaacggcagaagcttcaccacaaaagcgaaatgggcacaccacagggagaaaactggttgtcctggatgtttgaaaagtcggtcgttgtcatggtgtgttacttcatcctatctatcattaactccatggt

SEQ ID NO: 265 - DNA, 374 bp, Homo sapiens
tagaagagctaacctcacactcatcccactctaaactatgtgattcaacactgattttacatccaacaaagtgaaatcttgatagttgggtgtaaaaaggagagtaatggagatttcagagtagttggggttgcttacttttcatttttaattctttaggttttgtaagttacacacttcaagcattatagatgatcctctttttactactgaactaatgaagcctttttcattgcattgttctgcatttatttctacagggagaaaactggttgtcctggatgtttgaaaagttggtcgttgtcatggtgtgttacttcatcctatctatcattaactccatggcacaaagttatgccaaacgaatccagcag

SEQ ID NO: 266 - DNA, 360 bp, Homo sapiens
tttttttttttttttttttgtgcggtgggaattctctaattgtatcatgtgggccttttgaaagtaacaaacagaaggccagtctgctgcaagtttgctgctgaacatcacattccaccctaagaaaacacaaggtggattgcatcgagggtggataccttaccttagcacggaaggaaaaagtatgtcagtgcaaagtatggactaaactgctttcaggaaaaaagttgtaaaaattgatacaggttggaaaagggaattttccttcccggcttggagtcctcccaatttaaggcagaacccatccactccaatttctgcagtttaaaactttctctacttatttagttttctcctctg

SEQ ID NO: 267 - DNA, 247 bp, Homo sapiens
ctggaattgtcatctttggaacagtgattgcaacagcacttatgggattgacagagaaactgattttttccctgagagatcctgcatacagtacattcccgccagaaggtgttttcgtaaatacgcttggccttctgatcctggtgttcggggccctcattttttggatagtcaccagaccgcaatggaaacgtcctaaggagccaaattctaccattcttcatccaaatggaggcatgaacaggga

SEQ ID NO: 268 - DNA, 350 bp, Homo sapiens
taatggatttgtttggagatggcatgttggtagacgactgaatatggaaaggatatcaagttatctattttgttaattttatttttgttttttatcatctagatttttatcatggattagtctgaaatttaaagttctggccagtcggttttctttcatcttgtagtttttacagtatttccactgtgcatatgcaaaatgggtattacataactgtatcatatttggtattgataatttttttttttttttggaaacgggtttttgttttggcccagcccaaaaacatcccttggttaccccttccggggaaaaaaaaccaaacccctttttcggggaaaaaaaaaggg

SEQ ID NO: 269 - DNA, 455 bp, Homo sapiens
  misc_feature 81, 195, 231, 247, 298, 307, 317, 395, 427, 446, 451: n = A,T,C or G
ttttttttaaatcaaagagtagtttattaaaaaaggaatcaaacaggaaactctaagtaccagtgtgtacattgtacaatnttaaatgactcacgagaatgaagtttttttcaaatatattaagatcacaccaccttgttgtttatcgaaagatattcaaggagaaagatctgactctccaaactgcatctgagnattgccactttaaacaggacctcatttcaaacatgncaacaacgccactggntaataaaggctttgggaatggggtgctcattctattatttcactacaaacngcatagganaggcaggagnagttggggaatttattctaaaataggaatgggagggttgtccatctacagcaggcactccttcacttcctctgtttgncctttttaggcagtactccttggtcggtcttngaacggttttccaaccctnttcantggg

SEQ ID NO: 270 - DNA, 444 bp, Homo sapiens
  misc_feature 17, 20, 391, 430: n = A,T,C or G
ttttctgacgtctgttnctnaggctggaagaaatgagcagaaaacaagggatgagtactttttagagtatgtgcatgttacgtaatacctgtttctgggcaatgctgcttcttctgactcaacaaatggggagagcaaattgaaaatgcgtaaattggaaggcaagttctgaaattaaacgttgtactttggcctgatgttctgacctttaaggaagcaagagtttgtaaacttccaaatatttactattctgaactgccgtgtaaacctgacgtattcccaagtcaacataccagtataccaataggatgtgaataatgtttgtgttgagtttaaaaccatagcagttttgctctggcaagtaatggaaagcgttctcgcttcctgagtntgagctccagcagactgcagagtggccagtgccacagtngtagcctgactttc

SEQ ID NO: 271 - DNA, 502 bp, Homo sapiens
ggttctgcgctggtcggcggagtagcaagtggccatggggagcctcagcggtctgcgcctggcagcaggaagctgttttaggttatgtgaaagagatgtttcctcatctctaaggcttaccagaagctctgatttgaagagaataaatggattttgcacaaaaccacaggaaagtcccggagctccatcccgcacttacaacagagtgcctttacacaaacctacggattggcagaaaaagatcctcatatggtcaggtcgcttcaaaaaggaaggtgaaatcccagagactgtctcgttggagatgcttgatgctgcaaagaacaagatgcgagtgaagatcagctatctaatgattgccctgacggtggtaggatgcatcttcatggttattgagggcaagaaggctgcccaaagacacgagactttaacaagcttgaacttagaaaagaaagctcgtctgaaagaggaagcagctatgaaggccaaaacagagtagcag

SEQ ID NO: 272 - DNA, 377 bp, Homo sapiens
ggttctgcgctggtcggcggagtagcaagtggccatggggagcctcagcggtctgcgcctggcagcaggaagctgttttaggttatgtgaaagagatgtttcctcatctctaaggcttaccagaagctctgatttgaagagaataaatggattttgcacaaaaccacaggaaagtcccggagctccatcccgcacttacaacagagtgcctttacacaaacctacggattggcagaaaaagatcctcatatggtcaggtcgcttcaaaaaggaaggtgaaatcccagagactgtctcgttggagatgcttgatgctgcaaagaacaagatgcgagtgaagatcagctatctaatgattgccctgacggtggtaggaa

SEQ ID NO: 273 - DNA, 552 bp, Homo sapiens
agctcggaattcggctcgagtctgctcagcctggtgaacccacaggcccgagtttcacccagtccccactccacggtgcagctgcggcttatctctcagcccagcgagatgccagccttcctgtcccgggccagcgctctgacatgcagaaggtgaccctgggcctgcttgtgttcctggcaggctttcctgtcctggacgccaatgacctagaagataaaaacagtcctttctactatgactggcacagcctccaggttggcgggctcatctgcgctggggttctgtgcgccatgggcatcatcatcgtcatgagtgcaaaatgcaaatgcaagtttggccagaagtccggtcaccatccaggggagactccacctctcatcaccccaggctcagcccaaagctgatgaggacagaccagctgaaattgggtggaggaccgttctctgtccccaggtcctgtctctgcacagaaacttgaactccaggatggaattcttcctcctctgctgggactcctttgcatggcagggcctcatctcacctctcgca

SEQ ID NO: 274 - DNA, 186 bp, Homo sapiens
ctgctcagcctggtgaacacacagcccgatttacccagtccccactccaggtgcagctgcggcttatctctcagcccagcgagatgccagccttcctgtcccgggccagcgctctgacatgcagaaggtgaccctgggcctgcttgtgttcctggcaggctttcctgtcctggacgccaatgacct

SEQ ID NO: 275 - DNA, 121 bp, Homo sapiens
tctgctcagcctggtgaaccacacaggcccgagtttcacccagtccccactccacggtgcagctgcggcttatctctcagcccagcgagatgccagccttcctgtcccgggccagcgctct

SEQ ID NO: 276 - DNA, 336 bp, Homo sapiens
  misc_feature 336: n = A,T,C or G
agggacccgcagctcagctacagcacagatcagcaccatgaagcttctcacgggcctggttttctgctccttggtcctgagtgtcagcagccgaagcttcttttcgttccttggcgaggcttttgatggggctcgggacatgtggagagcctactctgacatgagagaagccaattacatcggctcagacaaatacttccatgctcgggggaactatgatgctgccaaaaggggacctgggggtgcctgggccgcagaagtgatcagcaatgccagagagaatatccagagactcacaggccatggtgcggaggactcgctggccgatcaggctgn

SEQ ID NO: 277 - DNA, 460 bp, Homo sapiens
tgcagacggaggtcaggtcttcctctttcctgagactggatctgttcaaacagcaaacgcccacagatggcccagaggtggtggtagtcagggtgtgtgggtgtttttagggttctttagtgttgtttctttcacccaggggtggtggtcccagccagtttggtgctgacggtgagaggaaattagaatctgtttgcaaattgtccaacccaccccctcaacatgaggggcttccattttctgtgttttgtaagggaactgtttccttcatgccgccatgttcctgatattagttctgatttctttttaacaaatgttatcatgattaagaaaatttccagcactttaatggccaattaactgagaatgtaagaaaattgatgctgtacaaggcaaataaagctgtttattaaccttgaaaaaaaaaaaaaaaaaaaaaaaaaaaaaaaattttttgggg

SEQ ID NO: 278 - DNA, 432 bp, Homo sapiens
  misc_feature 46, 151, 350, 362, 383, 403, 417: n = A,T,C or G
ggggttgcagacggaggtcaggtcttcctctttcctgagactgganctgttcaaacagcaaacgcccacagatggcccagaggtggtggtagtcagggtgtgtgggtgtttttagggttctttagtgttgtttctttcacccaggggtggntggtcccagccagtttggtgctgacggtgagaggaaattagaatctgtttgcaaattgtccaacccaccccctcaacatgaggggcttccattttctgtgttttgtaagggaactgtttccttcatgccgccatgttcctgatattagttctgatttctttttaacaaatgttatcatgattaagaaaatttccagcanttaatgggccanttaactgagaatgtaagaaantgatgctgttacaaggcaantaaagccgttttanttaaccctgaaaaaa

SEQ ID NO: 279 - DNA, 467 bp, Homo sapiens
acgtgacgcggggccaggcggccgtacagcagctgcaggcggagggcctgagcccgcgcttccaccagctggacatcgacgatctgcagagcatccgcgccctgcgcgacttcctgcgcaaggagtacgggggcctggacgtgctggtcaacaacacgggcatcgccttcaaggttgctgatcccacaccctttcatattcaagctgaagtgacgatgaaaacaaatttctctggtacccgagatgtgtgcacagaattactccctctaataaaaccccaagggagagtggtgaacgtacctagcatcatgagcgtcagagcccttaaaagctgcagcccagagctgcagcagaagttccgcagtgagaccatcactgaggaggagctggtggggctcatgaacaagtttgtggaggatacaaagaagggagtgcaccagaaggagggctggcccagcagcgcatac

SEQ ID NO: 280 - DNA, 626 bp, Homo sapiens
tacggccgggacgtgacgcggggccaggcggccgtacagcagctgcaggcggagggcctgagcccgcgcttccaccagctggacatcgacgatctgcagagcatccgcgccctgcgcgacttcctgcgcaaggagtacgggggcctggacgtgctggtcaacaacacgggcatcgccttcaaggttgctgatcccacaccctttcatattcaagctgaagtgacgatgaaaacaaatttctctggtacccgagatgtgtgcacagaattactccctctaataaaaccccaagggagagtggtgaacgtacctagcatcatgagcgtcagagcccttaaaagctgcagcccagagctgcagcagaagttccgcagtgagaccatcactgaggaggagctggtggggctcatgaacaagtttgtggaggatacaaagaagggagtgcaccagaaggagggctggcccagcagcgcatacggggtgacgaagattggcgtcaccgttctgtccaggatccacgccaggaaactgagtgagcagaggaaaggggacaagatcctcctgaatgcctgctgcccagggtgggtgagaactgacatggcgggacccaaggccaccaagagccc

SEQ ID NO: 281 - DNA, 487 bp, Homo sapiens
tggcctgttcctcagcgagggcctgaagctagtggataagtttttggaggatgttaaaaagttgtaccactcagaagccttcactgtcaacttcggggacaccgaagaggccaagaaacagatcaacgattacgtggagaagggtactcaagggaaaattgtggatttggtcaaggagcttgacagagacacagtttttgccctggtgaattacatcttctttaaaggcaaatgggagagaccctttgaagtcaaggacaccgaggaagaggacttccacgtggaccaggcgaccaccgtgaaggtgcctatgatgaagcgtttaggcatgtttaacatccagcactgtaagaagctgtccagctgggtgctgctgatgaaatacctgggcaatgccaccgccatcttcttcctgcctgatgaggggaaactacagcacctggaaaatgaactcacccacgatatcatcaccaagttcctggaaaat

SEQ ID NO: 282 - DNA, 345 bp, Homo sapiens
tggcctgttcctcagcgagggcctgaagctagtggataagtttttggaggatgttaaaaagttgtaccactcagaagccttcactgtcaacttcggggacaccgaagaggccaagaaacagatcaacgattacgtggagaagggtactcaagggaaaattgtggatttggtcaaggagcttgacagagacacagtttttgccctggtgaattacatcttctttaaaggcaaatgggagagaccctttgaagtcaaggacaccgaggaagaggacttccacgtggaccaggcgaccaccgtgaaggtgcctatgatgaagcgtttaggcatgtttaacatccagca

SEQ ID NO: 283 - DNA, 495 bp, Homo sapiens
cggccgcccttttttttttttttttttttttttttttttttttttttttttttttttttttttttttttcaaaaaaaaatttttttgggtttttttttttaaaactttttttttttttttttttggggggggccaaattcccccccaaaaaaaaaaaaaaaggggggggtttcccccccccccctttttttttttgggggggtttttttttttggggggggccccccccccctttttttttttttggaaaaaaatcccccccttggggggggtttctttttcccaaagggagttttttttcccccccccccgggggggggggggggtttttttttttttaaaaaaaaaacccccggaaaaaaaaaaacccccccccccccccccccccccaaaaaaaaaaaaggggggaaaaatgggggccccccctttttttttttttttttttttggggggggggaaaaaaaaccccccccccctttttgggggggttttttt

SEQ ID NO: 284 - DNA, 503 bp, Homo sapiens
attccgttgctgtcgagcatgaccaagcagctgggtgacttctggacacggatggaggagctccgccaccaagcccggcagcagggggcagaggcagtccaggcccagcagcttgcggaaggtgccagcgagcaggcattgagtgcccaagagggatttgagagaataaaacaaaagtatgctgagttgaaggaccggttgggtcagagttccatgctgggtgagcagggtgcccggatccagagtgtgaagacagaggcagaggagctgtttggggagaccatggagatgatggacaggatgaaagacatggagttggagctgctgcggggcagccaggccatcatgctgcgctcagcggacctgacaggactggagaagcgtgtggagcagatccgtgaccacatcaatgggcgcgtgctctactatgccacctgcaagtgatgctacagcttccagcccgttgccccactcatctgccgcctttgcttttggttgggggc

SEQ ID NO: 285 - DNA, 581 bp, Homo sapiens
agtggcactgcaggaagctcaggacaccatgcaaggcaccagccgctcccttcggcttatccaggacagggttgctgaggttcagcaggtactgcggccagcagaaaagctggtgacaagcatgaccaagcagctgggtgacttctggacacggatggaggagctccgccaccaagcccggcagcagggggcagaggcagtccaggcccagcagcttgcggaaggtgccagcgagcaggcattgagtgcccaagagggatttgagagaataaaacaaaagtatgctgagttgaaggaccggttgggtcagagttccatgctgggtgagcagggtgcccggatccagagtgtgaagacagaggcagaggagctgtttggggagaccatggagatgatggacaggatgaaagacatggagttggagctgctgcggggcagccaggccatcatgctgcgctcagcggacctgacaggactggagaagcgtgtggagcagatccgtgaccacatcaatgggcgcgtgctctactatgccacctgcaagtgatgctacagcttccagcccgttgccccactcatct

SEQ ID NO: 286 - DNA, 598 bp, Homo sapiens
agtggcactgcaggaagctcaggacaccatgcaaggcaccagccgctcccttcggcttatccaggacagggttgctgaggttcagcaggtactgcggccagcagaaaagctggtgacaagcatgaccaagcagctgggtgacttctggacacggatggaggagctccgccaccaagcccggcagcagggggcagaggcagtccaggcccagcagcttgcggaaggtgccagcgagcaggcattgagtgcccaagagggatttgagagaataaaacaaaagtatgctgagttgaaggaccggttgggtcagagttccatgctgggtgagcagggtgcccggatccagagtgtgaagacagaggcagaggagctgtttggggagaccatggagatgatggacaggatgaaagacatggagttggagctgctgcggggcagccaggccatcatgctgcgctcagcggacctgacaggactggagaagcgtgtggagcagatccgtgaccacatcaatgggcgcgtgctctactatgccacctgcaagtgatgctacagcttccagcccgttgccccactcatctgccgcctttgcttttgg

SEQ ID NO: 287 - DNA, 316 bp, Homo sapiens
ctgcccttcacctcgcagtggacctgcaaaatcctgacctggtgtcactcctgttgaagtgtggggctgatgtcaacagagttacctaccagggctattctccctaccagctcacctggggccgcccaagcacccggatacagcagcagctgggccagctgacactagaaaaccttcagatgctgccagagagtgaggatgaggagagctatgacacagagtcagagttcacggagttcacagaggacgagctgccctatgatgactgtgtgtttggaggccagcgtctgacgttatgagcgcaaaggggctgaaa

SEQ ID NO: 288 - DNA, 275 bp, Homo sapiens
atgattaggagaagtggtggccacagtcgaaaaatcccaaggcccaaacctgcaccactgactgctgaaatacagcaaaagattttgcatttgccaacatcttgggactggagaaatgttcatggtatcaattttgtcagtcctgttcgaaaccaagcatcctgtggcagctgctactcatttgcttctatgggtatgctagaagcgagaatccgtatactaaccaacaattctcagaccccaatcctaagccctcaggaggttgtgtcttgtag

SEQ ID NO: 289 - DNA, 522 bp, Homo sapiens
cagaagggaacaccagagctttgctaataattagtgtggtcaagagccgtctgagcctaatgagtcccagctgcattaggttaagagactcttccagagccatcgccaggtcgggaatggcacctctccctaggatacacagcctgcaggtccccaggacctggatgacacccgcctcactgtggcagtgtattgcctgttaattgctgctaattctaattctgatgatgactcctactccattgtttaccccaaagcatcagctaggctggagtgatttgttacaaatgagcaaaagatgagtccttgcttccctcagaaataaaaggagctcagctgcagcgttgcattgggcttcttggcctcccaactcttcccactcccagaatccagaagtaagctctgcatgttccccttcctgggaggaaaccagttgtcagaaggatgtatgatgaccccctcccctcccatccttcacctcctaagcagtcctggcttttcctcatcactcccctctacagt

SEQ ID NO: 290 - DNA, 331 bp, Homo sapiens
aacaccagagctttgctaataattagtgtggtcaagagccgtctgagcctaatgagtcccagctgcattaggttaagagactcttccagagccatcgccaggtcttgaatggcacctctccctaggatacacagcctgcaggtccccaggacctggatgacacccgcctcactgtggcagtgtattgcctgttaattgctgctaattctaattctgatgatgactcctactccattgtttaccccaaagcatcagctaggctggagtgatttgttacaaatgagcaaaagatgagtccttgcttccctcagaaataaaaggagctcagctg

SEQ ID NO: 291 - DNA, 228 bp, Homo sapiens
gagatgcaaagcaggattcaaaagaacatctttgcgttttctaccggctccccatcatcgtactagggaggaagaagcgggtgagaaacaaaacttctttccattgtcctgcccgtttctgcggacttgttctgaggccgaggcacctctaagatactgatggctctgcagaggacccattcattgcttctgcttttgctgctgaccctgctggggctggggctggtc

SEQ ID NO: 292 - DNA, 342 bp, Homo sapiens
ggagctgtcctgcaccgtggtggagctgaagtacacaggcaatgccagcgcactcttcatcctccctgatcaagacaagatggaggaagtggaagccatgctgctcccagagaccctgaagcggtggagagactctctggagttcagagagataggtgagctctacctgccaaagttttccatctcgagggactataacctgaacgacatacttctccagctgggcattgaggaagccttcaccagcaaggctgacctgtcagggatcacaggggccaggaaccctacagtctcccaggtggtccataaggctgtgcttgatgtatttgaggagggcacaga

SEQ ID NO: 293 - DNA, 311 bp, Homo sapiens
ggagctgtcctgcaccgtggtggagctgaagtacacaggcaatgccagcgcactcttcatcctccctgatcaagacaagatggaggaagtggaagccatgctgctcccagagaccctgaagcggtggagagactctctggagttcacagagataggtgagctctacctgccaaagttttccatctcgagggactataacctgaacgacatacttctccagctgggcattgaggaagccttcaccagcaaggctgacctgtcagggatcacaggggccaggaacctagcagtctcccaggtggtccataagg

SEQ ID NO: 294 - DNA, 402 bp, Homo sapiens
cggctgcgagaagacgacagaagggaagatggaggaagtggaagccatgctgctcccagagaccctgaagcggtggagagactctctggagttcagagagataggtgagctctacctgccaaagttttccatctcgagggactataacctgaacgacgacttctccagctgggcattgaggaagccttcaccagcaaggctgacctgtcagggatcacaggggccaggaacctagcagtctcccaggtggtccataaggctgtgcttgatgtatttgaggagggcacagaagcatctgctgccacagcagtcaaaatcaccctcctttctgcattagtggagacaaggaccattgtgcgtttcaacaggcccttcctgatgatcattgtgcctacagacacc

SEQ ID NO: 295 - DNA, 232 bp, Homo sapiens
ttccatctcgagggactataacctgaacgacgacttctccagctgggcattgaggaagccttcaccagcaaggctgacctgtcagggatcacaggggccaggaacctagcagtctcccaggtggtccataaggctgtgcttgatgtatttgaggagggcacagaagcatctgctgccacagcagtcaaaatcaccctcctttctgcattagtggagacaaggaccattgtgc

SEQ ID NO: 296 - DNA, 435 bp, Homo sapiens
tgactctgacttctgaggaagaggcccgtttgaagaagagtgcacatcactttgggggatccaaaaggagctgcaattttaaagtcttctgatgtcatatcatttcactgtctaggctacaacaggattctaggtggaggttgtgcatgttgtcctttttatctgatctgcgattaaagcagtaatattttaagatggactgggaaaaacatcaactcctgaagttagaaataagaatggtttgtaaaatccacagctatatcctgatgctggatggtattaatcttgtgtagtcttcaactggttagtgtgaaatagttctgccacctctgacgcaccactgccaatgctgtacgtactgcatttgccccttgagccaggtggatgtttaccgtgtgttatataacttactggctccttcactgaacatgccta

SEQ ID NO: 297 - DNA, 309 bp, Homo sapiens
atcatttcactgtctaggctacaacaggattctaaggggacgttgtgcatgttggcctttgtatctgatctgtgattaaagcagtaatattttaagatggactgggaaaaacatcaactcctgaagttagaaataagaatggtttgtaaaatccacagctgtatgctgaagctggatggtattaatcttgcgtagtcttcaactggttaggtgaaatagttctgccacctctgacgcaccactgccaatgctgtacgtactggatttggcccttgagccaggtggatgttcaccgggcgtgatataact

SEQ ID NO: 298 - DNA, 342 bp, Homo sapiens
  misc_feature 342: n = A,T,C or G
atcatttgactgtctaggctacaacaggattctaggtggaggttgtgcatgttgtcctttttatctgatctgtgattaaagcagtaatattttaacatggactgggaaaaacatcaactcctgaagttagaaataagaatggtttgtaaaatccacagctatatcctgatgctggatggtattaatcttgtgtagtcttcaactggttagttgaaatagttctgccacctctgacgcaccactgccaatgctgtacgtactgcatttgccccttgagccaggtggatgtttaccgtgtgttatataacttcctggctccttcactgaacatgcctagtccan

SEQ ID NO: 299 - DNA, 266 bp, Homo sapiens
gggacagaatggctatctcggaccttgtgaaggtgactctgacttctgaggaagacgcccgcttgaagaagagagcccatacactttgggggatccaaaacgagctgcgattttcaagtcttctgatgtcatatcattccactgtctaggctacaacaggattctagggggacgttgtgcatgttggcctttttatctgatctgtgactaaagcactaatattttaagatggactgggaaaaacatcaactcctgaagttagaaat

SEQ ID NO: 300 - DNA, 383 bp, Homo sapiens
ggacagaatggaatctcagaccttgtgaaggtgactctgacttctgaggaagaggcccgtttgaagaagagtgcagatacactttgggggatccaaaaggagctgcaattttaaagtcttctgatgtcatatcatttcactgtctaggctacaacaggattctaggtggaggttgtgcatgttgacctttttatctgatctgtgattaaagcagtaatattttaagatggactgggaaaaacatcaactcctgaagttagaaataagaatggtttgtaaaatccacagctatatcctgatgctggatggtattaatcttgtgtagtcttcaactggttagtgtgaaatagttctgccacctctgacgcaccactgccaatgct

SEQ ID NO: 301 - DNA, 453 bp, Homo sapiens
aaccgcttctccgttgaacaacatactagatggggacagaatggaatctcagaccttgtgaaggtgactctgacttctgaggaagaggcccgtttgaagaagagtgcagatacactttgggggatccaaaaggagctgcaattttaaagtcttctgatgtcatatcatttcactgtctaggctacaacaggattctaggtggaggttgtgcatgttgtcctttttatctgatctgtgattaaagcagtaatattttaagatggactgggaaaaacatcaactcctgaagttagaaataagaatggtttgtaaaatccacagctatatcctgatgctggatggtattaatcttgtgtagtcttcaactggttagtgtgaaatagttctgccacctctgacgcaccactgccaatgctgtacgtactgcatttgccccttgagccaggtggatgt

SEQ ID NO: 302 - DNA, 383 bp, Homo sapiens; identical to SEQ ID NO: 300

SEQ ID NO: 303 - DNA, 97 bp, Homo sapiens
gttgccttggagatgatcaaagtaactggtggctatccatttgaagcttacaaaaattgttttcttaacttagccattccaattgtagtatttacag

SEQ ID NO: 304 - DNA, 442 bp, Homo sapiens
gccctagttattataccatattacatcattactctatgtaattatctatgaagctatgtagttatttacccctgtattaagtgattttagactgttgttattttttgagttacagcatgtgctttcaaaatagggagactgtatggttgaattaatatttttttaaataactgttaacatgtatagagtaggttgaaagtttgaaagtataaaatatactaaaagtatacagacctgtaataagaaatttatattactatagtcccatagctgcttttactatccacagagaaatgcttgaaaacgtgaaagttgaatagatgcaattaaaatcacggatagttttaggctgtttatattatcagatcaccttcttttatctaggttgccttggagatgatcaaagtaactggtggctatccatttgaagcttacaaaaatt

SEQ ID NO: 305 - DNA, 380 bp, Homo sapiens
gagacgttcgcacacctgggtgccagcgccccagaggtcccgggacagcccgaggcgccgcgcccgccgccccgagctccccaagccttcgagagcggcgcacactcccggtctccactcgctcttccaacacccgctcgttttggcggcagctcgtgtcccagagaccgagttgccccagagaccgagacgccgccgctgcgaaggaccaatgagagccccgctgctaccgccggcgccggtggtgctgtcgctcttgatactcggctcaggccattatgctgctggattggacctcaatgacacctactctgggaagcgtgaaccattttctggggaccacagggctgatggatttgaggttacctccagaagggagg

SEQ ID NO: 306 - DNA, 133 bp, Homo sapiens
ccagtactgcctcctgtgctcgtgccaagacacagtgaatataacccccagctcagcctcctggccaagttccgcagcgcctccctgcacagtgagccactcatgccacacaacgccacctatcctgactctt

SEQ ID NO: 307 - DNA, 428 bp, Homo sapiens
tccagtactgcctcctgtgctcgtgccaagacacagtgaatataacccccagctcagcctcctggccaagttccgcagcgcctccctgcacagtgagccactcatgccacacaacgccacctatcctgactctttccagcagcctccgtgctctgcactccctccctcacccagccacgcgttctcccagtccccgtgcacggccagctaccctcactccccaggaagtccttctgagccagagagtccctatcaacactcagactttcgaccagtttgttacgaggagcccccactggtgctcggtcgcctactatgaactgaacaaccgagttggggagacattccaggcttcctcccgaagtgtgctcatagatgggttcaccgacccttcaaataacaggaacagattctgtcttggacttctt

SEQ ID NO: 308 - DNA, 497 bp, Homo sapiens
cggctgcgagaagacgacagaaggggggaatgtgtctggcccttcagcagtttctcttggcagcatcagctgggctgctttctttgtgtgtggccccaggtgtcaaaatgacaccagctgtctgtactagacaaggttaccaagtgcggaattggttaatactaacagagagatttgctccattctctttggaataacaggacatgctgtatagatacaggcagtaggtttgctctgtacccatgtgtacagcctacccatgcagggactgggattcgaggacttccaggcgcatagggtagaaccaaatgatagggtaggagcatgtgttctttagggccttgtaaggctgtttccttttgcatctggaactgactatataattgtcttcaatgaagactaattcaattttgcatatagaggagccaaagagagatttcagctctgtatttgtggtatcagtttggaaaaaaaaaatctgatactccatttgatta

SEQ ID NO: 309 - DNA, 356 bp, Homo sapiens
gggaatgtgtctggcccttcagcagtttctcttggcagcatcagctgggctgctttctttgtgtgtggccccaggtgtcaaaatgacaccagctgtctgtactagacaaggttaccaagtgcggaattggttaatactaacagagagatttgctccattctctttggaataacaggacatgctgtatagatacaggcagtaggtttgctctgtacccatgtgtacagcctacccatgcagggactgggattcgaggacttccaggcgcatagggtagaaccaaatgatagggtaggagcatgtgttctttaaggccttgtaaggctgtttccttttgcatctggaactgactatat

SEQ ID NO: 310 - DNA, 348 bp, Homo sapiens
gggaatgtgtctggcccttcagcagtttctcttggcagcatcagctgggctgctttctttgtgtgtggccccaggtgtcaaaatgacaccagctgtctgtactagacaaggttaccaagtgcggaattggttaatactaacagagagatttgctccattctctttggaataacaggacatgctgtatagatacaggcagtaggtttgctctgtacccatgtgtacagcctacccatgcagggactgggattcgaggacttccaggcgcatagggtagaaccaaatgatagggtaggagcatgtgttctttagggccttgtaaggctgtttccttttgcatctggaact

SEQ ID NO: 311 - DNA, 337 bp, Homo sapiens
aagttgtggtctgacacacactgctgtggttcccctggatttagtgaaatgccgtatgcaggtggacccccaaaagtacaagggcatatttaacggattctcagttacacttaaagaggatggtgttcgtggtttggctaaaggatgggctccgactttccttggctactccatgcagggactctgcaagtttggcttttatgaagtctttaaagtcttgtatagcaatatgcttggagaggagaatacttatctctggcgcacatcactatatttggctgcctctgccagtgctgaattctttgctgacattgccctggctcctatggaagctgct

SEQ ID NO: 312 - DNA, 252 bp, Homo sapiens
  misc_feature 144: n = A,T,C or G
agcccaagccctcagtggaacctgtcaagagcatcagcagcatggagctgaagaccgagccctttgatgacttcctgttcccagtgacacttcagagagctggtagttagtagcatgttgagccaggcctgggtctgtgtctcntttctctttctccttagtcttctcatagcattaactaatctattgggttcattattggaattaacctggtgctggatattttcaaattgtatctagtgcagctgattt

SEQ ID NO: 313 - DNA, 51 bp, Homo sapiens
actcccagctgcactggttacacgtcttccttcgtcttcacctaccccgag

SEQ ID NO: 314 - DNA, 348 bp, Homo sapiens
atggccacagagctggagcccctgtgcactccggtggtcacctgtactcccagctgcactgcttacacgtcttccttcgtcttcacctaccccgaggctgactccttccccagctgtgcagctgcccaccgcaagggcagcagcagcaatgagccttcctctgactcgctcagctcacccacgctgctggccctgtgagggggcagggaaggggaggcagccggcacccacaagtgccactgcccgagctggtgcattacagagaggagaaacacatcttccctagagggttcctgtagacctagggaggaccttatctgtgcgtgaaacacaccaggctgtgggccc

SEQ ID NO: 315 - DNA, 507 bp, Homo sapiens
ccggtggtcacctgtactcccagctgcactgcttacacgtcttccttcgtcttcacctaccccgaggctgactccttccccagctgtgcagctgcccaccgcaagggcagcagcagcaatgagccttcctctgactcgctcagctcacccacgctgctggccctgtgagggggcagggaaggggaggcagccggcacccacaagtgccactgcccgagctggtgcattacagagaggagaaacacatcttccctagagggttcctgtagacctagggaggaccttatctgtgcgtgaaacacaccaggctgtgggcctcaaggacttgaaagcatccatgtgtggactcaagtccttacctcttccggagatgtagcaaaacgcatggagtgtgtattgttcccagtgacacttcagagagctggtagttagtagcatgttgagccaggcctgggtctgtgtctcttttctctttctccttagtcttctcatagcattaactaatct

SEQ ID NO: 316 - DNA, 239 bp, Homo sapiens
  misc_feature 223: n = A,T,C or G
agactccaagccctactgggaggcacggagggtggcgaggcaggctcagctggaagctcagaaagccacgcaggacttccagagggccacagaggtgctccgcgccgccaaggagaccatctccctggccgagcagcggctgctggaggatgacaagcggcagttcgactccgcctggcaggagatgctgaatcacgccactcagagggtcatggaggcgganagaccaagaccaggag

SEQ ID NO: 317 - DNA, 313 bp, Homo sapiens
catcagtgatagggatgattcacaaacacaaagctggtcttttcaaaatgggaagaaaaaagatgcaattgatcccttactattcaagtataaagtgcaacccactaaaaaagaattacatgagtctgctattgttaaagcaacacaaatcagccggagaaaacacctattttctcgtgataaactaaagctttttctgaagcaacactgggaaccacaagatggagtcattaaaataaaggcatcatctctttcaacggataaaatagccgaacaagattttttcttatttcttccctgatgattccacccc

SEQ ID NO: 318 - DNA, 574 bp, Homo sapiens
aaataacatcaacagaacagcttcactttgggccaaacatttgaaaaactttttataaaaaattgtttgatatttcttaatgtctgctctgagccttaaaacacagattgaagaagaaaagaaagaaaaaacttaaatatttatttctatgctttgttgcctctgagaataatgacaatttatgaatttgtgtttcaaattgataaaatatttaggtacaaataacaagactaataatattttcttatttaaaaaaagcatgggaagatttttatttatcaaaatatagaggaaatgtagacaaaatggatataaatgaaaattaccatgttgtaaaaccttgaaaatcagattctaactggatttgtatgcaactaagtatttttctgaacacctatgcaggtcttatttacagtagttactaagggaacacacaaagaattacacaacgttttcctcaagaaaatggtacaaaacacaaccgaggagcgtatacagttgaaaacatttttgttttgattggaaggcagattattttatattagtattaaaaatcaaaccctatgtttctttc

SEQ ID NO: 319 - DNA, 518 bp, Homo sapiens
gaagggaaataacatcaacagaacaacttcactttgggccaaacatttgaaaaactttttataaaaaattgtttgatatttcttaatgtctgctctgagccttaaaacacagattgaagaagaaaagaaagaaaaaacttaaatatttatttctatgctttgttgcctctgagaataatgacaatttatgaatttgtgtttcaaattgataaaatatttaggtacaaataacaagactaataatattttcttatttaaaaaaagcatgggaagatttttatttatcaaaatatagaggaaatgtagacaaaatggatataaatgaaaattaccatgttgtaaaaccttgaaaatcagattctaactggatttgtatgcaactaagtatttttctgaacacctatgcaggtcttatttacagtagttactaagggaacacacaaagaattacacaacgttttcctcaagaaaatggtacaaaacacaaccgaggagcgtatacagttgaaaacattttt

SEQ ID NO: 320 - DNA, 353 bp, Homo sapiens
aaataacatcaacagaacaacttcactttgggccaaacatttaaaaaactttttataaaaaaatgtttgatatttcttaatgtctgctctgagccttacaacacagattgaagaagaaaagaaagaacaaacttagatatttatttctatgctttgttgcctctgagaataatgacaatttatgaatttgagtttcaaattgataaaatatttaggtactaataacaagactaataatattttcttatttataaaaagcatgggaagatttttatttatcaaaatatacaggaagtgtagacaaaatggatataaatgaaaattaccatgttgtaaaaccttgaaaatcagag

SEQ ID NO: 321 - DNA, 401 bp, Homo sapiens
gacctgcacacagagactccctcctgggctcctggcaccatggccccctgaagagctggccctggtcaccctcctcctgggggcttctctgcagcacatccacgcagctcgagggaccaatgtgggccgggagtgctgcctggagtacttcaagggagccattccccttagaaagctgaagacgtggtaccagacatctgaggactgctccagggatgccatcgtttttgtaactgtgcagggcagggccatctgttcggaccccaacaacaagagagtgaagaatgcagttaaatacctgcaaagccttgagaggtcttgaagcctcctcaccccagactcctgactgtctcccgggactacctgggacctccaccggtggtgttcaccgcccccaccct

SEQ ID NO: 322 - DNA, 547 bp, Homo sapiens
gacctgcacacagagactccctcctgggctcctggcaccatggccccactgaagatgctggccctggtcaccctcctcctgggggcttctctgcagcacatccacgcagctcgagggaccaatgtgggccgggagtgctgcctggagtacttcaagggagccattccccttagaaagctgaagacgtggtaccagacatctgaggactgctccagggatgccatcgtttttgtaactgtgcagggcagggccatctgttcggaccccaacaacaagagagtgaagaatgcagttaaatacctgcaaagccttgagaggtcttgaagcctcctcaccccagactcctgactgtctcccgggactacctgggacctccaccgttggtgttcaccgcccccaccctgagcgcctgggtccaggggaggccttccagggacgaagaagagccacagtgagggagatcccatccccttgtctgaactggagccatgggcacaaagggcccagattaaagtctttatcctcaaaaaaaaaaaaaaaaaaaaaa

SEQ ID NO: 323 - DNA, 283 bp, Homo sapiens
ctgagcagagggacctgcacacagagactccctcctgggctcctggcaccatggccccactgaagatgctggccctggtcaccctcctcctgggggcttctctgcagcacatccacgcagctcgagggaccaatgtgggccgggagtgctgcctggagtacttcaagggagccattccccttagaaagctgaagacgtggtaccagacatctgaggactgctccagggatgccatcgtttttgtaactgtgcagggcagggccatctgttcggaccccaacaa

SEQ ID NO: 324 - DNA, 160 bp, Homo sapiens
gcggtgacgacggggaccattttaccatcaccacccaccctgagagcaaccagggcatcctgacaaccaggaagggtttggattttgaggccaaaaaccagcacaccctgtacgttgaagtgaccaacgaggccccttttgtgctgaagctcccaacctc

SEQ ID NO: 325 - DNA, 300 bp, Homo sapiens
tttttttttggggccaattctttaatttaactaaattaggaacgcagcttttacagaacaataaacacaagggacggggccaccccaggatctaacagcttttcagggacctatgttgcaagctcaaaagtaatccactaacgaaccaagtcaaactccagtttttaataaaaaggggctgggggaggttgtcaaaccccttccaatataaatccccaatccgagggccaccaaatgaaaaagcaccaaaaatggaaggaaaactttcaaaaattctgcaaaaaatatgccccctttttt

SEQ ID NO: 326 - DNA, 394 bp, Homo sapiens
gtctattcttttattttactaaattaggaacgcagcatttacagaacaataaacacaagtgacgtggccaccccaggatctaacagctcttcagtgagctatgttgcaagctcagaagtaatccactaacgaaccaagtcagactccagttcttcatcaaaaggtgctggtggaggttgtcagacgccttccaatatagatccccaatccgatggccagcaaatgagagagcagcagagatggaaggaaaacttccagaaattctgcagagaatatgccccctttcttcatgacgctcgtgttcccccatgctgaaggtggccgtgcgcttccggtgtttaaagaagaacccttggggggaatatttcccggccatttgaccaatcccattcca

SEQ ID NO: 327 - DNA, 524 bp, Homo sapiens
gtctattcttttattttactaaattaggaacgcagcatttacagaacaaataaacacaagtgacgtggccaccccaggatctaacagctcttcagtgagctatgttgcaagctcagaagtaatccactaacgaaccaagtcagactccagttcttcatcaaaaggtgctggtggaggttgtcagacgccttccaatatagatccccaatccgatggccagcaaatgagagagcagcagagatggaaggaaaactttcagaaattctgcagagaatatgccccctttcttcatgacgctcgtgttcctcatgctgaggtggccgtgcgcttccggtgtttaaagaagaacccttggggggaatatttccggccgacttgaccaatcccatatccatctgatttttcttccagaagctttcacttccttcctccttcaatatcactccctcaactgtgactgttttccccccaatgctatggtttctgttcaaaaccccgttggttctgttgggtcgctactccgt

SEQ ID NO: 328 - DNA, 55 bp, Homo sapiens
ggccgcccttttttttttttttttttcgggggcgttttttgatttttaaaattgg

SEQ ID NO: 329 - DNA, 463 bp, Homo sapiens
tcactatagggaaagctggtacgcctgcaggtaccggtccggaattcccgggtcgacccacgcgtccgccgcccccgagacctgtgaagaaaaccatcttgtgaggggctgcctggactggtctggcaggttgggcctggatggggaggctctagcatctctcataggtgcaacctgagagtgggggagctaagccatgaggtaggggcaggcaagagagaggattcagacgctctgggagccagttcctagtcctcaactccagccacctgccccagctcgacggcactgggccagttccccctctgctctgcagctcggtttccttttctagaatggaaatagtgagggccaatgcccagggttggagggaggagggcgttcatagaagaacacacatgcgggcaccttcatcgtgtgtggcccactgtcagaacttaataaaagtcaactcatttgctgg

SEQ ID NO: 330 - DNA, 274 bp, Homo sapiens
  misc_feature 144, 218, 268: n = A,T,C or G
ccgcccccgagaccatgtgaagaaaaccatcttgtgaggggctgcctggactggtctggcaggttgggcctggatggggaggctctagcatctctcataggtgcaacctgagagtgggggagctaagccatgaggtaggggcangcaagagagaggattcagacgctctgggagccagttcctagtcctcaactccagccacctgccccagctcgacngcactgggccagttccccctctgctctgcagtcggtttccttttctagantggaaa

SEQ ID NO: 331 - DNA, 232 bp, Homo sapiens
cggctgtgagaatacgacagaagggtccggctgcgagaagacgacagaagggggatctcagcggggagccacgtctcttgcactgtggtctctgcatggaccccagggctgtggggacttgggggacagtaatcaagtaatccccttttccagaatgcattaacccactcccctgacctcacgctggggcaggtccccaagtgtgcaagctcagtattcatgatggtggggg

SEQ ID NO: 332 - DNA, 321 bp, Homo sapiens
gttgtgttgagatccagtgcagttgtgatttctgtggatcccagcttggttccaggaattttgtgtgattggtttaaatccagttttcaatcttcgacagctgggctggaacgtgaactcagtagctgaacctgtctgacccggtcacgttcttggatcctcagaactctttgctcttgtcggggtgggggtgggaactctcgtgaggagcgccagctgtgtaaatgccacgactccgtaattcttattcggtgggaccttgcttccctctgggagctggctcgttttgttggtgtctaacctttcgccgaatcgttaaag

SEQ ID NO: 333 - DNA, 344 bp, Homo sapiens
  misc_feature 265, 267, 272, 337: n = A,T,C or G
gtcctatttctcattttgttgataatttctgcatttaatggtctgtgctttaaatggtaacgctacggccccaggtcactgcgaggcacttaccatgtagatacgggctcaaaagtcacctctcagagacctacgtcatccactcaggaattcgcgcctctcatacttgcctgtctcattttatcttccttctagcagctgtctgaaattggttcgtctgttttcttgtttatggtattctcaagcccttgacagaccggctagngnggttntcccgtgcatcttcagcctggcacattatggacacttaaatactacgtattgatctaatattgangggttaa

SEQ ID NO: 334 - DNA, 405 bp, Homo sapiens
ggcacgagggatgaagggtgctgctcattttcattagatgtatgtgaaggcacagtgaaaatggaaatgttcttggagctacttcctcaaaatgtatccttagtcacctcagtgcaacagctgggagggggccgtgttaagattttttttgctacaaagaggaggtggcaatggtagatccacccttatgcttctcagtttagcataacctcttatggattttcatcaaattcagcgtgttggtcactggaaagagccttttccttctccttttcttactctcccctcatggggttcccctcttaaaggagaggagcttttaatttacacttaccacctcatttgcttttttggaggccatgccatataagcgggactaccgagttaatctcctttttacaaaag

SEQ ID NO: 335 - DNA, 227 bp, Homo sapiens
ggatgaactattcagatgctatcgtttggctaaaagaacatgatgtaaagaaagaagatggaactttctatgaatttggagaagatatcccagaagctcctgagagactgatgacagacaccattaatgaaccaatcttgctgtgtcgatttcctgtggagatcaagtccttctacatgcagcgatgtcctgaggattcccgtcttactgaatctgtcgacgtgttg

SEQ ID NO: 336 - DNA, 521 bp, Homo sapiens
tcgaattcggatgaactattcagatgctatcgtttggctaaaagaacatgatgtaaagaaagaagatggaactttctatgaatttggagaagatatcccagaagctcctgagagactgatgacagacaccattaatgaaccaatcttgctgtgtcgatttcctgtggagatcaagtccttctacatgcagcgatgtcctgaggattcccgtcttactgaatctgtcgacgtgttgatgcccaatgttggtgagattgtgggaggctcaatgcgtatctttgatagtgaagaaatactggcaggttataaaagggaagggattgaccccactccctattactggtatacggatcagagaaaatacggtacatgtccccatggaggatatggcttgggcttggaacgattcttaacgtggattctgaataggtatcacatccgagacgtgtgcttataccctcgatttgtccagcgttgcacgccataaccattttctccagaagcgtggaggaaagattatg

SEQ ID NO: 337 - DNA, 325 bp, Homo sapiens
ggactttcccgatcgccaggcaggagtttctctcggtgactactatcgctgtcatgtctggtcgtggcaagcaaggaggcaaggcccgcgccaaggccaagtcgcgctcgtcccgcgcgggccttcagttcccggtagggcgagtgcatcgcttgctgcgcaaaggcaactacgcggagcgagtgggggccggcgcgcccgtctacatggctgcgttcctcgagtatctgaccgctgagatcctggagctggcgggcaacgcggctcgggacaacaagaagacgcgcatcatccctcgtcacctccagctggccatccgcaacga

SEQ ID NO: 338 - DNA, 401 bp, Homo sapiens
  misc_feature 264: n = A,T,C or G
cgttgctgtcggttttaggaaacctggcatggtgctttcaggtctggggcttttagagccccccgtgtggcttacaaattctacagcatacagagcaggccacgctcaggcccggcatgcgggccaccaagttctggaaaccacgtggtgtccctgcgaatggggcgatcaagtccagagccggggcactttcagagtttgaaggtaactgagagcagatggtcctccatttcaactccagaagtggggctctgggagggatgntctaaccctccctggcatgtcacaaccaggctctggctggaggatccctccatccggctcctgtcatcccctacactttggcctagcaagaggtggaataaccacttgtgtgctcattactgttgggaggaacaaag

SEQ ID NO: 339 - DNA, 460 bp, Homo sapiens
catgcgggccaccaagttctggaaaccacgtggtgtccctgcgaatggggcgatcaagtccagagccggggcactttcagagtttgaaggtaactgagagcagatggtcctccatttcaactccagaagtggggctctgggagggatgttctagccctccctggcatgtcagaaccaggctctgcctggaggatccctccatccggctcctgtcatcccctacactttggccaagcaagaagtggtagaaccacttggctgctccttccttctggaggacacacagtctcagtccagatgccttcctgtctttctggccctttctggaccagatcctactcttcctttctaaatctgagatctccctccagggaatccgcctgcagaggacagagctggctgtcttcccccacccctaacctggcttattcccaactgctctgcccactgtgaaaccact

SEQ ID NO: 340 - DNA, 496 bp, Homo sapiens
tttttttttttttttttttttttttgggattcttaaatatagatgtatttttttcatctcatctccggacacactccaatcacacccctcctgccctcccctctcaactgcaaaccaagcggtgcagacacagcacagcacacatgaggggccctccctttcaccaaagctgaaggcagggcacagtttggggatggaagagcctcgaggtaaatgtgggggttctagaacccagtgacctcagttctggatcatgggaaagggatcagtatgcagtaacgtggtaaggttccagatctagaagccaggacctagaacctagtggtttcacagtgggcagagcagttgggaataagccaggttaggggtgggggaagacagccagctctgtcctctgcaggcggattccctggagggagatctcagatttagaaaggaagagtaggatctggtccagaaagggccagaaagacaggaaggcatctggactgagact

SEQ ID NO: 341 - DNA, 283 bp, Homo sapiens
ttttttttttttttttttttttttttttaggatttgaatacatttattgtgacaagaatgctgttataaatattcataagcaaaggccatctttttatctaggaattgtcaaagagaagattccaaattggaaggatacatcttttgtaaaatctgccaccaattcctgctttgagaataagcacctattgtaaaatttctactaacattataaatggtcacagcacatgccacttgatacaatccaaactttgaaatgtttgacttctcagtgggctgtccc

SEQ ID NO: 342 - DNA, 335 bp, Homo sapiens
tgtcgggcagcaggcgcagcccagcctcgaaatgcagaacgacgccggcgagttcgtggacctgtacgtgccgcggaaatgctccgctagcaatcgcatcatcggtgccaaggaccacgcatccatccagatgaacgtggccgaggttgacaaggtcacaggcaggtttaatggccagtttaaaacttatgctatctgcggggccattcgtaggatgggtgagtcagatgattccattctccgattggccaaggccgatggcatcgtctcaaagaacttttgactggagagaatcacagatgtggaatatttgtcataaataaataatgaaaacc

SEQ ID NO: 343 - DNA, 75 bp, Homo sapiens
gggtagagttcttaaatcgagatctggaggtagatggacgctttgtaaccctccagatctgggacactgcagggc

SEQ ID NO: 344 - DNA, 611 bp, Homo sapiens
gccggggggcagcggcgggcgcgagcggcagctgtcaggccaccgaggtccaagccgcacttgctgccccattgaggacgaggaggcagcaggagcagtgacggtgactctaaggagccggattcccggcacgcagagctgacctgcctggcacccgcggccctctcctgtttccttcccattgtgttggcaccctaaaaagaaagaataaaacaacaacaggaaaaaaaggaaaatatttaaattgtgacaaaaacccactgggttctcttggttacaaactccttcccttctggtgctacaaaaatgagtgggaaatccctgctcttaaaggtcattctcttgggtgatggtggagttgggaaaagttcgcttatgaaccgttacgtaaccaacaaatttgactcccaggcttttcacaccataggggtagagttcttaaatcgagatctggaggtagatggacgctttgtaaccctccagatctgggacactgcagggcaggaacgtttcaagagccttaggacacccttctacaggggagcagactgctgcctcttgaccttcagcgtggatgatcggcagagcttcgagaatcttggtaactggca

SEQ ID NO: 345 - DNA, 441 bp, Homo sapiens
ggcctttgcaagcctcaccggcgatgcaaggatagtcatcaacagggcccgggtggagtgccagagccaccggctgactgtggaggacccggtcactgtggagtacatcacccgctacatcgccagtctgaagcagcgttatacgcatagcactgggcgcaggcgtttggcatctctgccctcatcgtgggtttctactttgatggcactcctaggctctatcagactgacccctctgtcacataccatgcctggaaggccaatgccatacgccggggtgccaactcagtgcgtgagttcctggagaagaactatactgacgaagccattgtaacatatgatctgaccattaagctggtgatcaacgcactcctggaagtggttcactcaggtggcaaaaacattgaacttgctgtcatgaggcgagatcaatccctcaag

SEQ ID NO: 346 - DNA, 323 bp, Homo sapiens
ggcctttgcaggcctcaccgccgatgcaaggatagtcatcaacagggcccgggtggagtgccagagccaccggctgactgtggaggacccggtcactgtggagtacatcacccgctacatcgccagtctgaagcagcgttatacgcacagcaatgggcgcaggcgtttggcatctctgccctcatcgtgggtttcgactttgatggcactcctaggctctatcagactgacccctcgggcacataccatgcctggaaggccaatgccatatgccggggtgccaagtcagtgcgtgagttcctggagaagaactatactgacga

SEQ ID NO: 347 - DNA, 567 bp, Homo sapiens
ccagcggcctcttccccttcctggtgctgcttgccctgggaactctggcaccttgggctgtggaaggctctggaaagtccttcaaagctggagtctgtcctcctaagaaatctgcccagtgccttagatacaagaaacctgagtgccagagtgactggcagtgtccagggaagaagaaatgttgtcctgacacttgtggcatcaaatgcctggatcctgttgacaccccaaacccaacaaggaggaagcctgggaagtgcccagtgacttatggccaatgtttgatgcttaacccccccaatttctgtgagatggatggccagtgcaagcgtgacttgaagtgttgcatgggcatgtgtgggaaatcctgcgtttcccctgtgaaagcttgattcctgccatatggaggaggctctggagtcctgctctgtgtggtccaggtcctttccaccctgagacttggctccaccactgatatcctcctttggggaaaggcttggcacacagcaggctttcaagaagtgccagttgatcaatgaataaataaacgagcctatttctctttgc

SEQ ID NO: 348 - DNA, 314 bp, Homo sapiens
  misc_feature 48: n = A,T,C or G
atgaagtccagcggcctcttccccttcctggtgctgcttgccctgggnactctggcaccttgggctgtggaaggctctggaaagtccttcaaagctggagtctgtcctcctaagaaatctgcccagtgccttagatacaagaaacctgagtgccagagtgactggcagtgtccagggaagaagagatgttgtcctgacacttgtggcatcaaatgcctggatcctgttgacaccccaaacccaacaaggaggaagcctgggaagtgcccagtgacttatggccaatgtttgatgcttaacccccccaatttctg

SEQ ID NO: 349 - DNA, 611 bp, Homo sapiens
ggctctgctctgcagcacacccgtgggtgacccctcaccccagaagcagcagtggcagcttgggaaatgtgaggaagggaaggagggagagacgggagggaggagagagaggagaagggaggcaggggaggggcagcagaaccaaggcaaatatttcagctgggctatacccctctccccatccctgttatagaagcttagagagccagccagcaatggaaccttctggttcctgcgccaatcgccaccagtatcaattgtgtgagcttgggtgcgagtgcacgcgtgcgtgagtacggagagtatatatagatctctatctcttagcaaaggtgaatgccagatgtaaatggcgcctctgggcaaaggaggcttgtattttgcacattttataaaaacttgagagaatgagatttctgcttgtatatttctaaaaagaggaaggagcccaaaccatcctctccttaccactcccatccctgtgagccctaccttacccctctgcccctagccaaggagtgtgaatttatagatctaactttcataggcaaaacaaaagcttcgagctgttgcgtgtgtgagtctgttgtgtggatgtgcgtgtgtggtcc

SEQ ID NO: 350 - DNA, 370 bp, Homo sapiens
tggctggatgggcttggactgtggtcctgaaagcagcaagaagtatgctgaggctgtcactcgggctaagcagattgtgtggaatggtcctgtgggggtatttgaatgggaagcttttgcccggggaaccaaagctctcatggatgaggtggtgaaagccacttctaggggctgcatcaccatcataggtggtggagacactgccacttgctgtgccaaatggaacacggaggataaagtcagccatgtgagcactgggggtggtgccagtttggagctcctggaaggtaaagtccttcctggggtggatgctctcagcaatatttagtactttcctgccttttagttcctgtgcacagcccctaagtca

SEQ ID NO: 351 - DNA, 177 bp, Homo sapiens
gggctgcatcaccatcataggtggtggagacactgccacttgctgtgccaaatggaacacggaggataaagtcagccatgtgagcactgggggtggtgccagtttggagctcctggaagggaaagtccttcctggggtggatgctctcagcaatatttagtactttcctgcctttta

SEQ ID NO: 352 - DNA, 204 bp, Homo sapiens
  misc_feature 53, 55, 76, 86, 137: n = A,T,C or G
atggcttttaccttccttaaggtgctcaacaacatggagattgggcactttcncnggttgatgaagaaggaagccnagatttgtcnaagacctaatgtccaaaagctgagaagaatggtgtgaagattaccttgccntgttgacttgtcactgctgacaagtttgatgagaatgcccaagactggcccagccactggtggcttc

SEQ ID NO: 353 - DNA, 489 bp, Homo sapiens
cttttaccttccttaaggtgctcaggacatggagattggcacttctctgtttgatgaagagggagccaagattgtcaaagacctaatgtccaaagctgagaagaatggtgtgaagattaccttgcctgttgactttgtcactgctgacaagtttgatgagaatgccaagactggccaagccactgtggcttctggcatacctgctggctggatgggcttggactgtggtcctgaaagcagcaagaagtatgctgaggctgtcactcgggctaagcagattgtgtggaatggtcctgtgggggtatttgaatgggaagcttttgcccggggaaccaaagctctcatggatgaggtggtgaaagccacttctaggggctgcatcaccatcataggtggtggagacactgccacttgctgtgccaaatggaacacggaggataaagtcagccatgtgagcactgggggtggtgccagtttggagctcctgga

SEQ ID NO: 354 - DNA, 885 bp, Homo sapiens
tttttttttttcacggtttcaatggacacttttattgtttacttaatggatcatcaattttgtctcactacctacaaatggaatttcatcttgtttccatgctgagtagtgaaacagtgacaaagctaatcataataacctacatcaaaagagaactaagctaacactgctcactttctttttaacaggcaaaatataaatatatgcactctaaaatgcacaatggtttagtcactaaaaaattcaaatgggatcttgaagaatgtatgcaaatccagggtgcagtgaaaatgagctgagatgctgtgcaactgtttaagggttcctggcactgcatctcttggccactagctgaatcttgacatggaaggttttagctaatgcccaggggaaatgcaaaaaatgctaatttgacttagggcctgtgcacaggaactaaaaggcaggaaagtactaaatattgctgagagcatccaccccaggaaggactttaccttccaggagctccaaactggcaccacccccagtgctcacatggctgactttatcctccgtgttccatttggcacagcaagtggcagtgtctccaccacctatgatggtgatgcagccccctaaaagtggctttcaccacctcatccatgagagctttggttccccgggcaaaagcttcccattcaaatacccccacaggaccattccacacaatctgcttaacccgagtgacagcctcagcatacttcttgctggtttcaggaccacagtccaagccccatcccaccagcaggtatgcaagaaggcccagtgggcttgccagtcttggcatttctcatcaacttgtcagcagtgacaaagtcaaccgggaaggaatcttcacaccatctt

SEQ ID NO: 355 - DNA, 434 bp, Homo sapiens
cggctgcgagaagacgacagaaggggggagtggttgctataccttgacttcatttatatgaatttccactttattaaataatagaaaagaaaatcccggtgcttgcagtagagtgataggacattctatgcttacagaaaatatagccatgattgaaatcaaatagtaaaggctgttctggctttttatcttcttagctcatcttaaataagcagtacacttggatgcagtgcgtctgaagtgctaatcagttgtaacaatagcacaaatcgaacttaggatttgtttcttctcttctgtgtttcgatttttgatcaattctttaattttggaagcctataatacagttttctattcttggagataaaaattaaatggatcactgatattttagtcattctgcttctcatctaaatatttccatattctgtatt

SEQ ID NO: 356 - DNA, 318 bp, Homo sapiens
gggagtgggtgctataccttgacttcatttatatgaatttccactttattaaataatagaaaagaaaatcccggtgcttgcagtagagtgataggacattctatgcttacagaaaatatagccatgattgaaatcaaatagtaaaggctgttctggctttttatcttcttagctcatcttaaataagcagtacacttggatgcagtgcgtctgaagtgctaatcagttgtaacaatagcacaaatcgaacttaggatttgcttcttctcttctgtgttgcgatttttgatcaattctttaattttggaagcctataat

SEQ ID NO: 357 - DNA, 231 bp, Homo sapiens
cggctgcgagaagacaacagaagggggctcccgctcgggatctcgctccggatctcgctccgggtcccgcagtgggtcccggagaggaagctttgacgccacaaggaattcttcctactcttattcctactcatttagcagtagttctattgggcactattagtcagttgggagtgggtgctataccttgacttcatttatatgaatttccactttattaaataatagaaa

SEQ ID NO: 358 - DNA, 446 bp, Homo sapiens
atttgctgtatgccgagaatggaaaaattggaccacctaaactggatatcagaaaggaggagaagcaaatcatgattgacatatttcacccttcagtttttgtaaatggagacgagcaggaagtcgattatgatcccgaaactacctgttacattagggtgtacaatgtgtatgtgagaatgaacggaagtgagatccagtataaaatactcacgcagaaggaagatgattgtgacgagattcagtgccagttagcgattccagtatcctcactgaattctcagtactgtgtttcagcagaaggagtcttacatgtgtggggtgttacaactgaaaagtcaaaagaagtttgtattaccattttcaatagcagtataaaaggttctctttggattccagttgttgctgctttactactctttctagtgcttagcctggtattcatc

SEQ ID NO: 359 - DNA, 209 bp, Homo sapiens
  misc_feature 19, 185, 193: n = A,T,C or G
gagaatttgctgtatgccngagatggaaaaattggaccacctaaactggatatcagaaaggaggagaagcaaatcatgattgacatatttcacccttcaagtttttgtaaatggagacgagcaggaagtcgattatgatcccgaaactacctgttacattagggtgtacaatgtgtatgtgagantgaacggnagtgagatccagtata

SEQ ID NO: 360 - DNA, 521 bp, Homo sapiens
tgctgtcggtgactactgaagaaatattcctgacgtggtcccgggcagccatctgactccaatagagagagagagttcttcacctttaagtagtaaccagtctgaacctggcagcatcgctttaaactcgtatcactccagaaattgttctgagagtgatcactccagaaatggttttgatactgattccagctgtctggaatcacatagctccttatctgactcagaatttcccccaaataataaaggtgaaataaaaacagaaggacaagagctcataaccgtaataaaagcccccacctcctttggttatgataaaccacatgtgctagtggatctacttgtggatgatagcggtaaagagtccttgattggttatagaccaacagaagattccaaagaattttcatgagatcagctaagttgcaccaactttgaagtctgattttcctggacagttttctgctttaatttcatgaaaagattatgatctcagaaattgtatcttagttggtatcaac

SEQ ID NO: 361 - DNA, 522 bp, Homo sapiens
tggccctcgaggccaagaattcggcactaggggagaggagcttgaatttctgacacacataacatgtaaaaagtatttggcatttcataaggatttggggtggggtaaacgcaaggttagtctgttttaaaaaatgttttcattaacgagcacataactggtggttcctaatgggaatacttgacccaggcagaaactagaaaagtagcaagtaggaaacttccatttctctcccctaaacaaccccttaaggcactgtgagctggagacaggagaggtgttgcccaacctttgttcatatactcggtgacgatgtagatgggctcctcagacaccactgcatagagctggaccagcttgtcgtgcttcagcttcttcatgatctgcgcttcctcaaggaatgattcgggggacattgtgcctggtttaagagtctttatggctacttttgtgtttccattccaggtacctacaaacatcccagaatatgaagtcaaaccaaagatcttcttttgatggaaa

SEQ ID NO: 362 - DNA, 421 bp, Homo sapiens
  misc_feature 12, 331, 372: n = A,T,C or G
ttaatgagttanaaatcttaatatagccatcttagccataaccacaaataaactcattttttctgttaaaatacttgacagagtccttgcaattgaatgtctttgttcaacaaaaactgtattaagtgttttaaatttaaaatctaatcttatgcaaatagctggtggtcaaaacctttttccatcaaaagaagatctttggtttgacttcatattctgggatgtttgtaggtacctggaatggaaacacaaaagtagccataaagactcttaaaccaggcacaatgtcccccgaatcattccttgaggaagcgcagatcatgaagaagcngaagcacgacaagctggtccagctctatggcagtggtgtcngaggagcccatctacatcgtcaccgagtatatgaacaaaggttgggcaa

SEQ ID NO: 363 - DNA, 503 bp, Homo sapiens
cagaaggggtttccgaatgttttagttagccttttggtggagccgccagctgacaggacatcttacaagagaatttgcacatctctggaagcttagcaatcttattgcacactgttcgctggaagctttttgaagagcacattctcctcagtgagctcatgaggttttcatttttattcttccttccaacgtggtgctatctctgaaacgagcgttagagtgccgccttagacggaggcaggagtttcgttagaaagcggacgctgttctaaaaaaggtctcctgcagatctgtctgggctgtgatgacgaatattatgaaatgtgccttttctgaagagattgtgttagctccaaagcttttcctgtcgcagtgtttcagttctttattttcccttgtggatatgctgtgtgaaccgtcgtgtgagtgtggtatgcctgatcacagatggattttgttataagcatcaatgtgacacttgcaggacactacaacgtgggaca

SEQ ID NO: 364 - DNA, 365 bp, Homo sapiens
ggccgcccttttttttttttttgggggggaaaaaatttttttttaaaaaaaaaaaaacttcccccctggggaaaaaaaaaggttttttaaaaaaaaaaccaaacaaaattttcccgggccctttaggggttttaaattttcccccgggttgaaccccttttaaaaaaaaaggaatttttttggggggaaataatgggggaaaaaccaaaaaaaaaggggggttttttttttaaaacccttttttttttaaaaaaccttcccccaggggaaaaattcccaaaaccttttaaaaaaaaagggccgaaatttttaatccaaaggggaaaaaccccccccccaacaaaaaacccccaaaggggaaaaag

SEQ ID NO: 365 - DNA, 680 bp, Homo sapiens
  misc_feature 172, 173, 176, 186, 199, 200, 591, 625, 659, 670: n = A,T,C or G
aggacacagacaaggaacttgctgaaaggccaaccatttcaggatcagtcaaaggcagcaagcagatagactcaaggtgtgtgaaagatgttatacaccaggagctgccacttcatgtcccaaccagactgtgtctgtctgtgtctgcatgtaagagtgagggagggaaggnnggnactacaaganagtcggagatganncagcacacacacaattccccagcccacgtgatgcttgtgttgaccagatgttcctgagtctggagcaagcacccaggccagaataacagagctttcttagttggtgaagacttaaacatctgcctgaggtcaggaggcaatttgcctgccttgtacaaaagctcaggtgaaagactgagatgaatgtctttcctctccctgcctcccaccagacttcctcctggaaaacgctttggtagatttggccaggagctttcttttatgtaattggataaatacacacaccatacactatccacagatatagccaagtagatttgggtagaggatactatttccagaatagtgtttagctcacctagggggatatgttgtatcacatttgcatatnccacatggggacataagctaattttttacagacncgattctgtcatgctgttaatagccatggttaancccccattggnggggccggtg

SEQ ID NO: 366 - DNA, 570 bp, Homo sapiens
taagctcgggattcggctcgagcggctcgagtcaagagaaaacacaagaaggacatcagccagaacaagcgagccgtgaggcggctgcgcaccgcctgcgagagggccaagaggaccctgtcgtccagcacccaggccagcctggagatcgactccctgtttgagggcatcgacttctacacgtccatcaccagggcgaggttcgaggagctgtgctccgacctgttccgaagcaccctggagcccgtggagaaggctctgcgcgacgccaagctggacaaggcccagattcacgacctggtcctggtcgggggctccacccgcatccccaaggtgcagaagctgctgcaggacttcttcaacgggcgcgacctgaacaagagcatcaaccccgacgaggctgtggcctacggggcggcggtgcaggcggccatcctgatgggggacaagtccgagaacgtgcaggacctgctgctgctggacgtggctcccctgtcgctggggctggagacggccggaggcgtgatgactgccctgatcaagcgcaactccaccatccccaccaagcag

SEQ ID NO: 367 - DNA, 454 bp, Homo sapiens
gccgcccttttttttttttttttttttttttttttttttgtttttttttttttttcaaaaaaaaaaaatctttttagaaaaaaaaacccccccccaacaaaaaatggggggggggggggattttccctcccgggggaaggagaaaaagccgcagtaataaaaagggggggaaccaaaaaatttttttttttttttaaaaaaggtttttttgggggcccccccccccaaaaaaaaaaaaggtcccccccctttttttcccccctttttgggggggaaaaaaaaaaaaggggggggaaaaaaacagaaaattttccccaaaaatttaaaaaaaaaaggggggggggggggaaaaaaaaggtttttttacccccctgggggggaaaaaaaaaaaatttggggccaccaaaaaggggggggggccccccaaaaaagggggttttttttaaaaaaaaaa

SEQ ID NO: 368 - DNA, 651 bp, Homo sapiens
taagctcgggattcggctcgagtggtcttcgtctactccgggtctttcaggaggccaaaaggcagctccagaagattgacaaatctgagggccgcttccatgtccagaaccttagccaggtggagcaggatgggcggacggggcatggactccgcagatcttccaagttctgcttgaaggagcacaaagccctcaagacgttaggcatcatcatgggcactttcaccctctgctggctgcccttcttcatcgttaacattgtgcatgtgatccaggataacctcatccgtaaggaagtttacatcctcctaaattggataggctatgtcaattctggtttcaatccccttatctactgccggagcccagatttcaggattgccttccaggagcttctgtgcctgcgcaggtcttctttgaaggcctatgggaatggctactccagcaacggcaacacaggggagcagagtggatatcacgtggaacaggagaaagaaaataaactgctgtgtgaagacctcccaggcacggaagactttgtgggccatcaaggtactgtgcctagcgataacattgattcacaagggaggaattgtagtacaaatgactcactgctgtaaagcagtttttctacttttaaagacccccccc

SEQ ID NO: 369 - DNA, 280 bp, Homo sapiens
  misc_feature 112: n = A,T,C or G
tggtcttcgtctactccagggtctttcaggaggccaaaaggcagctccagaagattgacaaatctgagggccgcttccatgtccagaaccttagccaggtggagcaggatgngcggacggggcatggactccgcagatcttccaagttctgcttgaaggagcacaaagccctcaagacgttaggcatcatcatgggcactttcaccctctgctggctgcccttcttcatcgttaacattgtgcatgtgatccaggataacctcatccgtaagaagtttac

SEQ ID NO: 370 - DNA, 418 bp, Homo sapiens
ggccgcccttttttttttttttttttcccgggcttttttgggaaaaacccccctttcccataaaaaaatttttttgggggtttcccaatttttttttccaatttcaaataatttttttccaaaaaaaacccaaacccttggggcctttttttttttttttaaagggcctttttacttttccccaaggaggccttggggaaataaaaaaaacccggttggggggccccaaaaaaggggtgggcccccttgaatcccccattggtttgggggtaaaaaaggcccccccatgggcccccttcccccgggggggggaacccccccccaagacccccccgggggaaaccgggcccaaaaaaaaaaccctttaaaattttaaaaaacgggcccccccctaaaaaaacttttttttaaaaagggg

SEQ ID NO: 371 - DNA, 292 bp, Homo sapiens
ttagggtataagttgctgtaaaatttgtgtaaatttgtatccacacaaattcagtctctgaatacacagtattcagagtctctgatacacagtaattgtgacaatagggctaaatgtttaaagaaatcaaaagaatctattagattttagaaaaacatttaaactttttaaaatacttattaaaaaatttgtataagccacttgtcttgaaaactgtgcaactttttaaagtaaattattaagcagactggaaaagtgatgtattttcatagtgacctgtgtttcacttaat

SEQ ID NO: 372 - DNA, 415 bp, Homo sapiens
tccttatttatttaacttcacccgagttcctctgggtttctaagcagttatggtgatgacttagcgtcaagacatttgctgaactcagcacattcgggaccaatatatagtgggtacatcaagtccatctgacaaaatggggcagaagagaaaggactcagtgtgtgatccggtttctttttgctcgcccctgttttttgtagaatctcttcatgcttgacatacctaccagtattattcccgacgacacatatacatatgagaatataccttatttatttttgtgtaggtgtctgccttcacaaatgtcattgtctactcctagaagaaccaaatacctcaatttttgtttttgagtactgtactatcctgtaaatatatcttaagcaggtttgttttcagcactgatggaaaa

SEQ ID NO: 373 - DNA, 326 bp, Homo sapiens
tccttatttatttaacttcacccgagttcctctgggtttctaagcagttatggtgatgacttagcgtcaagacatttgctgaactcagcacattcgggaccaatatatagtgggtacatcaagtccatctgacaaaatggggcagaagagaaaggactcagtgtgtgatccggtttctttttgctcgcccctgttttttgtagaatctcttcatgcttgacatacctaccagtattattcccgacgacacatatacatatgagaatataccttatttatttttgtgtaggggtctgccttcacaaatgtcattgtctactcctaca

SEQ ID NO: 374 - DNA, 324 bp, Homo sapiens
tccttatttatttaacttcacccgagttcctctgggtttctaagcagttatggtgatgacttagcgtcaagacatttgctgaactcagcacattcgggaccaatatatagtgggtacatcaagttcatctgacaaaatggggcagaagagaaaggactcagtgtgtgatccggtttctttttgctcgcccctgttttttgtagaatcttttcatgcttgacatacctaccagtattattcccgacgacacatatacatatgagaatataccttatttatttttgagtaggtgtctgccttcacaaatggcattggctactccag

SEQ ID NO: 375 - DNA, 466 bp, Homo sapiens
taactctgggaggggctcgagagggctggtccttatttatttaacttcacccgagttcctctgggtttctaagcagttatggtgatgacttagcgtcaagacatttgctgaactcagcacattcgggaccaatatatagtgggtacatcaagtccatctgacaaaatggggcagaagagaaaggactcagtgtgtgatccggtttctttttgctcgcccctgttttttgtagaatctcttcatgcttgacatacctaccagtattattcccgacgacacatatacatatgagaatataccttatttatttttgtgtaggtgtctgccttcacaaatgtcattgtctactcctagaagaaccaaatacctcaatttttgtttttgagtactgtactatcctgtaaatatatcttaagcaggtttgttttcagcactgatggaaaataccagtgttgggttttttttt

SEQ ID NO: 376 - DNA, 324 bp, Homo sapiens; identical to SEQ ID NO: 374

SEQ ID NO: 377 - DNA, 326 bp, Homo sapiens; identical to SEQ ID NO: 373

SEQ ID NO: 378 - DNA, 494 bp, Homo sapiens
atgccccgcatagatgcggacctcaagctcgacttcaaggatgtcctgctccgacctaagcggagcagcctcaagagccgagccgaggtggatcttgaacgcaccttcacgtttcgaaattcaaagcagacctactcagggattcccatcatcgtggccaacatggacactgtgggcacgtttgagatggcagccgtgatgtcacagcactccatgtttacagcaattcataagcattactccctggatgactggaagctctttgccacaaatcacccagaatgcctgcagaatgtagccgtgagttcaggcagtgggcagaatgatctggaaaagatgaccagcatcctggaagctgtgccacaggttaagtttatttgcctggatgtggccaatgggtattcaaaacattttgtggaattcgtgaaacttgtccgtgccaaatttcctgaacacaccattatggcagggaacgtggtgacaggagaaatggt

SEQ ID NO: 379 - DNA, 243 bp, Homo sapiens
gccgctgcaccatgccccgcatagatgcggacctcaagctcgacttcaaggatgtcctgctccgacctaagcggacagcctcaagagccgagccgaggtggatcttgaacgcaccttcacgtttcgaaattcaaagcagacctactcagggattcccatcatcgtggccaacatggacactgtgggcacgtttgagatggcagccgtgatgtcacagcactccatgtttacagcaattcataa

SEQ ID NO: 380 - DNA, 804 bp, Homo sapiens
gcaaatgtttgattaattctgctcatatgcacatctgaaagcatgagacacactccacagacagcacgcactggagctggtggggcagatgggcactcgccgattaggtattaatgtcaataatacgtgcataaagtgctgataaaataacttaagtgttacaaaaacagacagtccacggtggctgcaggcacatgcaggcgggactgggtcagacactccagggctgcacatgttccagctggcctgagtccgacacgtcatagctggccttgtacttggccaggattttcatgaggggccgtagcttgagccaccactgttctttgggaatcctgtgctcaaaatccgtttgcttcttcagctctgccacaggtttgaaaaataacgtttcttttgcttattcccagcacacaaatggaatcatcggtggtaaatttttttcctctgccccgggcctccttgagttttgcagtgatccactccatagctctggcagagattttggttccaaagtttctatcaaatggagagggtgccccaccctgctgcatgtgacccagcacgttcttcctgcagtcaaacacgcctttgccctcttctgaatacagctggtaaatgaagtcggtggtgtagttttcactgcagctctcatttctgagcacaaggcctctctggatggtggtcttcattttctccgtcaggtgctccacgttggactgcagatcctgatgtcgaagggctcttcgaaatgtatgcggcatcagtccggccgcagccccccatgttggcaggtagcacagt

SEQ ID NO: 381 - DNA, 624 bp, Homo sapiens
  misc_feature 514: n = A,T,C or G
tggagttgtaggcaaatgtttaattaattctgctcatatgcacatctgaaagcatgagacacactccacagacagcacgcactggggctggtggggcagatgggcactcgccgattaggtattaatgtcaataatacgtgcataaagtgctgataaaataacttaagtgttacaaaaacagacagtccacggtggctgcaggcacatgcaggcgggactgggtcagacactccagggctgcacatgttccagctggcctgagtccgacacgtcatagctggccttgtacttggccaggattttcatgaggggccgtagcttgagccaccactgttctttgggaatcctgtgctcaaaatccgtttgcttcttcagctctgccacaggttgaaaaataacgtttcttttgcttattcccagcacacaaatggaatcatcggtggtaaatttttttcctctgccccgggcctccttgagttttgcagtgatccactccatagctctggcagagatnttggttccaaagtttctatcaaatggagaggtgccccaccctgctgatgtgaccccacacgttcttcctgagtcaaacacgcctttgccctcttctgaatacaagctggt

SEQ ID NO: 382 - DNA, 507 bp, Homo sapiens
  misc_feature 301, 460, 498: n = A,T,C or G
ttttttggagttgtaggaaatgtttaattctgctcatatgcacatctgaaagcatgagacacactccacaagacagcacgcactggggctggtggggcagatgggcactcgcgattaggtattaatgttaataatacgtgcataaagtgctgataaaataacttaagtgttacaaaaacagacagtccacggtggctgcaggcacatgcaggcgggactgggtcagacactccagggctgcacatgttccagctggcctgagtcccgacacgtcatagctggccttgtacttggccagggnttttcatgaggggccctagctttgagccaccacttgttctttggggaatcctgtgcttcaaaatcccgttttgcttctttcagctcttcccacaggtttgaaaaataacgttttctttttgcttatttcccagcacacaaatgggattcatcggtgggnaatttttttcctctgccccggggcttcttgagtttttncagtgattc

SEQ ID NO: 383 - DNA, 224 bp, Homo sapiens
  misc_feature 198, 219: n = A,T,C or G
atcagatcccaaagaccaattgcaacgtagctgtcatcaacgtgggggcacccgcggctgggatgaacgcggccgtacgctcagctgtgcgcgtgggcattgccgacggcacaggatgctcgccatctatgatggtttgacggcttcgcaagggccagatcaaagaaatcggctggacagatgtcgggggctggaccngccaaggaggctccattcttnggaca

SEQ ID NO: 384 - DNA, 507 bp, Homo sapiens; identical to SEQ ID NO: 382
  misc_feature 301, 460, 498: n = A,T,C or G

SEQ ID NO: 385 - DNA, 224 bp, Homo sapiens; identical to SEQ ID NO: 383
  misc_feature 198, 219: n = A,T,C or G

SEQ ID NO: 386 - DNA, 232 bp, Homo sapiens
acgacagaagggtacggctgcgagaagacgacagatgggtacggctgtgagaagacgactgatgggaacagctaaggactgctaaaccccactctgcatcaactgaacgcaaatcagccactttaattaagctaagcccttactagaccaatgggacttaaacccacaaacacttagttaacagctaagcaccctaatcaactggcttcaatgtacttctcccgccgtcggg

SEQ ID NO: 387 - DNA, 339 bp, Homo sapiens
tactggttttggagaacttgtctacaaccagggattgattttaaagatgtctttttttattttacttttttttaagcaccaaattttgttgtttttttttttttctcccttccccacaaatcccttttaaaatatttttgttaaccccctttccaacgggccgaggaaacttaaaacccctttttcctcggcctggttcctctttaatttttaatttttccccatcagtttaaaggttttggcatacttggcatcttttttcaaagggaaaactttttttgccattctttggacttccccttttttaaaggaaatgggggggccaaaaggggatttcaa

SEQ ID NO: 388 - DNA, 456 bp, Homo sapiens
tttttttttttttttttttttttaaccatcaaattcacagctatttttcgcttttagtgtgctcacagaaaattagaacaccttaagcaggagtttaatagcattttttgtaagcaaagttacattccatctctaagtcaaattggtcaaagcttctccagtatttacaaaacatgatagacaagatgctacacaaaaccattgcatctgaagattttgttttcctttattctcaaagacgactggaaaagaaagcattatctgctgtaatcaaaaacataccacagtataaacagttaccattccacttatcacagcttggttgagtttagaattagtgttttaaaaagtccaagatgactgcagttttacaaaaatgggcagggtggaaagttgcaaacttcatgtgcttctggatatcaagatttgtttttatacaatagtcacagttaaaaa

SEQ ID NO: 389 - DNA, 490 bp, Homo sapiens
ttacattgaatactacatatgtcgagggaatgcagaaagagttaaggaaggcaggttgtcctgctatggaggccactcttcgttttccatgtactgcatgctgtttgtggcactttatcttcaagccaggatgaagggagactgggcaagactcttaccccccacactgcaatttggtcttgttgccgtatccatttatgtgggcctttctcgagttgctgattataaacaccactggagcgatgtgttgactggactcattcagggagctctggttgcaatattagttgctgtatatgtatcggatttcttcaaagaaagaacttcttttaaagaaagaaaagaggaggactctcatacaactctgcatgaaacaccaacaactgggaatcactatccgagcaatcaccagccttgaaaggcagcagggtgcccaggtgaagctggcctgttttctaaaggaaaatgattgccacaaggcaagaggatg

SEQ ID NO: 390 - DNA, 334 bp, Homo sapiens
gaactcggtggtggccactgcgcagaccagacttcgctcgtactcgtgcgcctcgcttcgcttttcctccgcaaccatgtctgacaaacccgatatggctgagatcgagaaattcgataagtcgaaactgaagaagacagagacgcaagagaaaaatccactgccttccaaagaaacgattgaacaggagaagcaagcaggcgaatcgtaatgaggcgtgcgccgccaatatgcactgtacattccacaagcattgccttcttattttacttcttttagctgtttaactttgtaagatgcaaagaggttggatcaagattaaatgactgtgctg

SEQ ID NO: 391 - DNA, 377 bp, Homo sapiens
  misc_feature 349: n = A,T,C or G
gaactcggtggtggccactgcgcagaccagacttcgctcgtactcgtgcgcctcgcttcgcttttcctccgcaaccatgtctgacaaacccgatatggctgaggtcgagaaattcgataagtcgaaactgaagaagacagagacgcaagagaaaaatccactgccttccaaagaaacgattgaacaggagaagcaagcaggcgaatcgtaatgaggcgtgcgccgccaatatgcactgtacattccacaagcattgccttcttattttacttcttttagctgtttaactttgtaagacgcatagagggtggatcaagtttaaatgactgtgctgcccctttcacatcanagaactactgacaacgaaggccgcgcct

SEQ ID NO: 392 - DNA, 555 bp, Homo sapiens
ctcggtggtggccactgcgcagaccagacttcgctcgtactcgtgcgcctcgctttgcttttcctccgcaaccatgtctgacaaacccgatatggctgagatcgagaaattcgataagtcgaaactgaagaagacagagacgcaagagaaaaatccactgccttccaaagaaacgattgaacaggagaagcaagcaggcgaatcgtaatgaggcgtgcgccgccaatatgcactgtacattccacaagcattgccttcttattttacttcttttagctgtttaactttgtaagatgcaaagaggttggatcaagtttaaatgactgtgctgcccctttcacatcaaagaactactgacaacgaaggccgcgcctgcctttcccatctgtctatctatctggctggcagggaaggaaagaacttgcatgttggtgaaggaagaagtggggtggaagaagtggggtgggacgacagtgaaatctagagtaaaaccaagctggcccaaggtgtcctgcaggctgtaatgcagtttaatcagagtgccatttttttttt

SEQ ID NO: 393 - DNA, 300 bp, Homo sapiens
gctcaattggactatgttgacctctatcttattcattctccaatgtctctaaagccaggtgaggaactttcaccaacagatgaaaatggaaaagtaatatttgacatagtggatctctgtaccacctgggaggccatggagaagtgtaaggatgcatgattggccaagtccattggggtgtcaaacttcaaccgcaggcagctggagatgatcctcaacaagccaggactcaagtacaagcctggctgcaaccaggtagaaagtcattcgtatttcaaccggagtaaattgctagaatcg

SEQ ID NO: 394 - DNA, 344 bp, Homo sapiens
acagaagggtacggctgcgagaagacgacagaagggtacggctgcgagaagacgacagaagggtacggctgcgagaagacgacagaagggtaaaacactgaactgacaattaacagcccaatatctacaatcaaccgacaagtcattattaccctcactgtcaacccaacacaggcatgctcataaggaaaggttaaaaaaagtaaaaggaactcggcaaatcttaccccgcctgtttaccaaaaacatcacctgtagcatcaccagtattagaggcaccgcctgcccagtgacacatgtttaacggccgcggtaccctaaccgtgcaaaggtagcataatcac

SEQ ID NO: 395 - DNA, 507 bp, Homo sapiens
tgctcggtccttccgaggaagctaaggctgcgttggggtgaggccctcacttcatccggcgactagcaccgcgtccggcagcgccagccctacactcgcccgcgccatggcctctgtctccgagctcgcctgcatctactcggccctcattctgcacgacgatgaggtgacagtcacggaggataagatcaatgccctcattaaagcagccggtgtaaatgttgagcctttttggcctggcttgtttgcaaaggccctggccaacgtcaacattgggagcctcatctgcaatgtaggggccggtggacctgctccagcagctggtgctgcaccagcaggaggtcctgccccctccactgctgctgctccagttgaggagaagaaagtggaagcaaagaaagaagaatccgaggagtctgatgatgacatgggctttggtctttttgactaaacctcttttataacatgttcaataaaaagctgaactttaaaaaaaaaaaaaaaaaa

SEQ ID NO: 396 - DNA, 488 bp, Homo sapiens
gaggccctcacttcatccggcgactagcaccgcgtccggcagcgccagccctacactcgcccgcgccatggcctctgtctccgagctcgcctgcatctactcggccctcattctgcacgacgatgaggtgacagtcacggaggataagatcaatgccctcattaaagcagccggtgtaaatgttgagcctttttggcctggcttgtttgcaaaggccctggccaacgtcaacattgggagcctcatctgcaatgtaggggccggaggacctgctccagcagctggtgctgcaccagcaggaggtcctgccccctgcactgctgctgctccagttgaggagaagaaagtggaagcatagaaagaagaatccgacgagtctgatgatgacatgggctatggtctttttgactaaacctcttttataacatgttcaataaaaagctgaactttaaaaagaaaaaaaaaaaactcgagcctctagaactata

SEQ ID NO: 397 - DNA, 180 bp, Homo sapiens
ctgcgttggggtgaggccctcacttcatccggcgactagcaccgcgtccggcagcgccagccctacactcgcccgcgccatggcctctgtctccgagctcgcctgcatctactcggccctcattctgcacgacgatgaggtgacagtcacggaggataagatcaatgccctcattaaagc

SEQ ID NO: 398 - DNA, 491 bp, Homo sapiens
  misc_feature 12, 154, 255, 348, 368, 402, 409, 450, 471: n = A,T,C or G
tttttttttttntttcactgttcaaggtttattgggggttttagttggtataacacttggatagttggttgcattgtttgtatgtagatctttttacattatatggtaatgtacactactgatatagttcacaaaataagatcctttggaaganttatacacaagacatgatattggatttatacactggatcccagggatgtgactcactgggaaaaaatgttggactaggcatgttcagtgaaggagccaggnagttatataacacacggtaaacatccacctggctcaaggggcaaatgcagtacgtacagcattggcagtggtgcgtcagaggtggcagaactntttcacactaaccagttgangactacacaagattaataccatccagcatcaggntatagcntgtggattttacaaaccatttcttatttctaactttcaggngttgatgtttttcccagtccntcttaaaatttttactgctt

SEQ ID NO: 399 - DNA, 235 bp, Homo sapiens
tgatttctgtggatcccagcttggttccaggaattttgtgtgattggcttaaatccagttttcaatcttcgacagctgggctggaacgtgaactcagtagctgaacctgtctgacccggtcacgttcttggatcctcagaactctttgctcttgtcggggtgggggtgggaactcacgtggggagcggtggctgagaaaatgtaaggattctggaatacatattccatgggactt

SEQ ID NO: 400 - DNA, 465 bp, Homo sapiens
tacggctgcgagaagacgacagaagggtacggctgcgagaagacgacagaagggtacggctgcgagaagacgacagaagggtacggctgcgagaagacgacagaagggtgatttctgtggatcccagcttggttccaggaattttgtgtgattggcttaaatccagttttcaatcttcgacagctgggctggaacgtgaactcagtagctgaacctgtctgacccggtcacgttcttggatcctcagaactctttgctcttgtcggggtgggggtgggaactcacgtggggagcggtggctgagaaaatgtaaggattctggaatacatattccatgggactttccttccctctcctgcttcctcttttcctgctccctaacctttcgccgaatggggcagcaccactgacgtttctgggcggccagtgcggctgccaggttcctgtactactgccttgtacttt

SEQ ID NO: 401 - DNA, 243 bp, Homo sapiens
tgatttctgtggatcccagcttggttccaggaattttgtgtgattggcttaaatccagttttcaatcttcgacagctgggctggaacgtgaactcagtagctgaacctgtctgacccggtcacgttcttggatcctcacaactctttgctcttgtcggggtgggggtgggaactcacgtggggagcggtggctgagaaaatgtaaggattctggaatacatattccatgggactttccttccc

SEQ ID NO: 402 - DNA, 506 bp, Homo sapiens
ttctagcatcctcttaacgtgcagcaaaagcaggcgacaaaatctcctggctttacagacaaaaatatttcagcaaacgttgggcatcatggtttttgaaggctttagttctgctttctgcctctcctccacagccccaacctcccacccctgatacatgagccagtgattattcttgttcagggagaagatcatttagatttgttttgcattccttagaatggagggcaacattccacagctgccctggctgtgatgagtgtccttgcaggggccggagtaggagcactggggtgggggcggaattggggttactcgatgtaagggattccttgttgttgtgttgagatccagtgcagttgtgatttctgtggatcccagcttggttccaggaattttgtgtgattggcttaaatccagttttcaatcttcgacagctgggctggaacgtgaactcagtagctgaacctgtctgacccggtcacgttcttggatcctcagaactc

SEQ ID NO: 403 - DNA, 390 bp, Homo sapiens
gtagtcgcctctctttcagcagttacccagggtttttggagtctctggatgatttttacattcttagcagtggattgatattgctgcagaccacaaacagtgtgtttaataaaaccctgctaaagcaggtaatacccgagactctcctgtcctggcaaagagtccgtgtggccaatatgatggcagatagtggcaagaggtgggcagacatcttttcaaaatacaactctggcacctataacaatcaatacatggttctggacctgaagaaagtaaagctgaaccacagtcttgacaaaggcactctgtacattgtggagcaaattcctacatatgtagaatattctgaacaaactgatgttctacggaaaggatattggccctcctaca

SEQ ID NO: 404 - DNA, 372 bp, Homo sapiens
aggagattcagaagcacaaccacagcaagagcacctggctgatcctgcaccacaaggtgtacgatttgaccaaatttctggaagagcatcctggtggggaagaagttttaagggaacaagctggaggtgacgctactgagaactttgaggatgtcgggcactctacaaatgccagggaaatgtccaaaacattcatcattggggagctccatccagatgacagaccaaagttaaacaagcctccggaaactcttatcactactattgattctagttccagttggtggaccaactgggtgatccctgccatctctgcagtggccgtcgccttgatgtatcgcctatacatggcagaggactgaacacctcctc

SEQ ID NO: 405 - DNA, 619 bp, Homo sapiens
tcccgggtggagctggctgagtcgcgcgctctgctccacccgacggggctgtgtgtgctgggcctggctcgcggcgaaccgagatggcagagcagtcggacgaggccgtgaagtactacaccctagaggagattcagaagcacaaccacagcaagagcacctggctgatcctgcaccacaaggtgtacgatttgaccaaatttctggaagagcatcctggtggggaagaagttttaagggaacaagctggaggtgacgctactgagaactttgaggatgtcgggcactctacaaatgccagggaaatgtccaaaacattcatcattggggagctccatccagatgacagaccaaagttaaacaagcctccggaaactcttatcactactattgattctagttccagttggtggaccaactgggtgatccctgccatctctgcagtggccgtcgccttgatgtatcgcctatacatggcagaggactgaacacctcctcagaagtcagcgcaggaagagcctgctttggacacgggagaaaagaagccattgctaactacttcaactgacagaaaccttcacttgaaaacaatgattttaatatatctctttctttttct

SEQ ID NO: 406 - DNA, 499 bp, Homo sapiens
taagctcggaattcggctcgagggctccagctgagctcctgcttctactgaggacatacctcccagatgaggtggggcccccaaccccattccctgagcctggagcagagccccctctcactgtgggcttgctcaaagccctgctggagcagactggggctcaaggatggctgtcgggcccagttctaagcccatatgaggacatcctatgggaccccagcactccacccccgactccacctcgggacctatgactacccttcaggcatcagaacactcagggcctggaggcttgcttgggactggaggcttgcttggacagttcctctgtgtcactgacacaggaaatcatttctaggacacagtgatcagggaagggtgcctgggacttggagggtcccatgtatggacctgtgtatgcaatactgttctgtcatctggagctatttttaagatgtgtgtgttaaatatatacatagtttaatatataaaaaaaaaa

SEQ ID NO: 407 - DNA, 229 bp, Homo sapiens
ggctccagctgagctcctgcttctactgaggacatacctcccagatgaggtggggcccccaaccccattccctgagcctggagcagagccccctctcactgtgggcttgctcaaagccctgctggagcagactggggctcaaggatggctgtcgggcccagttctaagcccatatgaggacatcctatgggaccccagcactccacccccgactccacctcgggaccta

SEQ ID NO: 408 - DNA, 467 bp, Homo sapiens
ggaagttctgcgctggtcggcggagtatcaagtggccatggggagcctcagcggtctgcgcctggcagcaggaagctgttttaggttatgtgaaagagatgttggcctcatctctaaggcttaccagaagctctgatttgaagagaataaatggattttgcacaaaaccacaggaaagtcccggagctccatcccgcacttacaacagagtgcctttacacaaacctacggattggcagaaaaagatcctcatatggtcaggtcgcttcaaaaaggaagatgaaatcccagagactgtctcgttggagatgcttgatgctgcaaagaacaagatgcgagtgaagatcagctatctaatgattgccctgacggtggtaggatgcatcttcatggttattgagggcaagaaggctgcccaaagacacgagactttaacaagcttgaacttataaaagaaagctcgtctg

SEQ ID NO: 409 - DNA, 338 bp, Homo sapiens
ggaagttctgcgctggtcggcggagtagcaagtggccatggggagcctcagcggtctgcgcctggcagcaggaagctgttttaggttatgtgaaagagatgtttcctcatctctaaggcttaccagaagctctgatttgaagagaataaatggattttgcacaaaaccacaggaaagtcccggagctccatcccgcacttacaacagagtgcctttacacaaacctacggattggcagaaaaagatcctcatatggtcaggtcgcttcaaaaaggaagatgaaatcccagagactgtctcgttggagatgcttgatgctgcagagatcaagatgcgag

SEQ ID NO: 410 - DNA, 601 bp, Homo sapiens
tttgcacgatgccttccacatcccacggcgctgctgctgggggcagattggcctggggaggcagcacttgctctccagctcatctgggttgcttttccccgcagtggatatcacaggctaaaggggggggcagtccccaccatatttgagtctttctccaagttgcgccggacaaccaagaccaaaggacacagttacccacctggcccctctgaagtcagccggctcagacgatgcaggaagcgctgctccgagggccgagggcccacaactccattttctccacctccacctgctgatgtcacctgctttcctgtggaagaggcctcagcacctgccactttgccggcctccccagctgggaggctggagcctggccttagcagccccttttcagacctactgggccccttgggtgcccaggcagatgaagcaggctgcagcgcccagccttcaccagagcggcagccctcccctctcgaaccacggccagtctccccctcagcgtatatgctgcgcctgcccccacccgccggagcctacatccagaatgaacacagctaccaggtgggcagcgccttactctggaagcggcgagccg

SEQ ID NO: 411 - DNA, 52 bp, Homo sapiens
gccccttgggtgcccaggcagatgaagcaggctgcagcgcccagccttcacc

SEQ ID NO: 412 - DNA, 525 bp, Homo sapiens
cgtttcggtttctagggttgttacgaagctgcaggagcgagatggaggtggacgcaccgggtgttgatggtcgagatggtctccgggagcggcgaggctttagcgagggagggaggcagaacttcgatgtgaggcctcagtctggggcaaatgggcttcccaaacactcctactggttggacctctggcttttcatccttttcgatgtggtggtgtttctctttgtgtattttttgccatgacttgttcgctgatatctaaattaagaagttggttcttgagtgaattctgaaaatggctacaaacttcttgaataaagaagacaggactctcaatagaagaatttcacatctccaagggacccttcctttcattttacactttgttactaatttgcagaactctattaattgggtaggatttcacccattcctagctaagttcttaaaattaaaccctttggttcgtgtttaaaaactttcaaacatctgatggctttacaggggctgaatataaaagcatttg

SEQ ID NO: 413 - DNA, 604 bp, Homo sapiens
  misc_feature 12, 14, 18, 20, 24, 27, 29, 31, 33, 35, 54, 594, 595: n = A,T,C or G
ttcgaacccatncntttncnatcngananangntnctagttcttctgaagaccncatcgattcgtttcggtttctagggttgttacgaagctgcaggagcgagatggaggtggacgcaccgggtgttgatggtcgagatggtctccgggagcggcgaggctttagcgagggagggaggcagaacttcgatgtgaggcctcagtctggggcaaatgggcttcccaaacactcctactggttggacctctggcttttcatccttttcgatgtggtggtgtttctctttgtgtattttttgccatgacttgttcgctgatatctaaattaagaagttggttcttgagtgaattctgaaaatggctacaaacttcttgaataaagaagacaggactctcaatagaagaatttcacatctccaagggacccttcctttcattttacactttgttactaatttgcagaactctattaattgggtaggatttcacccattcctagctaagttcttaaaattaaaccctttggttcgtgtttaaaaactttcaaacatctgatggctttacaggggctgaatataaaagcatttgtacttannaaaaaaaaa

SEQ ID NO: 414 - DNA, 285 bp, Homo sapiens
ctctaacgtgggcaacagagaccctgtctcaaaaagaaaatattcctgttagccctaaaggctttacatgaggaatggtagaagtggtcttttgtttaaattagttgcattcagcatatatgaattgtcttaaatattttggggatactcccccgccttttaaacagggcataagatctggtaaactctctgtatatcttcctacctttcaaaatcgttcttagggttagtcaagtctggaatataattgctgactataaagttagcaattatgctttaaggtga

SEQ ID NO: 415 - DNA, 241 bp, Homo sapiens
atttacacttgatggctaataaagatggacagctaatgacagaattatttaatcgattagaaagtcagcatcatttccagatagaaaaggctctagttgagaaacttcagcaggattttgtagctgactggtgctctgagggagagtgcctagcagctattaactccacctataatacttcagggtatattttggatccacacactgctgttgcaaaagtggttgcagatagggtgcaaga

SEQ ID NO: 416 - DNA, 315 bp, Homo sapiens
cggcttctggaagagggggtgttgcggcagatccctgtagtgggcttcgtgctgaattggttttctccggtccaggcttcacagtagggaagaacttttaacttgacagcaggctctctggagtccacagaacccatatatgtctacaaagcacaaggtgcaggagtcacgctgcctccaacgccctcgggcagtcgcaccaagcagaggcttccaggccagaagccttttaaaaggtccctgcgaggttcagatgctttgagtgagaccagctcagtcagtcatattgaagacttagaaaaggtggagcgccta

SEQ ID NO: 417 - DNA, 164 bp, Homo sapiens
tggatcccccgggctgcaggaattcgaattctgtgtgtgtgtgtgtgtatgaatggtatatttattacattatttagaaagagaatgagtgtgttatgaggataatgttatatacagtctaagtggatgtttctgtttggcacagaatgtaggatttctgaaac

SEQ ID NO: 418 - DNA, 206 bp, Homo sapiens
tatatttattacattattttgaaagagaattagtgtgttatgtggataatgttatatacagccaaagtggatgtttctgtttggcaaggaaggtaggatttctgaaactcaggccttaaccaataggttggaagacaagaccaattgaagagttaggaaatgtgagtttttgttacttctgttattccagtcttggtttcattgtc

SEQ ID NO: 419 - DNA, 238 bp, Homo sapiens
  misc_feature 159, 227: n = A,T,C or G
agcagtgtacataatattccagtaggaaactgcttccaagtttaagcatgagctccccaaactggagaaaacatattttgctattctgagacaacaatcagaatacagactttggattccaggtcacagtttgctttttagacaaggtaaagcaaaganagccacattgtgccatcttcagctccagtggctttagcagtgactgtttgacataaaacatgtaaganttgcttgttgg

SEQ ID NO: 420 - DNA, 504 bp, Homo sapiens
cggcgtgcttgctgctggagggtgatggccctgcaaggctgtgggctccgacctcaccgggagtcgacagcgagaggttcgccgaagagcgaggttctgggcgagcgctgaacgccggccccaagcaccccgggtctttacacagtccgcgtccacagactctgacgaagacgtggatctgctctcgctttagctgctcgcggtcctccagatcatgtccgcgactcctgcgactccgcgcggaaaaaaaagtttgccaggcgtggactcaatgacctttccaagctgtgcgcctcgctgcctggaccgggtctgagcgcggctgcccaggttgacctttctgcgggagggctttctctacgtgctgttgtctcactgggtttttgtcggagccccacgccctccggcctctgattcctggaagaaagggttggtcccctcagcacccccagcatcccggaaaatggggagcaaggctctgccagcgcccatcccgctccaccc

SEQ ID NO: 421 - DNA, 814 bp, Homo sapiens
  misc_feature 38, 93, 94, 95, 422, 440, 467, 474, 508, 519, 529, 535,554, 557, 561, 565, 584, 594, 604, 619, 641, 655, 674, 679, 690,695, 702, 704, 706, 712, 716, 724, 734, 737, 740, 743, 780,781, 808, 813: n = A,T,C or G
cggggacggagctcggcgtgcttgctgctggagggttntggccctgcaaggctgtgggctccgacctcaccgggagtcgacagcgagaggttnnncgaagagcgaggttctgggcgagcgctgaacgccggccccaagcaccccgggtctttacacagtccgcgtccacagactctgacgaagacgtggatctgctctcgctttagctgctcgcggtcctccagatcatgtccgcgactcctgcgactccgcgcggaaaaaaaagtttgccaggcgtggactcaatgacctttccaagctgtgcgcctcgctgcctggaccgggtctgagcgcggctgcccaggttgaccttttctgcggaagggctttctctacgtgctgttgctcatgggtttttgtcggagccccaacgcccttccggncttttgattcctggaaanaaaaggggttggttccccttcaagcanccccaancattccccgggaaaaaatgggggagccaaagggnttttggccaanggccccaatncccggnttcaacccgttgggttggnaantttnaccnaaattaacttcctttcctncaaggcccgnggaaaaaacnttttcccgggccacngggggggaaccaaccttgcaanggggccttgtaccnggtcttcaaacggcgggtnccaanaacccttgccnccatngaaaccnantnggaacncctngggggttnttccccaatnggngcnccnaaaaaacaaccccggttccaaccatttaagggaaaannggcgggggggccccaagggcccttttnggacnt

SEQ ID NO: 422 - DNA, 375 bp, Homo sapiens
ctgacgaagacgtggatctgctctcgctttagctgctcgcggtcctccagatcatgtccgcgactcctgcgactccgcgcggaaaaaaaagtttgccaggcgtggactcaatgacctttccaagctgtgcgcctcgctgcctggaccgggtctgagcgcggctgcccaggttgacctttctgcgggagggctttctctacgtgctgttgtctcactgggtttttgtcggagccccacgccctccggcctctgattcctggaagaaagggttggtcccctcagcacccccagcatcccggaaaatggggagcaaggctctgcagcgcccatcccgctccaccgtcgctgcagctcccaattactcttctgcaggcg

SEQ ID NO: 423 - DNA, 405 bp, Homo sapiens
ggggacggagctcggcgtgcttgctgctggagggtgatggccctgcaaggctgtgggctccgacctcaccgggagtcgacagcgagaggttcgccgaagagcgaggttctgggcgagcgctgaacgccggccccaagcaccccgggtctttacacagtccgcgtccacagactctgacgaagacgtggatctgctctcgctttagctgctcgcggtcctccagatcatgtccgcgactcctgcgactccgcgcggaaaaaaaagtttgccaggcgtggactcaatgacctttccaagctgtgcgcctcgctgcctggaccgggtctgagcgcggctgcccaggttgacctttctgcgggagggctttctctacgtgctgttgtctcactgggtttttgtcggacc

SEQ ID NO: 424 - DNA, 139 bp, Homo sapiens
ctcgtgttcagctgtcagaataacagccaataaaaactacaggagcaaaacctctcaggaaggtgctttaaaaaagatgcatgaggaagaacaccatcaacaaatgtccatcttacaactgcaactgatacaaatgaat

SEQ ID NO: 425 - DNA, 273 bp, Homo sapiens
ttctggctgggaagcgcgattgtggctttaaaccaccatcatggtctagcaaagaggcaaagaccaagaccaccaagaagcgccctcagcgtgcaacatccaatgtgtttgccatgtttgaccagtcacagattcaggagttcaaagaggccttcaacatgattgatcagaacagagatggcttcatcgacaaggaagatttgcatgatatgcttgcttctctagggaagaatcccactgatgcataccttgatgccatgatgaatgaggccc

SEQ ID NO: 426 - DNA, 56 bp, Homo sapiens
gggaaccgccattctgcctgggaaccgccattctggccgggaaccgccattatgac

SEQ ID NO: 427 - DNA, 365 bp, Homo sapiens
ggcgcattcttacctgtcggggtgcggcgagtgtctcacctctctgcacttccaaggactcttgtcatctgccttaggcgggaaatgctgttgctggattgcaaccccgaggtggatggtctgaagcatttgctggagacaggggcctcggtcaacgcacccccggatccctgcaagcagtcgcctgtccacttagccgcaggaagcggccttgcttgctttcttctctggcagctgcaaacgggcgctgacctcaaccagcaggatgttttaggagaagctccactacacaaggcagcaaaagttggaagcctggagtgcctaagcctgcttgtagccagtgatgcccaaattgatttatgtag

SEQ ID NO: 428 - DNA, 119 bp, Homo sapiens
gagcggtggctgagaaatgtaaggattctggaatacatattccatgggactttccttccctctcctgcttcctcttttcctgctccctaacctttcgccgaatggggcagcaccactga

SEQ ID NO: 429 - DNA, 421 bp, Homo sapiens
  misc_feature 130, 185, 246, 256, 336, 361, 385, 412: n = A,T,C or G
tttttttttttttttttggaaataagtcaaagcattgtttatttatgacatatttacatatttacaaaactgattttactcaatacatcatcctgcgtaatatcataaaatgaacaccatatcctgggantaaaaatccatatttcttaataatttatgtatagcccaacttttagaacatagantattatcaatttggcttcccaaactacaaagtcctgtttataattttttctagccaaggancagagtaggntcaacaggcatattaaagtaatttagttaaccctgaggtaattactaacttggcataattttggaatggggtatatatancacactttccatctggcacttaggntacttattactattcacactaccnttttggtatttatccacctcaattttncaacttcct

SEQ ID NO: 430 - DNA, 481 bp, Homo sapiens
gggtagccgcttttcgtcgactcttaccggttggctgggccagctgcgccgcggctcacagctgacgatgggggaccccagcaagcaggacatcttgaccatcttcaagcgcctccgctcggtgcccactaacaaggtgtgttttgattgtggtgccaaaaaatcccagctgggcaagcataacctatggagtgttcctttgcattgattgctcagggtcccaccggtcacttggtgttcacttgagttttattcgatctacagagttggattccagctggtcatggtttcagttgcgatgcatgcaagtcggaggaaacgctagtgcatcttccttttttcatcaacatgggtgttccaccaatgacaccaatgccaagtacaacagtcgtgctgctcagctctatagggagaaaatcaaatcgctcgcctctcaagcaacacggaagcatggcactgatctgtggcttgatagttgtgt

SEQ ID NO: 431 - DNA, 136 bp, Homo sapiens
ggggtaagtttagaaatacggctgggcatgtccagccctgaccacggccagctctggagggctgtcctttggctgtacccacttggaagagaaagaaaaagaaaaaaaaaaaaaaaaaaaaaaatttttttttttt

SEQ ID NO: 432 - DNA, 578 bp, Homo sapiens
aaacaacaaacaccagaaaaattacctataccaatgatagcaaaaaaccttatgtgtgaactcgatgaagactgtgaaaagaatagtaagagggactacttaagttctagttttctatgttctgatgatgatagagcttctaaaaatatttctatgaactctgattcatcttttcctggaatttctataatggaaagtccattagaaagtcagcccttagattcagatagaagcattaaagaatcctcttttgaagaatcaaatattgaagatccacttattgtaacaccagattgccaagaaaagacctcaccaaaaggtgtcgagaaccctgctgtacaagagagtaaccaaaaaatgttaggtcctcctttggaggtgctgaaaacgttagcctctaaaagaaatgctgttgcttttcgaagttttaacagtcatattaatgcatccaataactcagaaccatccagaatgaacatgacttctttagatgccaatggatatttcgtgtgcctacagtggttcatatcccatggctataacccctactcaaaaaagaagatcctgtatgccacatc

SEQ ID NO: 433 - DNA, 229 bp, Homo sapiens
  misc_feature 35, 37: n = A,T,C or G
gcctaggtgcccaggctatgatgagtctgcttttnanggaggtagggaatgacatcttccttggacccaaagcttaaaagtaatgtatgctttgctgaccactgtttgttaggccttaaacaacattcactgtggtggtatcaggcacactgctatgtgcatcaattatttttttgctttccaaacagaatctctggggcacaagttttacactcaagctaagtataac

SEQ ID NO: 434 - DNA, 503 bp, Homo sapiens
tggtacgcctgcaggtaccggtccggaattcccgggtcgacccacgcgtccggcgtcatggagctgacctggttcccatctactcctttggagagaatgaagtgtacaagcaggtgatcttcgaggagggctcctggggccgatgggtccagaagaagttccagaaatacattggtttcgccccatgcatcttccatggtcgaggcctcttctcctccgacacctgggggctggtgccctactccaagcccatcaccactgttgtgggagagcccatcaccatccccaagctggagcacccaacccagcaagacatcgacctgtaccacaccatgtacatggaggccctggtgaagctcttcgacaagcacaagaccaagttcggcctcccggagactgaggtcctggaggtgaactgagccagccttcggggccaattccctggaggaaccagctgcaaatcacttttttgctctgtaaatttggaagtgtcatgggtgtct

SEQ ID NO: 435 - DNA, 248 bp, Homo sapiens
gcgtcatggagctgacctggttcccatctactcctttggagagaatgaagtgtacaagcaggtgatcttcgaggagggctcctggggccgatgggtccagaagaagttccagaaatacattggtttcgccccatgcatcttccatggtcgaggcctcttctcctccgacacctgggggctggtgcctactccaagcccatcaccactgttgtgggagagcccatcaccatccccaagctggagcacca

SEQ ID NO: 436 - DNA, 457 bp, Homo sapiens
atcttgtctcttttcatcgtgatggtgtgatgctgacgagaatatcttatgctttcttcagcctgttgcaatctgagccaatgattttctttgcactgatcctttctactctggagagaagctcttttgacacagatcctgccccgtttaatagactccagctgctggcactgccttctgagttctttcacttccgaattcttatcgtcctgcagccccaccacagtcaatgactaagttcctctggactttcacatggatcgtaatagacaacttcatcctgtttttcttaccagaccctaaaatgtgcctccaagacagtcgtgggaacagtatggagccagcagcagaagccactcacgaaccaatggaggagaacaactcagaaacagacccaagtcaatctaaggtttaacttttataagtctttcaagagagtccaactgtgtagtaagca

SEQ ID NO: 437 - DNA, 589 bp, Homo sapiens
gcttccaggtctccttccagcatccacacaagtacctgctccactacctggtttccctccagaactggctgaaccgccacagctggcagcggacccctgttgccgtcaccgcctgggccctgctgcgggacagctaccatggggcgctgtgcctccgcttccaggcccagcacatcgccgtggcggtgctctacctggccctgcaggtctacggagttgaggtgcccgccgaggtcgaggctgagaagccgtggtggcaggtgtttaatgacgaccttaccaagccaatcattgataatattgtgtctgatctcattcagatttataccatggacacagagatcccctaaggtcctggcccaggcctgcccaaagagaagcccaggatggtcggctgcctggggacattgtcaccacgtcgccatgacggctggtccccacaggaccagctgggaggactggttgtgctgctggagaagggctggagaaggcaatggcatgctgccgctttgccagtccctaaaagtcgcggtgcaggtgatggtgggagccgcgcctccagcgggcaggccgggagtgtactgtgtgc

SEQ ID NO: 438 - DNA, 241 bp, Homo sapiens
cgcttccaggtctccttccagcatccacacaagtacctgctccactacctggtttccctccagaactggctgaaccgccacagctggcagcggacccctgttgccgtcaccgcctgggccctgctgcgggacagctaccatggggcgctgtgcctccgcttccaggcccagcacatcgccgtggcggtgctctacctggccctgcaggtctacggagttgaggtgcccgccgaggtcgagg

SEQ ID NO: 439 - DNA, 221 bp, Homo sapiens
ttcagctctgcaaacactgtcacatcctttcctggaagggcactgaccatccgtgcactgccaataacccagagagctgctccgtttcactttcaccccaggactttatcaacttgttcaagttctgaatcccagcacatgacaacacttcagaagggtccccctgctgactggagagctgggaatatggcatttggacacttcatttgtaaatagtgtac

SEQ ID NO: 440 - DNA, 228 bp, Homo sapiens
  misc_feature 191: n = A,T,C or G
gagctttcttaataaccgtacttctcaaaatcagagttttactgtttcaataaatgttcaccctagattgtaagttttttgttgttgagccctagatttttttctactagtgtaaatctgtattccctccaagtatggtgataaggggactgagtcttatttacatttgtacaatcactactttacctgtngtatttgcagtaagtcttttgagccctattaaacctg

SEQ ID NO: 441 - DNA, 531 bp, Homo sapiens
tttcttaataaccgtacttctcaaaatcagagttttactgtttcaataaatgttcaccctagattgtaagttttttgttgttgagccctagatttttttctactagtgtaaatctgtattccctccaagtatggtgataaggggactgagtcttatttacatttgtacaatcactactttacctgttgtatttgcagtaagtcttttgagccctattaaacctgtcaattttcttgtcctgtcagaaaactgagattttggctcaaaaatggatgttattaacaaaggggaacaatatagatgtcttagtacaaagaaaatgaaatgtaagaggagattgtctggagttcaggggatagagtgtcaagtcttaaatggttacatctttttgctaagtgttactcagaatatagttacaaatatggtacttaaatatctagctgaaatttgtttgtcccatgagcttctcacatgagtctactgggcaattttatgtgagttttggtcaaaattggtaatctcttttatctt

SEQ ID NO: 442 - DNA, 147 bp, Homo sapiens
  misc_feature 112: n = A,T,C or G
aacttgttacccaataacaatttaatgttaaatttggctttcttctgtgtcccagcctcttaaattaatagatgggcctttccattatcattatgaccggacattgtaaagnacttaaggtaacacccagttttctattacttgccc

SEQ ID NO: 443 - DNA, 518 bp, Homo sapiens
acctgaagaatattagaagaaattgtgcaccctccacaaaacatacaaagtttaaaagtttggatctttttctcagcaggtatcagttgtaaataatgaattaggggccaaaatgcaaaacgaaaaatgaatcatctacatgtagttagtaatttctagtttgaactgtaattgaatattgtggcttcatatgtattattttatattgtacttttttcattattgatggtttggactttaataagagaaattccatagtttttaatatcccagaagtgagacaatttgaacagtgtattctggaaaacaacacactaactgaacagaagtgaatgcttatatatattatgatagccttaaacctttttcctctaatgccttaactgtcaaataattataaccttttaaagcataggactatagtcagcatgctagactgagaggtaaacactgatgcaattagaacaggtactgatgctgtcagtgtttaacactatgtttagctgtgtttatgctat

SEQ ID NO: 444 - DNA, 76 bp, Homo sapiens
gctgctcatgagcagcatggacgacctgatacgccactgtaacgggaagctgggcagctacaaaatcaatggccgg

SEQ ID NO: 445 - DNA, 308 bp, Homo sapiens
gagcattatgagcattatgtcagaatagaatagaattggggttcgatcttaacaggccagaaatgcctgggtttttttggtttgtttttgtttttgtttttttatcaaatcctgcctgactgtctgcttgttttgcctaccatcgtgacatctccatggctgtaccaccttgtcgggtagcttatcagactgatgttgactgttgaatctcatggcaacaccagtcgatgggctgtctgacattttggtatctttcatctgaccatccatatccaatgttctcatttaaacattacccagcatcattg

SEQ ID NO: 446 - DNA, 530 bp, Homo sapiens
tgtgttaatgttttctagcatgtactctggtttcaacagacacaaatttatatgttaacccagttttcttgccgttctgtaagtgttttattcttagtgtgatttttttccattgggatgtttttgattgaacttgttcattttgttttgcttgggaggaaaataaacaattttacttttttcctttaggagcattatgagcattatgtcagaatagaatagaattggggttcgatcttaacaggccagaaatgcctgggtttttttggtttgtttttgtttttgtttttttatcaaatcctgcctgactgtctgcttgttttgcctaccatcgtgacatctccatggctgtaccaccttgtcgggtagcttatcagactgatgttgactgttgaatctcatggcaacaccagtcgatgggctgtctgacattttggtatctttcatctgaccatccatatccaatgttctcatttaaacattacccagcatcattgtttataatcagaaactctggtccttctgtctgg

SEQ ID NO: 447 - DNA, 104 bp, Homo sapiens
ggacgtgcctggaaccacctcgtccacgtccacgtccacctgggggcctcgggaggctaggcccctcctcaaaggcccaccagcccggcgctcatgctgagccc

SEQ ID NO: 448 - DNA, 417 bp, Homo sapiens
tatctttcatctgaccatccatatccaatgttctcatttaaacattacccagcatcattgtttataatcagaaactctggtccttctgtctggtggcacttaaagtcttttgtgccataatgcagcagtatggagggaggattttatggagaaatggggatagtcttcatgaccacaaataaataaaggaaaactaagctgcactgtgggttttgaaaaggttattatacttcttaacaattctttttttcagggacttttctagctgtatgactgttacttgaccttctttgaaaagcattcccaaaatgctctattttagatagtttaacattaaccaacataattttttttagatcgagtcagcataaatttctaagtcagcctctagtcgtggttcatctctttcacctgcat

SEQ ID NO: 449 - DNA, 630 bp, Homo sapiens
ttttttttttttttttttttttggaatcgcaagaattcccaggccctctttttatttacagtgataccaaaccatccacttgcaaattctttggtctcccatcagctggaattaagtaggtactgtgtatctttgagatcatgtatttgtctccactttggtggatacaagaaaggaaggcacgaacagctgaaaaagaagggtatcacaccgctccagctggaatccagcaggaacctctgagcatgccacagctgaacacttaaaagaggaaagaaggacagctgctcttcatttattttgaaagcaaattcatttgaaagtgcataaatggtcatcataagtcaaacgtatcaattagaccttcaacctaggaaacaaaatttttttttctatttaataatacaccacactgaaattatttgccaatgaatcccaaagatttggtacaaatagtacaattcgtatttgctttcctctttcctttcttcagacaaacaccaaataaaatgcaggtgaaagagatgaaccacgactagaggctgacttagaaatttatgctgactcgatctaaaaaaaattatgttggttaatgttaaactatctaaaatagagcattttgggaatgct

SEQ ID NO: 450 - DNA, 596 bp, Homo sapiens
tttttttttttttttttttttttggggtaaaagttatatcttattgccatgctacaaaatgtatgaagttggcactgatagggagaaatagagaacaaagggtgggaagggatagagggaaaattatgttgttacatatacaacaaggttttattttaattaacagtggttacgttttgccaatattaaaaatgcaaaccaaaatttaaaatgctgatctgaaacagcattaagatacaatgtatgcatagtacagtatcacttatgtctttttattagagaaatatggaatgtttataaaagaaattaaccatgggggtaaaattcatatttcatatacaatttggcaatggtagtcccactgttggacaattttttataaaagaaaaaattaaaaatctaataagctacctttatacaaagttgctatatttatgcctttacgtaggaaaaaaacatttataatgcaaattaggacatacaatagtcttacaatactatacaatgtaatgaaaataaaacataacacaaagtttgtcctttataaaatgtatattttgcattactaatgcaaatgtggcacactggtgactact

SEQ ID NO: 451 - DNA, 559 bp, Homo sapiens
tggcgggttgctttccaaaatggcgcgggtgctgaaggctgcagccgcgaatgccgtagggcttttttccagacttcaagctcccattccaacagtaagagcttcttccacatcacagcccttggatcaagtgacaggttctgtgtggaacctgggtcgactcaaccatgtagccatagcagtgccagatttggaaaaggctgcagcattttataagaatattctgggggcccaggtaagtgaagcggtccctcttcctgaacatggagtatctgttgtttttgtcaacctgggaaataccaagatggaactgcttcatccattgggacgtgacagtccaattgcaggttttctgcagaaaaacaaggctggaggaatgcatcacatctgcatcgaggtggataatattaatgcagctgtgatggatttgaaaaaaaaagaagatccgcagtctaagtgaaggggtcaaaataggagcacatggaaaaccagtgatttttctccatcctaaagactgtggtggagtccttgtggaactggagcaagcttgacttatatt

SEQ ID NO: 452 - DNA, 638 bp, Homo sapiens
tggcgggttgcgttccaaatggcgcgggtgctgaaggctgcagccgcgaatgccgtagggcttttttccagacttcaagctcccattccaacagtaagagcttcttccacatcacagcccttggatcaagtgacaggttctgtgtggaacctgggtcgactcaaccatgtagccatagcagtgccagatttggaaaaggctgcagcattttataagaatattctgggggcccaggtaagtgaagcggtccctcttcctgaacatggagtatctgttgtttttgtcaacctgggaaataccaagatggaactgcttcatccattgggacgtgacagtccaattgcaggttttctgcagaaaaacaaggctggaggaatgcatcacatctgcatcgaggtggataatattaatgcagctgtgatggatttgaaaaaaaaagaagatccgcagtctaagtgaaggggtcaaaataggagcacatggaaaaccagtgatttttctccatcctaaagactgtggtggagtccttgtggaactggagcaagcttgacttatatttgcaagcaactaaattaattgacctgaaaaagcctatcaaatactatcaaaatgtactatgacattgagtccttcactgc

SEQ ID NO: 453 - DNA, 57 bp, Homo sapiens
gactacatttggggatgatgcattcctttaagattgaatgattctgcccttgggcag

SEQ ID NO: 454 - DNA, 538 bp, Homo sapiens
gccgggctgctaattctgtttaattgttcctgggctaaaaagaattagaaggaagctgtctgtttcccactgcggttatgtttcagtaaattagacgtactttctgatgaatactaattagccactgagcatttgcacccactgtctttgctggttgtgtgcagaacagctgccaagttgcccaagaccctcgctatcccatccccctctcttgctttccacttttgggcttcctttgcctagattagaagagatttcagttccgagaaagtaaaaggtgatccaaggaagtaatcaccgagtgtctcatggtttttccttgttgacaaaattcaaaactcacacatgtgtagtctaatgatagcgctaggatttaaagaaagtgttttagtgctgtgcttatttaggactacatttggggatgatgcattcctttaagattgaatgattctgcccttgggcagagctcccaattagggaggattaggtaagctttttgtggcgatgggtaataccattcttttcctcattgtgcctg

SEQ ID NO: 455 - DNA, 548 bp, Homo sapiens
tgaatcagtaggaatgtggggaagggagtgaggggagaccccctccttgactcagcagtggtgacggtcggtgtgtcctgcagacctgaagccaagatcaagggggcttgagcaccaggagcccccgcagttgctgaatgaccagcggagggcaggtgccagcctgtggcaaaataggaaagaaaaggacaggatggggacttcaccatttttttcagccttaaattgttccttaaaccttcatgtccttttctctaatgtgtgttcttgtttggtaaaataaaaaagtttgtaaccctgagttctctaaagatatacattcttttttactggtttgtgaagtcagaaggatgagagctgctatttcttggaaccgtgcaataaatattagcatattcagtctcggttctgcctagaggacctatttgcttttctttatctcgtaacccataactcacaggacattaaccagggtgtccaagaacagtctgggaaagttttgataattacttcagcattgctgtgtgatgggagacattgttttaaaa

SEQ ID NO: 456 - DNA, 354 bp, Homo sapiens
tcagtgggagtgaatcagtaggaatgtggggaagggagtgaggggagaccccctccttgactcagcagtggtgacggtcggtgtgtcctgcagacctgaagccaagatcaagggggcttgagcaccaggagcccccgcagttgctgaatgaccagcggagggcaggtgccagcctgtggcaaaataggaaagaaaaggacaggatggggacttcaccatttttttcagccttaaattgttccttaaaccttcatgtccttttctctaatgtgtgttcttgtttggtaaaataaaaaagtttgtaaccctgagttctctaaagatatacattcttttttactggtttgtgaagtc

SEQ ID NO: 457 - DNA, 570 bp, Homo sapiens
cttttataggattcatttaaaggtgaataaaataatgaatgtgaaactcatattagagcttaacatatagtagtaatgatttataaaatatttgcctcccttagaccagagcagctactaaatttgattttaataataagataaacaaattaataagatcacaaagttgttatgtaataacataaacagctgtgttaaaattagtagtgacccatatcaaagaaacacaattacaaagagattaagaaggataatatttaaagtgtagctttactcagtcttttgtgtgaaggtattcttagggataaaacaatgtatttggaagctgctggaagaatatggtgcaaagaatatttttaaatgcttgtgaatgttctgtaaccacaaacatagatacataacagatcaaagacatattttagactgccatgtggacttaaatcatgggaggcggaagagtggctccccaaagaggactatatcgtaataccagaacttgtgaatatattactttaagtggcaaaagggactttacagatgtgattaaaattaaggaccttgaaatggggg

SEQ ID NO: 458 - DNA, 540 bp, Homo sapiens
aactagacttcttttataggattcatttaaaggtgaataaaataatgaatgtgaaactcatattagagcttaacatatagtagtaatgatttataaaatatttgcctcccttagaccagagcagctactaaatttgattttaataataagataaacaaattaataagatcacaaagttgttatgtaataacataaacagctgtgttaaaattagtagtgacccatatcaaagaaacacaattacaaagagattaagaaggataatatttaaagtgtagctttactcagtcttttgtgtgaaggtattcttagggataaaacaatgtatttggaagctgctggaagaatatggtgcaaagaatatttttaaatgcttgtgaatgttctgtaaccacaaacatagatacataacagatcaaagacatatttgagactggcatgtggacttaaatcatgggaggcggaagagtggctccccaaagaggactatatcgtagtaccagaacttgtgaatatattactttaagtggcaaaagggac

SEQ ID NO: 459 - DNA, 622 bp, Homo sapiens
acttaagattttttcaatgtaagaaaaatgcaatgaaataatagctgcaaatacccactactaacaattgcttggccttcttatatagacctcccgaggttctcatcttttacatttcaggagtagaatcagttaaaaactaatctttatatgtaagggatgagagagagaaagaggagggtatgtgtatgcacacatgtgtgtgtgtgtggtgggtagtaattttaattcaatgatttactagagttcgatgtcgtttgctgataaatgaagcaggaggaagagccaggtttggaggggacgagagaatgagttccatttgtctcatatagaagttgaagtaactgagtgatgatgggtagagatgtccctcaggggtagccacagtattttatttactttttattcaccacatgcagcaaggagctttgttctccaaaatgctgtcaattatttttctaaattacaggtttgattgcttcactgtattttcatgtctcattactacctttacgcttaaaaccagaaactgtgccacagcgttaaagattctgctaacttttaaaatacagaactctggagatgccataattagattgcagatttatgagtcttctggata

SEQ ID NO: 460 - DNA, 378 bp, Homo sapiens
acaatgggtttgttctctgccttataaattgggggattctagaggagtctgcttttctcccaagaaggacctcttcttttcttgcttttcatatgctctccttgagatatcttgggtattctcatggctttaaatagcacttatatccagaagactcataaatctgcaatctaattatggcatctccagagttctgtattttaaaagttagcagaatctttaacgctgtggcaaagtttctggttttaagcgtaaaggtagtaatgagacatgaaaataccgtgaagccatcaaacctgtaatttaaaaaaataattgacagcattttggagaacaaagctccttgctgcatgtggggaataaaaagtaaataaaata

SEQ ID NO: 461 - DNA, 396 bp, Homo sapiens
ccttctgctctacgagaactatgggcagtcggaaacgggactaatttgtgccacctactggggaatgaagatcaagccgggtttcatggggaaggccactccaccctacgacgtccaggtcattgatgacaagggcagcatcctgccacctaacacagaaggaaacattggcatcagaatcaaacctgtcaggcctgtgagcctcttcatgtgctatgagggtgacccagagaagacagctaaagtggaatgtggggacttctacaacactggggacagaggaaagatggatgaagagggctacatttgtttcctggggaggagtgatgacatcattaatgcctctgggtatcgcatcgggcctgcagaggttgaaagtgctttggtggagcaccc

SEQ ID NO: 462 - DNA, 529 bp, Homo sapiens
ttttttttttttttttttttttttttcggtagaaatggggttttaccatgttgcccaggctagtctcgaactcctgggcttaagcaatccacacacctcgcttccaaaaaagctggggttacaggtgtgagccatcacacccagcctaatatacaatctcaaatattttgttttaaatcattacttactgaactataaagtaaaactaatttttagacagcattttaatacatattttactttttaaaggttataaagaaaacactaacaatatggaaaatgcatatttaaagaaaattgaaatcaaatataatcttatggctcaaaatcattagtgttaatattttgatacctaccttccccatcttttgcctacgaatactgggttaagagtttttaaatagttttgtccttgctttgtaattttcgtatgttctcacaaaagagaagctgaggaagcatttggctattgggaaaattaattaatagatgttaacttaccaagatatactataatagattagacagc

SEQ ID NO: 463 - DNA, 485 bp, Homo sapiens
tttaaagtaaatgactcatgttgaggaaagaggttattacctaaatctggactgcggcctaaggaaattcccttaacctctattctggtttcctatttcaaaatggttgtgtaggaggctaatggaagttagttggttgctatgatccaaaaactctatgggtgaaaatttaaagtacagatttcttatttaatcgttaaacagctttagttgtgagttctatgtcctggtataatggatcctgattattaatgcattaaatatgcattcagtgaattcaaatgttgctaattattcttttaccaatcaaagaaaactcaaagcatgggattaagaggggttggccaaaagtatttggaccaggttgcataccaggaccatgaagaaattgagaacagagcctacatcttttatactatggctcaaagcaagggctgttggaatgtgctgcttctccaaagtaggacttatgaaaaaatgagggt

SEQ ID NO: 464 - DNA, 576 bp, Homo sapiens
tatcagcatctgtagaggagaaagcagaataagcactggggtatttgatagacttgagaataagagaaccccaaagttgtcaataggtatttgctagaaagttcagtgggtcagggtgggaatagcagctgaaattggcagggattttgactattcaaataatgggtgagtagaagggatctgtggaatagccattatgacctcttgaaaccaggcaactagggggtcccttctagaatgatgctgcgtacctaagaaattcagtagggagtggagtcaaaatgatcagaaaagatagagatagttgtggcaaaagatgatctaagagtgtgtgtgtatgtgtgtgagtgagagagagaaatctcaagaaatagtggctatggtgttgaacactacatgaaagcaacctaaaacagctgtgtgaagttagaaaaggtactctggaccatattgccctgtaaaagctcaggaaaactaattttgcataaacataagcaacaggaaattattgctgtcaaatctcattcagagttattgtacaaaaaaagagacaagaatccctatagacaatgaaag

SEQ ID NO: 465 - DNA, 459 bp, Homo sapiens
ttatctaacgtttctaacaggggtgttaatgatattagcagcaagagctatgagaaataactttagacattatttcattgaaccttcccaactgaaattattttatgatgttataacatggatagtaactcaagtagcaataagttacacagttgtgccatttgtgcttctttctataaaaccatcactcacgttttacagctcctggtattattgcctgcacattcttggtatcttagtattatcgttgttgccagtgaaaaaaactcaaagaaggaagaatacacatgaaaacattcagctctcacaatccaaaaagtttgatgaaggagaaaattctttgggacagaacagtttttctacaacaaacaatgtttgcaatcagaatcaagaaatagcctcgagacattcatcactaaagcagtgatcgggaaggctctgagggctgttttttttttt

SEQ ID NO: 466 - DNA, 250 bp, Homo sapiens
tatacccaggatattatctaacgtgtctaacaggggtgttaatgatattagcagcaagagctatgagaaataactttagacattatttcattgaaccttcccaactgaaattattttatgatgttataacatggatagtaactcaagtagcaataagttacacagttgtgccatttgtgcttctttctataaaaccatcactcacgttttacagctcctggtattattgcctgcacattcttggtatctt

SEQ ID NO: 467 - DNA, 509 bp, Homo sapiens
atactttatctattttcgggcaacttgcttccctcatgaaccatggacatctcaatgtgccattacacacaggagttatatgttaggtattgttgtcccattttacagaagagaatccgcaaggttcacagagtgaatcataggcataaagtccttcaggtggtaaatggcaaggctggtgttccaaccagtcttctctggctccagggactggctccttcagactacatttcaccagctgcctccaggaacagaagacgggaattcacctttcatgcgacatataccagaaacgtggacctcagccaccctgggtcctatttgatccccagggccttcatttggccctcgaataaaaaccttatttttttatctccttacctttcccagaattcatagtaggacttggctggtgaaaggctggttgctgagaaggctacagtgtggctaggctgcagttccctgttattacattgccccaggtattaatattgtatatttaggcagct

SEQ ID NO: 468 - DNA, 554 bp, Homo sapiens
ggatttcaaatctgagatgatactttatctattttcgggcaacttgcttccctcatgaaccatggacatctcaatgtgccattacacacaggagttatatgttaggtattgttgtcccattttacagaagagaatccgcaaggttcacagagtgaatcataggcataaagtccttcaggtggtaaatggcaaggctggtgttccaaccagtcttctctggctccagggactggctccttcagactacatttcaccagctgcctccaggaacagaagacgggaattcacctttcatgcgacatataccagaaacgtggacctcagccaccctgggtcctatttgatccccagggccttcatttggccctcgaataaaaaccttatttttttatctccttacctttcccagaattcatagtaggacttggctggtgaaaggctggttgctgagaaggctacagtgtggctaggctgcagttccctgttattacattgccccaggtattaatattgtatatttaggcagctgttctcatccgtgcctggcagtgaaa

SEQ ID NO: 469 - DNA, 537 bp, Homo sapiens
attctgaccccattgtgcaccttagtcatggcaaactttccagttgctccttgccaaaactcaagaataaaagggcccaagctagagaggctgtcctcacaagcatcagctgctgggggcttccactcattttcctctgaaacaacagagaaagagaccatctctcattcgcagagcagcccaaggccttctgaggagactgtgagtctcctctaagtcatttctctctgctttgtagcagtggagctaccaagggtgagatgagcaggttgagaggcctctgaagcctgctgggcacaatgctctgtgataagtttcagctccactggagcttatcatccaccagcaatcgacttcatggctgctgctcagaggccctaggtgctgcgctgctcactgccctcacgtctctgggacttccacacataaagccatctctttccattgcactatggcacttgtagggaggatcccacacttagggcccaaaatgagaccatttgagtcaaatttctaattgtctttcaaattttatta

SEQ ID NO: 470 - DNA, 492 bp, Homo sapiens
attctgaccccattgtgcaccttagtcatggcaaactttccagttgctccttgccaaaactcaagaataaaagggcccaagctagagaggctgtcctcacaagcatcagctgctgggggcttccactcattttcctctgaaacaacagagaaagagaccatctctcattcgcagagcagcccaaggccttctgaggagactgtgagtctcctctaagtcatttctctctgctttgtagcagtggagctaccaagggtgagatgagcaggttgagaggcctctgaagcctgctgggcacaatgctctgtgataagtttcagctccactggagcttatcatccaccagcaatcgacttcatggctgctgctcagaggccctaggtgctgcgctgctcactgccctcacgtctctgggacttccacacataaagccatctctttccattgcactatggcacttgtagggaggatcccacacttagggcccaaatg

SEQ ID NO: 471 - DNA, 509 bp, Homo sapiens
aagacattcaaattagccaccactggagtagatgacctaaaagttcttacaactctcaattatacccagtgatgtctcgattagcacttattataaaaattaaaatttataattcaacatttataccatccagaaaaagttaaaatatattaatagcctatttctcttcaataaagcgtatatataactctatttgttaatgtttctattctccatgacattctgtttatagataagccctatgctatttctagtcaagtgctaatctcttgaatgaagctgaattaggtagtcaactactagatgtatcctgaaaagcaagtaatgtgtatatttcatttattttatacataagagctacagactgttgtcacaatcttttcaagggctattaaattcattattttaactaacatttttgaacatctgtcttatgttgttaattgaggacatttctgaatgtataacaacataagaataatagttgttaaacttcaaagagatgacag

SEQ ID NO: 472 - DNA, 649 bp, Homo sapiens
caaattagccaccactggagtagatgacctaaaagttcttacaactctcaattatacccagtgatgtctcgattagcacttattataaaaattaaaatttataattcaacatttataccatccagaaaaagttaaaatatattaatagcctatttctcttcaataaagcgtatatataactctatttgttaatgtttctattctccatgacattctgtttatagataagccctatgctatttctagtcaagtgctaatctcttgaatgaagctgaattaggtagtcaactactagatgtatcctgaaaagcaagtaatgtgtatatttcatttattttatacataagagctacagactgttgtcacaatcttttcaagggctattaaattcattattttaactaacatttttgaacatctgtcttatgttgttaattgaggacatttctgaatgtataacaacataagaataatagtttttaaacttcaaagagatgacaggttaatgagtaaaggagaaatatgaaatatcacagaattccttgacactaaatgatgttttgcaaatactgaacagaatgatgtttgtaaactttccactggttttcaagagtcccaaaacattaggaaaatgtacatcacctaactt

SEQ ID NO: 473 - DNA, 494 bp, Homo sapiens
atatcagaagtaaaacaatttttcttgttgactgctttggtaaaaaacagtttgatggatagttttacatttcactggactagataaaaaatggtgctaatatttatgtagcttgatgctatagttgctttggtatcaaacttaatacctaacccatataagatccttattatataattttgtgatcagtaaaatgatattttaaagagtgatcttaaaaatatgacctggtcattgcacaacgtttgcatttgaaatgaatttttgtactatagggtggatatggagttattcagtgcaagtgtgtgcttaatatcaaaccctatgcaaggagctatgtctagatttttggtccgaatttgcctccttcaagcctactagtgtgagatggaaaaaaatcgattgctcttttaatattatttccattttgaaattctcgacacttgaatgaaggcagtagaagcctctttttggatttctcttctaataacaaa

SEQ ID NO: 474 - DNA, 630 bp, Homo sapiens
aaaacatttttcttgttgactgctttggtaaaaaacagtttgatggatagttttacatttcactggactagataaaaaatggtgctaatatttatgtagcttgatgctatagttgctttggtatcaaacttaatacctaacccatataagatccttattatataattttgtgatcagtaaaatgatattttaaagagtgatcttaaaaatatgacctggtcattgcacaacgtttgcatttgaaatgaatttttgtactatagggtggatatggagttattcagtgcaagtgtgtgcttaatatcaaaccctatgcaaggagctatgtctagatttttggtccgaatttgcctccttcaagcctactagtgtgagatggaaaaaaatcgattgctcttttaatattatttccattttgaaattctcgacacttgaatgaaggcagtagaggcctctttttggatttctcttctaataacaaaactttatttagggaaggtttccctgtgctatcgtaagtttgttttgagcactgcattcactttaaaattctggaggaacaaaggctgggcacataatcacaaagcccaggccacacaataattccggggttgtattttctaagaacta

SEQ ID NO: 475 - DNA, 156 bp, Homo sapiens
gggggagataaggcaaagaggcacttttggatttctccatctgagcagctctgtgattcattatctgttctagaaagcagcacacgcagttccagcaaaaaaaaaaaaaaaaaaaaatttttttttttttccccccttttttttttttttttcccc

SEQ ID NO: 476 - DNA, 579 bp, Homo sapiens
attccgttgctgtcggcggccgggtcccgatgagcctcctgttgcctccgctggcgctgctgctgcttctcgcggcgcttgtggccccagccacagccgccactgcctaccggccggactggaaccgtctgagcggcctaacccgcgcccgggtagagacctgcgggggatgacagctgaaccgcctaaaggaggtgaaggctttcgtcacgcaggacattccattctatcacaacctggtgatgaaacacctccctggggccgaccctgagctcgtgctgctgggccgccgctacgaggaactagagcgcatcccactcagtgaaatgacccgcgaagagatcaatgcgctagtgcaggagctcggcttctaccgcaaggcggcgcccgacgcgcaggtgccccccgagtacgtgtgggcgcccgcgaagcccccagaggaaacttcggaccacgctgacctgtaggtccgggggcgcggcggagctgggacctacctgcctgagtcctggagacagaatgaagcgctcagcatcccgggaatacttctcttgctgagagccgatgcccgtccccggg

SEQ ID NO: 477 - DNA, 472 bp, Homo sapiens
ggcttagcggataacaatttcacacaggagctagcagacaccacaagataccaacagagcttctgaaacagatacccatagcattggagagaaaaacagctcacagtctgaggaagatgatattgaaagaaggaaagaagttgaaagcatcttgaagaaaaactcagattggatatgggattggtcaagtcggccggaaaatattccccccaaggagttcctctaaacacccgaagcgcacggccaccctcagcatgaggaacacgagcgtcatgaagaaagggggcatattctctgcagaatttctgaaagatttccttccatctctgctgctctctcatttgctggccatcggattggggatctatattggaaggcgtgtgacaacctccaccagcaccttttgatgaagaactggagtctgacttggttcgttagtggattacttctgagcttgcaacatagctcactg

SEQ ID NO: 478 - DNA, 355 bp, Homo sapiens
tctacacttaaagctttggagcaattcccatcgaccagagttggtccgaccagccttggaaaggtcactgaaaaatcttcaattggactatggtgacctctatcttatacattttccattgtctgcaaagccaggtgaggaagtgatcccaaaagatgacaatggaaaaatactatttgacacagtggatctctgtgccacatgggaggccatggagaagtgtaaagatgcacgattggccaagtccatcggggtgtccaacttcaaccacaggctgctggagatgatcctcaacaagccagggctcaagtacaagcctgtctgcaaccaggtggaatgtcatccttacttcaac

SEQ ID NO: 479 - DNA, 510 bp, Homo sapiens
aagactactgaatctgctaccaaaacagtgaatcagtgagtcgatgttctattttttgttttgtttcctcccctatctgtattcccaaaaattactttggggctaatttaacaagaactttaaattgtgttttaattgtaaaaatggcagggggtggaattattactctatacattcaacagagactgaatagatatgaaagctgattttttttaattaccatgcttcacaatgttaagttatatggggagcaacagcaaacaggtgctaatttgttttggatatagtataagcagtgtctgtgttttgaaagaatagaacacagtttgtagtgccactgttgttttgggggggcttttttcttttcggaaatcttaaaccttaagatactaaggacgttgttttggttgtactttggaattcttagtcacaaaatatattttgtttacaaaaatttctgtaaaacaggttataacagtgtttaaagtctcagtttcttgcttggggaac

SEQ ID NO: 480 - DNA, 371 bp, Homo sapiens
ttccgttgctgtcggaattgaggaagagctgggggatgaagctcgctttgccggacataacttccgtaatcccagtgtgctgtgattcctctgcttgcctggagacgtggaacctctgtctcatcctcctggaaccttgctgtcctgatctgtgatagttcaccccctgagatcccctgagccccagggtgcccagaacttccctgattgacctgctccgctgctccttggcttacctgacctcttgctgtctctgctcgccctcctttctgtgccctactcattggggttccgcactttccacttcttcctttctctttctctcttccctcaaaaactagaaatgtgaatgaggattattataaaagggg

SEQ ID NO: 481 - DNA, 543 bp, Homo sapiens
aattccgttgctgtcggtgtctggaggccatcctccagaactctcctgacgccaaaatcttctgcctggtgcacaaaatggatctggttcaggaggatcagcgtgacctgatttttaaagagcgagaggaagacctgaggcgtctgtctcgcccgctggagtgtgcttgttttcgaacgtccatctgggatgagacgctctacaaagcctggtccagcatcgtctaccagctgattcccaacgttcagcagctggagatgaacctcaggaattttgcccaaatcattgaggccgatgaagttctgctgttcgaaagagctacattcttggttatttcccactaccagtgcaaagagcagcgcgacgtccaccggtttgagaagatcagcaacatcatcaaacagttcaagctgagctgcagtaaattggccgcttccttccagagcatggaagttaggaattccaacttcgctgctttcatcgacatcttcacctcaaatacgtacgtgatggtggtcatgtcagatccgtcgatcccttctg

SEQ ID NO: 482 - DNA, 415 bp, Homo sapiens
ggcttactcactatagggcttttttttttttcgggtctattctttaattttactaaattaggaacgcagcttttacagaacaaataaccccaggggacggggcccccccaggatctaacagcttttcagggagctatgttgcaagctcaaaagtaatccactaacgaaccaagtcaaactccagtttttaataaaaaggggctgggggaggttgtcaaaccccttccaatataaatccccaatccgatggccaccaaatgaaaaagcaccagggatggaaggaaaactttcaaaaattctgcaaaaaatatgcccccttttttaatgaccctcgggttcctaatgctaaggggggccgcccccttcgggggttaaaaaaggaactccttggggggaatattttccggccgacttg

SEQ ID NO: 483 - DNA, 240 bp, Homo sapiens
tttttttttttaaagtcatggaggccatggggttggcttgaaaccacctttggggggtccaatcccttccttttttgcctaaattttatgtatacgggttcttcaaatgcgtggtaggggggggggcatccatatagtccctccaggtttatggagggttcttctactattaggacttttcgcttcaaaacaaaggcttttcaaatcatgaaaatttttaattttcctgctgttaaaaaa

SEQ ID NO: 484 - DNA, 293 bp, Homo sapiens
ttttttttttaataaatctcctaaggggatggctactttttctatctaaataataatatatagacctattcgatcagagatacaggggactaacaatcacaatcctgtgatcgacatccgaacataagtcactatctatcagaataaacaatgatccaacgaataatagaggagtaaggggacatgtccaaagcatcaggtatcgtcatgatcgaaaaccactgtcaagcaagacacaaacaaacaaaacagctttacacacaagtcagcagtccaagcgttcatgtcccaag

SEQ ID NO: 485 - DNA, 221 bp, Homo sapiens
tttttttttttcaagggacactttaatggttaacttaagggatcatcaattttgcctcactacctacaaagggaatttcatcttgtccccatgctgagtagggaaacagggacaaagttaatcataataccctacatcaaaaaaaaactaagctaacactgctaactttttttttaacaggcaaaatataaatatatgccctctaaaatgcccaagggttt

SEQ ID NO: 486 - DNA, 563 bp, Homo sapiens
ttccgttgctgtcgcctccgctctgctcttcgtggaacacgaccgtggtgcccggcccttgggagccttggggccagctggcctgctgctctccagtcaagtagcgaagctcctaccacccagacacccaaacagccgtggccccagaggtcctggccaaatatgggggcctgcctaggttggtggaacagtgctccttatgtaaactgagccctttgtttaaaaaacaattccaaatgtgaaactagaatgagagggaagagataacatggcatgcagcacacacggctgctccagttcatggcctcccaggggtgctggggatgcatccaaagtggttgtctgagacagagttggaaaccctcaccaactggcctcttcaccttccacattatcccgctgccaccggctgccctgtctcactgcagattcaggaccagcttgggctgcgtgcgttctgccttgccagtcagccgaggatgtagttgttgctgccgtcgtcccaccacctcagggaccagagggctaggttggcactgcggccctcaccaggtcctgggctc

SEQ ID NO: 487 - DNA, 271 bp, Homo sapiens
ctcatatggtcaggtcgcttcaaaaaggaagatgaaatcccagagactgtctcgttggagatgcttgatgctgcaaagaacaagatgcgagtgaagatcagctatctaatgattgccctgacggtggtaggatgcatcttcatggttattgagggcaagaaggctgcccaaagacacgagactttaacaagcttgaacttagaaaagaaagctcgtctgaaagaggaagcagctatgaaggccaaaacagagtagcagaggtatccgtgtt

SEQ ID NO: 488 - DNA, 342 bp, Homo sapiens
ggcttgtaatacgactcactatagggctttttttttttcgaattaaaaaaattccgttagccttttctccatctcctctaattctggtagcatctttggacccctaacacttggcatctgctacttcagacaaacaaaccctatgtaaatgacaaagaaggggcctcccaaccttctccctgtgttactatttcaaaagcactactcggggcacaggggtacaaatttcttatggccactagcatcttttttcaattttcaaaggaatcatcaaacatctgggtcaattatacttaaattacagaagcccggaattttaggcaacaggcccctcattttacc

SEQ ID NO: 489 - DNA, 326 bp, Homo sapiens
ttttttttttaaaaagtcatggaggccatggggttggcttgaaaccagctttggggggttcgattccttccttttttgtctaaattttatgtatacgggttcttcaaatgtgtggtagggtggggggcatccatatagtcactccaggtttatggagggttcttctactattaggacttttcgcttcgaagcgaaggcttctcaaatcatgaaaattattaatattactgctgttagaaaaatgaatgagcctaccgatgataggatgtttcatgtggtgtatgcatcggggtagtccgagtaacgtcggggcattccggataggc

SEQ ID NO: 490 - DNA, 55 bp, Homo sapiens
tttttttttttttttttttgagaaaccggggggggttttttttttaaaattgggg

SEQ ID NO: 491 - DNA, 558 bp, Homo sapiens
cgccgcgtccccttctcgctcctgcggggccccagctgggaccccttccgcgactggtacccgcatagccgcctcttcgaccaggccttcgggctgccccggctgccggaggagtggtcgcagtggttaggcggcagcagctggccaggctacgtgcgccccctgccccccgccgccatcgagagccccgcagtggccgcgcccgcctacagccgcgcgctcagccggcaactcagcagcggggtcttcggagatccggcacactgcggaccgctggcgcgtgtccctggatgtcaaccacttcgccccggacgagctgacggtcaagaccaaggatggcgtggtggagatcaccggcaagcacgaggagcggcaggacgagcatggctacatctcccggtgcttcacgcggaaatacacgctgccccccggtgtggaccccacccaagtttcctcctccctgtcccctgagggcacactgaccgtggaggcccccatgcccaagctagccacgcagtccaacgagatcaccatcccagtcaccttcgagtcgcgggc

SEQ ID NO: 492 - DNA, 370 bp, Homo sapiens
ggctagcggataacaatttcacacaggatggattggtcagagtgaattgaatattgtaagtcagccactgggacccgaggatttctgggaccccgcagttgggaggaggaagtagtccagccttccaggtggcgtgagaggcaatgactcgttacctgccgcccatcaccttggaggccttccctggccttgagtagaaaagtcggggatcggggcaagagaggctgagtacggatgggaaactattgtgcacaagtctttccagaggagtttcttaatgagatatttgtatttatttccagaccaataaatttgtaactttgcgaaaaaaaaaaagccctatagtgagtcgtattacaagccgaattcc

SEQ ID NO: 493 - DNA, 560 bp, Homo sapiens
cagccagcatgaccgagcgccgcgtccccttctcgctcctgcggggccccagctgggaccccttccgcgactggtacccgcatagccgcctcttcgaccaggccttcgggctgccccggctgccggaggagtggtcgcagtggttaggcggcagcagctggccaggctacgtgcgccccctgccccccgccgccatcgagagccccgcagtggccgcgcccgcctacagccgcgcgctcagccggcaactcagcagcggggtctcggagatccggcacactgcggaccgctggcgcgtgtccctggatgtcaaccacttcgccccggacgagctgacggtcaagaccaaggatggcgtggtggagatcaccggcaagcacgaggagcggcaggacgagcatggctacatctcccggtgcttcacgcggaaatacacgctgccccccggtgtggaccccacccaagtttcctcctccctgtcccctgagggcacactgaccgtggaggcccccatgcccaagctagccacgcagtccaacgagatcaccatcccagtcacc

SEQ ID NO: 494 - DNA, 443 bp, Homo sapiens
ggcttgtaatacgactcactatagggcttttttttttgcaagtgctgtgggaagaaagttagatttacgccgatgaatatgatagtgaaatggattttggcgtaggtttggtctagggtgtagcctgagaataggggaaatcagtgaatgaagcctcctatgatggcaaatacagctcctattgataggacatagtggaagtgagctacaacgtagtacgtgtcgtgtagtacgatgtctagtgatgagtttgctaatacaatgccagtcaggccacctacggtgaaaagaaagatgaatcctagggctcaaagcactgcagcagatcatttcatattgcctccgtggagtgtggcgagtcagctaaatactttgacgccggtggggatagcgatgattatggtagcatcatcctgtgtgaaattgttatccgctaagccgaa

SEQ ID NO: 495 - DNA, 249 bp, Homo sapiens
ttttttttttcgaaggatttggcaaagatttgtttttttttccatttccagttttttaaagtaaacacagatttgcttaaaataaagctgattttaaaagcccacaaaagttgaacacaaaggagaggattaaattccccaatgcagagtgataaaaaggaaaagatcctgagtaggtgccttcagcaaaaaactgatcatccagggtgatcacctaataatcggagacttaattccttataatgcaaa

SEQ ID NO: 496 - DNA, 434 bp, Homo sapiens
tttccgtatctgcttcgggcttccacctcatttttttcgctttgcccattctgtttcagccagtcgccaagaatcatgaaagtcgccagtggcagcaccgccaccgccgccgcgggccccagctgcgcgctgaaggccggcaagacagcgagcggtgcgggcgaggtggtgcgctgtctgtctgagcagagcgtggccatctcgcgctgcgccgggggcgccggggcgcgcctgcctgccctgctggacgagcagcaggtaaacgtgctgctctacgacatgaacggctgttactcacgcctcaaggagctggtgcccaccctgccccagaaccgcaaggtgagcaaggtggagattctccagcacgtcatcgactacatcagggaccttcagttggagctgaactcggaatccgaagttggaacccccggggg

SEQ ID NO: 497 - DNA, 368 bp, Homo sapiens
tttttttttgcttatggagggttcctctactattaggacttttcgcttcgaagcgaaggcttctcaaatcatgaaaattattaatattactgctgttagagaagtgaatgaccctacagatgataggatgtttcatgtggtgtatgcatcggggtagtccgagtaacgtcggggcattccggataggccgaaaaagtgttgtgggaaaaaagttagatttaccccgatgaatatgatagtgaaatggattttggcgtaggtttggtctagggtgtaccctgagaataggggaaatcagtgaatgaagcctcctatgatggcaaatacagctcctattgataggacatagtggaagtgagctacaacgt

SEQ ID NO: 498 - DNA, 482 bp, Homo sapiens
ccagccttcctgtcccgggccagcgctctgacatgcagaaggtgaccctgggcctgcttgtgttcctggcaggctttcctgtcctggacgccaatgacctagaagataaaaacagtcctttctactatgactggcacagcctccaggttggcgggctcatctgcgctggggttctgtgcgccatgggcatcatcatcgtcatgagtgcaaaatgcaaatgcaagtttggccagaagtccggtcaccatccaggggagactccacctctcatcaccccaggctcagcccaaagctgatgaggacagaccagctgaaattgggtggaggaccgttctctgtccccaggtcctgtctctgcacagaaacttgaactccaggatggaattcttcctcctctgctgggactcctttgcatggcagggcctcatctcacctctcgcaagagggtctctttgttcaatttttttttatctaaaatgatt

SEQ ID NO: 499 - DNA, 489 bp, Homo sapiens
tggcgagcagtttcccacttgccaaagatcccttttaaccaacactagcccttgtttttaacacacgctccagcccttcatcagcctgggcagtcttaccaaaatgtttaaagtgatctcagaggggcccatggattaacgccctcatcccaaggtccgtcccatgacataacactccacacccgccccagccaacttcatgggtcactttttctggaaaataatgatctgtacagacaggacagaatgaaactcctgcggctctttggcctgaaagttgggaatggttgggggagagaagggcagcagcttattggtggtcttttcaccattggcagaaacagtgagagctgtgtggtgcagaaatccagaaatgaggtgtagggaattttgcctgccttcctgcagacctgagctggctttggaatgaggttaaagtgtcagggacgttgcctgagcccaaatgtgtagtgtggtctgggcaggcag

SEQ ID NO: 500 - DNA, 25 bp, Artificial Sequence
  Oligonucleotide primer
ggaatcaccgctttgccatcttcaa

SEQ ID NO: 501 - DNA, 25 bp, Artificial Sequence
  Oligonucleotide primer
aacttctaccgtttcgccactaagg

SEQ ID NO: 502 - DNA, 23 bp, Artificial Sequence
  Oligonucleotide primer
gaccgtgtactgcgtgtcgtgcg

SEQ ID NO: 503 - DNA, 23 bp, Artificial Sequence
  Oligonucleotide primer
gcgtgctgtgcgtcatgtgccag

SEQ ID NO: 504 - DNA, 24 bp, Artificial Sequence
  Oligonucleotide primer
gccgtcttcaggcaacaactccca

SEQ ID NO: 505 - DNA, 24 bp, Artificial Sequence
  Oligonucleotide primer
tgctggacgaggctgtcatcttgc

SEQ ID NO: 506 - DNA, 25 bp, Artificial Sequence
  Oligonucleotide primer
acagggagaaaactggttgtcctgg

SEQ ID NO: 507 - DNA, 23 bp, Artificial Sequence
  Oligonucleotide primer
aaggcagaacccatccactccaa

SEQ ID NO: 508 - DNA, 25 bp, Artificial Sequence
  Oligonucleotide primer
gctgctggattcgtttggcataact

SEQ ID NO: 509 - DNA, 25 bp, Artificial Sequence
  Oligonucleotide primer
tcaatacggtttgcttaggtcgtcg

SEQ ID NO: 510 - DNA, 24 bp, Artificial Sequence
  Oligonucleotide primer
tctcctctgagttcaaccgctgct

SEQ ID NO: 511 - DNA, 24 bp, Artificial Sequence
  Oligonucleotide primer
tcgtcgccaacttgagtctcctct

SEQ ID NO: 512 - protein, 406 aa, Homo sapiens
MAENGKNCDQRRVAMNKEHHNGNFTDPSSVNEKKRREREERQNIVLWRQPLITLQYFSLEILVILKEWTSKLWHRQSIVVSFLLLLAVLIATYYVEGVHQQYVQRIEKQFLLYAYWIGLGILSSVGLGTGLHTFLLYLGPHIASVTLAAYECNSVNFPEPPYPDQIICPDEEGTEGTISLWSIISKVRIEACMWGIGTAIGELPPYFMARAARLSGAEPDDEEYQEFEEMLEHAESAQDFASRAKLAVQKLVQKVGFFGILACASIPNPLFDLAGITCGHFLVPFWTFFGATLIGKAIIKMHIQKIFVIITFSKHIVEQMVAFIGAVPGIGPSLQKPFQEYLEAQRQKLHHKSEMGTPQGENWLSWMFEKLVVVMVCYFILSIINSMAQSYAKRIQQRLNSEEKTK

SEQ ID NO: 513 - DNA, 1221 bp, Homo sapiens
atggcagagaatggaaaaaattgtgaccagagacgtgtagcaatgaacaaggaacatcataatggaaatttcacagacccctcttcagtgaatgaaaagaagaggagggagcgggaagaaaggcagaatattgtcctgtggagacagccgctcattaccttgcagtatttttctctggaaatccttgtaatcttgaaggaatggacctcaaaattatggcatcgtcaaagcattgtggtgtcttttttactgctgcttgctgtgcttatagctacgtattatgttgaaggagtgcatcaacagtatgtgcaacgtatagagaaacagtttcttttgtatgcctactggataggcttaggaattttgtcttctgttgggcttggaacagggctgcacacctttctgctttatctgggtccacatatagcctcagttacattagctgcttatgaatgcaattcagttaattttcccgaaccaccctatcctgatcagattatttgtccagatgaagagggcactgaaggaaccatttctttgtggagtatcatctcaaaagttaggattgaagcctgcatgtggggtatcggtacagcaatcggagagctgcctccatatttcatggccagagcagctcgcctctcaggtgctgaaccagatgatgaagagtatcaggaatttgaagagatgctggaacatgcagagtctgcacaagactttgcctcccgggccaaactggcagttcaaaaactagtacagaaagttggattttttggaattttggcctgtgcttcaattccaaatcctttatttgatctggctggaataacgtgtggacactttctggtacctttttggaccttctttggtgcaaccctaattggaaaagcaataataaaaatgcatatccagaaaatttttgttataataacattcagcaagcacatagtggagcaaatggtggctttcattggtgctgtccccggcataggtccatctctgcagaagccatttcaggagtacctggaggctcaacggcagaagcttcaccacaaaagcgaaatgggcacaccacagggagaaaactggttgtcctggatgtttgaaaagttggtcgttgtcatggtgtgttacttcatcctatctatcattaactccatggcacaaagttatgccaaacgaatccagcagcggttgaactcagaggagaaaactaaataa

SEQ ID NO: 514 - DNA, 338 bp, Homo sapiens
gtgctgtccccggcataggtccatctctgcagaagccatttcaggagtacctggaggctcaacggcagaagcttcaccacaaaagcgaaatgggcacaccacagggagaaaactgcttgtcctggatgtttgaaaagtcggtcgatgtcatggtgtgttacttcatcctatctatcattaactccatggcacaaagttatgccaaacgaatccagcagcggttgaactcagaggagaaaactaaataagtagagaaagttttaaactgcagaaattggagtggatgggttctgccttatattgggaggactccaagccgggaaggaaaattccctttt

SEQ ID NO: 515 - DNA, 186 bp, Homo sapiens
tgtgttaatgttttctagcatgtactctggtttcaacagacacaaatttatatgttaacccagttttcttgccgttctgtaagtgttttattcttagtgtgatttttttccattgggatgtttttgattgaacttgttcattttgttttgcttgggaggaaaataaacaattttacttttttcctt

SEQ ID NO: 516 - DNA, 118 bp, Homo sapiens
acagggagaaaactggttgtcctggatgtttgaaaagttggtcgttgtcatggtgtgttacttcatcctatctatcattaactccatggcacaaagttatgccaaacgaatccagcag

(19) United States
(12) Patent Application Publication
(10) Pub. No.: US 2006/0141455 A1
(43) Pub. Date: Jun. 29, 2006

(54) Gene products differentially expressed in cancerous breast cells and their methods of use

(76) Inventor: Rhonda Hansen, Greenbrae, CA (US)

Correspondence Address: Chiron Corporation;Intellectual Property - R440, P.O. Box 8097, Emeryville, CA, 94662-8097, US

(21) Appl. No.: 10/501,187

(22) PCT Filed: Jan. 8, 2003
(86) PCT No.: PCT/US03/00657

§ 371 Date: Jan. 13, 2006

Related U.S. Application Data
(60) Provisional application No. 60/345,637, filed on Jan. 8, 2002.

(51) Int. Cl. A61K 48/00; C12Q 1/68; G01N 33/574; A61K 39/395

(52) U.S. Cl. 435/6; 435/7.23; 514/44; 424/155.1

(57) ABSTRACT

The present invention provides polynucleotides, as well as polypeptides encoded thereby, that are differentially expressed in breast cancer cells. These polynucleotides are useful in a variety of diagnostic and therapeutic methods. The present invention further provides methods of reducing growth of breast cancer cells. These methods are useful for treating breast cancer.

### CROSS-REFERENCING

This patent application claims the benefit of U.S. provisional application Ser. No. 60/345,637 filed Jan. 8, 2002, which application is incorporated by reference in its entirety.

### STATEMENT OF RIGHTS OF INVENTION

The United States Government has certain rights in this invention pursuant to a CRADA (BG98-053) between Chiron Corporation and the University of California, which operates the Lawrence Berkeley National Laboratory for the United States Department of Energy under Contract No. DE-AC03-76SF00098.

### FIELD OF THE INVENTION

The present invention relates to polynucleotides of human origin in substantially isolated form and gene products that are differentially expressed in breast cancer cells, and uses thereof.

### BACKGROUND OF THE INVENTION

Cancer, like many diseases, is not the result of a single, well-defined cause, but rather can be viewed as several diseases, each caused by different aberrations in informational pathways, that ultimately result in apparently similar pathologic phenotypes. Identification of polynucleotides that correspond to genes that are differentially expressed in cancerous, pre-cancerous, or low metastatic potential cells relative to normal cells of the same tissue type, provides the basis for diagnostic tools, facilitates drug discovery by providing for targets for candidate agents, and further serves to identify therapeutic targets for cancer therapies that are more tailored for the type of cancer to be treated.

Identification of differentially expressed gene products also furthers the understanding of the progression and nature of complex diseases such as cancer, and is key to identifying the genetic factors that are responsible for the phenotypes associated with development of, for example, the metastatic phenotype. Identification of gene products that are differentially expressed at various stages, and in various types of cancers, can both provide for early diagnostic tests, and further serve as therapeutic targets. Additionally, the product of a differentially expressed gene can be the basis for screening assays to identify chemotherapeutic agents that modulate its activity (e.g. its expression, biological activity, and the like).

Early disease diagnosis is of central importance to halting disease progression, and reducing morbidity. Analysis of a patient's tumor to identify the gene products that are differentially expressed, and administration of therapeutic agent(s) designed to modulate the activity of those differentially expressed gene products, provides the basis for more specific, rational cancer therapy that may result in diminished adverse side effects relative to conventional therapies. Furthermore, confirmation that a tumor poses less risk to the patient (e.g., that the tumor is benign) can avoid unnecessary therapies. In short, identification of genes and the encoded gene products that are differentially expressed in cancerous cells can provide the basis of therapeutics, diagnostics, prognostics, therametrics, and the like.

Breast cancer is a leading cause of death among women. One of the priorities in breast cancer research is the discovery of new biochemical markers that can be used for diagnosis, prognosis and monitoring of breast cancer. The prognostic usefulness of these markers depends on the ability of the marker to distinguish between patients with breast cancer who require aggressive therapeutic treatment and patients who should be monitored.

While the pathogenesis of breast cancer is unclear, transformation of non-tumorigenic breast epithelium to a malignant phenotype may be the result of genetic factors, especially in women under 30 (Miki, et al., Science, 266: 66-71, 1994). However, it is likely that other, non-genetic factors are also significant in the etiology of the disease. Regardless of its origin, breast cancer morbidity increases significantly if a lesion is not detected early in its progression. Thus, considerable effort has focused on the elucidation of early cellular events surrounding transformation in breast tissue. Such effort has led to the identification of several potential breast cancer markers.

Thus, the identification of new markers associated with breast cancer, and the identification of genes involved in transforming cells into the cancerous phenotype, remains a significant goal in the management of this disease. In exemplary aspects, the invention described herein provides breast cancer diagnostics, prognostics, therametrics, and therapeutics based upon polynucleotides and/or their encoded gene products.

### SUMMARY OF THE INVENTION

The present invention provides methods and compositions useful in detection of cancerous cells, identification of agents that modulate the phenotype of cancerous cells, and identification of therapeutic targets for chemotherapy of cancerous cells. Cancerous breast cells are of particular interest in each of these aspects of the invention. More specifically, the invention provides polynucleotides in substantially isolated form, as well as polypeptides encoded thereby, that are differentially expressed in breast cancer cells. Also provided are antibodies that specifically bind the encoded polypeptides. These polynucleotides, polypeptides and antibodies are thus useful in a variety of diagnostic, therapeutic, and drug discovery methods. In some embodiments, a polynucleotide that is differentially expressed in breast cancer cells can be used in diagnostic assays to detect breast cancer cells. In other embodiments, a polynucleotide that is differentially expressed in breast cancer cells, and/or a polypeptide encoded thereby, is itself a target for therapeutic intervention.

Accordingly, in one aspect the invention provides a method for detecting a cancerous breast cell. In general, the method involves contacting a test sample obtained from a cell that is suspected of being a breast cancer cell with a probe for detecting a gene product differentially expressed in breast cancer. Many embodiments of the invention involve a gene identifiable or comprising a sequence selected from the group consisting of SEQ ID NOS: 1-499, contacting the probe and the gene product for a time sufficient for binding of the probe to the gene product; and comparing a level of binding of the probe to the sample with a level of probe binding to a control sample obtained from a control breast cell of known cancerous state. A modulated (i.e. increased or decreased) level of binding of the probe in the test breast cell sample relative to the level of binding in a control sample is indicative of the cancerous state of the test breast cell. In certain embodiments, the level of binding of the probe in the test cell sample, usually in relation to at least one control gene, is similar to binding of the probe to a cancerous cell sample. In certain other embodiments, the level of binding of the probe in the test cell sample, usually in relation to at least one control gene, is different, i.e. opposite, to binding of the probe to a non-cancerous cell sample. In specific embodiments, the probe is a polynucleotide probe and the gene product is nucleic acid. In other specific embodiments, the gene product is a polypeptide. In further embodiments, the gene product or the probe is immobilized on an array.

In another aspect, the invention provides a method for assessing the cancerous phenotype (e.g., metastasis, metastatic potential, aberrant cellular proliferation, and the like) of a breast cell comprising detecting expression of a gene product in a test breast cell sample, wherein the gene comprises a sequence selected from the group consisting of SEQ ID NOS: 1-499; and comparing a level of expression of the gene product in the test breast cell sample with a level of expression of the gene in a control cell sample. Comparison of the level of expression of the gene in the test cell sample relative to the level of expression in the control cell sample is indicative of the cancerous phenotype of the test cell sample. In specific embodiments, detection of gene expression is by detecting a level of an RNA transcript in the test cell sample. In other specific embodiments detection of expression of the gene is by detecting a level of a polypeptide in a test sample.

In another aspect, the invention provides a method for suppressing or inhibiting a cancerous phenotype of a cancerous cell, the method comprising introducing into a mammalian cell an expression modulatory agent (e.g. an antisense molecule, small molecule, antibody, neutralizing antibody, inhibitory RNA molecule, etc.) to inhibition of expression of a gene identified by a sequence selected from the group consisting of SEQ ID NOS: 1-499. Inhibition of expression of the gene inhibits development of a cancerous phenotype in the cell. In specific embodiments, the cancerous phenotype is metastasis, aberrant cellular proliferation relative to a normal cell, or loss of contact inhibition of cell growth. In the context of this invention “expression” of a gene is intended to encompass the expression of an activity of a gene product, and, as such, inhibiting expression of a gene includes inhibiting the activity of a product of the gene.

In another aspect, the invention provides a method for assessing the tumor burden of a subject, the method comprising detecting a level of a differentially expressed gene product in a test sample from a subject suspected of or having a tumor, the differentially expressed gene product comprising a sequence selected from the group consisting of SEQ ID NOS: 1-499. Detection of the level of the gene product in the test sample is indicative of the tumor burden in the subject.

In another aspect, the invention provides a method for identifying a gene product as a target for a cancer therapeutic, the method comprising contacting a cancerous cell expressing a candidate gene product with an anti-cancer agent, wherein the candidate gene product corresponds to a sequence selected from the group consisting of SEQ ID NOS: 1-499; and analyzing the effect of the anti-cancer agent upon a biological activity of the candidate gene product and/or upon a cancerous phenotype of the cancerous cell. Modulation of the biological activity of the candidate gene product and modulation of the cancerous phenotype of the cancerous cell indicates the candidate gene product is a target for a cancer therapeutic. In specific embodiments, the cancerous cell is a cancerous breast cell. In other specific embodiments, the inhibitor is an antisense oligonucleotide. In further embodiments, the cancerous phenotype is aberrant cellular proliferation relative to a normal cell, or colony formation due to loss of contact inhibition of cell growth.

In another aspect, the invention provides a method for identifying agents that modulate (i.e. increase or decrease) the biological activity of a gene product differentially expressed in a cancerous cell, the method comprising contacting a candidate agent with a differentially expressed gene product, the differentially expressed gene product corresponding to a sequence selected from the group consisting of SEQ ID NOS: 1-499; and detecting a modulation in a biological activity of the gene product relative to a level of biological activity of the gene product in the absence of the candidate agent. In specific embodiments, the detecting is by identifying an increase or decrease in expression of the differentially expressed gene product. In other specific embodiments, the gene product is mRNA or cDNA prepared from the mRNA gene product. In further embodiments, the gene product is a polypeptide.

In another aspect, the invention provides a method of inhibiting growth of a tumor cell by modulating expression of a gene product, where the gene product is encoded by a gene identified by a sequence selected from the group consisting of: SEQ ID NOS: 1-499.

The invention provides a method of determining the cancerous state of a cell, comprising detecting a level of a product of a gene in a test cell wherein said gene is defined by a sequence selected from a group consisting of SEQ ID NOS:1-499 wherein the cancerous state of the test cell is indicated by detection of said level and comparison to a control level of said gene product. In certain embodiments of this method, the gene product is a nucleic acid or a polypeptide. In certain embodiments of this method, the gene product is immobilized on an array. In one embodiment of this method, the control level is a level of said gene product associated with a control cell of known cancerous state. In other embodiments of this method, the known cancerous state is a non-cancerous state. In another embodiment of this method, the level differs from the control level by at least two fold, indicating the test cell is not of the same cancerous state as that indicated by the control level.

The invention also provides a method for detecting a cancerous breast cell in a sample. This method involves detecting a level of a product of a gene in a test sample obtained from a subject wherein said gene is defined by a sequence selected from a group consisting of SEQ ID NOS: 1-499, wherein the presence of a cancerous breast cell or metastasized breast cancer cell is indicated by detection of said level and comparison to a control level of said gene product. In certain embodiments of this method, the sample is a sample of breast tissue. In certain other embodiments of this method, the sample is selected from the group consisting of a liver, brain, kidney, lung, bone and skin sample. In one embodiment of this method, the cancerous breast cell is a metastasized cancerous breast cell. In certain other embodiments of the method, the control level is a level of said gene product associated with a control sample containing cells of known cancerous state. In another embodiment, the known cancerous state is a non-cancerous state.

The invention also provides a method for suppressing a cancerous phenotype of a cancerous mammalian cell comprising introducing into said cancerous cell an antisense polynucleotide for inhibition of expression of a gene defined by a sequence selected from the group consisting of SEQ ID NOS: 1-499, wherein inhibition of expression of said gene suppresses development of said cancerous phenotype. In many embodiments of the method, the cancerous phenotype is metastatic. In certain embodiments of the method, the cancerous phenotype is aberrant cellular proliferation relative to a normal cell. In other embodiments of the method, the cancerous phenotype is loss of contact inhibition of cell growth. In certain other embodiments of the method, the cancerous phenotype is aberrant cellular proliferation of a metastasized breast cancer cell relative to a normal cell.

The invention further provides a method for assessing the tumor burden of a subject. This method involves detecting a level of a differentially expressed gene product in a test sample from a subject suspected of having a tumor, the differentially expressed gene product comprising a sequence selected from the group consisting of SEQ ID NOS: 1-499; wherein detection of the level of the gene product in the test sample is indicative of the tumor burden in the subject.

The method further provides a method for identifying a gene product as a target for a cancer therapeutic. This method involves contacting a cancerous cell expressing a candidate gene product with an anti-cancer agent, wherein the candidate gene product corresponds to a sequence selected from the group consisting of SEQ ID NOS: 1-499; and analyzing the effect of the anti-cancer agent upon a biological activity of the candidate gene product and upon a cancerous phenotype of the cancerous cell; wherein modulation of the biological activity of the candidate gene product and modulation of the cancerous phenotype of the cancerous cell indicates the candidate gene product is a target for a cancer therapeutic. In certain embodiments of this method, the cancerous cell is a cancerous breast cell. In other embodiments of this method, the inhibitor is an antisense oligonucleotide. In certain other embodiments of this method, the cancerous phenotype is aberrant cellular proliferation relative to a normal cell. In another embodiment of this method, the cancerous phenotype is colony formation due to loss of contact inhibition of growth.

The invention further provides a method for identifying agents that decrease biological activity of a gene product differentially expressed in a cancerous cell, the method comprising: contacting a candidate agent with a differentially expressed gene product, the differentially expressed gene product corresponding to a sequence selected from the group consisting of SEQ ID NOS: 1-499; and detecting a decrease in a biological activity of the gene product relative to a level of biological activity of the gene product in the absence of the candidate agent. In certain embodiments of this method, the detecting step is by detection of a decrease in expression of the differentially expressed gene product. In certain other embodiments of this method, the gene product is mRNA or a cDNA prepared from the mRNA gene product. In other embodiments of the invention, the gene product is a polypeptide.

The invention further provides a method of inhibiting growth of a tumor cell by modulating expression of a gene product, the gene product being encoded by a gene identified by a sequence selected from the group consisting of: SEQ ID NOS: 1-499.

These and other objects, advantages, and features of the invention will become apparent to those persons skilled in the art upon reading the details of the invention as more fully described below.

### BRIEF DESCRIPTION OF THE FIGURES

FIG. 1 is three panels of autoradiographs showing expression of GAK polypeptide in different cell lines.

FIG. 2 is a graph of a hydropathy plot and a table showing the hydrophobic regions of DKFZp566I133.

FIG. 3 is six panels of photographs of MDA-231 cells exposed to C180-7, C180-8 and positive control antisense (AS) and control (RC) oligonucleotides.

FIG. 4 is an alignment of spot ID 22793 and spot ID 26883.

FIG. 5 is a figure of three sequence alignments showing the mapping of each of three sequences onto VMPI (DKFZ).

### DETAILED DESCRIPTION OF THE INVENTION

The present invention provides polynucleotides, as well as polypeptides encoded thereby, that are differentially expressed in breast cancer cells. Methods are provided in which these polynucleotides and polypeptides are used for detecting and reducing the growth of breast cancer cells. Also provided are methods in which the polynucleotides and polypeptides of the invention are used in a variety of diagnostic and therapeutic applications for breast cancer.

Before the present invention is described, it is to be understood that this invention is not limited to particular embodiments described, as such may, of course, vary. It is also to be understood that the terminology used herein is for the purpose of describing particular embodiments only, and is not intended to be limiting.

Unless defined otherwise, all technical and scientific terms used herein have the same meaning as commonly understood by one of ordinary skill in the art to which this invention belongs. Although any methods and materials similar or equivalent to those described herein can be used in the practice or testing of the present invention, the preferred methods and materials are now described. All publications and patent applications mentioned herein are incorporated herein by reference to disclose and describe the methods and/or materials in connection with which the publications are cited.

It must be noted that as used herein and in the appended claims, the singular forms “a”, “and”, and “the” include plural referents unless the context clearly dictates otherwise. Thus, for example, reference to “a polynucleotide” includes a plurality of such polynucleotides and reference to “the breast cancer cell” includes reference to one or more cells and equivalents thereof known to those skilled in the art, and so forth.

The publications and applications discussed herein are provided solely for their disclosure prior to the filing date of the present application. Nothing herein is to be construed as an admission that the present invention is not entitled to antedate such publication by virtue of prior invention. Further, the dates of publication provided may be different from the actual publication dates which may need to be independently confirmed.

Definitions

The terms “polynucleotide” and “nucleic acid”, used interchangeably herein, refer to polymeric forms of nucleotides of any length, either ribonucleotides or deoxynucleotides. Thus, these terms include, but are not limited to, single-, double-, or multi-stranded DNA or RNA, genomic DNA, cDNA, DNA-RNA hybrids, or a polymer comprising purine and pyrimidine bases or other natural, chemically or biochemically modified, non-natural, or derivatized nucleotide bases. These terms further include, but are not limited to, mRNA or cDNA that comprise intronic sequences (see, e.g., Niwa et al. (1999) Cell 99(7):691-702). The backbone of the polynucleotide can comprise sugars and phosphate groups (as may typically be found in RNA or DNA), or modified or substituted sugar or phosphate groups. Alternatively, the backbone of the polynucleotide can comprise a polymer of synthetic subunits such as phosphoramidites and thus can be an oligodeoxynucleoside phosphoramidate or a mixed phosphoramidate-phosphodiester oligomer. Peyrottes et al. (1996) Nucl. Acids Res. 24:1841-1848; Chaturvedi et al. (1996) Nucl. Acids Res. 24:2318-2323. A polynucleotide may comprise modified nucleotides, such as methylated nucleotides and nucleotide analogs, uracyl, other sugars, and linking groups such as fluororibose and thioate, and nucleotide branches. The sequence of nucleotides may be interrupted by non-nucleotide components. A polynucleotide may be further modified after polymerization, such as by conjugation with a labeling component. Other types of modifications included in this definition are caps, substitution of one or more of the naturally occurring nucleotides with an analog, and introduction of means for attaching the polynucleotide to proteins, metal ions, labeling components, other polynucleotides, or a solid support. The term “polynucleotide” also encompasses peptidic nucleic acids (Pooga et al Curr Cancer Drug Targets. (2001) 1:231-9).

A “gene product” is a biopolymeric product that is expressed or produced by a gene. A gene product may be, for example, an unspliced RNA, an mRNA, a splice variant mRNA, a polypeptide, a post-translationally modified polypeptide, a splice variant polypeptide etc. Also encompassed by this term is biopolymeric products that are made using an RNA gene product as a template (i.e. cDNA of the RNA). A gene product may be made enzymatically, recombinantly, chemically, or within a cell to which the gene is native. In many embodiments, if the gene product is proteinaceous, it exhibits a biological activity. In many embodiments, if the gene product is a nucleic acid, it can be translated into a proteinaceous gene product that exhibits a biological activity.

A composition (e.g. a polynucleotide, polypeptide, antibody, or host cell) that is “isolated” or “in substantially isolated form” refers to a composition that is in an environment different from that in which the composition naturally occurs. For example, a polynucleotide that is in substantially isolated form is outside of the host cell in which the polynucleotide naturally occurs, and could be a purified fragment of DNA, could be part of a heterologous vector, or could be contained within a host cell that is not a host cell from which the polynucleotide naturally occurs. The term “isolated” does not refer to a genomic or cDNA library, whole cell total protein or mRNA preparation, genomic DNA preparation, or an isolated human chromosome. A composition which is in substantially isolated form is usually substantially purified.

As used herein, the term “substantially purified” refers to a compound (e.g., a polynucleotide, a polypeptide or an antibody, etc.,) that is removed from its natural environment and is usually at least 60% free, preferably 75% free, and most preferably 90% free from other components with which it is naturally associated. Thus, for example, a composition containing A is “substantially free of” B when at least 85% by weight of the total A+B in the composition is A. Preferably, A comprises at least about 90% by weight of the total of A+B in the composition, more preferably at least about 95% or even 99% by weight. In the case of polynucleotides, “A” and “B” may be two different genes positioned on different chromosomes or adjacently on the same chromosome, or two isolated cDNA species, for example.

The terms “polypeptide” and “protein”, interchangeably used herein, refer to a polymeric form of amino acids of any length, which can include coded and non-coded amino acids, chemically or biochemically modified or derivatized amino acids, and polypeptides having modified peptide backbones. The term includes fusion proteins, including, but not limited to, fusion proteins with a heterologous amino acid sequence, fusions with heterologous and homologous leader sequences, with or without N-terminal methionine residues; immunologically tagged proteins; and the like.

“Heterologous” refers to materials that are derived from different sources (e.g., from different genes, different species, etc.).

As used herein, the terms “a gene that is differentially expressed in a breast cancer cell,” and “a polynucleotide that is differentially expressed in a breast cancer cell” are used interchangeably herein, and generally refer to a polynucleotide that represents or corresponds to a gene that is differentially expressed in a cancerous breast cell when compared with a cell of the same cell type that is not cancerous, e.g., mRNA is found at levels at least about 25%, at least about 50% to about 75%, at least about 90%, at least about 1.5-fold, at least about 2-fold, at least about 5-fold, at least about 10-fold, or at least about 50-fold or more, different (e.g., higher or lower). The comparison can be made in tissue, for example, if one is using in situ hybridization or another assay method that allows some degree of discrimination among cell types in the tissue. The comparison may also or alternatively be made between cells removed from their tissue source.

“Differentially expressed polynucleotide” as used herein refers to a nucleic acid molecule (RNA or DNA) comprising a sequence that represents a differentially expressed gene, e.g., the differentially expressed polynucleotide comprises a sequence (e.g., an open reading frame encoding a gene product; a non-coding sequence) that uniquely identifies a differentially expressed gene so that detection of the differentially expressed polynucleotide in a sample is correlated with the presence of a differentially expressed gene in a sample. “Differentially expressed polynucleotides” is also meant to encompass fragments of the disclosed polynucleotides, e.g., fragments retaining biological activity, as well as nucleic acids homologous, substantially similar, or substantially identical (e.g., having about 90% sequence identity) to the disclosed polynucleotides.

By “cyclin G associated kinase”, or “GAK” is meant any polypeptide composition that exhibits cyclin G associated kinase activity. Examples of cyclin G associated kinase include the polypeptide defined by NCBI accession number XM_{—}003450, NM_{—}005255, NP_{—}005246 and NM_{—}031030. Assays for determining whether a polypeptide has cyclin G associated kinase activity are described in Ausubel et al., eds., 1998, Current Protocols in Molecular Biology, John Wiley & Sons, NY. Variants of the human cyclin G associated kinase that retain biological activity may be produced by, inter alia, substituting amino acids that are in equivalent positions between two cyclin G associated kinases, such as the cyclin G associated kinases from rat and humans.

With regard to cyclin G associated kinases, further references of interest include: Kanaoka et al, FEBS Lett. 1997 Jan. 27; 402(1):73-80; Kimura et al, Genomics. 1997 Sep. 1; 44(2):179-87; Greener et al, J Biol Chem. 2000 Jan 14;275(2):1365-70; and Korolchuk et al, Traffic. 2002 June; 3(6):428-39.

“DKFZp566I133” and “DKFZ” are used interchangeably herein to refer to a polypeptide composition that exhibits DKFZp566I133 activity. Assays for determining whether a polypeptide has DKFZp566I133 activity (i.e. for determining whether DKFZp566I133 may have intracytoplasmatic vacuole promoting activity) are described in Dusetti et al, (Biochem Biophys Res Commun. 2002 Jan. 18; 290(2):641-9). Variants of the DKFZp566I133 that retain biological activity may be produced by, inter alia, substituting amino acids that are in equivalent positions between two DKFZp566I133, such as the DKFZp566I133 from rat and humans. DKFZ is also known as VMPI, or vacuole membrane protein 1.

Alternatively, “DKFZp566I133”, or “DKFZ” refers to an amino acid sequence defined by NCBI accession number NP_{—}112200, AAH09758, NM_{—}138839, and NM_{—}030938, polynucleotides encoding the amino acid sequences set forth in these accession numbers (SEQ ID NO:512 and SEQ ID NO:513, respectively).

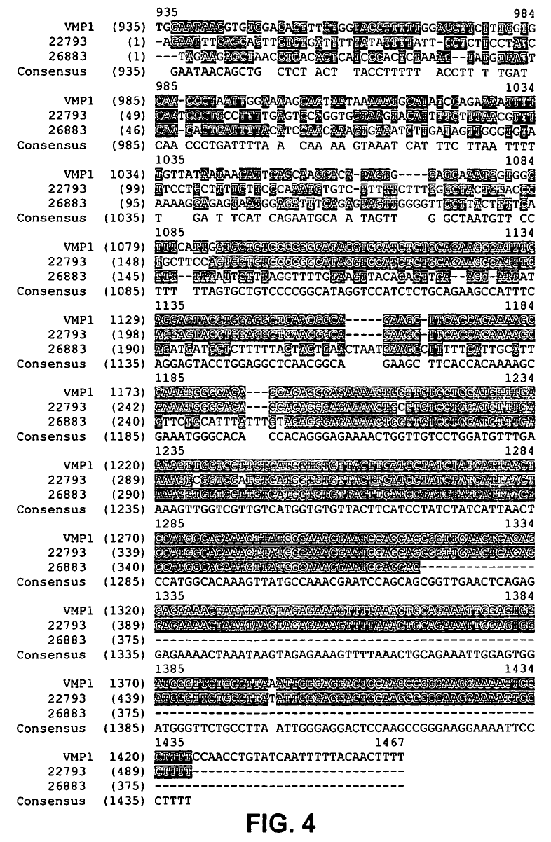

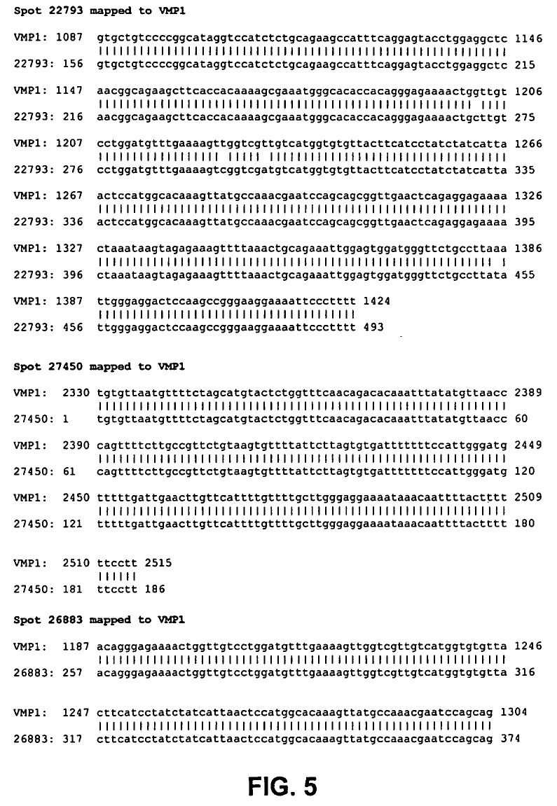

In addition, “DKFZp566I133”, or “DKFZ” refers to the polynucleotide sequences represented by Spot ID NOS 22793, 26883 and 27450 (SEQ ID NOS: 274-275 and SEQ ID NOS: 276-277 and SEQ ID NOS:459-460, respectively). FIG. 4 shows an alignment between Spot ID NOS: 22793, 26883 and VMP1 (NM_{—}030938) (i.e. DKFZ), identifying a VMPI or DKFZ gene product as corresponding to these spot IDs. FIG. 5 depicts fragments of Spot ID NOS 22793, 26883, 27450 which align with VMP1 (SEQ ID NOS 514, 515, and 516 respectively). These fragments, or their encoded products, may also be used as a DKFZ identifying sequence.

“Corresponds to” or “represents” when used in the context of, for example, a polynucleotide or sequence that “corresponds to” or “represents” a gene means that at least a portion of a sequence of the polynucleotide is present in the gene or in the nucleic acid gene product (e.g., mRNA or cDNA). A subject nucleic acid may also be “identified” by a polynucleotide if the polynucleotide corresponds to or represents the gene. Several genes identified by the polynucleotides of the sequence listing may be found in table 1. Genes identified by a polynucleotide may have all or a portion of the identifying sequence wholly present within an exon of a genomic sequence of the gene, or different portions of the sequence of the polynucleotide may be present in different exons (e.g., such that the contiguous polynucleotide sequence is present in an mRNA, either pre- or post-splicing, that is an expression product of the gene). In some embodiments, the polynucleotide may represent or correspond to a gene that is modified in a cancerous cell relative to a normal cell. The gene in the cancerous cell may contain a deletion, insertion, substitution, or translocation relative to the polynucleotide and may have altered regulatory sequences, or may encode a splice variant gene product, for example. The gene in the cancerous cell may be modified by insertion of an endogenous retrovirus, a transposable element, or other naturally occurring or non-naturally occurring nucleic acid. In most cases, a polynucleotide corresponds to or represents a gene if the sequence of the polynucleotide is most identical to the sequence of a gene or its product (e.g. mRNA or cDNA) as compared to other genes or their products. In most embodiments, the most identical gene is determined using a sequence comparison of a polynucleotide to a database of polynucleotides (e.g. GenBank) using the BLAST program at default settings For example, if the most similar gene in the human genome to an exemplary polynucleotide is the protein kinase C gene, the exemplary polynucleotide corresponds to protein kinase C. In most cases, the sequence of a fragment of an exemplary polynucleotide is at least 95%, 96%, 97%, 98%, 99% or up to 100% identical to a sequence of at least 15, 20, 25, 30, 35, 40, 45, or 50 contiguous nucleotides of a corresponding gene or its product (mRNA or cDNA), when nucleotides that are “N” represent G, A, T or C.

An “identifying sequence” is a minimal fragment of a sequence of contiguous nucleotides that uniquely identifies or defines a polynucleotide sequence or its complement. In many embodiments, a fragment of a polynucleotide uniquely identifies or defines a polynucleotide sequence or its complement. In some embodiments, the entire contiguous sequence of a gene, cDNA, EST, or other provided sequence is an identifying sequence. “Diagnosis” as used herein generally includes determination of a subject's susceptibility to a disease or disorder, determination as to whether a subject is presently affected by a disease or disorder, prognosis of a subject affected by a disease or disorder (e.g., identification of pre-metastatic or metastatic cancerous states, stages of cancer, or responsiveness of cancer to therapy), and use of therametrics (e.g., monitoring a subject's condition to provide information as to the effect or efficacy of therapy).

As used herein, the term “a polypeptide associated with breast cancer” refers to a polypeptide encoded by a polynucleotide that is differentially expressed in a breast cancer cell. Several examples of polypeptides associated with breast cancer are shown in Table 1.

The term “biological sample” encompasses a variety of sample types obtained from an organism and can be used in a diagnostic or monitoring assay. The term encompasses blood and other liquid samples of biological origin, solid tissue samples, such as a biopsy specimen or tissue cultures or cells derived therefrom and the progeny thereof. The term encompasses samples that have been manipulated in any way after their procurement, such as by treatment with reagents, solubilization, or enrichment for certain components. The term encompasses a clinical sample, and also includes cells in cell culture, cell supernatants, cell lysates, serum, plasma, biological fluids, and tissue samples.

The terms “treatment”, “treating”, “treat” and the like are used herein to generally refer to obtaining a desired pharmacologic and/or physiologic effect. The effect may be prophylactic in terms of completely or partially preventing a disease or symptom thereof and/or may be therapeutic in terms of a partial or complete stabilization or cure for a disease and/or adverse effect attributable to the disease. “Treatment” as used herein covers any treatment of a disease in a mammal, particularly a human, and includes: (a) preventing the disease or symptom from occurring in a subject which may be predisposed to the disease or symptom but has not yet been diagnosed as having it; (b) inhibiting the disease symptom, i.e., arresting its development; or (c) relieving the disease symptom, i.e., causing regression of the disease or symptom.

The terms “individual,” “subject,” “host,” and “patient,” used interchangeably herein and refer to any mammalian subject for whom diagnosis, treatment, or therapy is desired, particularly humans. Other subjects may include cattle, dogs, cats, guinea pigs, rabbits, rats, mice, horses, and the like.

A “host cell”, as used herein, refers to a microorganism or a eukaryotic cell or cell line cultured as a unicellular entity which can be, or has been, used as a recipient for a recombinant vector or other transfer polynucleotides, and include the progeny of the original cell which has been transfected. It is understood that the progeny of a single cell may not necessarily be completely identical in morphology or in genomic or total DNA complement as the original parent, due to natural, accidental, or deliberate mutation.

The terms “cancer”, “neoplasm”, “tumor”, and “carcinoma”, are used interchangeably herein to refer to cells which exhibit relatively autonomous growth, so that they exhibit an aberrant growth phenotype characterized by a significant loss of control of cell proliferation. In general, cells of interest for detection or treatment in the present application include precancerous (e.g., benign), malignant, pre-metastatic, metastatic, and non-metastatic cells. Detection of cancerous cells is of particular interest.

The term “normal” as used in the context of “normal cell,” is meant to refer to a cell of an untransformed phenotype or exhibiting a morphology of a non-transformed cell of the tissue type being examined.

“Cancerous phenotype” generally refers to any of a variety of biological phenomena that are characteristic of a cancerous cell, which phenomena can vary with the type of cancer. The cancerous phenotype is generally identified by abnormalities in, for example, cell growth or proliferation (e.g., uncontrolled growth or proliferation), regulation of the cell cycle, cell mobility, cell-cell interaction, or metastasis, etc.

“Therapeutic target” generally refers to a gene or gene product that, upon modulation of its activity (e.g., by modulation of expression, biological activity, and the like), can provide for modulation of the cancerous phenotype.

As used throughout, “modulation” is meant to refer to an increase or a decrease in the indicated phenomenon (e.g., modulation of a biological activity refers to an increase in a biological activity or a decrease in a biological activity).

Polynucleotide Compositions

The present invention provides isolated polynucleotides that represent genes that are differentially expressed in breast cancer cells. The polynucleotides, as well as polypeptides encoded thereby, find use in a variety of therapeutic and diagnostic methods.

The scope of the invention with respect to compositions containing the isolated polynucleotides useful in the methods described herein includes, but is not necessarily limited to, polynucleotides having a sequence set forth in any one of the polynucleotide sequences provided herein; polynucleotides obtained from the biological materials described herein or other biological sources (particularly human sources) by hybridization under stringent conditions (particularly conditions of high stringency); genes corresponding to the provided polynucleotides; cDNAs corresponding to the provided polynucleotides; variants of the provided polynucleotides and their corresponding genes, particularly those variants that retain a biological activity of the encoded gene product (e.g., a biological activity ascribed to a gene product corresponding to the provided polynucleotides as a result of the assignment of the gene product to a protein family(ies) and/or identification of a functional domain present in the gene product). Other nucleic acid compositions contemplated by and within the scope of the present invention will be readily apparent to one of ordinary skill in the art when provided with the disclosure here. “Polynucleotide” and “nucleic acid” as used herein with reference to nucleic acids of the composition is not intended to be limiting as to the length or structure of the nucleic acid unless specifically indicated.

The invention features polynucleotides that represent genes that are expressed in human tissue, specifically human breast tissue, particularly polynucleotides that are differentially expressed in cancerous breast cells. Nucleic acid compositions described herein of particular interest are at least about 15 bp in length, at least about 30 bp in length, at least about 50 bp in length, at least about 100 bp, at least about 200 bp in length, at least about 300 bp in length, at least about 500 bp in length, at least about 800 bp in length, at least about 1 kb in length, at least about 2.0 kb in length, at least about 3.0 kb in length, at least about 5 kb in length, at least about 10 kb in length, at least about 50 kb in length and are usually less than about 200 kb in length. These polynucleotides (or polynucleotide fragments) have uses that include, but are not limited to, diagnostic probes and primers as starting materials for probes and primers, as discussed herein.

The subject polynucleotides usually comprise a sequence set forth in any one of the polynucleotide sequences provided herein, for example, in the sequence listing, incorporated by reference in a table (e.g. by an NCBI accession number), a cDNA deposited at the A.T.C.C., or a fragment or variant thereof. A “fragment” or “portion” of a polynucleotide is a contiguous sequence of residues at least about 10 nt to about 12 nt, 15 nt, 16 nt, 18 nt or 20 nt in length, usually at least about 22 nt, 24 nt, 25 nt, 30 nt, 40 nt, 50 nt, 60nt, 70 nt, 80 nt, 90 nt, 100 nt to at least about 150 nt, 200 nt, 250 nt, 300 nt, 350 nt, 400 nt, 500 nt, 800 nt or up to about 1000 nt, 1500 or 2000 nt in length. In some embodiments, a fragment of a polynucleotide is the coding sequence of a polynucleotide. A fragment of a polynucleotide may start at position 1 (i.e. the first nucleotide) of a nucleotide sequence provided herein, or may start at about position 10, 20, 30, 50, 75, 100, 150, 200, 250, 300, 350, 400, 450, 500, 600, 700, 800, 900, 1000, 1500 or 2000, or an ATG translational initiation codon of a nucleotide sequence provided herein. In this context “about” includes the particularly recited value or a value larger or smaller by several (5, 4, 3, 2, or 1) nucleotides. The described polynucleotides and fragments thereof find use as hybridization probes, PCR primers, BLAST probes, or as an identifying sequence, for example.

The subject nucleic acids may be variants or degenerate variants of a sequence provided herein. In general, a variants of a polynucleotide provided herein have a fragment of sequence identity that is greater than at least about 65%, greater than at least about 70%, greater than at least about 75%, greater than at least about 80%, greater than at least about 85%, or greater than at least about 90%, 95%, 96%, 97%, 98%, 99% or more (i.e. 100%) as compared to an identically sized fragment of a provided sequence. as determined by the Smith-Waterman homology search algorithm as implemented in MPSRCH program (Oxford Molecular). For the purposes of this invention, a preferred method of calculating percent identity is the Smith-Waterman algorithm. Global DNA sequence identity should be greater than 65% as determined by the Smith-Waterrnan homology search algorithm as implemented in MPSRCH program (Oxford Molecular) using an gap search with the following search parameters: gap open penalty, 12; and gap extension penalty, 1.

The subject nucleic acid compositions include full-length cDNAs or mRNAs that encompass an identifying sequence of contiguous nucleotides from any one of the polynucleotide sequences provided herein.

As discussed above, the polynucleotides useful in the methods described herein also include polynucleotide variants having sequence similarity or sequence identity. Nucleic acids having sequence similarity are detected by hybridization under low stringency conditions, for example, at 50° C. and 10×SSC (0.9 M saline/0.09 M sodium citrate) and remain bound when subjected to washing at 55° C. in 1×SSC. Sequence identity can be determined by hybridization under high stringency conditions, for example, at 50° C. or higher and 0.1×SSC (9 mM saline/0.9 mM sodium citrate). Hybridization methods and conditions are well known in the art, see, e.g., U.S. Pat. No. 5,707,829. Nucleic acids that are substantially identical to the provided polynucleotide sequences, e.g. allelic variants, genetically altered versions of the gene, etc., bind to the provided polynucleotide sequences under stringent hybridization conditions. By using probes, particularly labeled probes of DNA sequences, one can isolate homologous or related genes. The source of homologous genes can be any species, e.g. primate species, particularly human; rodents, such as rats and mice; canines, felines, bovines, ovines, equines, yeast, nematodes, etc.

In one embodiment, hybridization is performed using a fragment of at least 15 contiguous nucleotides (nt) of at least one of the polynucleotide sequences provided herein. That is, when at least 15 contiguous nt of one of the disclosed polynucleotide sequences is used as a probe, the probe will preferentially hybridize with a nucleic acid comprising the complementary sequence, allowing the identification and retrieval of the nucleic acids that uniquely hybridize to the selected probe. Probes from more than one polynucleotide sequence provided herein can hybridize with the same nucleic acid if the cDNA from which they were derived corresponds to one mRNA.

Polynucleotides contemplated for use in the invention also include those having a sequence of naturally occurring variants of the nucleotide sequences (e.g., degenerate variants (e.g., sequences that encode the same polypeptides but, due to the degenerate nature of the genetic code, different in nucleotide sequence), allelic variants, etc.). Variants of the polynucleotides contemplated by the invention are identified by hybridization of putative variants with nucleotide sequences disclosed herein, preferably by hybridization under stringent conditions. For example, by using appropriate wash conditions, variants of the polynucleotides described herein can be identified where the allelic variant exhibits at most about 25-30% base pair (bp) mismatches relative to the selected polynucleotide probe. In general, allelic variants contain 15-25% bp mismatches, and can contain as little as even 5-15%, or 2-5%, or 1-2% bp mismatches, as well as a single bp mismatch.

The invention also encompasses homologs corresponding to any one of the polynucleotide sequences provided herein, where the source of homologous genes can be any mammalian species, e.g., primate species, particularly human; rodents, such as rats; canines, felines, bovines, ovines, equines, yeast, nematodes, etc. Between mammalian species, e.g., human and mouse, homologs generally have substantial sequence similarity, e.g., at least 75% sequence identity, usually at least 80%%, at least 85, at least 90%, at least 95%, at least 96%, at least 97%, at least 98%, at least 99% or even 100% identity between nucleotide sequences. Sequence similarity is calculated based on a reference sequence, which may be a subset of a larger sequence, such as a conserved motif, coding region, flanking region, etc. A reference sequence will usually be at least about a fragment of a polynucleotide sequence and may extend to the complete sequence that is being compared. Algorithms for sequence analysis are known in the art, such as gapped BLAST, described in Altschul, et al. Nucleic Acids Res. (1997) 25:3389-3402, or TeraBLAST available from TimeLogic Corp. (Crystal Bay, Nev.).

Moreover, representative examples of polynucleotide fragments of the invention (useful, for example, as probes), include, for example, fragments comprising, or alternatively consisting of, a sequence from about nucleotide number 1-50, 51-100, 101-150, 151-200, 201-250, 251-300, 301-350, 351-400, 401-450, 451-500, 501-550, 551-600, 651-700, 701-750, 751-800, 800-850, 851-900, 901-950, 951-1000, 1001-1050, 1051-1100, 1101-1150, 1151-1200, 1201-1250, 1251-1300, 1301-1350, 1351-1400, 1401-1450, 1451-1500, 1501-1550, 1551-1600, 1601-1650, 1651-1700, 1701-1750, 1751-1800, 1801-1850, 1851-1900, 1901-1950, 1951-2000, 2001-2050, 2051-2100, 2101-2150, 2151-2200, 2201-2250, 2251-2300, 2301-2350, 2351-2400, 2401-2450, 2451-2500, 2501-2550, 2551-2600, 2601-2650, 2651-2700, 2701-2750, 2751-2800, 2801-2850, 2851-2900, 2901-2950, 2951-3000, 3001-3050, 3051-3100, 3101-3150, 3151-3200, 3201-3250, 3251-3300, 3301-3350, 3351-3400, 3401-3450, 3451-3500, 3501-3550, 3551-3600, 3601-3650, 3651-3700, 3701-3750, 3751-3800, 3801-3850, 3851-3900, 3901-3950, 3951-4000, 4001-4050, 4051-4100, 4101-4150, 4151-4200, 4201-4250, 4251-4300, 4301-4350, 4351-4400, 4401-4450, 4451-4500, 4501-4550, 4551-4600, 4601-4650, 4651-4700, 4701-4750, 4751-4800, 4801-4850, 4851-4900, 4901-4950, 4951-5000, 5001-5050, 5051-5100, 5101-5150, 5151-5200, 5201-5250, 5251-5300, 5301-5350, 5351-5400, 5401-5450, 5451-5500, 5501-5550, 5551-5600, 5601-5650, 5651-5700, 5701-5750, 5751-5800, 5801-5850, 5851-5900, 5901-5950, 5951-6000, 6001-6050, 6051-6100, 6101-6150, and 6151 of a subject nucleic acid, or the complementary strand thereto. In this context “about” includes the particularly recited range or a range larger or smaller by several (5, 4, 3, 2, or 1) nucleotides, at either terminus or at both termini. In some embodiments, these fragments encode a polypeptide which has a functional activity (e.g., biological activity) whereas in other embodiments, these fragments are probes, or starting materials for probes. Polynucleotides which hybridize to one or more of these nucleic acid molecules under stringent hybridization conditions or alternatively, under lower stringency conditions, are also encompassed by the invention, as are polypeptides encoded by these polynucleotides or fragments.

The subject nucleic acids can be cDNAs or genomic DNAs, as well as fragments thereof, particularly fragments that encode a biologically active gene product and/or are useful in the methods disclosed herein (e.g., in diagnosis, as a unique identifier of a differentially expressed gene of interest, etc.). The term “cDNA” as used herein is intended to include all nucleic acids that share the arrangement of sequence elements found in native mature mRNA species, where sequence elements are exons and 3′ and 5′ non-coding regions. Normally mRNA species have contiguous exons, with the intervening introns, when present, being removed by nuclear RNA splicing, to create a continuous open reading frame encoding a polypeptide. mRNA species can also exist with both exons and introns, where the introns may be removed by alternative splicing. Furthermore it should be noted that different species of mRNAs encoded by the same genomic sequence can exist at varying levels in a cell, and detection of these various levels of mRNA species can be indicative of differential expression of the encoded gene product in the cell.

A genomic sequence of interest comprises the nucleic acid present between the initiation codon and the stop codon, as defined in the listed sequences, including all of the introns that are normally present in a native chromosome. It can further include the 3′ and 5′ untranslated regions found in the mature mRNA. It can further include specific transcriptional and translational regulatory sequences, such as promoters, enhancers, etc., including about 1 kb, but possibly more, of flanking genomic DNA at either the 5′ and 3′ end of the transcribed region. The genomic DNA can be isolated as a fragment of 100 kbp or smaller; and substantially free of flanking chromosomal sequence. The genomic DNA flanking the coding region, either 3′ and 5′, or internal regulatory sequences as sometimes found in introns, contains sequences required for proper tissue, stage-specific, or disease-state specific expression.

The nucleic acid compositions of the subject invention can encode all or a part of the naturally-occurring polypeptides. Double or single stranded fragments can be obtained from the DNA sequence by chemically synthesizing oligonucleotides in accordance with conventional methods, by restriction enzyme digestion, by PCR amplification, etc.

Probes specific to the polynucleotides described herein can be generated using the polynucleotide sequences disclosed herein. The probes are usually a fragment of a polynucleotide sequences provided herein. The probes can be synthesized chemically or can be generated from longer polynucleotides using restriction enzymes. The probes can be labeled, for example, with a radioactive, biotinylated, or fluorescent tag. Preferably, probes are designed based upon an identifying sequence of any one of the polynucleotide sequences provided herein. More preferably, probes are designed based on a contiguous sequence of one of the subject polynucleotides that remain unmasked following application of a masking program for masking low complexity (e.g., XBLAST, RepeatMasker, etc.) to the sequence., i.e., one would select an unmasked region, as indicated by the polynucleotides outside the poly-n stretches of the masked sequence produced by the masking program.

The polynucleotides of interest in the subject invention are isolated and obtained in substantial purity, generally as other than an intact chromosome. Usually, the polynucleotides, either as DNA or RNA, will be obtained substantially free of other naturally-occurring nucleic acid sequences that they are usually associated with, generally being at least about 50%, usually at least about 90% pure and are typically “recombinant”, e.g., flanked by one or more nucleotides with which it is not normally associated on a naturally occurring chromosome.

The polynucleotides described herein can be provided as a linear molecule or within a circular molecule, and can be provided within autonomously replicating molecules (vectors) or within molecules without replication sequences. Expression of the polynucleotides can be regulated by their own or by other regulatory sequences known in the art. The polynucleotides can be introduced into suitable host cells using a variety of techniques available in the art, such as transferrin polycation-mediated DNA transfer, transfection with naked or encapsulated nucleic acids, liposome-mediated DNA transfer, intracellular transportation of DNA-coated latex beads, protoplast fusion, viral infection, electroporation, gene gun, calcium phosphate-mediated transfection, and the like.

The nucleic acid compositions described herein can be used to, for example, produce polypeptides, as probes for the detection of mRNA in biological samples (e.g., extracts of human cells) or cDNA produced from such samples, to generate additional copies of the polynucleotides, to generate ribozymes or antisense oligonucleotides, and as single stranded DNA probes or as triple-strand forming oligonucleotides. The probes described herein can be used to, for example, determine the presence or absence of any one of the polynucleotide provided herein or variants thereof in a sample. These and other uses are described in more detail below.

Polypeptides and Variants Thereof

The present invention further provides polypeptides encoded by polynucleotides that represent genes that are differentially expressed in breast cancer cells. Such polypeptides are referred to herein as “polypeptides associated with breast cancer.” The polypeptides can be used to generate antibodies specific for a polypeptide associated with breast cancer, which antibodies are in turn useful in diagnostic methods, prognostics methods, therametric methods, and the like as discussed in more detail herein. Polypeptides are also useful as targets for therapeutic intervention, as discussed in more detail herein.

The polypeptides contemplated by the invention include those encoded by the disclosed polynucleotides and the genes to which these polynucleotides correspond, as well as nucleic acids that, by virtue of the degeneracy of the genetic code, are not identical in sequence to the disclosed polynucleotides. Further polypeptides contemplated by the invention include polypeptides that are encoded by polynucleotides that hybridize to polynucleotide of the sequence listing. Thus, the invention includes within its scope a polypeptide encoded by a polynucleotide having the sequence of any one of the polynucleotide sequences provided herein, or a variant thereof.

In general, the term “polypeptide” as used herein refers to both the full length polypeptide encoded by the recited polynucleotide, the polypeptide encoded by the gene represented by the recited polynucleotide, as well as portions or fragments thereof. “Polypeptides” also includes variants of the naturally occurring proteins, where such variants are homologous or substantially similar to the naturally occurring protein, and can be of an origin of the same or different species as the naturally occurring protein (e.g., human, murine, or some other species that naturally expresses the recited polypeptide, usually a mammalian species). In general, variant polypeptides have a sequence that has at least about 80%, usually at least about 90%, and more usually at least about 98% sequence identity with a differentially expressed polypeptide described herein, as measured by BLAST 2.0 using the parameters described above. The variant polypeptides can be naturally or non-naturally glycosylated, i.e., the polypeptide has a glycosylation pattern that differs from the glycosylation pattern found in the corresponding naturally occurring protein.

The invention also encompasses homologs of the disclosed polypeptides (or fragments thereof) where the homologs are isolated from other species, i.e. other animal or plant species, where such homologs, usually mammalian species, e.g. rodents, such as mice, rats; domestic animals, e.g., horse, cow, dog, cat; and humans. By “homolog” is meant a polypeptide having at least about 35%, usually at least about 40% and more usually at least about 60% amino acid sequence identity to a particular differentially expressed protein as identified above, where sequence identity is determined using the BLAST 2.0 algorithm, with the parameters described supra.

In general, the polypeptides of interest in the subject invention are provided in a non-naturally occurring environment, e.g. are separated from their naturally occurring environment. In certain embodiments, the subject protein is present in a composition that is enriched for the protein as compared to a cell or extract of a cell that naturally produces the protein. As such, isolated polypeptide is provided, where by “isolated” or “in substantially isolated form” is meant that the protein is present in a composition that is substantially free of other polypeptides, where by substantially free is meant that less than 90%, usually less than 60% and more usually less than 50% of the composition is made up of other polypeptides of a cell that the protein is naturally found.

Also within the scope of the invention are variants; variants of polypeptides include mutants, fragments, and fusions. Mutants can include amino acid substitutions, additions or deletions. The amino acid substitutions can be conservative amino acid substitutions or substitutions to eliminate non-essential amino acids, such as to alter a glycosylation site, a phosphorylation site or an acetylation site, or to minimize misfolding by substitution or deletion of one or more cysteine residues that are not necessary for function. Conservative amino acid substitutions are those that preserve the general charge, hydrophobicity/hydrophilicity, and/or steric bulk of the amino acid substituted.

Variants can be designed so as to retain or have enhanced biological activity of a particular region of the protein (e.g., a functional domain and/or, where the polypeptide is a member of a protein family, a region associated with a consensus sequence). For example, muteins can be made which are optimized for increased antigenicity, i.e. amino acid variants of a polypeptide may be made that increase the antigenicity of the polypeptide. Selection of amino acid alterations for production of variants can be based upon the accessibility (interior vs. exterior) of the amino acid (see, e.g., Go et al, Int. J. Peptide Protein Res. (1980) 15:211), the thermostability of the variant polypeptide (see, e.g., Querol et al., Prot. Eng. (1996) 9:265), desired glycosylation sites (see, e.g., Olsen and Thomsen, J. Gen. Microbiol. (1991) 137:579), desired disulfide bridges (see, e.g., Clarke et al., Biochemistry (1993) 32:4322; and Wakarchuk et al., Protein Eng. (1994) 7:1379), desired metal binding sites (see, e.g., Toma et al., Biochemistry (1991) 30:97, and Haezerbrouck et al., Protein Eng. (1993) 6:643), and desired substitutions with in proline loops (see, e.g., Masul et al., Appl. Env. Microbiol. (1994) 60:3579). Cysteine-depleted muteins can be produced as disclosed in U.S. Pat. No. 4,959,314.Variants also include fragments of the polypeptides disclosed herein, particularly biologically active fragments and/or fragments corresponding to functional domains. Fragments of interest will typically be at least about 10 aa to at least about 15 aa in length, usually at least about 50 aa in length, and can be as long as 300 aa in length or longer, but will usually not exceed about 1000 aa in length, where the fragment will have a stretch of amino acids that is identical to a polypeptide encoded by a polynucleotide having a sequence of any one of the polynucleotide sequences provided herein, or a homolog thereof. The protein variants described herein are encoded by polynucleotides that are within the scope of the invention. The genetic code can be used to select the appropriate codons to construct the corresponding variants.

A fragment of a subject polypeptide is, for example, a polypeptide having an amino acid sequence which is a portion of a subject polypeptide e.g. a polypeptide encoded by a subject polynucleotide that is identified by any one of the sequence of SEQ ID NOS 1-499 or its complement. The polypeptide fragments of the invention are preferably at least about 9 aa, at least about 15 aa, and more preferably at least about 20 aa, still more preferably at least about 30 aa, and even more preferably, at least about 40 aa, at least about 50 aa, at least about 75 aa, at least about 100 aa, at least about 125 aa or at least about 150 aa in length. A fragment “at least 20 aa in length,” for example, is intended to include 20 or more contiguous amino acids from, for example, the polypeptide encoded by a cDNA, in a cDNA clone contained in a deposited library, or a nucleotide sequence shown in SEQ ID NOS:1-499 or the complementary stand thereof. In this context “about” includes the particularly recited value or a value larger or smaller by several (5, 4, 3, 2, or 1) amino acids. These polypeptide fragments have uses that include, but are not limited to, production of antibodies as discussed herein. Of course, larger fragments (e.g., at least 150, 175, 200, 250, 500, 600, 1000, or 2000 amino acids in length) are also encompassed by the invention.

Moreover, representative examples of polypeptides fragments of the invention (useful in, for example, as antigens for antibody production), include, for example, fragments comprising, or alternatively consisting of, a sequence from about amino acid number 1-10, 5-10, 10-20,21-31, 31-40, 41-61, 61-81,91-120, 121-140, 141-162, 162-200, 201-240, 241-280,281- 320, 321-360, 360-400, 400-450, 451-500, 500-600, 600-700, 700-800, 800-900 and the like. In this context “about” includes the particularly recited range or a range larger or smaller by several (5, 4, 3, 2, or 1) amino acids, at either terminus or at both termini. In some embodiments, these fragments has a functional activity (e.g., biological activity) whereas in other embodiments, these fragments may be used to make an antibody.

Further polypeptide variants may are described in PCT publications WO/00-55173, WO/01-07611 and WO/02-16429

Vectors, Host Cells and Protein Production

The present invention also relates to vectors containing the polynucleotide of the present invention, host cells, and the production of polypeptides by recombinant techniques. The vector may be, for example, a phage, plasmid, viral, or retroviral vector. Retroviral vectors may be replication competent or replication defective. In the latter case, viral propagation generally will occur only in complementing host cells.

The polynucleotides of the invention may be joined to a vector containing a selectable marker for propagation in a host. Generally, a plasmid vector is introduced in a precipitate, such as a calcium phosphate precipitate, or in a complex with a charged lipid. If the vector is a virus, it may be packaged in vitro using an appropriate packaging cell line and then transduced into host cells.

The polynucleotide insert should be operatively linked to an appropriate promoter, such as the phage lambda PL promoter, the E. coli lac, trp, phoA and tac promoters, the SV40 early and late promoters and promoters of retroviral LTRs, to name a few. Other suitable promoters will be known to the skilled artisan. The expression constructs will further contain sites for transcription initiation, termination, and, in the transcribed region, a ribosome binding site for translation. The coding portion of the transcripts expressed by the constructs will preferably include a translation initiating codon at the beginning and a termination codon (UAA, UGA or UAG) appropriately positioned at the end of the polypeptide to be translated.

As indicated, the expression vectors will preferably include at least one selectable marker. Such markers include dihydrofolate reductase, G418 or neomycin resistance for eukaryotic cell culture and tetracycline, kanamycin or ampicillin resistance genes for culturing in E. coli and other bacteria.

Representative examples of appropriate hosts include,but are not limited to, bacterial cells, such as E. coli, Streptomyces and Salmonella typhimurium cells; fungal cells, such as yeast cells (e.g., Saccharomyces cerevisiae or Pichia pastoris (ATCC Accession No. 201178)); insect cells such as Drosophila S2 and Spodoptera Sf9 cells; animal cells such as CHO, COS, 293, and Bowes melanoma cells; and plant cells. 5 Appropriate culture mediums and conditions for the above-described host cells are known in the art.

Among vectors preferred for use in bacteria include pQE70, pQE60 and pQE-9, available from QIAGEN, Inc.; pBluescript vectors, Phagescript vectors, pNHSA, pNH16a, pNH18A, pNH46A, available from Stratagene Cloning Systems, Inc.; and ptrc99a, pKK223-3, pKK233-3, pDR540, pRITS available from Pharmacia Biotech, Inc. Among preferred eukaryotic vectors are pWLNEO, pSV2CAT, pOG44, pXTI and pSG available from Stratagene; and pSVK3, pBPV, pMSG and pSVL available from Pharmacia. Preferred expression vectors for use in yeast systems include, but are not limited to pYES2, pYDI, pTEFI/Zeo, pYES2/GS, pPICZ, pGAPZ, pGAPZalph, pPIC9, pPIC3.5, pHIL-D2, pHIL-S1, pPIC3.5K, pPIC9K, and PAO815 (all available from Invitrogen, Carload, Calif.). Other suitable vectors will be readily apparent to the skilled artisan.

Nucleic acids of interest may be cloned into a suitable vector by route methods. Suitable vectors include plasmids, cosmids, recombinant viral vectors e.g. retroviral vectors, YACs, BACs and the like, phage vectors.

Introduction of the construct into the host cell can be effected by calcium phosphate transfection, DEAE-dextran mediated transfection, cationic lipid-mediated transfection, electroporation, transduction, infection, or other methods. Such methods are described in many standard laboratory manuals, such as Davis et al., Basic Methods In Molecular Biology (1986). It is specifically contemplated that the polypeptides of the present invention may in fact be expressed by a host cell lacking a recombinant vector.

A polypeptide of this invention can be recovered and purified from recombinant cell cultures by well-known methods including ammonium sulfate or ethanol precipitation, acid extraction, anion or cation exchange chromatography, phosphocellulose chromatography, hydrophobic interaction chromatography, affinity chromatography, hydroxylapatite chromatography and lectin chromatography. Most preferably, high performance liquid chromatography (“HPLC”) is employed for purification.

Polypeptides of the present invention can also be recovered from: products purified from natural sources, including bodily fluids, tissues and cells, whether directly isolated or cultured; products of chemical synthetic procedures; and products produced by recombinant techniques from a prokaryotic or eukaryotic host, including, for example, bacterial, yeast higher plant, insect, and mammalian cells. Depending upon the host employed in a recombinant production procedure, the polypeptides of the present invention may be glycosylated or may be non-glycosylated. In addition, polypeptides of the invention may also include an initial modified methionine residue, in some cases as a result of host mediated processes. Thus, it is well known in the art that the N-terminal methionine encoded by the translation initiation codon generally is removed with high efficiency from any protein after translation in all eukaryotic cells. While the N-terminal methionine on most proteins also is efficiently removed in most prokaryotes, for some proteins, this prokaryotic removal process is inefficient, depending on the nature of the amino acid to which the N-terminal methionine is covalently linked.

Suitable methods and compositions for polypeptide expression may be found in PCT publications WO/00-55173, WO/01-07611 and WO/02-16429.

In addition, polypeptides of the invention can be chemically synthesized using techniques known in the art (e.g., see Creighton, 1983, Proteins: Structures and Molecular Principles, W.H. Freeman & Co., N.Y., and Hunkapiller et al., Nature, 310:105-111 (1984)). For example, a polypeptide corresponding to a fragment of a polypeptide can be synthesized by use of a peptide synthesizer. Furthermore, if desired, nonclassical amino acids or chemical amino acid analogs can be introduced as a substitution or addition into the polypeptide sequence. Non-classical amino acids include, but are not limited to, to the D-isomers of the common amino acids, 2,4-diaminobutyric acid, α-amino isobutyric acid, 4-aminobutyric acid, Abu, 2-amino butyric acid, g-Abu, e-Ahx, β-amino hexanoic acid, Aib, 2-amino isobutyric acid, 3-amino propionic acid, ornithine, norleucine, norvaline, 5 hydroxyproline, sarcosine, citrulline, homocitrulline, cysteic acid, t-butylglycine, t-butylalanine, phenylglycine, cyclohexylalanine, b-alanine, fluoro-amino acids, designer amino acids such as b-methyl amino acids, Ca-methyl amino acids, Na-methyl amino acids, and amino acid analogs in general. Furthermore, the amino acid can be D (dextrorotary) or L (levorotary).

Non-naturally occurring variants may be produced using art-known mutagenesis techniques, which include, but are not limited to oligonucleotide mediated mutagenesis, alanine scanning, PCR mutagenesis, site directed mutagenesis (see, e.g., Carter et al., Nucl. Acids Res. 73:4331 (1986); and Zoller et al., Nucl. Acids Res. 70:6487 (1982)), cassette mutagenesis (see, e.g., Wells et al., Gene 34:315 (1985)), restriction selection mutagenesis (see, e.g., Wells et al., Philos. Trans. R. Soc. London SerA 377:415 (1986)).

Scanning amino acid analysis can also be employed to identify one or more amino acids along a contiguous sequence. Among the preferred scanning amino acids are relatively small neutral amino acids. Such amino acids include alanine, glycine, serine and cysteine. Alanine is typically a preferred scanning amino acid among this group because it eliminates the side-chain beyond the beta-carbon and is less likely to alter the main-chain conformation of the variant. Alanine is also typically preferred because it is the most common amino acid. If alanine substituting does not yield adequate amounts of variant, an isoteric amino acid can be used.

Any cyseine reside not involved in maintaining the proper conformation of a polypeptide may also be substituted, generally with serine, to improve the oxidative stability of the molecule and prevent aberrant crosslinking. Conversely, cysteine bonds may be added to the polypeptide to improve its stability.

The invention additionally, encompasses polypeptides of the present invention which are differentially modified during or after translation, e.g., by glycosylation, acetylation, phosphorylation, amidation, derivatization by known protecting/blocking groups, proteolytic cleavage, linkage to an antibody molecule or other cellular ligand, etc. Any of numerous chemical modifications may be carried out by known techniques, including but not limited, to specific chemical cleavage by cyanogen bromide, trypsin, chymotrypsin, papain, V8 protease, NaBH4; acetylation, formylation, oxidation, reduction; metabolic synthesis in the presence of tunicamycin; etc.

Additional post-translational modifications encompassed by the invention include, for example, e.g., N-linked or O-linked carbohydrate chains, processing of N-terminal or C-terminal ends), attachment of chemical moieties to the amino acid backbone, chemical modifications of N-linked or O-linked carbohydrate chains, and addition or deletion of an N-terminal methionine residue as a result of procaryotic host cell expression. The polypeptides may also be modified with a detectable label, such as an enzymatic, fluorescent, isotopic or affinity label to allow for detection and isolation of the protein.

Also provided by the invention are chemically modified derivatives of the polypeptides of the invention which may provide additional advantages such as increased solubility, stability and circulating time of the polypeptide, or decreased immunogenicity (see U.S. Pat. No. 4,179,337). The chemical moieties for derivitization may be selected from water soluble polymers such as polyethylene glycol, ethylene glycol/propylene glycol copolymers, carboxymethylcellulose, dextran, polyvinyl alcohol and the like. The 5 polypeptides may be modified at random positions within the molecule, or at predetermined positions within the molecule and may include one, two, three or more attached chemical moieties.

Suitable methods and compositions for production of modified polypeptides may be found in PCT publications WO/00-55173, WO/01-0761 1 and WO/02-16429.

Antibodies and Other Polypeptide or Polynucleotied Binding Molecules

The present invention further provides antibodies, which may be isolated antibodies, that are specific for a polypeptide encoded by a polynucleotide described herein and/or a polypeptide of a gene that corresponds to a polynucleotide described herein. Antibodies can be provided in a composition comprising the antibody and a buffer and/or a pharmaceutically acceptable excipient. Antibodies specific for a polypeptide associated with breast cancer are useful in a variety of diagnostic and therapeutic methods, as discussed in detail herein.

Gene products, including polypeptides, mRNA (particularly mRNAs having distinct secondary and/or tertiary structures), cDNA, or complete gene, can be prepared and used for raising antibodies for experimental, diagnostic, and therapeutic purposes. Antibodies may be used to identify a gene corresponding to a polynucleotide. The polynucleotide or related cDNA is expressed as described above, and antibodies are prepared. These antibodies are specific to an epitope on the polypeptide encoded by the polynucleotide, and can precipitate or bind to the corresponding native protein in a cell or tissue preparation or in a cell-free extract of an in vitro expression system.

Antibodies

Further polypeptides of the invention relate to antibodies and T-cell antigen receptors (TCR) which immunospecifically bind a subject polypeptide, subject polypeptide fragment, or variant thereof, and/or an epitope thereof (as determined by immunoassays well known in the art for assaying specific antibody-antigen binding). Antibodies of the invention include, but are not limited to, polyclonal, monoclonal, multispecific, human, humanized or chimeric antibodies, single chain antibodies, Fab fragments, F(ab′) fragments, fragments produced by a Fab expression library, anti-idiotypic (anti-Id) antibodies (including, e.g., anti-Id antibodies to antibodies of the invention), and epitope-binding fragments of any of the above. The term “antibody,” as used herein, refers to immunoglobulin molecules and immunologically active portions of immunoglobulin molecules, i.e., molecules that contain an antigen binding site that immunospecifically binds an antigen. The immunoglobulin molecules of the invention can be of any type (e.g., IgG, IgE, IgM, IgD, IgA and IgY), class (e.g., IgG1, IgG2, IgG3, IgG4, IgA1 and IgA2) or subclass of immunoglobulin molecule.

Most preferably the antibodies are human antigen-binding antibody fragments of the present invention and include, but are not limited to, Fab. Fab′ and F(ab′)2, Fd, single-chain Fvs (scFv), single-chain antibodies, disulfide-linked Fvs (sdFv) and fragments comprising either a V_{L} or V_{H} domain. Antigen-binding antibody fragments, including single-chain antibodies, may comprise the variable region(s) alone or in combination with the entirety or a portion of the following: hinge region, C_{H}1, C_{H}², and C_{H}3 domains. Also included in the invention are antigen-binding fragments also comprising any combination of variable region(s) with a hinge region, C_{H}1, C_{H}2, and C_{H}3 domains. The antibodies of the invention may be from any animal origin including birds and mammals. Preferably, the antibodies are human, murine (e.g., mouse and rat), donkey, ship rabbit, goat, guinea pig, camel, horse, or chicken. As used herein, “human” antibodies include antibodies having the amino acid sequence of a human immunoglobulin and include antibodies isolated from, human immunoglobulin libraries or from animals transgenic for one or more human immunoglobulin and that do not express endogenous immunoglobulins, as described infra and, for example in, U.S. Pat. No. 5,939,598 by Kucherlapati et al.

The antibodies of the present invention may be monospecific, bispecific, trispecific or of greater multispecificity. Multispecific antibodies may be specific for different epitopes of a polypeptide of the present invention or may be specific for both a polypeptide of the present invention as well as for a heterologous epitope, such as a heterologous polypeptide or solid support material. See, e.g., PCT publications WO 93/17715; WO 92/08802; WO 91/00360; WO 92/05793; Tutt, et al., J. Immunol. 147:60-69 (1991); U.S. Pat. Nos. 4,474,893; 4,714,681; 4,925,648; 5,573,920; 5,601,819; Kostelny et al., J. Immunol. 148:1547-1553 (1992).

Antibodies of the present invention may be described or specified in terms of the epitope(s) or portion(s) of a polypeptide of the present invention which they recognize or specifically bind. The epitope(s) or polypeptide portion(s) may be specified as described herein, e.g., by N-terminal and C-terminal positions, or by size in contiguous amino acid residues. Antibodies which specifically bind any epitope or polypeptide of the present invention may also be excluded. Therefore, the present invention includes antibodies that specifically bind polypeptides of the present invention, and allows for the exclusion of the same.

Antibodies of the present invention may also be described or specified in terms of their cross-reactivity. Antibodies that do not bind any other analog, ortholog, or homolog of a polypeptide of the present invention are included. Antibodies that bind polypeptides with at least 95%, at least 90%, at least 85%, at least 80%, at least 75%, at least 70%, at least 65%, at least 60%, at least 55%, and at least 50% identity (as calculated using methods known in the art and described herein) to a polypeptide of the present invention are also included in the present invention. In specific embodiments, antibodies of the present invention cross-react with murine, rat and/or rabbit homologs of human proteins and the corresponding epitopes thereof Antibodies that do not bind polypeptides with less than 95%, less than 90%, less than 85%, less than 80%, less than 75%, less than 70%, less than 65%, less than 60%, less than 55%, and less than 50% identity (as calculated using methods known in the art and described herein) to a polypeptide of the present invention are also included in the present invention. In a specific embodiment, the above-described cross-reactivity is with respect to any single specific antigenic or immunogenic polypeptide, or combination(s) of 2, 3, 4, 5, or more of the specific antigenic and/or immunogenic polypeptides disclosed herein. Further included in the present invention are antibodies which bind polypeptides encoded by polynucleotides which hybridize to a polynucleotide of the present invention under stringent hybridization conditions (as described herein). Antibodies of the present invention may also be described or specified in terms of their binding affinty to a polypeptide of the invention. Preferred binding affinities include those with a dissociation constant or Kd less than 5×10⁻² M, 10⁻² M, 5×10⁻³ M, 10⁻³ M, 5×10⁻⁴ M, 10⁻⁴ M, 5×10⁻⁵ M, 10⁻⁵ M, 5×10⁻⁶ M, 10⁻⁶ M, 5×10⁻⁷ M, 10⁻⁷ M, 5×1D⁻⁸ M, 10⁻⁸ M, 5×10⁻⁹ M, 10⁻⁹ M, 5×1O⁻¹⁰M, 10⁻¹⁰ M, 5×1O⁻¹¹ M, 1O^{×11} M, 5×10⁻¹² M, 10⁻¹² M, 5×10⁻¹³ M, 1O⁻¹³ M, 5×10⁻¹⁴ M, 10⁻¹⁴ M, 5×10⁻¹⁵ M, or 10⁻¹⁵ M.

The invention also provides antibodies that competitively inhibit binding of an antibody to an epitope of the invention as determined by any method known in the art for determining competitive binding, for example, the immunoassays described herein. In preferred embodiments, the antibody competitively inhibits binding to the epitope by at least 95%, at least 90%, at least 85 %, at least 80%, at least 75%, at least 70%, at least 60%, or at least 50%.

Methods for making screening, humanzing, and modifying different types of antibody are well known in the art and may be found in PCT publications WO/00-5 5173, WO/01-07611 and WO/02-16429.

Antibodies of the present invention may act as agonists or antagonists of the polypeptides of the present invention. For example, the present invention includes antibodies which disrupt the receptor/ligand interactions with the polypeptides of the invention either partially or fully. Preferably, antibodies of the present invention bind an antigenic epitope disclosed herein, or a portion thereof. The invention features both receptor-specific antibodies and ligand-specific antibodies. The invention also features receptor-specific antibodies which do not prevent ligand binding but prevent receptor activation. Receptor activation (i.e., signaling) may be determined by techniques described herein or otherwise known in the art. For example, receptor activation can be determined by detecting the phosphorylation (e.g., tyrosine or serine/threonine) of the receptor or its substrate by immunoprecipitation followed by western blot analysis (for example, as described supra). In specific embodiments, antibodies are provided that inhibit ligand activity or receptor activity by at least 95%, at least 90%, at least 85%, at least 80%, at least 75%, at least 70%, at least 60%, or at least 50% of the activity in absence of the antibody.

The invention also features receptor-specific antibodies which both prevent ligand binding and receptor activation as well as antibodies that recognize the receptor-ligand complex, and, preferably, do not specifically recognize the unbound receptor or the unbound ligand. Likewise, included in the invention are neutralizing antibodies which bind the ligand and prevent binding of the ligand to the receptor, as well as antibodies which bind the ligand, thereby preventing receptor activation, but do not prevent the ligand from binding the receptor. Further included in the invention are antibodies which activate the receptor. These antibodies may act as receptor agonists, i.e., potentiate or activate either all or a subset of the biological activities of the ligand-mediated receptor activation, for example, by inducing dimerization of the receptor. The antibodies may be specified as agonists, antagonists or inverse agonists for biological activities comprising the specific biological activities of the peptides of the invention disclosed herein. The above antibody agonists can be made using methods known in the art. See, e.g., PCT publication WO 96/40281; U.S. Pat. No. 5,811,097; Deng et al., Blood 92(6): 1981-1988 (1998); Chen et al., Cancer Res. 58(16):36684998 (1998); Harrop et al., J. Immunol. 161(4): 1786-1794 (1998); Zhu et al., Cancer Res. 58(15):3209-3214 (1998): Yoon et al., J. Immunol. 160(7):3 170-3179 (1998); Prat et al., J. Cell. Sci. 11 l(Pt2):237-247 (1998); Pitard et al., J. Immunol. Methods 205(2): 177-190 (1997); Liautard et al., Cytokine 9(4):233-241 (1997); Carlson et al., J. Biol. Chem. 272(17): 11295-1 1301 (1997); Taryman et al., Neuron 14(4):755-762 (1995); Muller et al., Structure 6(9): 1153-1167 (1998); Bartunek et al., Cytokine 8(1):14-20 (1996) (which are all incorporated by reference herein in their entireties).

Antibodies of the present invention may be used, for example, but not limited to, to puri, detect, and target the polypeptides of the present invention, including both in vitro and in vivo diagnostic and therapeutic methods. For example, the antibodies have use in immunoassays for qualitatively and quantitatively measuring levels of the polypeptides of the present invention in biological samples. See, e.g., Harlow et al., Antibodies: A Laboratory Manual, (Cold Spring Harbor Laboratory Press, 2nd ed. 1988) (incorporated by reference herein in its entirety).

Further methods and compositions involving antibodies may be found in PCT publications WO/00-55173, WO/01-07611 and WO/02-16429.

Polynucleotides Encoding Antibodies

The invention further provides polynucleotides comprising a nucleotide sequence encoding an antibody of the invention and fragments thereof. The invention also encompasses polynucleotides that hybridize under stringent or alternatively, under lower stringency hybridization conditions, e.g., as defined supra, to polynucleotides that encode an antibody, preferably, that specifically binds to a polypeptide of the invention, preferably, an antibody that binds to a subject polypeptide.

The polynucleotides may be obtained, and the nucleotide sequence of the polynucleotides determined, by any method known in the art. For example, if the nucleotide sequence of the antibody is known, a polynucleotide encoding the antibody may be assembled from chemically synthesized oligonucleotides (e.g., as described in Kutmeier et al., BioTechniques 17:242 (1994)). which, briefly, involves the synthesis of overlapping oligonucleotides containing portions of the sequence encoding the antibody, annealing and ligating of those oligonucleotides, and then amplification of the ligated oligonucleotides by PCR.

Alternatively, a polynucleotide encoding an antibody may be generated from nucleic acid from a suitable source. If a clone containing a nucleic acid encoding a particular antibody is not available, but the sequence of the antibody molecule is known, a nucleic acid encoding the immunoglobulin may be chemically synthesized or obtained from a suitable source (e.g., an antibody cDNA library, or a cDNA library generated from, or nucleic acid, preferably poly A+RNA, isolated from, any tissue or cells expressing the antibody, such as hybridoma cells selected to express an antibody of the invention) by PCR amplification using synthetic primers hybridizable to the 3′ and 5′ ends of the sequence or by cloning using an oligonucleotide probe specific for the particular gene sequence to identify, e.g., a cDNA clone from a cDNA library that encodes the antibody. Amplified nucleic acids generated by PCR may then be cloned into replicable cloning vectors using any method well known in the art.

Once the nucleotide sequence and corresponding amino acid sequence of the antibody is determined, the nucleotide sequence of the antibody may be manipulated using methods well known in the art for the manipulation of nucleotide sequences, e.g., recombinant DNA techniques, site directed mutagenesis, PCR, etc. (see, for example, the techniques described in Sambrook et al., 1990, Molecular Cloning, A Laboratory Manual, 2d Ed., Cold Spring Harbor Laboratory, Cold Spring Harbor, N.Y. and Ausubel et al., eds., 1998, Current Protocols in Molecular Biology, John Wiley & Sons, NY, which are both incorporated by reference herein in their entireties ), to generate antibodies having a different amino acid sequence, for example to create amino acid substitutions, deletions, and/or insertions.

Methods of Producing Antibodies

The antibodies of the invention can be produced by any method known in the art for the synthesis of antibodies, in particular, by chemical synthesis or preferably, by recombinant expression techniques. Recombinant expression of an antibody of the invention, or fragment, derivative or analog thereof, (e.g., a heavy or light chain of an antibody of the invention or a single chain antibody of the invention), requires construction of an expression vector containing a polynucleotide that encodes the antibody. Once a polynucleotide encoding an antibody molecule or a heavy or light chain of an antibody, or portion thereof (preferably containing the heavy or light chain variable domain), of the invention has been obtained, the vector for the production of the antibody molecule may be produced by recombinant DNA technology using techniques well known in the art. Thus, methods for preparing a protein by expressing a polynucleotide containing an antibody encoding nucleotide sequence are described herein. Methods which are well known to those skilled in the art can be used to construct expression vectors containing antibody coding sequences and appropriate transcriptional and translational control signals. These methods include, for example, in vitro recombinant DNA techniques, synthetic techniques, and in vivo genetic recombination. The invention, thus, provides replicable vectors comprising a nucleotide sequence encoding an antibody molecule of the invention, or a heavy or light chain thereof, or a heavy or light chain variable domain, operably linked to a promoter. Such vectors may include the nucleotide sequence encoding the constant region of the antibody molecule (see, e.g., PCT Publication WO 86/05807; PCT Publication WO 89/01036; and U.S. Pat. No. 5,122,464) and the variable domain of the antibody may be cloned into such a vector for expression of the entire heavy or light chain.

The expression vector is transferred to a host cell by conventional techniques and the transfected cells are then cultured by conventional techniques to produce an antibody of the invention. Thus, the invention includes host cells containing a polynucleotide encoding an antibody of the invention, or a heavy or light chain thereof, or a single chain antibody of the invention, operably linked to a heterologous promoter. In preferred embodiments for the expression of double-chained antibodies, vectors encoding both the heavy and light chains may be co-expressed in the host cell for expression of the entire immunoglobulin molecule, as detailed below.

A variety of host-expression vector systems may be utilized to express the antibody molecules of the invention. Such host-expression systems represent vehicles by which the coding sequences of interest may be produced and subsequently purified, but also represent cells which may, when transformed or transfected with the appropriate nucleotide coding sequences, express an antibody molecule of the invention in situ. These include but are not limited to microorganisms such as bacteria (e.g., E. coli, B. subtilis) transformed with recombinant bacteriophage DNA, plasmid DNA or cosmid DNA expression vectors containing antibody coding sequences; yeast (e.g., Saccharomyces, Pichia) transformed with recombinant yeast expression vectors containing antibody coding sequences; insect cell systems infected with recombinant virus expression vectors (e.g., baculovirus) containing antibody coding sequences; plant cell systems infected with recombinant virus expression vectors (e.g., cauliflower mosaic virus, CaMV; tobacco mosaic virus, TMV) or transformed with recombinant plasmid expression vectors (e.g., Ti plasmid) containing antibody coding sequences; or mammalian cell systems (e.g., COS, CHO, BHK, 293, 3T3 cells) harboring recombinant expression constructs containing promoters derived from the genome of mammalian cells (e.g., metallothionein promoter) or from mammalian viruses (e.g., the adenovirus late promoter; the vaccinia virus 7.5K promoter). Preferably, bacterial cells such as Escherichia coli, and more preferably, eukaryotic cells, especially for the expression of whole recombinant antibody molecule, are used for the expression of a recombinant antibody molecule. For example, mammalian cells such as Chinese hamster ovary cells (CHO), in conjunction with a vector such as the major intermediate early gene promoter element from human cytomegalovirus is an effective expression system for antibodies (Foecking et al., Gene 45:101 (1986); Cockett etal., Bio/Technology 8:2 (1990)).

In bacterial systems, a number of expression vectors may be advantageously selected depending upon the use intended for the antibody molecule being expressed. For example, when a large quantity of such a protein is to be produced, for the generation of pharmaceutical compositions of an antibody molecule, vectors which direct the expression of high levels of fusion protein products that are readily purified may be desirable. Such vectors include, but are not limited, to the E. coli expression vector pUR278 (Ruther et al., EMBO J. 2:1791 (1983)), in which the antibody coding sequence may be ligated individually into the vector in frame with the lac Z coding region so that a fusion protein is produced; prN vectors (Inouye & Inouye, Nucleic Acids Res. 13:3101-3109 (1985); Van Heeke & Schuster, J. Biol. Chem. 24:5503-5509 (1989)); and the like. pGEX vectors may also be used to express foreign polypeptides as fusion proteins with glutathione S-transferase (GST). In general, such fusion proteins are soluble and can easily be purified from lysed cells by adsorption and binding to matrix glutathione-agarose beads followed by elution in the presence of free glutathione. The pGEX vectors are designed to include thrombin or factor Xa protease cleavage sites so that the cloned target gene product can be released from the GST moiety.

In an insect system, Autographa californica nuclear polyhedrosis virus (AcNPV) is used as a vector to express foreign genes. The virus grows in Spodoptera frigiperda ceils. The antibody coding sequence may be cloned individually into non-essential regions (for example the polyhedrin gene) of the virus and placed under control of an AcNPV promoter (for example the polyhedrin promoter).

In mammalian host cells, a number of viral-based expression systems may be utilized. In cases where an adenovirus is used as an expression vector, the antibody coding sequence of interest may be ligated to an adenovirus transcription/tanslation control complex, e.g., the late promoter and tripartite leader sequence. This chimeric gene may then be inserted in the adenovirus genome by in vitro or in vivo recombination. Insertion in a non- essential region of the viral genome (e.g., region E1 or E3) will result in a recombinant virus that is viable and capable of expressing the antibody molecule in infected hosts, (e.g., see Logan & Shenk, Proc. Natl. Acad. Sci. USA 81:355-359 (1984)). Specific initiation signals may also be required for efficient translation of inserted antibody coding sequences. These signals include the ATG initiation codon and adjacent sequences. Furthermore, the initiation codon must be in phase with the reading frame of the desired coding sequence to ensure translation of the entire insert. These exogenous translational control signals and initiation codons can be of a variety of origins, both natural and synthetic. The efficiency of expression may be enhanced by the inclusion of appropriate transcription enhancer elements, transcription terminators, etc. (see Bittner et al., Methods in Enzymol. 153:51-544 (1987)).

Antibodies production is well known in the art. Exemplary methods and compositions for making antibodies may be found in PCT publications WO/00-55173, WO/01-07611 and WO/02-16429.

Immunophenotyping

The antibodies of the invention may be utilized for immunophenotyping of cell lines and biological samples. The translation product of the gene of the present invention may be useful as a cell specific marker, or more specifically as a cellular marker that is differentially expressed at various stages of differentiation and/or maturation of particular cell types. Monoclonal antibodies directed against a specific epitope, or combination of epitopes, will allow for the screening of cellular populations expressing the marker. Various techniques can be utilized using monoclonal antibodies to screen for cellular populations expressing the marker(s), and include magnetic separation using antibody-coated magnetic beads, “panning” with antibody attached to a solid matrix (i.e., plate), and flow cytometry (See, e.g., U.S. Pat. No. 5,985,660; and Morrison et al. Cell, 96:73749 (1999)).

These techniques allow for the screening of particular populations of cells, such as might be found with hematological malignancies (i.e. minimal residual disease (MRD) in acute leukemic patients) and “non-self cells in transplantations to prevent Graft-versus-Host Disease (GVHD). Alternatively, these techniques allow for the screening of hematopoietic stem and progenitor cells capable of undergoing proliferation and/or differentiation, as might be found in human umbilical cord blood.

Assays For Antibody Binding

The antibodies of the invention may be assayed for immunospecific binding by any method known in the art. The immunoassays which can be used include but are not limited to competitive and non-competitive assay systems using techniques such as western blots, radioimmunoassays, ELISA (enzyme linked immunosorbent assay), “sandwich” immunoassays, immunoprecipitation assays, precipitin reactions, gel diffusion precipitin reactions, immunodiffusion assays, agglutination assays, complement-fixation assays, immunoradiometric assays, fluorescent immunoassays, protein A immunoassays, to name but a few. Such assays are routine and well known in the art (see, e.g., Ausubel et al, eds, 1994, Current Protocols in Molecular Biology, Vol. 1, John Wiley & Sons, Inc., New York, which is incorporated by reference herein in its entirety). Exemplary immunoassays are described briefly below (but are not intended by way of limitation).

Immunoprecipitation protocols generally comprise lysing a population of cells in a lysis buffer such as RIPA buffer (1% NP40 or Triton X-100, 1% sodium deoxycholate, 0.1% SDS, 0.15 M NaCl, 0.01 M sodium phosphate at pH 7.2, 1% Trasylol) supplemented with protein phosphatase and/or protease inhibitors (e.g., EDTA, PMSF, aprotinin, sodium vanadate), adding the antibody of interest to the cell lysate, incubating for a period of time (e.g., 1-4 hours) at 4° C., adding protein A and/or protein G sepharose beads to the cell lysate, incubating for about an hour or more at 4° C., washing the beads in lysis buffer and resuspending the beads in SDS/sample buffer. The ability of the antibody of interest to immunoprecipitate a particular antigen can be assessed by, e.g., western blot analysis. One of skill in the art would be knowledgeable as to the parameters that can be modified to increase the binding of the antibody to an antigen and decrease the background (e.g., pre-clearing the cell lysate with sepharose beads). For further discussion regarding immunoprecipitation protocols see, e.g., Ausubel et al, eds, 1994, Current Protocols in Molecular Biology, Vol. 1, John Wiley & Sons, Inc., New York at 10.16.1.

Western blot analysis generally comprises preparing protein samples, electrophoresis of the protein samples in a polyacrylamide gel (e.g., 8%-20% SDS-PAGE depending on the molecular weight ofthe antigen), transferring the protein sample from the polyacrylamide gel to a membrane such as nitrocellulose, PVDF or nylon, blocking the membrane in blocking solution (e.g., PBS with 3% BSA or non-fat milk), washing the membrane in washing buffer (e.g., PBS-Tween 20), blocking the membrane with primary antibody (the antibody of interest) diluted in blocking buffer, washing the membrane in washing buffer, blocking the membrane with a secondary antibody (which recognizes the primary antibody, e.g., an anti-human antibody) conjugated to an enzymatic substrate (e.g., horseradish peroxidase or alkaline phosphatase) or radioactive molecule (e.g., 32P or 1251) diluted in blocking buffer, washing the membrane in wash buffer, and detecting the presence of the antigen. One of skill in the art would be knowledgeable as to the parameters that can be modified to increase the signal detected and to reduce the background noise. For further discussion regarding western blot protocols see, e.g., Ausubel et al, eds, 1994, Current Protocols in Molecular Biology, Vol. 1, John Wiley & Sons, Inc., New York at 10.8.1.

ELISAs comprise preparing antigen, coating the well of a 96 well microtiter plate with the antigen, adding the antibody of interest conjugated to a detectable compound such as an enzymatic substrate (e.g., horseradish peroxidase or alkaline phosphatase) to the well and incubating for a period of time, and detecting the presence of the antigen. In ELISAs the antibody of interest does not have to be conjugated to a detectable compound; instead, a second antibody (which recognizes the antibody of interest) conjugated to a detectable compound may be added to the well. Further, instead of coating the well with the antigen, the antibody may be coated to the well. In this case, a second antibody conjugated to a detectable compound may be added following the addition of the antigen of interest to the coated well. One of skill in the art would be knowledgeable as to the parameters that can be modified to increase the signal detected as well as other variations of ELISAs known in the art. For further discussion regarding ELISAs see, e.g., Ausubel et al, eds, 1994, Current Protocols in Molecular Biology, Vol. 1, John Wiley & Sons, Inc., New York at 11.2.1.

The binding affinity of an antibody to an antigen and the off-rate of an antibody-antigen interaction can be determined by competitive binding assays. One example of a competitive binding assay is a radioimmunoassay comprising the incubation of labeled antigen (e.g., 3H or 1251) with the antibody of interest in the presence of increasing amounts of unlabeled antigen, and the detection of the antibody bound to the labeled antigen. The affinity of the antibody of interest for a particular antigen and the binding off-rates can be determined from the data by scatchard plot analysis. Competition with a second antibody can also be determined using radioimmunoassays. In this case, the antigen is incubated with antibody of interest conjugated to a labeled compound (e.g., 3H or 1251) in the presence of increasing amounts of an unlabeled second antibody.

Therapeutic Uses

The present invention is further directed to antibody-based therapies which involve administering antibodies of the invention to an animal, preferably a mammal, and most preferably a human, patient for treating one or more of the disclosed diseases, disorders, or conditions. Therapeutic compounds of the invention include, but are not limited to, antibodies of the invention (including fragments, analogs and derivatives thereof as described herein) and nucleic acids encoding antibodies of the invention (including fragments, analogs and derivatives thereof and anti-idiotypic antibodies as described herein). The antibodies of the invention can be used to treat, inhibit or prevent diseases, disorders or conditions associated with aberrant expression and/or activity of a polypeptide of the invention, including, but not limited to, any one or more of the diseases, disorders, or conditions described herein. The treatment and/or prevention of diseases, disorders, or conditions associated with aberrant expression and/or activity of a polypeptide of the invention includes, but is not limited to, alleviating symptoms associated with those diseases, disorders or conditions. Antibodies of the invention may be provided in pharmaceutically acceptable compositions as known in the art or as described herein.

A summary of the ways in which the antibodies of the present invention may be used therapeutically includes binding polynucleotides or polypeptides of the present invention locally or systemically in the body or by direct cytotoxicity of the antibody, e.g. as mediated by complement (CDC) or by effector cells (ADCC). Some of these approaches are described in more detail below. Armed with the teachings provided herein, one of ordinary skill in the art will know how to use the antibodies of the present invention for diagnostic, monitoring or therapeutic purposes without undue experimentation.

The antibodies of this invention may be advantageously utilized in combination with other monoclonal or chimeric antibodies, or with lymphokines or hematopoietic growth factors (such as, e.g., IL-2, IL-3 and IL-7), for example, which serve to increase the number or activity of effector cells which interact with the antibodies.

The antibodies of the invention may be administered alone or in combination with other types of treatments (e.g., radiation therapy, chemotherapy, hormonal therapy, immunotherapy and anti-tumor agents). Generally, administration of products of a species origin or species reactivity (in the case of antibodies) that is the same species as that of the patient is preferred. Thus, in a preferred embodiment, human antibodies, fragments derivatives, analogs, or nucleic acids, are administered to a human patient for therapy or prophylaxis.

It is preferred to use high affinity and/or potent in vivo inhibiting and/or neutralizing antibodies against polypeptides or polynucleotides of the present invention, fragments or regions thereof, for both immunoassays directed to and therapy of disorders related to polynucleotides or polypeptides, including fragments thereof, of the present invention. Such antibodies, fragments, or regions, will preferably have an affinity for polynucleotides or polypeptides of the invention, including fragments thereof Preferred binding affinities include those with a dissociation constant or Kd less than 5×10⁻² M, 10⁻² M, 5×10⁻³ M, 10⁻³ M, 5×10⁻⁴ M, 10⁻⁴ M, 5×10⁻⁵ M, 10⁻⁵ M, 5×10⁻⁶ M, 10⁻⁶ M, 5×10⁻⁷ M, 10⁻⁷ M, 5×1D⁻⁸ M, 10⁻⁸ M, 5×10⁻⁹ M, 10⁻⁹ M, 5×1O⁻¹⁰M, 10⁻¹⁰ M, 5×1O⁻¹¹ M, 1O^{×11} M, 5×10⁻¹² M, 10⁻¹² M, 5×10⁻¹³ M, 1O⁻¹³ M, 5×10⁻¹⁴ M, 10⁻¹⁴ M, 5×10⁻¹⁵ M, or 10⁻¹⁵ M.

Kits

Also provided by the subject invention are kits for practicing the subject methods, as described above. The subject kits include at least one or more of: a subject nucleic acid, isolated polypeptide or an antibody thereto. Other optional components of the kit include: restriction enzymes, control primers and plasmids; buffers, cells, carriers adjuvents etc. The nucleic acids of the kit may also have restrictions sites, multiple cloning sites, primer sites, etc to facilitate their ligation other plasmids. The various components of the kit may be present in separate containers or certain compatible components may be precombined into a single container, as desired. In many embodiments, kits with unit doses of the active agent, e.g. in oral or injectable doses, are provided. In certain embodiments, controls, such as samples from a cancerous or non-cancerous cell are provided by the invention. Further embodiments of the kit include an antibody for a subject polypeptide and a chemotherapeutic agent to be used in combination with the polypeptide as a treatment.

In addition to above-mentioned components, the subject kits typically further include instructions for using the components of the kit to practice the subject methods. The instructions for practicing the subject methods are generally recorded on a suitable recording medium. For example, the instructions may be printed on a substrate, such as paper or plastic, etc. As such, the instructions may be present in the kits as a package insert, in the labeling of the container of the kit or components thereof (i.e., associated with the packaging or subpackaging) etc. In other embodiments, the instructions are present as an electronic storage data file present on a suitable computer readable storage medium, e.g. CD-ROM, diskette, etc. In yet other embodiments, the actual instructions are not present in the kit, but means for obtaining the instructions from a remote source, e.g. via the internet, are provided. An example of this embodiment is a kit that includes a web address where the instructions can be viewed and/or from which the instructions can be downloaded. As with the instructions, this means for obtaining the instructions is recorded on a suitable substrate.

### COMPUTER-RELATED EMBODIMENTS

In general, a library of polynucleotides is a collection of sequence information, which information is provided in either biochemical form (e.g., as a collection of polynucleotide molecules), or in electronic form (e.g., as a collection of polynucleotide sequences stored in a computer-readable form, as in a computer system and/or as part of a computer program). The sequence information of the polynucleotides can be used in a variety of ways, e.g., as a resource for gene discovery, as a representation of sequences expressed in a selected cell type (e.g., cell type markers), and/or as markers of a given disease or disease state. For example, in the instant case, the sequences of polynucleotides and polypeptides corresponding to genes differentially expressed in cancer, particular in breast cancer, as well as the nucleic acid and amino acid sequences of the genes themselves, can be provided in electronic form in a computer database.

In general, a disease marker is a representation of a gene product that is present in all cells affected by disease either at an increased or decreased level relative to a normal cell (e.g., a cell of the same or similar type that is not substantially affected by disease). For example, a polynucleotide sequence in a library can be a polynucleotide that represents an mRNA, polypeptide, or other gene product encoded by the polynucleotide, that is either overexpressed or underexpressed in a cancerous breast cell affected by cancer relative to a normal (ie., substantially disease-free) breast cell.

The nucleotide sequence information of the library can be embodied in any suitable form, e.g., electronic or biochemical forms. For example, a library of sequence information embodied in electronic form comprises an accessible computer data file (or, in biochemical form, a collection of nucleic acid molecules) that contains the representative nucleotide sequences of genes that are differentially expressed (e.g., overexpressed or underexpressed) as between, for example, i) a cancerous cell and a normal cell; ii) a cancerous cell and a dysplastic cell; iii) a cancerous cell and a cell affected by a disease or condition other than cancer; iv) a metastatic cancerous cell and a normal cell and/or non-metastatic cancerous cell; v) a malignant cancerous cell and a non-malignant cancerous cell (or a normal cell) and/or vi) a dysplastic cell relative to a normal cell. Other combinations and comparisons of cells affected by various diseases or stages of disease will be readily apparent to the ordinarily skilled artisan. Biochemical embodiments of the library include a collection of nucleic acids that have the sequences of the genes in the library, where the nucleic acids can correspond to the entire gene in the library or to a fragment thereof, as described in greater detail below.

The polynucleotide libraries of the subject invention generally comprise sequence information of a plurality of polynucleotide sequences, where at least one of the polynucleotides has a sequence of any of sequence described herein. By plurality is meant at least 2, usually at least 3 and can include up to all of the sequences described herein. The length and number of polynucleotides in the library will vary with the nature of the library, e.g., if the library is an oligonucleotide array, a cDNA array, a computer database of the sequence information, etc.

Where the library is an electronic library, the nucleic acid sequence information can be present in a variety of media. “Media” refers to a manufacture, other than an isolated nucleic acid molecule, that contains the sequence information of the present invention. Such a manufacture provides the genome sequence or a subset thereof in a form that can be examined by means not directly applicable to the sequence as it exists in a nucleic acid. For example, the nucleotide sequence of the present invention, e.g. the nucleic acid sequences of any of the polynucleotides of the sequences described herein, can be recorded on computer readable media, e.g. any medium that can be read and accessed directly by a computer. Such media include, but are not limited to: magnetic storage media, such as a floppy disc, a hard disc storage medium, and a magnetic tape; optical storage media such as CD-ROM; electrical storage media such as RAM and ROM; and hybrids of these categories such as magnetic/optical storage media.

One of skill in the art can readily appreciate how any of the presently known computer readable mediums can be used to create a manufacture comprising a recording of the present sequence information. “Recorded” refers to a process for storing information on computer readable medium, using any such methods as known in the art. Any convenient data storage structure can be chosen, based on the means used to access the stored information. A variety of data processor programs and formats can be used for storage, e.g. word processing text file, database format, etc. In addition to the sequence information, electronic versions of libraries comprising one or more sequence described herein can be provided in conjunction or connection with other computer-readable information and/or other types of computer-readable files (e.g., searchable files, executable files, etc, including, but not limited to, for example, search program software, etc.).

By providing the nucleotide sequence in computer readable form, the information can be accessed for a variety of purposes. Computer software to access sequence information (e.g. the NCBI sequence database) is publicly available. For example, the gapped BLAST (Altschul et al., Nucleic Acids Res. (1997) 25:3389-3402) and BLAZE (Brutlag et al, Comp. Chem. (1993) 17:203) search algorithms on a Sybase system, or the TeraBLAST (TimeLogic, Crystal Bay, Nev.) program optionally running on a specialized computer platform available from TimeLogic, can be used to identify open reading frames (ORFs) within the genome that contain homology to ORFs from other organisms.

As used herein, “a computer-based system” refers to the hardware means, software means, and data storage means used to analyze the nucleotide sequence information of the present invention. The minimum hardware of the computer-based systems of the present invention comprises a central processing unit (CPU), input means, output means, and data storage means. A skilled artisan can readily appreciate that any one of the currently available computer-based system are suitable for use in the present invention. The data storage means can comprise any manufacture comprising a recording of the present sequence information as described above, or a memory access means that can access such a manufacture.

“Search means” refers to one or more programs implemented on the computer-based system, to compare a target sequence or target structural motif, or expression levels of a polynucleotide in a sample, with the stored sequence information. Search means can be used to identify fragments or regions of the genome that match a particular target sequence or target motif. A variety of known algorithms are publicly known and commercially available, e.g. MacPattern (EMBL), TeraBLAST (TimeLogic), BLASTN and BLASTX (NCBI). A “target sequence” can be any polynucleotide or amino acid sequence of six or more contiguous nucleotides or two or more amino acids, preferably from about 10 to 100 amino acids or from about 30 to 300 nt. A variety of means for comparing nucleic acids or polypeptides may be used to compare accomplish a sequence comparison (e.g., to analyze target sequences, target motifs, or relative expression levels) with the data storage means. A skilled artisan can readily recognize that any one of the publicly available homology search programs can be used to search the computer based systems of the present invention to compare of target sequences and motifs. Computer programs to analyze expression levels in a sample and in controls are also known in the art.

A “target structural motif,” or “target motif,” refers to any rationally selected sequence or combination of sequences in which the sequence(s) are chosen based on a three-dimensional configuration that is formed upon the folding of the target motif, or on consensus sequences of regulatory or active sites. There are a variety of target motifs known in the art. Protein target motifs include, but are not limited to, enzyme active sites and signal sequences, kinase domains, receptor binding domains, SH2 domains, SH3 domains, phosphorylation sites, protein interaction domains, transmembrane domains, etc. Nucleic acid target motifs include, but are not limited to, hairpin structures, promoter sequences and other expression elements such as binding sites for transcription factors.

A variety of structural formats for the input and output means can be used to input and output the information in the computer-based systems of the present invention. One format for an output means ranks the relative expression levels of different polynucleotides. Such presentation provides a skilled artisan with a ranking of relative expression levels to determine a gene expression profile. A gene expression profile can be generated from, for example, a cDNA library prepared from mRNA isolated from a test cell suspected of being cancerous or pre-cancerous, comparing the sequences or partial sequences of the clones against the sequences in an electronic database, where the sequences of the electronic database represent genes differentially expressed in a cancerous cell, e.g., a cancerous breast cell. The number of clones having a sequence that has substantial similarity to a sequence that represents a gene differentially expressed in a cancerous cell is then determined, and the number of clones corresponding to each of such genes is determined. An increased number of clones that correspond to differentially expressed gene is present in the cDNA library of the test cell (relative to, for example, the number of clones expected in a cDNA of a normal cell) indicates that the test cell is cancerous.

As discussed above, the “library” as used herein also encompasses biochemical libraries of the polynucleotides of the sequences described herein, e.g., collections of nucleic acids representing the provided polynucleotides. The biochemical libraries can take a variety of forms, e.g., a solution of cDNAs, a pattern of probe nucleic acids stably associated with a surface of a solid support (ie., an array) and the like. Of particular interest are nucleic acid arrays in which one or more of the genes described herein is represented by a sequence on the array. By array is meant an article of manufacture that has at least a substrate with at least two distinct nucleic acid targets on one of its surfaces, where the number of distinct nucleic acids can be considerably higher, typically being at least 10 nt, usually at least 20 nt and often at least 25 nt. A variety of different array formats have been developed and are known to those of skill in the art. The arrays of the subject invention find use in a variety of applications, including gene expression analysis, drug screening, mutation analysis and the like, as disclosed in the above-listed exemplary patent documents.

In addition to the above nucleic acid libraries, analogous libraries of polypeptides are also provided, where the polypeptides of the library will represent at least a portion of the polypeptides encoded by a gene corresponding to a sequence described herein.

Diagnostic and Other Methods Involving Detection of Differentially Expressed Genes

The present invention provides methods of using the polynucleotides described herein in, for example, diagnosis of cancer and classification of cancer cells according to expression profiles. In specific non-limiting embodiments, the methods are useful for detecting breast cancer cells, facilitating diagnosis of cancer and the severity of a cancer (e.g., tumor grade, tumor burden, and the like) in a subject, facilitating a determination of the prognosis of a subject, and assessing the responsiveness of the subject to therapy (e.g., by providing a measure of therapeutic effect through, for example, assessing tumor burden during or following a chemotherapeutic regimen). Detection can be based on detection of a polynucleotide that is differentially expressed in a breast cancer cell, and/or detection of a polypeptide encoded by a polynucleotide that is differentially expressed in a breast cancer cell (“a polypeptide associated with breast cancer”). The detection methods of the invention can be conducted in vitro or in vivo, on isolated cells, or in whole tissues or a bodily fluid, e.g., blood, plasma, serum, urine, and the like).

In general, methods of the invention involving detection of a gene product (e.g., mRNA, cDNA generated from such mRNA, and polypeptides) involve contacting a sample with a probe specific for the gene product of interest. “Probe” as used herein in such methods is meant to refer to a molecule that specifically binds a gene product of interest (e.g., the probe binds to the target gene product with a specificity sufficient to distinguish binding to target over non-specific binding to non-target (background) molecules). “Probes” include, but are not necessarily limited to, nucleic acid probes (e.g., DNA, RNA, modified nucleic acid, and the like), antibodies (e.g., antibodies, antibody fragments that retain binding to a target epitope, single chain antibodies, and the like), or other polypeptide, peptide, or molecule (e.g., receptor ligand) that specifically binds a target gene product of interest.

The probe and sample suspected of having the gene product of interest are contacted under conditions suitable for binding of the probe to the gene product. For example, contacting is generally for a time sufficient to allow binding of the probe to the gene product (e.g., from several minutes to a few hours), and at a temperature and conditions of osmolarity and the like that provide for binding of the probe to the gene product at a level that is sufficiently distinguishable from background binding of the probe (e.g., under conditions that minimize non-specific binding). Suitable conditions for probe-target gene product binding can be readily determined using controls and other techniques available and known to one of ordinary skill in the art.

In this embodiment, the probe can be an antibody or other polypeptide, peptide, or molecule (e.g., receptor ligand) that specifically binds a target polypeptide of interest.

The detection methods can be provided as part of a kit. Thus, the invention further provides kits for detecting the presence and/or a level of a polynucleotide that is differentially expressed in a breast cancer cell (e.g., by detection of an mRNA encoded by the differentially expressed gene of interest), and/or a polypeptide encoded thereby, in a biological sample. Procedures using these kits can be performed by clinical laboratories, experimental laboratories, medical practitioners, or private individuals. The kits of the invention for detecting a polypeptide encoded by a polynucleotide that is differentially expressed in a breast cancer cell comprise a moiety that specifically binds the polypeptide, which may be a specific antibody. The kits of the invention for detecting a polynucleotide that is differentially expressed in a breast cancer cell comprise a moiety that specifically hybridizes to such a polynucleotide. The kit may optionally provide additional components that are useful in the procedure, including, but not limited to, buffers, developing reagents, labels, reacting surfaces, means for detection, control samples, standards, instructions, and interpretive information.

Detecting a Polypeptide Encoded by a Polynucleotide that is Differentially Expressed in a Breast Cancer Cell

In some embodiments, methods are provided for a detecting breast cancer cell by detecting in a cell, particularly a breast cell, a polypeptide encoded by a gene differentially expressed in a breast cancer cell. Any of a variety of known methods can be used for detection, including, but not limited to, immunoassay, using an antibody specific for the encoded polypeptide, e.g., by enzyme-linked immunosorbent assay (ELISA), radioimmunoassay (RIA), and the like; and functional assays for the encoded polypeptide, e.g., binding activity or enzymatic activity.

For example, an immunofluorescence assay can be easily performed on cells without first isolating the encoded polypeptide. The cells are first fixed onto a solid support, such as a microscope slide or microtiter well. This fixing step can permeabilize the cell membrane. The permeablization of the cell membrane permits the polypeptide-specific probe (e.g, antibody) to bind. Alternatively, where the polypeptide is secreted or membrane-bound, or is otherwise accessible at the cell-surface (e.g., receptors, and other molecule stably-associated with the outer cell membrane or otherwise stably associated with the cell membrane, such permeabilization may not be necessary.

Next, the fixed cells are exposed to an antibody specific for the encoded polypeptide. To increase the sensitivity of the assay, the fixed cells may be further exposed to a second antibody, which is labeled and binds to the first antibody, which is specific for the encoded polypeptide. Typically, the secondary antibody is detectably labeled, e.g., with a fluorescent marker. The cells which express the encoded polypeptide will be fluorescently labeled and easily visualized under the microscope. See, for example, Hashido et al. (1992) Biochem. Biophys. Res. Comm. 187:1241-1248.

As will be readily apparent to the ordinarily skilled artisan upon reading the present specification, the detection methods and other methods described herein can be varied. Such variations are within the intended scope of the invention. For example, in the above detection scheme, the probe for use in detection can be immobilized on a solid support, and the test sample contacted with the immobilized probe. Binding of the test sample to the probe can then be detected in a variety of ways, e.g., by detecting a detectable label bound to the test sample.

The present invention further provides methods for detecting the presence of and/or measuring a level of a polypeptide in a biological sample, which polypeptide is encoded by a polynucleotide that represents a gene differentially expressed in cancer, particularly in a polynucleotide that represents a gene differentially cancer cell, using a probe specific for the encoded polypeptide. In this embodiment, the probe can be a an antibody or other polypeptide, peptide, or molecule (e.g., receptor ligand) that specifically binds a target polypeptide of interest.

The methods generally comprise: a) contacting the sample with an antibody specific for a differentially expressed polypeptide in a test cell; and b) detecting binding between the antibody and molecules of the sample. The level of antibody binding (either qualitative or quantitative) indicates the cancerous state of the cell. For example, where the differentially expressed gene is increased in cancerous cells, detection of an increased level of antibody binding to the test sample relative to antibody binding level associated with a normal cell indicates that the test cell is cancerous.

Suitable controls include a sample known not to contain the encoded polypeptide; and a sample contacted with an antibody not specific for the encoded polypeptide, e.g., an anti-idiotype antibody. A variety of methods to detect specific antibody-antigen interactions are known in the art and can be used in the method, including, but not limited to, standard immunohistological methods, immunoprecipitation, an enzyme immunoassay, and a radioimmunoassay.

In general, the specific antibody will be detectably labeled, either directly or indirectly. Direct labels include radioisotopes; enzymes whose products are detectable (e.g., luciferase, β-galactosidase, and the like); fluorescent labels (e.g., fluorescein isothiocyanate, rhodamine, phycoerythrin, and the like); fluorescence emitting metals, e.g., ¹⁵²Eu, or others of the lanthanide series, attached to the antibody through metal chelating groups such as EDTA; chemiluminescent compounds, e.g., luninol, isoluminol, acridinium salts, and the like; bioluminescent compounds, e.g., luciferin, aequorin (green fluorescent protein), and the like.

The antibody may be attached (coupled) to an insoluble support, such as a polystyrene plate or a bead. Indirect labels include second antibodies specific for antibodies specific for the encoded polypeptide (“first specific antibody”), wherein the second antibody is labeled as described above; and members of specific binding pairs, e.g., biotin-avidin, and the like. The biological sample may be brought into contact with and immobilized on a solid support or carrier, such as nitrocellulose, that is capable of immobilizing cells, cell particles, or soluble proteins. The support may then be washed with suitable buffers, followed by contacting with a detectably-labeled first specific antibody. Detection methods are known in the art and will be chosen as appropriate to the signal emitted by the detectable label. Detection is generally accomplished in comparison to suitable controls, and to appropriate standards.

In some embodiments, the methods are adapted for use in vivo, e.g., to locate or identify sites where breast cancer cells are present. In these embodiments, a detectably-labeled moiety, e.g., an antibody, which is specific for a breast cancer-associated polypeptide is administered to an individual (e.g., by injection), and labeled cells are located using standard imaging techniques, including, but not limited to, magnetic resonance imaging, computed tomography scanning, and the like. In this manner, breast cancer cells are differentially labeled.

Detecting a Polynucleotide that Represents a Gene Differentially Expressed in a Breast Cancer Cell

In some embodiments, methods are provided for detecting a breast cancer cell by detecting expression in the cell of a transcript or that is differentially expressed in a breast cancer cell. Any of a variety of known methods can be used for detection, including, but not limited to, detection of a transcript by hybridization with a polynucleotide that hybridizes to a polynucleotide that is differentially expressed in a breast cancer cell; detection of a transcript by a polymerase chain reaction using specific oligonucleotide primers; in situ hybridization of a cell using as a probe a polynucleotide that hybridizes to a gene that is differentially expressed in a breast cancer cell and the like.

In many embodiments, the levels of a subject gene product are measured. By measured is meant qualitatively or quantitatively estimating the level of the gene product in a first biological sample either directly (e.g. by determining or estimating absolute levels of gene product) or relatively by comparing the levels to a second control biological sample. In many embodiments the second control biological sample is obtained from an individual not having not having breast cancer. As will be appreciated in the art, once a standard control level of gene expression is known, it can be used repeatedly as a standard for comparison. Other control samples include samples of cancerous breast tissue.

The methods can be used to detect and/or measure mRNA levels of a gene that is differentially expressed in a breast cancer cell. In some embodiments, the methods comprise: a) contacting a sample with a polynucleotide that corresponds to a differentially expressed gene described herein under conditions that allow hybridization; and b) detecting hybridization, if any. Detection of differential hybridization, when compared to a suitable control, is an indication of the presence in the sample of a polynucleotide that is differentially expressed in a breast cancer cell. Appropriate controls include, for example, a sample that is known not to contain a polynucleotide that is differentially expressed in a breast cancer cell. Conditions that allow hybridization are known in the art, and have been described in more detail above.

Detection can also be accomplished by any known method, including, but not limited to, in situ hybridization, PCR (polymerase chain reaction), RT-PCR (reverse transcription-PCR), and “Northern” or RNA blotting, arrays, microarrays, etc, or combinations of such techniques, using a suitably labeled polynucleotide. A variety of labels and labeling methods for polynucleotides are known in the art and can be used in the assay methods of the invention. Specific hybridization can be determined by comparison to appropriate controls.

Polynucleotide generally comprising at least 12 contiguous nt of a polynucleotide provided herein, as shown in the Sequence Listing or of the sequences of the genes corresponding to the polynucleotides of the Sequence Listing, are used for a variety of purposes, such as probes for detection of and/or measurement of, transcription levels of a polynucleotide that is differentially expressed in a breast cancer cell. Additional disclosure about preferred regions of the disclosed polynucleotide sequences is found in the Examples. A probe that hybridizes specifically to a polynucleotide disclosed herein should provide a detection signal at least 2-, 5-, 10-, or 20-fold higher than the background hybridization provided with other unrelated sequences. It should be noted that “probe” as used in this context of detection of nucleic acid is meant to refer to a polynucleotide sequence used to detect a differentially expressed gene product in a test sample. As will be readily appreciated by the ordinarily skilled artisan, the probe can be detectably labeled and contacted with, for example, an array comprising immobilized polynucleotides obtained from a test sample (e.g., mRNA). Alternatively, the probe can be immobilized on an array and the test sample detectably labeled. These and other variations of the methods of the invention are well within the skill in the art and are within the scope of the invention.

Labeled nucleic acid probes may be used to detect expression of a gene corresponding to the provided polynucleotide. In Northern blots, mRNA is separated electrophoretically and contacted with a probe. A probe is detected as hybridizing to an mRNA species of a particular size. The amount of hybridization can be quantitated to determine relative amounts of expression, for example under a particular condition. Probes are used for in situ hybridization to cells to detect expression. Probes can also be used in vivo for diagnostic detection of hybridizing sequences. Probes are typically labeled with a radioactive isotope. Other types of detectable labels can be used such as chromophores, fluorophores, and enzymes. Other examples of nucleotide hybridization assays are described in W092/02526 and U.S. Pat. No. 5,124,246.

PCR is another means for detecting small amounts of target nucleic acids, methods for which may be found in Sambrook, et al. Molecular Cloning: A Laboratory Manual, CSH Press 1989, pp.14.2-14.33.

A detectable label may be included in the amplification reaction. Suitable detectable labels include fluorochromes,(e.g. fluorescein isothiocyanate (FITC), rhodamine, Texas Red, phycoerythrin, allophycocyanin, 6-carboxyfluorescein (6-FAM), 2′,7′-dimethoxy-4′,5′-dichloro-6-carboxyfluorescein, 6-carboxy-X-rhodamine (ROX), 6-carboxy-2′,4′,7′,4,7-hexachlorofluorescein (HEX), 5-carboxyfluorescein (5-FAM) or N,N,N′,N′-tetramethyl-6-carboxyrhodamine (TAMRA)), radioactive labels, (e.g. ³²P, ³⁵S, ³H, etc.), and the like. The label may be a two stage system, where the polynucleotides is conjugated to biotin, haptens, etc. having a high affinity binding partner, e.g. avidin, specific antibodies, etc., where the binding partner is conjugated to a detectable label. The label may be conjugated to one or both of the primers. Alternatively, the pool of nucleotides used in the amplification is labeled, so as to incorporate the label into the amplification product.

Arrays

Polynucleotide arrays provide a high throughput technique that can assay a large number of polynucleotides or polypeptides in a sample. This technology can be used as a tool to test for differential expression.

A variety of methods of producing arrays, as well as variations of these methods, are known in the art and contemplated for use in the invention. For example, arrays can be created by spotting polynucleotide probes onto a substrate (e.g., glass, nitrocellulose, etc.) in a two-dimensional matrix or array having bound probes. The probes can be bound to the substrate by either covalent bonds or by non-specific interactions, such as hydrophobic interactions.

Samples of polynucleotides can be detectably labeled (e.g., using radioactive or fluorescent labels) and then hybridized to the probes. Double stranded polynucleotides, comprising the labeled sample polynucleotides bound to probe polynucleotides, can be detected once the unbound portion of the sample is washed away. Alternatively, the polynucleotides of the test sample can be immobilized on the array, and the probes detectably labeled. Techniques for constructing arrays and methods of using these arrays are described in, for example, Schena et al. (1996) Proc Natl Acad Sci USA. 93(20):10614-9; Schena et al. (1995) Science 270(5235):467-70; Shalon et al. (1996) Genome Res. 6(7):639-45, U.S. Pat. No. 5,807,522, EP 799 897; WO 97/29212; WO 97/27317; EP 785 280; WO 97/02357; U.S. Pat. No. 5,593,839; U.S. Pat. No. 5,578,832; EP 728 520; U.S. Pat. No. 5,599,695; EP 721 016; U.S. Pat. No. 5,556,752; WO 95/22058; and U.S. Pat. No. 5,631,734. In most embodiments, the “probe” is detectably labeled. In other embodiments, the probe is immobilized on the array and not detectably labeled.

Arrays can be used, for example, to examine differential expression of genes and can be used to determine gene function. For example, arrays can be used to detect differential expression of a gene corresponding to a polynucleotide described herein, where expression is compared between a test cell and control cell (e.g., cancer cells and normal cells). For example, high expression of a particular message in a cancer cell, which is not observed in a corresponding normal cell, can indicate a cancer specific gene product. Exemplary uses of arrays are further described in, for example, Pappalarado et al., Sem. Radiation Oncol. (1998) 8:217; and Ramsay, Nature Biotechnol. (1998) 16:40. Furthermore, many variations on methods of detection using arrays are well within the skill in the art and within the scope of the present invention. For example, rather than immobilizing the probe to a solid support, the test sample can be immobilized on a solid support which is then contacted with the probe.

Diagnosis, Prognosis, Assessment of Therapy (Therametrics), and Management of Cancer

The polynucleotides described herein, as well as their gene products and corresponding genes and gene products, are of particular interest as genetic or biochemical markers (e.g., in blood or tissues) that will detect the earliest changes along the carcinogenesis pathway and/or to monitor the efficacy of various therapies and preventive interventions.

For example, the level of expression of certain polynucleotides can be indicative of a poorer prognosis, and therefore warrant more aggressive chemo- or radio-therapy for a patient or vice versa. The correlation of novel surrogate tumor specific features with response to treatment and outcome in patients can define prognostic indicators that allow the design of tailored therapy based on the molecular profile of the tumor. These therapies include antibody targeting, antagonists (e.g., small molecules), and gene therapy.

Determining expression of certain polynucleotides and comparison of a patient's profile with known expression in normal tissue and variants of the disease allows a determination of the best possible treatment for a patient, both in terms of specificity of treatment and in terms of comfort level of the patient. Surrogate tumor markers, such as polynucleotide expression, can also be used to better classify, and thus diagnose and treat, different forms and disease states of cancer. Two classifications widely used in oncology that can benefit from identification of the expression levels of the genes corresponding to the polynucleotides described herein are staging of the cancerous disorder, and grading the nature of the cancerous tissue.

The polynucleotides that correspond to differentially expressed genes, as well as their encoded gene products, can be useful to monitor patients having or susceptible to cancer to detect potentially malignant events at a molecular level before they are detectable at a gross morphological level. In addition, the polynucleotides described herein, as well as the genes corresponding to such polynucleotides, can be useful as therametrics, e.g., to assess the effectiveness of therapy by using the polynucleotides or their encoded gene products, to assess, for example, tumor burden in the patient before, during, and after therapy.

Furthermore, a polynucleotide identified as corresponding to a gene that is differentially expressed in, and thus is important for, one type of cancer can also have implications for development or risk of development of other types of cancer, e.g., where a polynucleotide represents a gene differentially expressed across various cancer types. Thus, for example, expression of a polynucleotide corresponding to a gene that has clinical implications for breast cancer can also have clinical implications for metastatic breast cancer, colon cancer, or ovarian cancer.

Staging. Staging is a process used by physicians to describe how advanced the cancerous state is in a patient. Staging assists the physician in determining a prognosis, planning treatment and evaluating the results of such treatment. Staging systems vary with the types of cancer, but generally involve the following “TNM” system: the type of tumor, indicated by T; whether the cancer has metastasized to nearby lymph nodes, indicated by N; and whether the cancer has metastasized to more distant parts of the body, indicated by M. Generally, if a cancer is only detectable in the area of the primary lesion without having spread to any lymph nodes it is called Stage I. If it has spread only to the closest lymph nodes, it is called Stage II. In Stage III, the cancer has generally spread to the lymph nodes in near proximity to the site of the primary lesion. Cancers that have spread to a distant part of the body, such as the liver, bone, brain or other site, are Stage IV, the most advanced stage.

The polynucleotides and corresponding genes and gene products described herein can facilitate fine-tuning of the staging process by identifying markers for the aggressiveness of a cancer, e.g. the metastatic potential, as well as the presence in different areas of the body. Thus, a Stage II cancer with a polynucleotide signifying a high metastatic potential cancer can be used to change a borderline Stage II tumor to a Stage III tumor, justifying more aggressive therapy. Conversely, the presence of a polynucleotide signifying a lower metastatic potential allows more conservative staging of a tumor.

One type of breast cancer is ductal carcinoma in situ (DCIS): DCIS is when the breast cancer cells are completely contained within the breast ducts (the channels in the breast that carry milk to the nipple), and have not spread into the surrounding breast tissue. This may also be referred to as non-invasive or intraductal cancer, as the cancer cells have not yet spread into the surrounding breast tissue and so usually have not spread into any other part of the body.

Lobular carcinoma in situ breast cancer (LCIS) means that cell changes are found in the lining of the lobules of the breast. It can be present in both breasts. It is also referred to as non-invasive cancer as it has not spread into the surrounding breast tissue.

Invasive breast cancer can be staged as follows: Stage 1 tumours: these measure less than two centimetres. The lymph glands in the armpit are not affected and there are no signs that the cancer has spread elsewhere in the body; Stage 2 tumours: these measure between two and five centimetres, or the lymph glands in the armpit are affected, or both. However, there are no signs that the cancer has spread further; Stage 3 tumours: these are larger than five centimetres and may be attached to surrounding structures such as the muscle or skin. The lymph glands are usually affected, but there are no signs that the cancer has spread beyond the breast or the lymph glands in the armpit; Stage 4 tumours: these are of any size, but the lymph glands are usually affected and the cancer has spread to other parts of the body. This is secondary breast cancer.

Grading of cancers. Grade is a term used to describe how closely a tumor resembles normal tissue of its same type. The microscopic appearance of a tumor is used to identify tumor grade based on parameters such as cell morphology, cellular organization, and other markers of differentiation. As a general rule, the grade of a tumor corresponds to its rate of growth or aggressiveness, with undifferentiated or high-grade tumors generally being more aggressive than well-differentiated or low-grade tumors.

The polynucleotides of the Sequence Listing, and their corresponding genes and gene products, can be especially valuable in determining the grade of the tumor, as they not only can aid in determining the differentiation status of the cells of a tumor, they can also identify factors other than differentiation that are valuable in determining the aggressiveness of a tumor, such as metastatic potential.

Low grade means that the cancer cells look very like the normal cells of the breast. They are usually slowly growing and are less likely to spread. In high grade tumors the cells look very abnormal. They are likely to grow more quickly and are more likely to spread.

Assessment of proliferation of cells in tumor. The differential expression level of the polynucleotides described herein can facilitate assessment of the rate of proliferation of tumor cells, and thus provide an indicator of the aggressiveness of the rate of tumor growth. For example, assessment of the relative expression levels of genes involved in cell cycle can provide an indication of cellular proliferation, and thus serve as a marker of proliferation.

Detection of Breast Cancer.

The polynucleotides corresponding to genes that exhibit the appropriate expression pattern can be used to detect breast cancer in a subject. Breast cancer is one of the most common neoplasms in women, and prevention and early detection are key factors in controlling and curing breast cancer. The expression of appropriate polynucleotides can be used in the diagnosis, prognosis and management of breast cancer. Detection of breast cancer can be determined using expression levels of any of these sequences alone or in combination with the levels of expression of other known cancer genes. Determination of the aggressive nature and/or the metastatic potential of a breast cancer can be determined by comparing levels of one or more gene products of the genes corresponding to the polynucleotides described herein, and comparing total levels of another sequence known to vary in cancerous tissue, e.g., expression of p53, DCC, ras, FAP (see, e.g., Fearon E R, et al., Cell (1990) 61(5):759; Hamilton S R et al., Cancer (1993) 72:957; Bodmer W, et al, Nat Genet. (1994) 4(3):217; Fearon E R, Ann NY Acad Sci. (1995) 768:101). For example, development of breast cancer can be detected by examining the level of expression of a gene corresponding to a polynucleotides described herein to the levels of oncogenes (e.g. ras) or tumor suppressor genes (e.g. FAP or p53). Thus expression of specific marker polynucleotides can be used to discriminate between normal and cancerous breast tissue, to discriminate between breast cancers with different cells of origin, to discriminate between breast cancers with different potential metastatic rates, etc. For a review of other markers of cancer, see, e.g., Hanahan et al. (2000) Cell 100:57-70.

Treatment of Breast Cancer

The invention further provides methods for reducing growth of breast cancer cells. The methods provide for decreasing the expression of a gene that is differentially expressed in a breast cancer cell or decreasing the level of and/or decreasing an activity of a breast cancer-associated polypeptide. In general, the methods comprise contacting a breast cancer cell with a substance that modulates (1) expression of a gene that is differentially expressed in breast cancer; or (2) a level of and/or an activity of a breast cancer-associated polypeptide.

“Reducing growth of breast cancer cells” includes, but is not limited to, reducing proliferation of breast cancer cells, and reducing the incidence of a non-cancerous breast cell becoming a cancerous breast cell. Whether a reduction in breast cancer cell growth has been achieved can be readily determined using any known assay, including, but not limited to, [³]H-thymidine incorporation; counting cell number over a period of time; detecting and/or measuring a marker associated with breast cancer (e.g., PSA).

The present invention provides methods for treating breast cancer, generally comprising administering to an individual in need thereof a substance that reduces breast cancer cell growth, in an amount sufficient to reduce breast cancer cell growth and treat the breast cancer. Whether a substance, or a specific amount of the substance, is effective in treating breast cancer can be assessed using any of a variety of known diagnostic assays for breast cancer, including, but not limited to, proctoscopy, rectal examination, biopsy, contrast radiographic studies, CAT scan, and detection of a tumor marker associated with breast cancer in the blood of the individual (e.g., PSA (breast-specific antigen)). The substance can be administered systemically or locally. Thus, in some embodiments, the substance is administered locally, and breast cancer growth is decreased at the site of administration. Local administration may be useful in treating, e.g., a solid tumor.

A substance that reduces breast cancer cell growth can be targeted to a breast cancer cell. Thus, in some embodiments, the invention provides a method of delivering a drug to a breast cancer cell, comprising administering a drug-antibody complex to a subject, wherein the antibody is specific for a breast cancer-associated polypeptide, and the drug is one that reduces breast cancer cell growth, a variety of which are known in the art. Targeting can be accomplished by coupling (e.g., linking, directly or via a linker molecule, either covalently or non-covalently, so as to form a drug-antibody complex) a drug to an antibody specific for a breast cancer-associated polypeptide. Methods of coupling a drug to an antibody are well known in the art and need not be elaborated upon herein.

Tumor Classification and Patient Stratification

The invention further provides for methods of classifying tumors, and thus grouping or “stratifying” patients, according to the expression profile of selected differentially expressed genes in a tumor. Differentially expressed genes can be analyzed for correlation with other differentially expressed genes in a single tumor type or across tumor types. Genes that demonstrate consistent correlation in expression profile in a given cancer cell type (e.g., in a breast cancer cell or type of breast cancer) can be grouped together, e.g., when one gene is overexpressed in a tumor, a second gene is also usually overexpressed. Tumors can then be classified according to the expression profile of one or more genes selected from one or more groups.

The tumor of each patient in a pool of potential patients can be classified as described above. Patients having similarly classified tumors can then be selected for participation in an investigative or clinical trial of a cancer therapeutic where a homogeneous population is desired. The tumor classification of a patient can also be used in assessing the efficacy of a cancer therapeutic in a heterogeneous patient population. In addition, therapy for a patient having a tumor of a given expression profile can then be selected accordingly.

In another embodiment, differentially expressed gene products (e.g., polypeptides or polynucleotides encoding such polypeptides) may be effectively used in treatment through vaccination. The growth of cancer cells is naturally limited in part due to immune surveillance. Stimulation of the immune system using a particular tumor-specific antigen enhances the effect towards the tumor expressing the antigen. An active vaccine comprising a polypeptide encoded by the cDNA of this invention would be appropriately administered to subjects having an alteration, e.g., overabundance, of the corresponding RNA, or those predisposed for developing cancer cells with an alteration of the same RNA. Polypeptide antigens are typically combined with an adjuvant as part of a vaccine composition. The vaccine is preferably administered first as a priming dose, and then again as a boosting dose, usually at least four weeks later. Further boosting doses may be given to enhance the effect. The dose and its timing are usually determined by the person responsible for the treatment.

The invention also encompasses the selection of a therapeutic regimen based upon the expression profile of differentially expressed genes in the patient's tumor. For example, a tumor can be analyzed for its expression profile of the genes corresponding to SEQ ID NOS:1-499 as described herein, e.g., the tumor is analyzed to determine which genes are expressed at elevated levels or at decreased levels relative to normal cells of the same tissue type. The expression patterns of the tumor are then compared to the expression patterns of tumors that respond to a selected therapy. Where the expression profiles of the test tumor cell and the expression profile of a tumor cell of known drug responsivity at least substantially match (e.g., selected sets of genes at elevated levels in the tumor of known drug responsivity and are also at elevated levels in the test tumor cell), then the therapeutic agent selected for therapy is the drug to which tumors with that expression pattern respond.

Pattern Matching in Diagnosis Using Arrays

In another embodiment, the diagnostic and/or prognostic methods of the invention involve detection of expression of a selected set of genes in a test sample to produce a test expression pattern (TEP). The TEP is compared to a reference expression pattern (REP), which is generated by detection of expression of the selected set of genes in a reference sample (e.g., a positive or negative control sample). The selected set of genes includes at least one of the genes of the invention, which genes correspond to the polynucleotide sequences described herein. Of particular interest is a selected set of genes that includes gene differentially expressed in the disease for which the test sample is to be screened.

Identification of Therapeutic Targets and Anti-Cancer Therapeutic Agents

The present invention also encompasses methods for identification of agents having the ability to modulate activity of a differentially expressed gene product, as well as methods for identifying a differentially expressed gene product as a therapeutic target for treatment of cancer, especially breast cancer.

Identification of compounds that modulate activity of a differentially expressed gene product can be accomplished using any of a variety of drug screening techniques. Such agents are candidates for development of cancer therapies. Of particular interest are screening assays for agents that have tolerable toxicity for normal, non-cancerous human cells. The screening assays of the invention are generally based upon the ability of the agent to modulate an activity of a differentially expressed gene product and/or to inhibit or suppress phenomenon associated with cancer (e.g., cell proliferation, colony formation, cell cycle arrest, metastasis, and the like).

Screening of Candidate Agents

Screening assays can be based upon any of a variety of techniques readily available and known to one of ordinary skill in the art. In general, the screening assays involve contacting a cancerous cell (preferably a cancerous cell such as a cancerous breast cell) with a candidate agent, and assessing the effect upon biological activity of a differentially expressed gene product. The effect upon a biological activity can be detected by, for example, detection of expression of a gene product of a differentially expressed gene (e.g., a decrease in mRNA or polypeptide levels, would in turn cause a decrease in biological activity of the gene product). Alternatively or in addition, the effect of the candidate agent can be assessed by examining the effect of the candidate agent in a functional assay. For example, where the differentially expressed gene product is an enzyme, then the effect upon biological activity can be assessed by detecting a level of enzymatic activity associated with the differentially expressed gene product. The functional assay will be selected according to the differentially expressed gene product. In general, where the differentially expressed gene is increased in expression in a cancerous cell, agents of interest are those that decrease activity of the differentially expressed gene product.

Assays described infra can be readily adapted in the screening assay embodiments of the invention. Exemplary assays useful in screening candidate agents include, but are not limited to, hybridization-based assays (e.g., use of nucleic acid probes or primers to assess expression levels), antibody-based assays (e.g., to assess levels of polypeptide gene products), binding assays (e.g., to detect interaction of a candidate agent with a differentially expressed polypeptide, which assays may be competitive assays where a natural or synthetic ligand for the polypeptide is available), and the like. Additional exemplary assays include, but are not necessarily limited to, cell proliferation assays, antisense knockout assays, assays to detect inhibition of cell cycle, assays of induction of cell death/apoptosis, and the like. Generally such assays are conducted in vitro, but many assays can be adapted for in vivo analyses, e.g., in an animal model of the cancer.

Identification of Therapeutic Targets

In another embodiment, the invention contemplates identification of differentially expressed genes and gene products as therapeutic targets. In some respects, this is the converse of the assays described above for identification of agents having activity in modulating (e.g., decreasing or increasing) activity of a differentially expressed gene product.

In this embodiment, therapeutic targets are identified by examining the effect(s) of an agent that can be demonstrated or has been demonstrated to modulate a cancerous phenotype (e.g., inhibit or suppress or prevent development of a cancerous phenotype). Such agents are generally referred to herein as an “anti-cancer agent”, which agents encompass chemotherapeutic agents. For example, the agent can be an antisense oligonucleotide that is specific for a selected gene transcript. For example, the antisense oligonucleotide may have a sequence corresponding to a sequence of a differentially expressed gene described herein, e.g., a sequence of one of SEQ ID NOS:1-499.

Assays for identification of therapeutic targets can be conducted in a variety of ways using methods that are well known to one of ordinary skill in the art. For example, a test cancerous cell that expresses or overexpresses a differentially expressed gene is contacted with an anti-cancer agent, the effect upon a cancerous phenotype and a biological activity of the candidate gene product assessed. The biological activity of the candidate gene product can be assayed be examining, for example, modulation of expression of a gene encoding the candidate gene product (e.g., as detected by, for example, an increase or decrease in transcript levels or polypeptide levels), or modulation of an enzymatic or other activity of the gene product. The cancerous phenotype can be, for example, cellular proliferation, loss of contact inhibition of growth (e.g., colony formation), tumor growth (in vitro or in vivo), and the like. Alternatively or in addition, the effect of modulation of a biological activity of the candidate target gene upon cell death/apoptosis or cell cycle regulation can be assessed.

Inhibition or suppression of a cancerous phenotype, or an increase in cell death or apoptosis as a result of modulation of biological activity of a candidate gene product indicates that the candidate gene product is a suitable target for cancer therapy. Assays described infra can be readily adapted for assays for identification of therapeutic targets. Generally such assays are conducted in vitro, but many assays can be adapted for in vivo analyses, e.g., in an appropriate, art-accepted animal model of the cancer.

Candidate Agents

The term “agent” as used herein describes any molecule, e.g. protein or pharmaceutical, with the capability of modulating a biological activity of a gene product of a differentially expressed gene. Generally a plurality of assay mixtures are run in parallel with different agent concentrations to obtain a differential response to the various concentrations. Typically, one of these concentrations serves as a negative control, i.e. at zero concentration or below the level of detection.

Candidate agents encompass numerous chemical classes, though typically they are organic molecules, preferably small organic compounds having a molecular weight of more than 50 and less than about 2,500 daltons. Candidate agents comprise functional groups necessary for structural interaction with proteins, particularly hydrogen bonding, and typically include at least an amine, carbonyl, hydroxyl or carboxyl group, preferably at least two of the functional chemical groups. The candidate agents often comprise cyclical carbon or heterocyclic structures and/or aromatic or polyaromatic structures substituted with one or more of the above functional groups. Candidate agents are also found among biomolecules including, but not limited to: peptides, saccharides, fatty acids, steroids, purines, pyrimidines, derivatives, structural analogs or combinations thereof Candidate agents are obtained from a wide variety of sources including libraries of synthetic or natural compounds. For example, numerous means are available for random and directed synthesis of a wide variety of organic compounds and biomolecules, including expression of randomized oligonucleotides and oligopeptides. Alternatively, libraries of natural compounds in the form of bacterial, fungal, plant and animal extracts (including extracts from human tissue to identify endogenous factors affecting differentially expressed gene products) are available or readily produced. Additionally, natural or synthetically produced libraries and compounds are readily modified through conventional chemical, physical and biochemical means, and may be used to produce combinatorial libraries. Known pharmacological agents may be subjected to directed or random chemical modifications, such as acylation, alkylation, esterification, amidification, etc. to produce structural analogs.

Exemplary candidate agents of particular interest include, but are not limited to, antisense and RNAi polynucleotides, and antibodies, soluble receptors, and the like. Antibodies and soluble receptors are of particular interest as candidate agents where the target differentially expressed gene product is secreted or accessible at the cell-surface (e.g., receptors and other molecule stably-associated with the outer cell membrane).

For method that involve RNAi (RNA interference), a double stranded RNA (dsRNA) molecule is usually used. The dsRNA is prepared to be substantially identical to at least a segment of a subject polynucleotide (e.g. a cDNA or gene). In general, the dsRNA is selected to have at least 70%, 75%, 80%, 85% or 90% sequence identity with the subject polynucleotide over at least a segment of the candidate gene. In other instances, the sequence identity is even higher, such as 95%, 97% or 99%, and in still other instances, there is 100% sequence identity with the subject polynucleotide over at least a segment of the subject polynucleotide. The size of the segment over which there is sequence identity can vary depending upon the size of the subject polynucleotide. In general, however, there is substantial sequence identity over at least 15, 20, 25, 30, 35, 40 or 50 nucleotides. In other instances, there is substantial sequence identity over at least 100, 200, 300, 400, 500 or 1000 nucleotides; in still other instances, there is substantial sequence identity over the entire length of the subject polynucleotide, i.e., the coding and non-coding region of the candidate gene.

Because only substantial sequence similarity between the subject polynucleotide and the dsRNA is necessary, sequence variations between these two species arising from genetic mutations, evolutionary divergence and polymorphisms can be tolerated. Moreover, as described further infra, the dsRNA can include various modified or nucleotide analogs.

Usually the dsRNA consists of two separate complementary RNA strands. However, in some instances, the dsRNA may be formed by a single strand of RNA that is self-complementary, such that the strand loops back upon itself to form a hairpin loop. Regardless of form, RNA duplex formation can occur inside or outside of a cell.

The size of the dsRNA that is utilized varies according to the size of the subject polynucleotide whose expression is to be suppressed and is sufficiently long to be effective in reducing expression of the subject polynucleotide in a cell. Generally, the dsRNA is at least 10-15 nucleotides long. In certain applications, the dsRNA is less than 20, 21, 22, 23, 24 or 25 nucleotides in length. In other instances, the dsRNA is at least 50, 100, 150 or 200 nucleotides in length. The dsRNA can be longer still in certain other applications, such as at least 300, 400, 500 or 600 nucleotides. Typically, the dsRNA is not longer than 3000 nucleotides. The optimal size for any particular subject polynucleotide can be determined by one of ordinary skill in the art without undue experimentation by varying the size of the dsRNA in a systematic fashion and determining whether the size selected is effective in interfering with expression of the subject polynucleotide.

dsRNA can be prepared according to any of a number of methods that are known in the art, including in vitro and in vivo methods, as well as by synthetic chemistry approaches.

In vitro methods. Certain methods generally involve inserting the segment corresponding to the candidate gene that is to be transcribed between a promoter or pair of promoters that are oriented to drive transcription of the inserted segment and then utilizing an appropriate RNA polymerase to carry out transcription. One such arrangement involves positioning a DNA fragment corresponding to the candidate gene or segment thereof into a vector such that it is flanked by two opposable polymerase-specific promoters that can be same or different. Transcription from such promoters produces two complementary RNA strands that can subsequently anneal to form the desired dsRNA. Exemplary plasmids for use in such systems include the plasmid (PCR 4.0 TOPO) (available from Invitrogen). Another example is the vector pGEM-T (Promega, Madison, Wis.) in which the oppositely oriented promoters are T7 and SP6; the T3 promoter can also be utilized.

In a second arrangement, DNA fragments corresponding to the segment of the subject polynucleotide that is to be transcribed is inserted both in the sense and antisense orientation downstream of a single promoter. In this system, the sense and antisense fragments are cotranscribed to generate a single RNA strand that is self-complementary and thus can form dsRNA.

Various other in vitro methods have been described. Examples of such methods include, but are not limited to, the methods described by Sadher et al. (Biochem. Int. 14:1015, 1987); by Bhattacharyya (Nature 343:484, 1990); and by Livache, et al. (U.S. Pat. No. 5,795,715), each of which is incorporated herein by reference in its entirety.

Single-stranded RNA can also be produced using a combination of enzymatic and organic synthesis or by total organic synthesis. The use of synthetic chemical methods enable one to introduce desired modified nucleotides or nucleotide analogs into the dsRNA.

In vivo methods. dsRNA can also be prepared in vivo according to a number of established methods (see, e.g., Sambrook, et al. (1989) Molecular Cloning: A Laboratory Manual, 2^{nd} ed.; Transcription and Translation (B. D. Hames, and S. J. Higgins, Eds., 1984); DNA Cloning, volumes I and II (D. N. Glover, Ed., 1985); and Oligonucleotide Synthesis (M. J. Gait, Ed., 1984, each of which is incorporated herein by reference in its entirety).

Once the single-stranded RNA has been formed, the complementary strands are allowed to anneal to form duplex RNA. Transcripts are typically treated with DNAase and further purified according to established protocols to remove proteins. Usually such purification methods are not conducted with phenol:chloroform. The resulting purified transcripts are subsequently dissolved in RNAase free water or a buffer of suitable composition.

dsRNA is generated by annealing the sense and anti-sense RNA in vitro. Generally, the strands are initially denatured to keep the strands separate and to avoid self-annealing. During the annealing process, typically certain ratios of the sense and antisense strands are combined to facilitate the annealing process. In some instances, a molar ratio of sense to antisense strands of 3:7 is used; in other instances, a ratio of 4:6 is utilized; and in still other instances, the ratio is 1:1.

The buffer composition utilized during the annealing process can in some instances affect the efficacy of the annealing process and subsequent transfection procedure. While some have indicated that the buffered solution used to carry out the annealing process should include a potassium salt such as potassium chloride (e.g. at a concentration of about 80 mM). In some embodiments, the buffer is substantially postassium free. Once single-stranded RNA has annealed to form duplex RNA, typically any single-strand overhangs are removed using an enzyme that specifically cleaves such overhangs (e.g., RNAase A or RNAase T).

Once the dsRNA has been formed, it is introduced into a reference cell, which can include an individual cell or a population of cells (e.g., a tissue, an embryo and an entire organism). The cell can be from essentially any source, including animal, plant, viral, bacterial, fungal and other sources. If a tissue, the tissue can include dividing or nondividing and differentiated or undifferentiated cells. Further, the tissue can include germ line cells and somatic cells. Examples of differentiated cells that can be utilized include, but are not limited to, neurons, glial cells, blood cells, megakaryocytes, lymphocytes, macrophages, neutrophils, eosinophils, basophils, mast cells, leukocytes, granulocytes, keratinocytes, adipocytes, osteoblasts, osteoclasts, hepatocytes, cells of the endocrine or exocrine glands, fibroblasts, myocytes, cardiomyocytes, and endothelial cells. The cell can be an individual cell of an embryo, and can be a blastocyte or an oocyte.

Certain methods are conducted using model systems for particular cellular states (e.g., a disease). For instance, certain methods provided herein are conducted with a cancer cell lines that serves as a model system for investigating genes that are correlated with various cancers.

A number of options can be utilized to deliver the dsRNA into a cell or population of cells such as in a cell culture, tissue or embryo. For instance, RNA can be directly introduced intracellularly. Various physical methods are generally utilized in such instances, such as administration by microinjection (see, e.g., Zernicka-Goetz, et al. (1997) Development 124:1133-1137; and Wianny, et al. (1998) Chromosoma 107: 430-439).

Other options for cellular delivery include permeabilizing the cell membrane and electroporation in the presence of the dsRNA, liposome-mediated transfection, or transfection using chemicals such as calcium phosphate. A number of established gene therapy techniques can also be utilized to introduce the dsRNA into a cell. By introducing a viral construct within a viral particle, for instance, one can achieve efficient introduction of an expression construct into the cell and transcription of the RNA encoded by the construct.

If the dsRNA is to be introduced into an organism or tissue, gene gun technology is an option that can be employed. This generally involves immobilizing the dsRNA on a gold particle which is subsequently fired into the desired tissue. Research has also shown that mammalian cells have transport mechanisms for taking in dsRNA (see, e.g., Asher, et al. (1969) Nature 223:715-717). Consequently, another delivery option is to administer the dsRNA extracellularly into a body cavity, interstitial space or into the blood system of the mammal for subsequent uptake by such transport processes. The blood and lymph systems and the cerebrospinal fluid are potential sites for injecting dsRNA. Oral, topical, parenteral, rectal and intraperitoneal administration are also possible modes of administration.

The composition introduced can also include various other agents in addition to the dsRNA. Examples of such agents include, but are not limited to, those that stabilize the dsRNA, enhance cellular uptake and/or increase the extent of interference. Typically, the dsRNA is introduced in a buffer that is compatible with the composition of the cell into which the RNA is introduced to prevent the cell from being shocked. The minimum size of the dsRNA that effectively achieves gene silencing can also influence the choice of delivery system and solution composition.

Sufficient dsRNA is introduced into the tissue to cause a detectable change in expression of a taget gene (assuming the candidate gene is in fact being expressed in the cell into which the dsRNA is introduced) using available detection methodologies. Thus, in some instances, sufficient dsRNA is introduced to achieve at least a 5-10% reduction in candidate gene expression as compared to a cell in which the dsRNA is not introduced. In other instances, inhibition is at least 20, 30, 40 or 50%. In still other instances, the inhibition is at least 60, 70, 80, 90 or 95%. Expression in some instances is essentially completely inhibited to undetectable levels.

The amount of dsRNA introduced depends upon various factors such as the mode of administration utilized, the size of the dsRNA, the number of cells into which dsRNA is administered, and the age and size of an animal if dsRNA is introduced into an animal. An appropriate amount can be determined by those of ordinary skill in the art by initially administering dsRNA at several different concentrations for example, for example. In certain instances when dsRNA is introduced into a cell culture, the amount of dsRNA introduced into the cells varies from about 0.5 to 3 μg per 10⁶ cells.

A number of options are available to detect interference of candidate gene expression (i.e., to detect candidate gene silencing). In general, inhibition in expression is detected by detecting a decrease in the level of the protein encoded by the candidate gene, determining the level of mRNA transcribed from the gene and/or detecting a change in phenotype associated with candidate gene expression.

Use of Polypeptides to Screen for Peptide Analogs and Antagonists

Polypeptides encoded by differentially expressed genes identified herein can be used to screen peptide libraries to identify binding partners, such as receptors, from among the encoded polypeptides. Peptide libraries can be synthesized according to methods known in the art (see, e.g., U.S. Pat. No. 5,010,175 and WO 91/17823).

Agonists or antagonists of the polypeptides of the invention can be screened using any available method known in the art, such as signal transduction, antibody binding, receptor binding, mitogenic assays, chemotaxis assays, etc. The assay conditions ideally should resemble the conditions under which the native activity is exhibited in vivo, that is, under physiologic pH, temperature, and ionic strength. Suitable agonists or antagonists will exhibit strong inhibition or enhancement of the native activity at concentrations that do not cause toxic side effects in the subject. Agonists or antagonists that compete for binding to the native polypeptide can require concentrations equal to or greater than the native concentration, while inhibitors capable of binding irreversibly to the polypeptide can be added in concentrations on the order of the native concentration.

Such screening and experimentation can lead to identification of a polypeptide binding partner, such as a receptor, encoded by a gene or a cDNA corresponding to a polynucleotide described herein, and at least one peptide agonist or antagonist of the binding partner. Such agonists and antagonists can be used to modulate, enhance, or inhibit receptor function in cells to which the receptor is native, or in cells that possess the receptor as a result of genetic engineering. Further, if the receptor shares biologically important characteristics with a known receptor, information about agonist/antagonist binding can facilitate development of improved agonists/antagonists of the known receptor.

Vaccines and Uses

The differentially expressed nucleic acids and polypeptides produced by the nucleic acids of the invention can also be used to modulate primary immune response to prevent or treat cancer. Every immune response is a complex and intricately regulated sequence of events involving several cell types. It is triggered when an antigen enters the body and encounters a specialized class of cells called antigen-presenting cells (APCs). These APCs capture a minute amount of the antigen and display it in a form that can be recognized by antigen-specific helper T lymphocytes. The helper (Th) cells become activated and, in turn, promote the activation of other classes of lymphocytes, such as B cells or cytotoxic T cells. The activated lymphocytes then proliferate and carry out their specific effector functions, which in many cases successfully activate or eliminate the antigen. Thus, activating the immune response to a particular antigen associated with a cancer cell can protect the patient from developing cancer or result in lymphocytes eliminating cancer cells expressing the antigen.

Gene products, including polypeptides, mRNA (particularly mRNAs having distinct secondary and/or tertiary structures), cDNA, or complete gene, can be prepared and used in vaccines for the treatment or prevention of hyperproliferative disorders and cancers. The nucleic acids and polypeptides can be utilized to enhance the immune response, prevent tumor progression, prevent hyperproliferative cell growth, and the like. Methods for selecting nucleic acids and polypeptides that are capable of enhancing the immune response are known in the art. Preferably, the gene products for use in a vaccine are gene products which are present on the surface of a cell and are recognizable by lymphocytes and antibodies.

The gene products may be formulated with pharmaceutically acceptable carriers into pharmaceutical compositions by methods known in the art. The composition is useful as a vaccine to prevent or treat cancer. The composition may further comprise at least one co-immunostimulatory molecule, including but not limited to one or more major histocompatibility complex (MHC) molecules, such as a class I or class II molecule, preferably a class I molecule. The composition may further comprise other stimulator molecules including B7.1, B7.2, ICAM-1, ICAM-2, LFA-1, LFA-3, CD72 and the like, immunostimulatory polynucleotides (which comprise an 5′-CG-3′ wherein the cytosine is unmethylated), and cytokines which include but are not limited to IL-1 through IL-15, TNF-α, IFN-γ, RANTES, G-CSF, M-CSF, IFN-α, CTAP III, ENA-78, GRO, 1-309, PF-4, IP-10, LD-78, MGSA, MIP-1α, MIP-1β, or combination thereof, and the like for immunopotentiation. In one embodiment, the immunopotentiators of particular interest are those that facilitate a Th1 immune response.

The gene products may also be prepared with a carrier that will protect the gene products against rapid elimination from the body, such as a controlled release formulation, including implants and microencapsulated delivery systems. Biodegradable polymers can be used, such as ethylene vinyl acetate, polyanhydrides, polyglycolic acid, collagen, polyorthoesters, polylactic acid, and the like. Methods for preparation of such formulations are known in the art.

In the methods of preventing or treating cancer, the gene products may be administered via one of several routes including but not limited to transdermal, transmucosal, intravenous, intramuscular, subcutaneous, intradermal, intraperitoneal, intrathecal, intrapleural, intrauterine, rectal, vaginal, topical, intratumor, and the like. For transmucosal or transdermal administration, penetrants appropriate to the barrier to be permeated are used in the formulation. Such penetrants are generally known in the art, and include, for example, administration bile salts and fusidic acid derivatives. In addition, detergents may be used to facilitate permeation. Transmucosal administration may be by nasal sprays or suppositories. For oral administration, the gene products are formulated into conventional oral administration form such as capsules, tablets, elixirs and the like.

The gene product is administered to a patient in an amount effective to prevent or treat cancer. In general, it is desirable to provide the patient with a dosage of gene product of at least about 1 pg per Kg body weight, preferably at least about 1 ng per Kg body weight, more preferably at least about 1 μg or greater per Kg body weight of the recipient. A range of from about 1 ng per Kg body weight to about 100 mg per Kg body weight is preferred although a lower or higher dose may be administered. The dose is effective to prime, stimulate and/or cause the clonal expansion of antigen-specific T lymphocytes, preferably cytotoxic T lymphocytes, which in turn are capable of preventing or treating cancer in the recipient. The dose is administered at least once and may be provided as a bolus or a continuous administration. Multiple administrations of the dose over a period of several weeks to months may be preferable. Subsequent doses may be administered as indicated.

In another method of treatment, autologous cytotoxic lymphocytes or tumor infiltrating lymphocytes may be obtained from a patient with cancer. The lymphocytes are grown in culture, and antigen-specific lymphocytes are expanded by culturing in the presence of the specific gene products alone or in combination with at least one co-immunostimulatory molecule with cytokines. The antigen-specific lymphocytes are then infused back into the patient in an amount effective to reduce or eliminate the tumors in the patient. Cancer vaccines and their uses are further described in U.S. Pat. No. 5,961,978; U.S. Pat. No. 5,993,829; U.S. Pat. No. 6,132,980; and WO 00/38706.

Pharmaceutical Compositions and Uses

Pharmaceutical compositions can comprise polypeptides, receptors that specifically bind a polypeptide produced by a differentially expressed gene (e.g., antibodies, or polynucleotides (including antisense nucleotides and ribozymes) of the claimed invention in a therapeutically effective amount. The compositions can be used to treat primary tumors as well as metastases of primary tumors. In addition, the pharmaceutical compositions can be used in conjunction with conventional methods of cancer treatment, e.g., to sensitize tumors to radiation or conventional chemotherapy.

Where the pharmaceutical composition comprises a receptor (such as an antibody) that specifically binds to a gene product encoded by a differentially expressed gene, the receptor can be coupled to a drug for delivery to a treatment site or coupled to a detectable label to facilitate imaging of a site comprising breast cancer cells. Methods for coupling antibodies to drugs and detectable labels are well known in the art, as are methods for imaging using detectable labels.

The term “therapeutically effective amount” as used herein refers to an amount of a therapeutic agent to treat, ameliorate, or prevent a desired disease or condition, or to exhibit a detectable therapeutic or preventative effect. The effect can be detected by, for example, chemical markers or antigen levels. Therapeutic effects also include reduction in physical symptoms, such as decreased body temperature.

The precise effective amount for a subject will depend upon the subject's size and health, the nature and extent of the condition, and the therapeutics or combination of therapeutics selected for administration. Thus, it is not useful to specify an exact effective amount in advance. However, the effective amount for a given situation is determined by routine experimentation and is within the judgment of the clinician. For purposes of the present invention, an effective dose will generally be from about 0.01 mg/ kg to 50 mg/kg or 0.05 mg/kg to about 10 mg/kg of the DNA constructs in the individual to which it is administered.

A pharmaceutical composition can also contain a pharmaceutically acceptable carrier. The term “pharmaceutically acceptable carrier” refers to a carrier for administration of a therapeutic agent, such as antibodies or a polypeptide, genes, and other therapeutic agents. The term refers to any pharmaceutical carrier that does not itself induce the production of antibodies harmful to the individual receiving the composition, and which can be administered without undue toxicity. Suitable carriers can be large, slowly metabolized macromolecules such as proteins, polysaccharides, polylactic acids, polyglycolic acids, polymeric amino acids, amino acid copolymers, lipid aggregates and inactive virus particles. Such carriers are well known to those of ordinary skill in the art. Pharmaceutically acceptable carriers in therapeutic compositions can include liquids such as water, saline, glycerol and ethanol. Auxiliary substances, such as wetting or emulsifying agents, pH buffering substances, and the like, can also be present in such vehicles.

Typically, the therapeutic compositions are prepared as injectables, either as liquid solutions or suspensions; solid forms suitable for solution in, or suspension in, liquid vehicles prior to injection can also be prepared. Liposomes are included within the definition of a pharmaceutically acceptable carrier. Pharmaceutically acceptable salts can also be present in the pharmaceutical composition, e.g., mineral acid salts such as hydrochlorides, hydrobromides, phosphates, sulfates, and the like; and the salts of organic acids such as acetates, propionates, malonates, benzoates, and the like. A thorough discussion of pharmaceutically acceptable excipients is available in Remington: The Science and Practice of Pharmacy (1995) Alfonso Gennaro, Lippincott, Williams, & Wilkins.

Delivery Methods

Once formulated, the compositions contemplated by the invention can be (1) administered directly to the subject (e.g., as polynucleotide, polypeptides, small molecule agonists or antagonists, and the like); or (2) delivered ex vivo, to cells derived from the subject (e.g., as in ex vivo gene therapy). Direct delivery of the compositions will generally be accomplished by parenteral injection, e.g., subcutaneously, intraperitoneally, intravenously or intramuscularly, intratumoral or to the interstitial space of a tissue. Other modes of administration include oral and pulmonary administration, suppositories, and transdermal applications, needles, and gene guns or hyposprays. Dosage treatment can be a single dose schedule or a multiple dose schedule.

Methods for the ex vivo delivery and reimplantation of transformed cells into a subject are known in the art and described in e.g., International Publication No. WO 93/14778. Examples of cells useful in ex vivo applications include, for example, stem cells, particularly hematopoetic, lymph cells, macrophages, dendritic cells, or tumor cells. Generally, delivery of nucleic acids for both ex vivo and in vitro applications can be accomplished by, for example, dextran-mediated transfection, calcium phosphate precipitation, polybrene mediated transfection, protoplast fusion, electroporation, encapsulation of the polynucleotide(s) in liposomes, and direct microinjection of the DNA into nuclei, all well known in the art.

Once differential expression of a gene corresponding to a polynucleotide described herein has been found to correlate with a proliferative disorder, such as neoplasia, dysplasia, and hyperplasia, the disorder can be amenable to treatment by administration of a therapeutic agent based on the provided polynucleotide, corresponding polypeptide or other corresponding molecule (e.g., antisense, ribozyme, etc.). In other embodiments, the disorder can be amenable to treatment by administration of a small molecule drug that, for example, serves as an inhibitor (antagonist) of the function of the encoded gene product of a gene having increased expression in cancerous cells relative to normal cells or as an agonist for gene products that are decreased in expression in cancerous cells (e.g., to promote the activity of gene products that act as tumor suppressors).

The dose and the means of administration of the inventive pharmaceutical compositions are determined based on the specific qualities of the therapeutic composition, the condition, age, and weight of the patient, the progression of the disease, and other relevant factors. For example, administration of polynucleotide therapeutic composition agents includes local or systemic administration, including injection, oral administration, particle gun or catheterized administration, and topical administration. In general, the therapeutic polynucleotide composition contains an expression construct comprising a promoter operably linked to a polynucleotide of at least 12, 22, 25, 30, or 35 contiguous nt of the polynucleotide disclosed herein. Various methods can be used to administer the therapeutic composition directly to a specific site in the body. For example, a small metastatic lesion is located and the therapeutic composition injected several times in several different locations within the body of the tumor. Alternatively, arteries which serve a tumor are identified, and the therapeutic composition injected into such an artery, in order to deliver the composition directly into the tumor. A tumor that has a necrotic center is aspirated and the composition injected directly into the now empty center of the tumor. The antisense composition is directly administered to the surface of the tumor, for example, by topical application of the composition. X-ray imaging is used to assist in certain of the above delivery methods.

Targeted delivery of therapeutic compositions containing an antisense polynucleotide, subgenomic polynucleotides, or antibodies to specific tissues can also be used. Receptor-mediated DNA delivery techniques are described in, for example, Findeis et al., Trends Biotechnol. (1993) 11:202; Chiou et al., Gene Therapeutics: Methods And Applications Of Direct Gene Transfer (J. A. Wolff, ed.) (1994); Wu et al., J. Biol. Chem. (1988) 263:621; Wu et al., J. Biol. Chem. (1994) 269:542; Zenke et al., Proc. Natl. Acad. Sci. (USA) (1990) 87:3655; Wu et al., J. Biol. Chem. (1991) 266:338. Therapeutic compositions containing a polynucleotide are administered in a range of about 100 ng to about 200 mg of DNA for local administration in a gene therapy protocol. Concentration ranges of about 500 ng to about 50 mg, about 1 μg to about 2 mg, about 5 μg to about 500 μg, and about 20 μg to about 100 μg of DNA can also be used during a gene therapy protocol. Factors such as method of action (e.g., for enhancing or inhibiting levels of the encoded gene product) and efficacy of transformation and expression are considerations that will affect the dosage required for ultimate efficacy of the antisense subgenomic polynucleotides.

Where greater expression is desired over a larger area of tissue, larger amounts of antisense subgenomic polynucleotides or the same amounts readministered in a successive protocol of administrations, or several administrations to different adjacent or close tissue portions of, for example, a tumor site, may be required to effect a positive therapeutic outcome. In all cases, routine experimentation in clinical trials will determine specific ranges for optimal therapeutic effect. For polynucleotide related genes encoding polypeptides or proteins with anti-inflammatory activity, suitable use, doses, and administration are described in U.S. Pat. No. 5,654,173.

The therapeutic polynucleotides and polypeptides of the present invention can be delivered using gene delivery vehicles. The gene delivery vehicle can be of viral or non-viral origin (see generally, Jolly, Cancer Gene Therapy (1994) 1:51; Kimura, Human Gene Therapy (1994) 5:845; Connelly, Human Gene Therapy (1995) 1:185; and Kaplitt, Nature Genetics (1994) 6:148). Expression of such coding sequences can be induced using endogenous mammalian or heterologous promoters. Expression of the coding sequence can be either constitutive or regulated.

Viral-based vectors for delivery of a desired polynucleotide and expression in a desired cell are well known in the art. Exemplary viral-based vehicles include, but are not limited to, recombinant retroviruses (see, e.g., WO 90/07936; WO 94/03622; WO 93/25698; WO 93/25234; U.S. Pat. No. 5,219,740; WO 93/11230; WO 93/10218; U.S. Pat. No. 4,777,127; GB Patent No. 2,200,651; EP 0 345 242; and WO 91/02805), alphavirus-based vectors (e.g., Sindbis virus vectors, Semliki forest virus (ATCC VR-67; ATCC VR-1247), Ross River virus (ATCC VR-373; ATCC VR-1246) and Venezuelan equine encephalitis virus (ATCC VR-923; ATCC VR-1250; ATCC VR 1249; ATCC VR-532), and adeno-associated virus (AAV) vectors (see, e.g., WO 94/12649, WO 93/03769; WO 93/19191; WO 94/28938; WO 95/11984 and WO 95/00655). Administration of DNA linked to killed adenovirus as described in Curiel, Hum. Gene Ther. (1992) 3:147 can also be employed.

Non-viral delivery vehicles and methods can also be employed, including, but not limited to, polycationic condensed DNA linked or unlinked to killed adenovirus alone (see, e.g., Curiel, Hum. Gene Ther. (1992) 3:147); ligand-linked DNA (see, e.g., Wu, J. Biol. Chem. (1989) 264:16985); eukaryotic cell delivery vehicles cells (see, e.g., U.S. Pat. No. 5,814,482; WO 95/07994; WO 96/17072; WO 95/30763; and WO 97/42338) and nucleic charge neutralization or fusion with cell membranes. Naked DNA can also be employed. Exemplary naked DNA introduction methods are described in WO 90/11092 and U.S. Pat. No. 5,580,859. Liposomes that can act as gene delivery vehicles are described in U.S. Pat. No. 5,422,120; WO 95/13796; WO 94/23697; WO 91/14445; and EP 0524968. Additional approaches are described in Philip, Mol. Cell Biol. (1994) 14:2411, and in Woffendin, Proc. Natl. Acad Sci. (1994) 91:1581.

Further non-viral delivery suitable for use includes mechanical delivery systems such as the approach described in Woffendin et al., Proc. Natl. Acad. Sci. USA (1994) 91(24):11581. Moreover, the coding sequence and the product of expression of such can be delivered through deposition of photopolymerized hydrogel materials or use of ionizing radiation (see, e.g., U.S. Pat. No. 5,206,152 and WO 92/11033). Other conventional methods for gene delivery that can be used for delivery of the coding sequence include, for example, use of hand-held gene transfer particle gun (see, e.g., U.S. Pat. No. 5,149,655); use of ionizing radiation for activating transferred gene (see, e.g., U.S. Pat. No. 5,206,152 and WO 92/11033).

### EXAMPLES

The following examples are put forth so as to provide those of ordinary skill in the art with a complete disclosure and description of how to make and use the present invention, and are not intended to limit the scope of what the inventors regard as their invention nor are they intended to represent that the experiments below are all or the only experiments performed. Efforts have been made to ensure accuracy with respect to numbers used (e.g. amounts, temperature, etc.) but some experimental errors and deviations should be accounted for. Unless indicated otherwise, parts are parts by weight, molecular weight is weight average molecular weight, temperature is in degrees Centigrade, and pressure is at or near atmospheric.

### Example 1

### Source of Biological Materials

The cells used for detecting differential expression of breast cancer related genes were those previously described for the HMT-3522 tumor reversion model, disclosed in U.S. Pat. Nos. 5,846,536 and 6,123,941, herein incorporated by reference. The model utilizes both non-tumorigenic (HMT-3522 SI) and tumorigenic (HMT-3522 T4-2) cells derived by serial passaging from a single reduction mammoplasty. In two dimensional (2D) monolayers on plastic, both S1 and T4-2 cells display similar morphology. But in three dimensional (3D) matrigel cultures, S1 form phenotypically normal mammary tissue structures while T4-2 cells fail to organize into these structures and instead disseminate into the matrix. This assay was designated as a tumor reversion model, in that the T4-2 cells can be induced to form S1-like structures in 3D by treatment with beta-1 integrin or EGFR blocking antibodies, or by treating with a chemical inhibitor of the EGFR signaling pathway (tyrophostin AG 1478). These treated T4-2 cells, called T4R cells, are non-tumorigenic.

### Example 2

### Cell Growth and RNA Isolation

Growth of Cells 2D and 3D for Microarray Experiments: HMT3522 S1 and T4-2 cells were grown 2D and 3D and T4-2 cells reverted with anti-EGFR, anti-beta 1 integrin, or tyrophostin AG 1478 as previously described (Weaver et al J Cell Biol. 137:231-45, 1997; and Wang et al PNAS 95:14821-14826, 1998). Anti-EGFR (mAb 225) was purchased from Oncogene and introduced into the matrigel at the time of gelation at a concentration of 4 ug/ml purified mouse IgG1. Anti-beta 1 integrin (mAb AIIB2) was a gift from C. Damsky at the University of California at San Francisco and was also introduced into the matrigel at the time of gelation at a concentration of 100 ug/ml ascites protein (which corresponds to 4-10 ug/ml purified rat IgG1). Tyrophostin AG 1478 was purchased from Calbiochem and used at a concentration of 100 nM.

Isolation of RNA for Microarray Experiments: RNA was prepared from: S1 passage 60 2D cultures; T4-2 passage 41 2D cultures; S1 passage 59 3D cultures; and T4-2 and T4-2 revertant (with anti-EGFR, anti-beta 1 integrin, and tyrophostin) passage 35 3D cultures.

All RNA for microarray experiments was isolated using the commercially available RNeasy Mini Kit from Qiagen. Isolation of total RNA from cells grown 2D was performed as instructed in the kit handbook. Briefly, media was aspirated from the cells and kit Buffer RLT was added directly to the flask. The cell lysate was collected with a rubber cell scraper, and the lysate passed 5 times through a 20-G needle fitted to a syringe. One volume of 70% ethanol was added to the homogenized lysate and mixed well by pipetting. Up to 700 ul of sample was applied to an RNeasy mini spin column sitting in a 2-ml collection tube and centrifuged for 15 seconds at >8000× g. 700 ul Buffer RW1 was added to the column and centrifuged for 15 seconds at >8000× g to wash. The column was transferred to a new collection tube. 500 ul Buffer RPE was added to the column and centrifuged for 15 seconds at >8000× g to wash. Another 500 ul Buffer RPE was added to the column for additional washing, and the column centrifuged for 2 minutes at maximum speed to dry. The column was transferred to a new collection tube and RNA eluted from the column with 30 ul RNase-free water by centrifuging for I minute at >8000× g.

Isolation of total RNA from cells grown 3D was performed as described above, except cells were isolated from matrigel prior to RNA isolation. The cells were isolated as colonies from matrigel using ice-cold PBS/EDTA (0.01 M sodium phosphate pH 7.2 containing 138 mM sodium chloride and 5 mM EDTA). See Weaver et al, J Cell Biol 137:231-245, 1997; and Wang et al. PNAS 95:14821-14826, 1998.

### Example 3

### Detection and Identification of Genes Exhibiting Differential Expression

The relative expression levels of a selected sequence (which in turn is representative of a single transcript) were examined in the tumorigenic versus non-tumorigenic cell lines described above, following culturing of the cells (S1, T4-2 and T4R) in either two-dimensional (2D) monolayers or three-dimensional (3D) matrigel cultures as described above. Differential expression for a selected sequence was assessed by hybridizing mRNA from SI and T4-2 2D cultures, and S1, T4-2 and T4R 3D cultures to microarray chips as described below, as follows: Exp1=T4-2 2D/S1 2D; Exp2=T4-2 3D/S1 3D; Exp3=S1 3D/S1 2D; Exp4=T4-2 3D/T4-2 2D; Exp5=T4-2 3D/T4R (anti-EGFR) 3D; Exp6=T4-2 3D/T4R (anti-betal integrin) 3D; and Exp7=T4-2 3D/T4R (tyrophostin AG 1478) 3D.

Each array used had an identical spatial layout and control spot set. Each microarray was divided into two areas, each area having an array with, on each half, twelve groupings of 32×12 spots for a total of about 9,216 spots on each array. The two areas are spotted identically which provide for at least two duplicates of each clone per array. Spotting was accomplished using PCR amplified products from 0.5 kb to 2.0 kb and spotted using a Molecular Dynamics Gen III spotter according to the manufacturer's recommendations. The first row of each of the 24 regions on the array had about 32 control spots, including 4 negative control spots and 8 test polynucleotides.

The test polynucleotides were spiked into each sample before the labeling reaction with a range of concentrations from 2-600 pg/slide and ratios of 1:1. For each array design, two slides were hybridized with the test samples reverse-labeled in the labeling reaction. This provided for about 4 duplicate measurements for each clone, two of one color and two of the other, for each sample.

Identification Of Differentially Expressed Genes: “Differentially expressed” in the context of the present example meant that there was a difference in expression of a particular gene between tumorigenic vs. non-tumorigenic cells, or cells grown in three-dimensional culture vs. cells grown in two-dimensional culture. To identify differentially expressed genes, total RNA was first reverse transcribed into cDNA using a primer containing a T7 RNA polymerase promoter, followed by second strand DNA synthesis. cDNA was then transcribed in vitro to produce antisense RNA using the T7 promoter-mediated expression (see, e.g., Luo et al. (1999) Nature Med 5:117-122), and the antisense RNA was then converted into cDNA. The second set of cDNAs were again transcribed in vitro, using the T7 promoter, to provide antisense RNA. Optionally, the RNA was again converted into cDNA, allowing for up to a third round of T7-mediated amplification to produce more antisense RNA. Thus the procedure provided for two or three rounds of in vitro transcription to produce the final RNA used for fluorescent labeling.

Fluorescent probes were generated by first adding control RNA to the antisense RNA mix, and producing fluorescently labeled cDNA from the RNA starting material. Fluorescently labeled cDNAs prepared from tumorigenic RNA sample were compared to fluorescently labeled cDNAs prepared from non-tumorigenic cell RNA sample. For example, the cDNA probes from the non-tumorigenic cells were labeled with Cy3 fluorescent dye (green) and the cDNA probes prepared from the tumorigenic cells were labeled with Cy5 fluorescent dye (red).

The differential expression assay was performed by mixing equal amounts of probes from tumorigenic cells and non-tumorigenic cells, and/or cells grown in 3D vs. those grown in 2D. The arrays were prehybridized by incubation for about 2 hrs at 60° C. in 5×SSC/0.2% SDS/1 mM EDTA, and then washed three times in water and twice in isopropanol. Following prehybridization of the array, the probe mixture was then hybridized to the array under conditions of high stringency (overnight at 42° C. in 50% formamide, 5×SSC, and 0.2% SDS). After hybridization, the array was washed at 55° C. three times as follows: 1) first wash in 1×SSC/0.2% SDS; 2) second wash in 0.1×SSC/0.2% SDS; and 3) third wash in 0.1×SSC.

The arrays were then scanned for green and red fluorescence using a Molecular Dynamics Generation III dual color laser-scanner/detector. The images were processed using BioDiscovery Autogene software, and the data from each scan set normalized to provide for a ratio of expression relative to non-tumorigenic or tumorigenic cells grown two-dimensionally or three-dimensionally. Data from the microarray experiments was analyzed according to the algorithms described in U.S. application Ser. No. 60/252,358, filed Nov. 20, 2000, by E. J. Moler, M. A. Boyle, and F. M. Randazzo, and entitled “Precision and accuracy in cDNA microarray data,” which application is specifically incorporated herein by reference.

The experiment was repeated, this time labeling the two probes with the opposite color in order to perform the assay in both “color directions.” Each experiment was sometimes repeated with two more slides (one in each color direction). The level fluorescence for each sequence on the array expressed as a ratio of the geometric mean of 8 replicate spots/genes from the four arrays or 4 replicate spots/gene from 2 arrays or some other permutation. The data were normalized using the spiked positive controls present in each duplicated area, and the precision of this normalization was included in the final determination of the significance of each differential. The fluorescence intensity of each spot was also compared to the negative controls in each duplicated area to determine which spots have detected significant expression levels in each sample.

A statistical analysis of the fluorescent intensities was applied to each set of duplicate spots to assess the precision and significance of each differential measurement, resulting in a p-value testing the null hypothesis that there is no differential in the expression level between the tumorigenic and non-tumorigenic cells or cells grown two-dimensionally versus three-dimensionally. During initial analysis of the microarrays, the hypothesis was accepted if p>10⁻³, and the differential ratio was set to 1.000 for those spots. All other spots have a significant difference in expression between the two samples compared. For example, if the tumorigenic sample has detectable expression and the non-tumorigenic does not, the ratio is truncated at 1000 since the value for expression in the non-tumorigenic sample would be zero, and the ratio would not be a mathematically useful value (e.g., infinity). If the non-tumorigenic sample has detectable expression and the tumorigenic does not, the ratio is truncated to 0.001, since the value for expression in the tumor sample would be zero and the ratio would not be a mathematically useful value. These latter two situations are referred to herein as “on/off.” Database tables were populated using a 95% confidence level (p>0.05).

In general, a polynucleotide is said to represent a significantly differentially expressed gene between two samples when there is detectable levels of expression in at least one sample and the ratio value is greater than at least about 1.2 fold, at least about 1.5 fold, or at least about 2 fold, where the ratio value is calculated using the method described above.

A differential expression ratio of I indicates that the expression level of the gene in tumorigenic cells was not statistically different from expression of that gene in the specific non-tumorigenic cells compared. A differential expression ratio significantly greater than 1 in tumorigenic breast cells relative to non-tumorigenic breast cells indicates that the gene is increased in expression in tumorigenic cells relative to non-tumorigenic cells, suggesting that the gene plays a role in the development of the tumorigenic phenotype, and may be involved in promoting metastasis of the cell. Detection of gene products from such genes can provide an indicator that the cell is cancerous, and may provide a therapeutic and/or diagnostic target. Likewise, a differential expression ratio significantly less than 1 in tumorigenic breast cells relative to non-tumorigenic breast cells indicates that, for example, the gene is involved in suppression of the tumorigenic phenotype. Increasing activity of the gene product encoded by such a gene, or replacing such activity, can provide the basis for chemotherapy. Such gene can also serve as markers of cancerous cells, e.g., the absence or decreased presence of the gene product in a breast cell relative to a non-tumorigenic breast cell indicates that the cell is cancerous.

Using the above methodology, three hundred and sixty-seven (367) genes or products thereof were identified from 20,000 chip clones analyzed as being overexpressed 2-fold or more in one or more of these experiments, with a p-value of 0.001 or less. These identified genes or products thereof are listed in Table 1, according to the Spot ID of the spotted polynucleotide, the Sample ID, the corresponding GenBank Accession Number (No.), the GenBank description (if available) for the corresponding Genbank Accession Number, and the GenBank score (p-value; the probability that the association between the SEQ ID NO. and the gene or product thereof occurred by chance). The polynucleotide and polypeptide sequences, as provided by any disclosed Genbank entries are herein incorporated by reference to the corresponding Genbank accession number. The differential hybridization results from the seven differential expression microarray experiments listed above are provided in Table 2, where sequences have a measurement corresponding to its ratio of expression in the 7 experiments, e.g. spot ID 10594 is 2.2-fold overexpressed in 3D T4-2 cells as compared to 3D S1 cells. SEQ ID NOS: 1-499, representing the sequences corresponding to the spot Ids listed in Tables 1 and 2 are provided in the sequence listing. Table 11 is a lookup table showing the relationship between the spot Ids (i.e. the nucleic acids spotted on the microarray) and the sequences provided in the sequence listing.

### Example 4

### Cycling G Associated Kinase (GAK)

A gene or product thereof called cyclin G associated kinase, or GAK, was identified as being overexpressed in 3D T4-2 cultures relative to both 3D S1 cultures (ratio: 7.9296) and 2D T4-2 cultures (ratio: 34.6682) (Sample ID RG: 1056692:10012:C11, Spot ID 19990). GAK corresponds to Genbank Accession number XM_{—}003450.

### Example 5

### Antisense Regulation of GAK Expression

Additional functional information on GAK was generated using antisense knockout technology. A number of different oligonucleotides complementary to GAK mRNA were designed (AS) with corresponding controls (RC): GGAATCACCGCTTTGCCATCTTCAA (SEQ ID NO:500; CHIR159-1AS, gak:P1868AS), AACTTCTACCGTTTCGCCACTAAGG (SEQ ID NO:501; CHIR159-1RC, gak:P1868RC); GACCGTGTACTGCGTGTCGTGCG (SEQ ID NO:502; CHIR159-7AS, gak:P0839AS) and GCGTGCTGTGCGTCATGTGCCAG (SEQ ID NO:502; CHIR159-7RC, gak:P0839RC), and tested for their ability to suppress expression of GAK in human malignant colorectal carcinoma SW620 cells, human breast cancer MDA231 cells, and human breast cancer T4-2 cells. For each transfection mixture, a carrier molecule, preferably a lipitoid or cholesteroid, was prepared to a working concentration of 0.5 mM in water, sonicated to yield a uniform solution, and filtered through a 0.45 μm PVDF membrane. The antisense or control oligonucleotide was then prepared to a working concentration of 100 μM in sterile Millipore water. The oligonucleotide was further diluted in OptiMEM™ (Gibco/BRL), in a microfuge tube, to 2 μM, or approximately 20 μg oligo/ml of OptiMEM™. In a separate microfuge tube, lipitoid or cholesteroid, typically in the amount of about 1.5-2 nmol lipitoid/μg antisense oligonucleotide, was diluted into the same volume of OptiMEM™ used to dilute the oligonucleotide. The diluted antisense oligonucleotide was immediately added to the diluted lipitoid and mixed by pipetting up and down. Oligonucleotide was added to the cells to a final concentration of 300 nM.

The level of target mRNA (GAK) in the transfected cells was quantitated in the cancer cell lines using the methods using primers CHIRI 59_{—}2896 (GCCGTCTTCAGGCAACAACTCCCA; SEQ ID NO: 504; forward) and CHIR159_{—}3089 (TGCTGGACGAGGCTGTCATCTTGC; SEQ ID NO:505; reverse). RNA was extracted as above according to manufacturer's directions.

Quantitative PCR (qPCR) was performed by first isolating the RNA from the above mentioned tissue/cells using a Qiagen RNeasy mini prep kit. A total of 0.5 micrograms of RNA was used to generate a first strand cDNA using Stratagene MuLV Reverse Transcriptase, using recommended concentrations of buffer, enzyme, and Rnasin. Concentrations and volumes of dNTP, and oligo dT, or random hexamers were lower than recommended to reduce the level of background primer dimerization in the qPCR.

The cDNA is then used for qPCR to determine the levels of expression of GAK using the GeneAmp 7000 by ABI as recommended by the manufacturer. Primers for actin were also used in order to normalized the values, and eliminate possible variations in cDNA template concentrations, pipetting error, etc.

For each 20 μl reaction, extracted RNA (generally 0.2-1 μg total) was placed into a sterile 0.5 or 1.5 ml microcentrifuge tube, and water was added to a total volume of 12.5 μl. To each tube was added 7.5 μl of a buffer/enzyme mixture, prepared by mixing (in the order listed) 2.5 μl H₂O, 2.0 μl 10× reaction buffer, 10 μl oligo dT (20 pmol), 1.0 μl dNTP mix (10 mM each), 0.5 μl RNAsin® (20 u) (Ambion, Inc., Hialeah, Fla.), and 0.5 μl MMLV reverse transcriptase (50 u) (Ambion, Inc.). The contents were mixed by pipetting up and down, and the reaction mixture was incubated at 42° C. for 1 hour. The contents of each tube were centrifuged prior to amplification.

An amplification mixture was prepared by mixing in the following order: 1×PCR buffer II, 3 mM MgCl₂, 140 μM each dNTP, 0.175 pmol each oligo, 1:50,000 dil of SYBR® Green, 0.25 mg/ml BSA, 1 unit Taq polymerase, and H₂O to 20 μl. (PCR buffer II is available in 10× concentration from Perkin-Elmer, Norwalk, Conn.). In 1× concentration it contains 10 mM Tris pH 8.3 and 50 mM KCl. SYBR® Gree (Molecular Probes, Eugene, Oreg.) is a dye which fluoresces when bound to double stranded DNA. As double stranded PCR product is produced during amplification, the fluorescence from SYBR® Green increases. To each 20 μl aliquot of amplification mixture, 2 μl of template RT was added, and amplification was carried out according to standard protocols.

Table 3 shows that the antisense oligonucleotides described above reduced expression of GAK mRNA as compared to controls in all three cell lines. GAK mRNA reduction ranged from about 50% to about 90%, as compared to cells transfected with reverse (i.e. sense) control oligonucleotides.

TABLE 3
antisense regulation of GAK mRNA

| | | Gene | Actin | | Percent |
|---|---|---|---|---|---|
| Oligo | Cell Line | Message | Message | Ratio | KO |
| CHIR159-1AS | SW620 | 0.0923 | 0.669 | 0.138 | 90.7 |
| CHIR159-1RC | SW620 | 1.01 | 0.680 | 1.49 | |
| CHIR159-7AS | SW620 | 0.0555 | 0.678 | 0.082 | 85.4 |
| CHIR159-7RC | SW620 | 0.335 | 0.598 | 0.560 | |
| CHIR159-1AS | MDA231 | 0.358 | 0.687 | 0.521 | 59.3 |
| CHIR159-1RC | MDA231 | 1.00 | 0.784 | 1.28 | |
| CHIR159-7AS | MDA231 | 0.262 | 0.674 | 0.389 | 69.4 |
| CHIR159-7RC | MDA231 | 0.840 | 0.659 | 1.27 | |
| CHIR159-1AS | T4-2 | 0.307 | 0.707 | 0.434 | 72.9 |
| CHIR159-1RC | T4-2 | 1.23 | 0.770 | 1.60 | |
| CHIR159-7AS | T4-2 | 0.214 | 0.649 | 0.330 | 49.8 |
| CHIR159-7RC | T4-2 | 0.506 | 0.770 | 0.657 | |

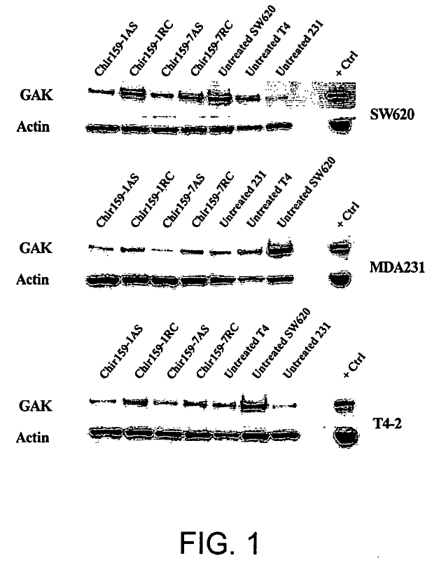

Reduction of GAK protein by antisense polynucleotides in SW620, MDA231 and T4-2 was confirmed using an antibody that specifically recognizes GAK. FIG. 1 shows a western (i.e. protein) blot of protein extracts of the above cell lines decorated with anti-GAK antibodies. GAK protein expression is reduced in cell lines receiving GAK antisense oligonucleotides.

### Example 6

### Role of GAK in Anchorage of Independent Cell Growth

The effect of GAK gene expression upon anchorage-independent cell growth of SW620 and MBA-231 cells was measured by colony formation in soft agar. Soft agar assays were performed by first coating a non-tissue culture treated plate with PolyHEMA to prevent cells from attaching to the plate. Non-transfected cells were harvested using 0.05% trypsin and washing twice in media. The cells are counted using a hemacytometer and resuspended to 10⁴ per ml in media. 50 μl aliquots are placed in poly-HEMA coated 96-well plates and transfected. For each transfection mixture, a carrier molecule, preferably a lipitoid or cholesteroid, was prepared to a working concentration of 0.5 mM in water, sonicated to yield a uniform solution, and filtered through a 0.45 μm PVDF membrane. The antisense or control oligonucleotide was then prepared to a working concentration of 100 μM in sterile Millipore water. The oligonucleotide was further diluted in OptiMEM™ (Gibco/BRL), in a microfuge tube, to 2 μM, or approximately 20 μg oligo/ml of OptiMEMM™. In a separate microfuge tube, lipitoid or cholesteroid, typically in the amount of about 1.5-2 nmol lipitoid/μg antisense oligonucleotide, was diluted into the same volume of OptiMEM™ used to dilute the oligonucleotide. The diluted antisense oligonucleotide was immediately added to the diluted lipitoid and mixed by pipetting up and down. Oligonucleotide was added to the cells to a final concentration of 300 nM. Following transfection (˜30 minutes), 3% GTG agarose is added to the cells for a final concentration of 0.35% agarose. After the cell layer agar solidifies, 100 μl of media is dribbled on top of each well. Colonies form in 7 days. For a read-out of growth, 20 μl of Alamar Blue is added to each well and the plate is shaken for 15 minutes. Fluorecence readings (530nm excitation 590 nm emission) are taken after incubation for 6-24 hours.

The data presented in Table 4 shows that the application of GAK antisense oligonucleotides to SW620 and MDA 231 cells results in inhibition of colony formation and shows that GAK plays a role in production anchorage-independent cell growth. Table 4 shows the average fluorescence reading for several experiments. The standard deviation (St. Dev) of the fluorescence reading and coefficient of variation (% CV) is also shown.

TABLE 4
GAK and anchorage-independent cell growth.

| Oligo | Cell Line | Average | St. Dev | % CV |
|---|---|---|---|---|
| Blank | SW620 | 12868.17 | 208.78 | 1.78 |
| Untreated | SW620 | 31075.17 | 1944.36 | 7.66 |
| Pos Control | SW620 | 5717.17 | 1108.71 | 23.75 |
| Neg Control | SW620 | 7576.17 | 465.95 | 7.63 |
| Chir159-1AS | SW620 | 9701.5 | 2281.36 | 28.8 |
| Chir159-1RC | SW620 | 17765.5 | 1958.45 | 13.5 |
| Blank | MDA231 | 12726.83 | 232.45 | 2 |
| Untreated | MDA231 | 87272.17 | 0 | 0 |
| Pos Control | MDA231 | 10645.17 | 1591.08 | 18.31 |
| Neg Control | MDA231 | 24159.5 | 2850.58 | 14.45 |
| Chir159-1AS | MDA231 | 8613.5 | 4852.76 | 69 |
| Chir159-1RC | MDA231 | 17859.17 | 1535.55 | 10.53 |

### Example 7

### DKFZp566I133 (DKFZ)

Several previously uncharacterized genes were identified as being induced in these experiments. One such gene was represented by two spots, Spot ID Nos 22793 and 26883 (gene assignment DKFZp566I133). This gene was expressed at a ratio of about 2.2 in two 2-dimensional (2D) T4-2 vs. 2D S1 experiments, and also at a ratio of about 2 when 3-dimensional (3D) T4-2 cells were compared to the various tumor reversion cultures. However, the ratio of expression increased to an average of 3.2 when 3-dimensional (3D) T4-2 cultures were compared to 2D S1 cultures. In contrast, there was essentially no difference in expression levels when 3D S1 cultures were compared to 2D S1 cultures, suggesting that expression of this gene is specifically elevated in the tumorigenic cell line T4-2, and even further elevated when the tumorigenic cell line is grown in three dimensional cultures (see Table 5).

TABLE 5

| | | | | | | 3D T4-2/ | |
|---|---|---|---|---|---|---|---|
| Spot | 2D T4-2/ | 3D T42/ | 3D S1/ | 3D T4-2/ | 3D T4-2/ | B1 integrin | 3D T4-2/ |
| ID | 2D S1 | 3D S1 | 2D S1 | 2D T4-2 | EGFRAb | Ab | Tyr |
| 22893 | 1.90387 | 2.64711 | 0.522161 | 1 | 2.17956 | 1.75287 | 2.055538 |
| 26883 | 2.43428 | 3.74613 | 0.524466 | 1 | 2.467573 | 2.029468 | 2.002817 |

These array data were confirmed by qPCR using the methods described above and the gene specific PCR primers CHIR180_{—}1207 ACAGGGAGAAAACTGGTTGTCCTGG (SEQ ID NO:506; Forward) and CHIR180_{—}1403 AAGGCAGAACCCATCCACTCCAA (SEQ ID NO:507; Reverse). Independent cultures were used for these experiments, and data was normalized to B-catenin. These data are shown in Table 6.

TABLE 6

| | | | | | 3D B1 | |
|---|---|---|---|---|---|---|
| | | | | 3D EGFR | Integrin | |
| 2D S1 | 2D T4-2 | 3D S1 | 3D T4-2 | Ab | Ab | 3D Tyr |
| 0.165 | 0.421 | 0.14 | 0.475 | 0.231 | 0.175 | 0.174 |

DKFZ corresponds to Genbank Accession numbers NP_{—}112200, AAH09758, and NM_{—}030938. Orthologs of DKFZ are identified in species other than Homo sapiens include NM_{—}138839 from Ratus norvegicus.

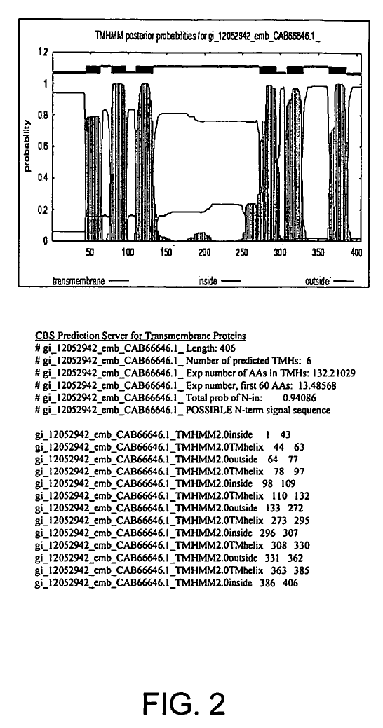

Analysis of the sequence of DKFZ using a transmembrane helix prediction algorithm (Sonhammer, et al, A hidden Markov model for predicting transmembrane helices in protein sequences, In Proc. of Sixth Int. Conf. on Intelligent Systems for Molecular Biology, p. 175-82, Ed. J. Glasgow, T. Littlejohn, F. Major, R. Lathrop, D. Sankoff, and C. Sensen, Menlo Park, Calif.: AAAI Press, 1998) indicates that the DKFZ protein has six transmembrane regions (FIG. 2), and, as such, is likely to be a transmembrane protein.

### Example 8

### Antisense Regulation of DKFZ Expression

Additional functional information on DKFZ was generated using antisense knockout technology. A number of different oligonucleotides complementary to DKFZ mRNA were designed (AS) with corresponding controls (RC): GCTGCTGGATTCGTTTGGCATAACT (SEQ ID NO: 508; CHIR180-7AS, DKFZp56611:P1301AS), TCAATACGGMGCTTAGGTCGTCG (SEQ ID NO:509; CHIR180-7RC, DKFZp56611:P1301RC), TCTCCTCTGAGTTCAACCGCTGCT (SEQ ID NO:510; CHIR180-8AS, DKFZp56611:P1320AS) and TCGTCGCCAACTTGAGTCTCCTCT (SEQ ID NO:511; CHIR180-8RC, DKFZp56611:P1320AS), and tested for their ability to suppress expression of DKFZ in human malignant colorectal carcinoma SW620 cells, human breast cancer MDA231 cells, and human breast cancer T4-2 cells, as described above.

Table 7 shows that the antisense (AS) oligonucleotides described above reduced expression of DKFZ mRNA as compared to controls in all three cell lines. DKFZ mRNA reduction ranged from about 95% to about 99%, as compared to cells transfected with reverse (i.e. sense) control (RC) oligonucleotides.

TABLE 7
antisense regulation of DKFZ mRNA

| | | Gene | Actin | | Percent |
|---|---|---|---|---|---|
| Oligo | Cell Line | Message | Message | Ratio | KO |
| CHIR180-7AS | SW620 | 0.0157 | 0.772 | 0.020 | 99.3 |
| CHIR180-7RC | SW620 | 1.99 | 0.736 | 2.70 | |
| CHIR180-8AS | SW620 | 0.0387 | 0.681 | 0.057 | 97.9 |
| CHIR180-8RC | SW620 | 1.89 | 0.703 | 2.69 | |
| CHIR180-7AS | MDA231 | 0.0471 | 3.58 | 0.013 | 98.5 |
| CHIR180-7RC | MDA231 | 1.99 | 2.33 | 0.854 | |
| CHIR180-8AS | MDA231 | 0.00935 | 1.74 | 0.00537 | 99.5 |
| CHIR180-8RC | MDA231 | 1.14 | 1.01 | 1.13 | |
| CHIR180-7AS | T4-2 | 0.119 | 0.667 | 0.178 | 95.4 |
| CHIR180-7RC | T4-2 | 2.8 | 0.728 | 3.85 | |
| CHIR180-8AS | T4-2 | 0.0852 | 0.751 | 0.113 | 95.6 |
| CHIR180-8RC | T4-2 | 1.6 | 0.620 | 2.58 | |

### Example 9

### Effect of DFKZ Expression on Cell Proliferation

The effect of gene expression on the inhibition of cell proliferation was assessed in metastatic breast cancer cell line MDA-231 and breast cancer cell line T4-2.

Cells were plated to approximately 60-80% confluency in 96-well dishes. Antisense or reverse control oligonucleotide was diluted to 2 μM in OptiMEM™ and added to OptiMEM™ into which a delivery vehicle, preferably a lipitoid or cholesteroid, had been diluted. The oligo/delivery vehicle mixture was then further diluted into medium with serum on the cells. The final concentration of oligonucleotide for all experiments was 300 nM, and the final ratio of oligo to delivery vehicle for all experiments was 1.5 nmol lipitoid/μg oligonucleotide.

Antisense oligonucleotides were prepared. Cells were transfected for 4 hours or overnight at 37° C. and the transfection mixture was replaced with fresh medium. Plates are incubated for 4 days, with a plate harvested for each day0-day4. To determine differences in cell number, a CyQuant Cell Proliferation Assay kit (Molecular Probes) was used per manufacturer's instructions. Fluorecence readings (480nm excitation 520 rn emission) are taken after incubation for 5 minutes.

The results of these assays are shown in Tables 7A and 8. The data show that DKFZ antisense polynucleotides significantly reduce cell proliferation as compared to controls, and, as such, DKFZ plays a role in production or maintenance of the cancerous phenotype in cancerous breast cells.

TABLE 7A
Cell proliferation

| | | Ave | | | | |
|---|---|---|---|---|---|---|
| | | Day | Ave | Ave | Av3 | Ave |
| Oligo | Cell Line | 0 | Day 1 | Day 2 | Day 3 | Day 4 |
| Untreated | MDA231 | 4233 | 4858 | 9544 | 10981 | 16776 |
| Untreated | MDA231 | 3849 | 4036 | 8686 | 9855 | 14865 |
| Pos Control | MDA231 | 3630 | 2236 | 3564 | 4536 | 7477 |
| Neg Control | MDA231 | 4913 | 5127 | 8331 | 8887 | 13620 |
| CHIR180-7AS | MDA231 | 3848 | 3476 | 6942 | 8715 | 11925 |
| CHIR180-7RC | MDA231 | 4895 | 4700 | 8484 | 10318 | 14226 |
| Untreated | T4-2 | 4062 | 3389 | 5438 | 10579 | 15617 |
| Untreated | T4-2 | 4209 | 3802 | 6346 | 11802 | 16275 |
| Pos Control | T4-2 | 3985 | 2712 | 4081 | 6404 | 9685 |
| Neg Control | T4-2 | 4051 | 3901 | 4356 | 9425 | 12964 |
| CHIR180-7AS | T4-2 | 3792 | 3201 | 3849 | 7376 | 10911 |
| CHIR180-7RC | T4-2 | 3967 | 3840 | 4321 | 8382 | 12293 |

TABLE 8

| | Standard Deviations | | | | | P-Value of T-Test | | | | |
|---|---|---|---|---|---|---|---|---|---|---|
| Oligo | Day 0 | Day 1 | Day 2 | Day 3 | Day 4 | Day 0 | Day 1 | Day 2 | Day 3 | Day 4 |
| Untreated | 337 | 269 | 299 | 697 | 1333 | 0.1306 | 0.1063 | 0.1804 | 0.0926 | 0.1225 |
| Untreated | 99 | 631 | 867 | 547 | 1047 | 0.1306 | 0.1063 | 0.1804 | 0.0926 | 0.1225 |
| Pos | 94 | 118 | 89 | 441 | 974 | 0.0000 | 0.0001 | 0.0003 | 0.0001 | 0.0010 |
| Control | | | | | | | | | | |
| Neg | 2 | 252 | 697 | 195 | 780 | 0.0000 | 0.0001 | 0.0003 | 0.0001 | 0.0010 |
| Control | | | | | | | | | | |
| CHIR180-7AS | 292 | 16 | 435 | 398 | 418 | 0.0072 | 0.0276 | 0.0059 | 0.0140 | 0.0028 |
| CHIR180-7RC | 208 | 6 | 244 | 533 | 440 | 0.0072 | 0.0276 | 0.0059 | 0.0140 | 0.0028 |
| Untreated | 64 | 283 | 789 | 1593 | 1226 | 0.2550 | 0.0921 | 0.1257 | 0.2794 | 0.4352 |
| Untreated | 22 | 158 | 205 | 577 | 478 | 0.2550 | 0.0921 | 0.1257 | 0.2794 | 0.4352 |
| Pos | 122 | 213 | 6 | 475 | 957 | 0.4320 | 0.0065 | 0.2624 | 0.0051 | 0.0293 |
| Control | | | | | | | | | | |
| Neg | 47 | 335 | 464 | 809 | 1417 | 0.4320 | 0.0065 | 0.2624 | 0.0051 | 0.0293 |
| Control | | | | | | | | | | |
| CHIR180-7AS | 170 | 679 | 263 | 127 | 1330 | 0.2638 | 0.0976 | 0.3516 | 0.0040 | 0.0039 |
| CHIR180-7RC | 22 | 453 | 646 | 579 | 884 | 0.2638 | 0.0976 | 0.3516 | 0.0040 | 0.0039 |

### Example 10

### Role of DKFZ in Anchorage Independent Cell Growth

The effect of DKFZ gene expression upon anchorage-independent cell growth of MDA435 and MCF7 human breast cancer cells was measured by colony formation in soft agar. Soft agar assays were conducted by the method described for GAK, above.

The data presented in Table 9 shows that the application of DKFZ antisense oligonucleotides to MDA435 and MCF7 cells results in inhibition of colony formation and shows that DKFZ plays a role in anchorage-independent cell growth of cancer cells. Table 9 shows the average fluorescence reading for several experiments. The standard deviation (St. Dev) of the fluorescence reading and coefficient of variation (% CV) and probability (P-value) is also shown.

TABLE 9

| Oligo | Cell Line | Average | St. Dev | % CV | P-Value |
|---|---|---|---|---|---|
| Untreated | MDA435 | 31190 | 5838 | 19 | 0.1342 |
| Untreated | MDA435 | 38623 | 3620 | 9 | 0.1342 |
| Pos Control | MDA435 | 4776 | 818 | 17 | 0.0156 |
| Neg Control | MDA435 | 16315 | 481 | 3 | 0.0156 |
| Chir180-7AS | MDA435 | 21161 | 3439 | 16 | 0.0274 |
| Chir180-7RC | MDA435 | 28868 | 1902 | 7 | 0.0274 |
| Untreated | MCF7 | 18954 | 1478 | 8 | 0.1476 |
| Untreated | MCF7 | 14383 | 4163 | 29 | 0.1476 |
| Pos Control | MCF7 | 1036 | 194 | 19 | 0.0036 |
| Neg Control | MCF7 | 9478 | 2382 | 25 | 0.0036 |
| Chir180-7AS | MCF7 | 4752 | 2002 | 42 | 0.0139 |
| Chir180-7RC | MCF7 | 9570 | 18 | 0 | 0.0139 |

The effect of DKFZ gene expression upon invasiveness of MDA231 human breast cancer cells was measured by a matrigel assay. A 3-dimensional reconstituted basement membrane culture of cells was generated as described previously (Peterson et al., (1992) Proc. Natl. Acad. Sci. USA 89:9064-9068) using a commercially prepared reconstituted basement membrane (Matrigel; Collaborative Research, Waltham, Mass.) and examined using methods well known in the art.

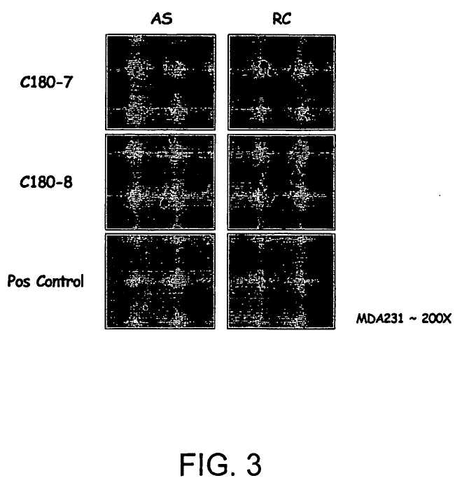

Table 10 (quantitated using Alamar Blue similar to the soft agar assay) and FIG. 3 provides exemplary results of the Matrigel invasion/motility assay to test the invasiveness of MDA231 cells with reduced expression of DKFZ. In general, these data show that a reduction in the expression of DKFZ significantly decreases the invasiveness of MDA231 cells.

TABLE 10

| Oligo | Cell Line | Average | St. Dev | % CV | P-Value |
|---|---|---|---|---|---|
| Untreated | MDA231 | 28316 | 13663 | 48 | 0.9080 |
| Untreated | MDA231 | 26840 | 15669 | 58 | 0.9080 |
| Pos Control | MDA231 | 2756 | 487 | 18 | 0.0002 |
| Neg Control | MDA231 | 14301 | 1386 | 10 | 0.0002 |
| Chir180-7AS | MDA231 | 10508 | 1963 | 19 | 0.0287 |
| Chir180-7RC | MDA231 | 14310 | 153 | 1 | 0.0287 |

### Example 11

### Expression of DKFZ in Cancer Tissues

The following peptides were used for polyclonal antibody production: peptide 809: gvhqqyvqriek (SEQ ID NO:380), corresponding to amino acids 97-108 of the DKFZ protein and peptide 810: sgaepddeeyqef (SEQ ID NO:381), corresponding to amino acids 215-227 of the DKFZ protein.

Antibodies specific for DKFZ are used in FACS and immunolocalization analysis to show that DKFZ is associated with membrane, and up-regulated in cancer tissues of biopsies from cancer patients.

Further, antibodies specific for DKFZ are used to modulate DKFZ activity in cancerous breast, and is further used, alone or conjugated to a toxic moiety, as a treatment for breast cancer.

While the present invention has been described with reference to the specific embodiments thereof, it should be understood by those skilled in the art that various changes may be made and equivalents may be substituted without departing from the true spirit and scope of the invention. In addition, many modifications may be made to adapt a particular situation, material, composition of matter, process, process step or steps, to the objective, spirit and scope of the present invention. All such modifications are intended to be within the scope of the claims appended hereto

TABLE 1

| | | GENBANK | | GENBANK |
|---|---|---|---|---|
| SPOTID | SAMPLE ID | NO | GENBANK DESCRIPTION | SCORE |
| 10594 | I:1871362:05B01:A04 | M62994 | Homo sapiens thyroid autoantigen (truncated actin- | 8.6E−36 |
| | | | binding protein) mRNA, complete cds | |
| 21851 | M00055153A:A12 | | | |
| 20990 | I:1986550:13B02:G12 | XM_005667 | Homo sapiens lipocalin 2 (oncogene 24p3) (LCN2). | 0 |
| | | | mRNA | |
| 18641 | I:3473302:09A01:A09 | AB046098 | Macaca fascicularis brain cDNA, clone: QccE-15843 | 5.8E−57 |
| 17229 | I:1506962:09A01:G01 | AL365454 | Homo sapiens mRNA full length insert cDNA clone | 2.6E−110 |
| | | | EUROIMAGE 926491 | |
| 25930 | 035JN020.F01 | AJ010446 | Homo sapiens mRNA for immunoglobulin kappa | 0 |
| | | | light chain, anti-RhD, therad 24 | |
| 20701 | RG:730349:10010:G12 | U28387 | Human hexokinase II pseudogene, complete cds | 0 |
| 20346 | RG:1839794:10015:E11 | U28387 | Human hexokinase II pseudogene, complete cds | 0 |
| 21247 | M00054680C:A06 | U28387 | Human hexokinase II pseudogene, complete cds | 9.9E−80 |
| 23062 | M00056353C:E10 | XM_011013 | Homo sapiens filamin B, beta (actin-binding protein- | 0 |
| | | | 278) (FLNB), mRNA | |
| 25666 | 035Jn031.B01 | AF191633 | Homo sapiens filamin (FLNB) gene, exon 48 and | 0 |
| | | | complete cds | |
| 19001 | I:2171401:09A02:E09 | AF123887 | Homo sapiens ERO1L (ERO1L) mRNA, partial cds | 3.3E−104 |
| 10897 | I:1852047:02A01:A10 | U22384 | Human lysyl oxidase gene, partial cds | 0 |
| 1960 | M00023297B:A10 | M33376 | Human pseudo-chlordecone reductase mRNA, | 0 |
| | | | complete cds | |
| 26381 | 035JN029.H02 | AB037838 | Homo sapiens mRNA for KIAA1417 protein, partial | 0 |
| | | | cds | 0 |
| 26719 | 035JN030.A02 | X68277 | H. sapiens CL 100 mRNA for protein tyrosine | 0 |
| | | | phosphatase | |
| 27152 | 037XN007.A09 | XM_048479 | Homo sapiens hypothetical protein FLJ14642 | 7.3E−58 |
| | | | (FLJ14642), mRNA | |
| 10926 | I:2047770:08B02:G04 | AK000969 | Homo sapiens cDNA FLJ10107 fis, clone | 3.8E−94 |
| | | | HEMBA1002583 | |
| 28980 | 035JN003.C12 | XM_027456 | Homo sapiens hypothetical gene supported by | 0 |
| | | | AK000584 (LOC89942), mRNA | |
| 1236 | M00022024A:F02 | | | |
| 29350 | 035JN008.D06 | XM_043864 | Homo sapiens phosphoinositide-3-kinase, | 0 |
| | | | regulatory subunit, polypeptide 1 (p85 alpha) | |
| | | | (PIK3R1), mRNA | |
| 26242 | 035JN015.B02 | AL137717 | Homo sapiens mRNA; cDNA DKFZp434J1630 | 2.6E−70 |
| | | | (from clone DKFZp434J1630) | |
| 4098 | M00001439D:C09 | BC002446 | Homo sapiens, MRJ gene for a member of the | 0 |
| | | | DNAJ protein family, clone MGC: 1152 | |
| | | | IMAGE: 3346070, mRNA, complete cds | |
| 17432 | I:1965049:16B02:D07 | XM_051165 | Homo sapiens DKFZP586A0522 protein | 0 |
| | | | (DKFZP586A0522), mRNA | |
| 1785 | SL198 | XM_051165 | Homo sapiens DKFZP586A0522 protein | 0 |
| | | | (DKFZP586A0522), mRNA | |
| 28856 | 035JN032.E11 | X62996 | H. sapiens mitochondrial genome (consensus | 0 |
| | | | sequence) | |
| 18791 | RG:229957:10007:D03 | D42042 | Human mRNA for KIAA0085 gene, partial cds | 0 |
| 22950 | M00056922C:C09 | | | |
| 1882 | M00022196B:D09 | Z29083 | H. sapiens 5T4 gene for 5T4 Oncofetal antigen | 0 |
| 23886 | M00055408A:F10 | | | |
| 24995 | M00055215C:E11 | XM_012880 | Homo sapiens hypothetical protein MGC1936 | 0 |
| | | | (MGC1936), mRNA | |
| 24477 | M00055510B:F08 | AF240697 | Homo sapiens retinol dehydrogenase homolog | 0 |
| | | | isoform-2 (RDH) mRNA, complete cds | |
| 21681 | M00056771C:A12 | X02152 | Human mRNA for lactate dehydrogenase-A (LDH- | 0 |
| | | | A, EC 1.1.1.27) | |
| 9557 | I:1335140:05A02:C08 | X02152 | Human mRNA for lactate dehydrogenase-A (LDH- | 0 |
| | | | A, EC 1.1.1.27) | |
| 22033 | M00056574B:A07 | | | |
| 873 | M00007979C:C05 | X00663 | Human mRNA fragment for epidermal growth factor | 0 |
| | | | (EGF) receptor | |
| 17144 | RG:25254:10004:D07 | M97675 | Human transmembrane receptor (ror1) mRNA, | 0 |
| | | | complete cds | |
| 26970 | 035JN015.F09 | AF097514 | Homo sapiens stearoyl-CoA desaturase (SCD) | 0 |
| | | | mRNA, complete cds | |
| 21402 | M00054507C:D07 | | | |
| 27074 | 035Jn031.B03 | AF061741 | Homo sapiens retinal short-chain | 0 |
| | | | dehydrogenase/reductase retSDR1 mRNA, | |
| | | | complete cds | |
| 10963 | I:1258790:05A02:B10 | AF072752 | Homo sapiens ten integrin EGF-like repeat domains | 0 |
| | | | protein precursor (ITGBL1) mRNA, complete cds | |
| 29525 | 035JN026.D12 | | | |
| 25514 | 035JN011.F01 | U62961 | Human succinyl CoA: 3-oxoacid CoA transferase | 0 |
| | | | precursor (OXCT) mRNA, complete cds | |
| 26612 | 035JN016.C08 | NM_000240 | Homo sapiens monoamine oxidase A (MAOA), | 0 |
| | | | nuclear gene encoding mitochondrial protein, | |
| | | | mRNA | |
| 24600 | M00055490C:G11 | U57059 | Homo sapiens Apo-2 ligand mRNA, complete cds | 0 |
| 9741 | I:3126828:12A02:G02 | U37518 | Human TNF-related apoptosis inducing ligand | 0 |
| | | | TRAIL mRNA, complete cds | |
| 23689 | M00054752A:E11 | XM_001468 | Homo sapiens S100 calcium-binding protein A10 | 0 |
| | | | (annexin II ligand, calpactin I, light polypeptide | |
| | | | (p11)) (S100A10), mRNA | |
| 22352 | M00042842B:E02 | XM_001468 | Homo sapiens S100 calcium-binding protein A10 | 0 |
| | | | (annexin II ligand, calpactin I, light polypeptide | |
| | | | (p11)) (S100A10), mRNA | |
| 23806 | RG:2007319:20003:G10 | | | |
| 12285 | I:1404669:04A01:G12 | BC002517 | Homo sapiens, Pirin, clone MGC: 2083 | 0 |
| | | | IMAGE: 3140037, mRNA, complete cds | |
| 27638 | 035JN011.D10 | AK002155 | Homo sapiens cDNA FLJ11293 fis, clone | 0 |
| | | | PLACE1009670, highly similar to Homo sapiens | |
| | | | genethonin 1 mRNA | |
| 9663 | I:2488567:11A02:H08 | XM_006027 | Homo sapiens brain-derived neurotrophic factor | 0 |
| | | | (BDNF), mRNA | |
| 26850 | 035JN003.B03 | XM_031551 | Homo sapiens similar to carbohydrate (N- | 0 |
| | | | acetylglucosamine-6-O) sulfotransferase 2 (H. sapiens) | |
| | | | (LOC90414), mRNA | |
| 10204 | I:1491445:02B01:F09 | AF131765 | Homo sapiens clone 24833 nonsyndromic hearing | 0 |
| | | | impairment protein mRNA sequence, complete cds | |
| 1318 | 2192-6 | | | |
| 25922 | 035JN020.B01 | AB020673 | Homo sapiens mRNA for KIAA0866 protein, | 0 |
| | | | complete cds | |
| 26347 | 035JN025.G02 | | | |
| 20361 | I:395116:17A02:E05 | | | |
| 28672 | 035JN012.A05 | AF126181 | Homo sapiens breast cancer-associated gene 1 | 0 |
| | | | protein (BCG1) mRNA, complete cds | |
| 25520 | 035JN011.A07 | D86956 | Human mRNA for KIAA0201 gene, complete cds | 0 |
| 1723 | M00005694A:A09 | BC001980 | Homo sapiens, clone IMAGE: 3462291, mRNA | 0 |
| 28863 | 037XN002.A05 | | | |
| 25526 | 035JN011.D07 | AF086281 | Homo sapiens full length insert cDNA clone | 0 |
| | | | ZD45G11 | |
| 27936 | 035JN008.A04 | X59445 | H. sapiens mRNA for colon carcinoma Manganese | 0 |
| | | | Superoxide Dismutase | |
| 26851 | 035JN001.C03 | XM_033944 | Homo sapiens superoxide dismutase 2, | 0 |
| | | | mitochondrial (SOD2), mRNA | |
| 25107 | M00054825A:E04 | AF075061 | Homo sapiens full length insert cDNA YP07G10 | 0 |
| 24912 | M00054505D:D06 | AF075061 | Homo sapiens full length insert cDNA YP07G10 | 0 |
| 25169 | M00055510D:D04 | M11167 | Human 28S ribosomal RNA gene | 1.2E−76 |
| 25600 | 035JN023.A01 | BC003107 | Homo sapiens, inhibitor of DNA binding 3, dominant | 0 |
| | | | negative helix-loop-helix protein, clone MGC: 1988 | |
| | | | IMAGE: 3543936, mRNA, complete | |
| 28706 | 035JN016.B05 | X55181 | Human ETS2 gene. 3′end | 0 |
| 26377 | 035JN029.F02 | Y14436 | Homo sapiens mRNA for phosphatidic acid | 0 |
| | | | phosphatase type 2 | |
| 19460 | I:438655:14B02:B04 | AF007133 | Homo sapiens clone 23764 mRNA sequence | 4.5E−113 |
| 25243 | RG:1667183:10014:F12 | BC000013 | Homo sapiens, insulin-like growth factor binding | 0 |
| | | | protein 3, clone MGC: 2305 IMAGE: 3506666, | |
| | | | mRNA, complete cds | |
| 20018 | I:1213574:17B01:A11 | AB037925 | Homo sapiens MAIL mRNA, complete cds | 3.7E−106 |
| 918 | M00026895D:H03 | BC006433 | Homo sapiens, Ras-related GTP-binding protein, | 0 |
| | | | clone MGC: 13077 IMAGE: 3835186, mRNA, | |
| | | | complete cds | |
| 25027 | RG:1983823:20002:B06 | | | |
| 29089 | 035JN017.B06 | XM_037534 | Homo sapiens phosphodiesterase 7A (PDE7A), | 0 |
| | | | mRNA | |
| 9141 | I:1347384:02A02:C07 | U78579 | Human type I phosphatidylinositol-4-phosphate 5- | 0 |
| | | | kinase beta (STM7) mRNA, partial cds | |
| 12005 | I:1259230:05A01:C06 | D87075 | Human mRNA for KIAA0238 gene, partial cds | 0 |
| 12148 | I:3360476:03B01:B12 | XM_040922 | Homo sapiens interleukin 13 receptor, alpha 2 | 0 |
| | | | (IL13RA2), mRNA | |
| 17394 | RG:1943755:10016:A07 | AF346607 | Homo sapiens interleukin-1 receptor associated | 0 |
| | | | kinase 1b (IRAK) mRNA, complete cds, | |
| | | | alternatively spliced | |
| 27017 | 035JN021.F03 | XM_051742 | Homo sapiens spermine synthase (SMS), mRNA | 0 |
| 25809 | 035JN002.B07 | XM_009699 | Homo sapiens nuclear receptor interacting protein 1 | 0 |
| | | | (NRIP1), mRNA | |
| 8719 | I:2600080:10A01:H01 | XM_009665 | Homo sapiens Kreisler (mouse) maf-related leucine | 0 |
| | | | zipper homolog (KRML), mRNA | |
| 21030 | RG:1714832:10015:C06 | XM_029957 | Homo sapiens Rab acceptor 1 (prenylated) | 0 |
| | | | (RABAC1), mRNA | |
| 11436 | I:1470085:03B01:F05 | XM_038976 | Homo sapiens N-ethylmaleimide-sensitive factor | 0 |
| | | | attachment protein, alpha (NAPA), mRNA | |
| 10374 | I:1513989:03B02:C03 | XM_009010 | Homo sapiens complement component 3 (C3), | 1.4E−96 |
| | | | mRNA | |
| 19037 | I:417827:15A01:G10 | X79538 | H. sapiens nuk_34 mRNA for translation initiation | 1.9E−28 |
| | | | factor | |
| 398 | M00027016A:C05 | XM_031470 | Homo sapiens aldolase C, fructose-bisphosphate | 4E−62 |
| | | | (ALDOC), mRNA | |
| 18773 | I:1211682:14A02:C09 | XM_008477 | Homo sapiens aldolase C, fructose-bisphosphate | 0 |
| | | | (ALDOC), mRNA | |
| 3583 | M00023407B:C10 | | | |
| 3418 | M00001470A:C03 | XM_043951 | Homo sapiens CDP-diacylglycerol-inositol 3- | 0 |
| | | | phosphatidyltransferase (phosphatidylinositol | |
| | | | synthase) (CDIPT), mRNA | |
| 18985 | I:1402615:09A02:E03 | AF191148 | Homo sapiens type I transmembrane protein Fn14 | 7.9E−64 |
| | | | mRNA, complete cds | |
| 25861 | 035JN010.D01 | XM_047975 | Homo sapiens hydroxyacyl glutathione hydrolase | 0 |
| | | | (HAGH), mRNA | |
| 3317 | M00003974D:E04 | AF136185 | Homo sapiens collagen type XVII (COL17A1) gene, | 0 |
| | | | 3′ UTR, long form | |
| 8743 | I:1858905:04A01:D01 | U36775 | Human ribonuclease 4 gene, partial cds | 2.1E−57 |
| 26240 | 035JN015.A02 | XM_007493 | Homo sapiens ribonuclease, RNase A family, 4 | 0 |
| | | | (RNASE4), mRNA | |
| 28562 | 037XN007.B11 | X00947 | Human alpha 1-antichymotrypsin gene fragment | 0 |
| 16877 | I:2362945:15A01:C07 | XM_029378 | Homo sapiens checkpoint suppressor 1 (CHES1), | 1.9E−91 |
| | | | mRNA | |
| 25955 | 035JN022.C01 | AF035620 | Homo sapiens BRCA1-associated protein 2 | 0 |
| | | | (BRAP2) mRNA, complete cds | |
| 26308 | 035JN023.C02 | XM_041470 | Homo sapiens zinc finger protein 145 (Kruppel-like, | 0 |
| | | | expressed in promyelocytic leukemia) (ZNF145), | |
| | | | mRNA | |
| 4140 | 2239-4 | X03083 | Human lactate dehydrogenase-A gene exon 7 and | 0 |
| | | | 3′ flanking region | |
| 3436 | 2239-1 | X03083 | Human lactate dehydrogenase-A gene exon 7 and | 0 |
| | | | 3′ flanking region | |
| 25612 | 035JN023.G01 | M94856 | Human fatty acid binding protein homologue (PA- | 0 |
| | | | FABP) mRNA, complete cds | |
| 12257 | I:1448135:04A01:A06 | X15535 | H. sapiens lysosomal acid phosphatase gene (EC | 0 |
| | | | 3.1.3.2) Exon 11 | |
| 9111 | I:1958902:04A02:D07 | D87258 | Homo sapiens mRNA for serin protease with IGF- | 0 |
| | | | binding motif, complete cds | |
| 17620 | I:875567:15B01:B08 | XM_045326 | Homo sapiens MAX-interacting protein 1 (MXI1), | 0 |
| | | | mRNA | |
| 26025 | 035JN030.F01 | XM_032511 | Homo sapiens procollagen-proline, 2-oxoglutarate 4- | 0 |
| | | | dioxygenase (proline 4-hydroxylase), alpha | |
| | | | polypeptide I (P4HA1), mRNA | |
| 19271 | RG:686684:10010:D04 | AF005216 | Homo sapiens receptor-associated tyrosine kinase | 0 |
| | | | (JAK2) mRNA, complete cds | |
| 4151 | 2035-1 | D87953 | Human mRNA for RTP, complete cds | 0 |
| 26569 | 035JN010.F02 | AB004788 | Homo sapiens mRNA for BNIP3L, complete cds | 0 |
| 10344 | I:2859338:11B02:D03 | XM_005052 | Homo sapiens angiopoietin 1 (ANGPT1), mRNA | 1.3E−97 |
| 832 | M00021649B:D05 | XM_004628 | Homo sapiens hypoxia-inducible protein 2 (HIG2), | 0 |
| | | | mRNA | |
| 12071 | I:1798283:06A01:D06 | S72481 | pantophysin [human, keratinocyte line HaCaT, | 0 |
| | | | mRNA, 2106 nt] | |
| 12271 | I:1445767:04A01:H06 | X12701 | H. sapiens mRNA for endothelial plasminogen | 1.8E−130 |
| | | | activator inhibitor PAI | |
| 11433 | I:1526282:03A01:E05 | XM_033627 | Homo sapiens glycoprotein (transmembrane) nmb | 3.7E−117 |
| | | | (GPNMB), mRNA | |
| 20917 | RG:222350:10007:C12 | X00663 | Human mRNA fragment for epidermal growth factor | 1.7E−122 |
| | | | (EGF) receptor | |
| 25810 | 035JN004.B07 | X00588 | Human mRNA for precursor of epidermal growth | 0 |
| | | | factor receptor | |
| 12039 | I:3506985:07A01:D06 | M24795 | Human CD36 antigen mRNA, complete cds | 0 |
| 25499 | 035JN005.G07 | XM_028224 | Homo sapiens N-acetylglucosamine-phosphate | 0 |
| | | | mutase (AGM1), mRNA | |
| 25557 | 035JN013.D07 | BC010135 | Homo sapiens, cyclin C, clone IMAGE: 4106819, | 0 |
| | | | mRNA | |
| 9917 | I:1283532:05A01:G09 | XM_004148 | Homo sapiens 5T4 oncofetal trophoblast | 2.4E−70 |
| | | | glycoprotein (5T4), mRNA | |
| 19505 | RG:204653:10007:A10 | XM_003789 | Homo sapiens colony stimulating factor 1 receptor, | 0 |
| | | | formerly McDonough feline sarcoma viral (v-fms) | |
| | | | oncogene homolog (CSF1R), mRNA | |
| 17491 | RG:277866:10008:B07 | XM_003789 | Homo sapiens colony stimulating factor 1 receptor, | 0 |
| | | | formerly McDonough feline sarcoma viral (v-fms) | |
| | | | oncogene homolog (CSF1R), mRNA | |
| 10683 | I:1686726:06A01:F10 | XM_003789 | Homo sapiens colony stimulating factor 1 receptor, | 0 |
| | | | formerly McDonough feline sarcoma viral (v-fms) | |
| | | | oncogene homolog (CSF1R), mRNA | |
| 1936 | M00008020C:H09 | X68277 | H. sapiens CL 100 mRNA for protein tyrosine | 0 |
| | | | phosphatase | |
| 828 | M00021638B:F03 | X68277 | H. sapiens CL 100 mRNA for protein tyrosine | 0 |
| | | | phosphatase | |
| 9558 | I:1824443:05B02:C08 | XM_003708 | Homo sapiens gamma-aminobutyric acid (GABA) A | 0 |
| | | | receptor, pi (GABRP), mRNA | |
| 20164 | I:1997963:14B02:B05 | XM_003631 | Homo sapiens solute carrier family 25 | 0 |
| | | | (mitochondrial carrier, adenine nucleotide | |
| | | | translocator), member 4 (SLC25A4), mRNA | |
| 969 | NIH50_40026 | BC008664 | Homo sapiens, clone MGC: 9281 IMAGE: 3871960, | 0 |
| | | | mRNA, complete cds | |
| 9910 | I:1805840:05B01:C09 | XM_003399 | Homo sapiens mannosidase, beta A, lysosomal | 0 |
| | | | (MANBA), mRNA | |
| 2427 | M00005767D:B03 | XM_047441 | Homo sapiens RAP1, GTP-GDP dissociation | 0 |
| | | | stimulator 1 (RAP1GDS1), mRNA | |
| 19990 | RG:1056692:10012:C11 | XM_003450 | Homo sapiens cyclin G associated kinase (GAK), | 0 |
| | | | mRNA | |
| 20605 | I:690313:16A01:G12 | XM_011152 | Homo sapiens insulin-like growth factor binding | 0 |
| | | | protein 7 (IGFBP7), mRNA | |
| 10650 | I:2456393:07B01:E10 | AK001580 | Homo sapiens cDNA FLJ10718 fis, clone | 0 |
| | | | NT2RP3001096, weakly similar to Rattus | |
| | | | norvegicus leprecan mRNA | |
| 25963 | 035JN022.G01 | X53002 | Human mRNA for integrin beta-5 subunit | 0 |
| 25562 | 035JN015.F07 | X53002 | Human mRNA for integrin beta-5 subunit | 0 |
| 9377 | I:2782593:12A01:A02 | X60656 | H. sapiens mRNA for elongation factor 1-beta | 1.4E−46 |
| 17618 | I:707667:15B01:A08 | XM_002273 | Homo sapiens inhibitor of DNA binding 2, dominant | 3.5E−117 |
| | | | negative helix-loop-helix protein (ID2), mRNA | |
| 12136 | I:3208994:03B01:D06 | U16267 | Human AMP deaminase isoform L, alternatively | 0 |
| | | | spliced (AMPD2) mRNA, exons 1A, 2 and 3, partial | |
| | | | cds | |
| 17373 | I:1538189:14A02:G07 | XM_046818 | Homo sapiens similar to receptor tyrosine kinase- | 8.3E−123 |
| | | | like orphan receptor 1 (H. sapiens) (LOC92711), | |
| | | | mRNA | |
| 18577 | RG:503209:10010:A09 | XM_049305 | Homo sapiens Lysosomal-associated multispanning | 0 |
| | | | membrane protein-5 (LAPTM5), mRNA | |
| 3143 | M00001605D:C02 | BC003107 | Homo sapiens, inhibitor of DNA binding 3, dominant | 1.7E−88 |
| | | | negative helix-loop-helix protein, clone MGC: 1988 | |
| | | | IMAGE: 3543936, mRNA, complete | |
| 17737 | RG:155066:10006:E02 | AL050147 | Homo sapiens mRNA; cDNA DKFZp586E0820 | 0 |
| | | | (from clone DKFZp586E0820): partial cds | |
| 20029 | I:1923613:17A01:G11 | AF113123 | Homo sapiens carbonyl reductase mRNA, complete | 0 |
| | | | cds | |
| 18537 | NIH50_40304 | BC001380 | Homo sapiens, succinate dehydrogenase complex, | 0 |
| | | | subunit A, flavoprotein (Fp), clone MGC: 1484 | |
| | | | IMAGE: 3051442, mRNA, complete cds | |
| 10090 | NIH50_40304 | | | |
| 12102 | I:2832414:11B01:C06 | XM_048045 | Homo sapiens katanin p80 (WD40-containing) | 0 |
| | | | subunit B 1 (KATNB1), mRNA | |
| 8487 | I:1375115:05A01:D01 | BC001174 | Homo sapiens, exostoses (multiple) 1, clone | 0 |
| | | | MGC: 2129 IMAGE: 3502232, mRNA, complete cds | |
| 9252 | I:1673876:06B01:B02 | BC000917 | Homo sapiens, clone MGC: 5184 IMAGE: 3048750, | 0 |
| | | | mRNA, complete cds | |
| 25605 | 035JN021.D01 | BC000671 | Homo sapiens, claudin 4, clone MGC: 1778 | 0 |
| | | | IMAGE: 3349211, mRNA, complete cds | |
| 29652 | M00001610C:D05 | BC000588 | Homo sapiens, HIRA-interacting protein 3, clone | 0 |
| | | | MGC: 1814 IMAGE: 3345739, mRNA, complete cds | |
| 10858 | I:2458933:04B01:E04 | X97544 | H. sapiens mRNA for TIM17 preprotein translocase | 8.7E−62 |
| 1261 | M00023419C:B06 | U89606 | Human pyridoxal kinase mRNA, complete cds | 0 |
| 4156 | 2243-4 | X93334 | Homo sapiens mitochondrial DNA, complete | 0 |
| | | | genome | |
| 3452 | 2243-1 | X93334 | Homo sapiens mitochondrial DNA, complete | 0 |
| | | | genome | |
| 2748 | 2242-6 | X93334 | Homo sapiens mitochondrial DNA, complete | 0 |
| | | | genome | |
| 2046 | 2248-3 | X93334 | Homo sapiens mitochondrial DNA, complete | 0 |
| | | | genome | |
| 2044 | 2242-4 | X93334 | Homo sapiens mitochondrial DNA, complete | 0 |
| | | | genome | |
| 1342 | 2248-2 | X93334 | Homo sapiens mitochondrial DNA, complete | 0 |
| | | | genome | |
| 1326 | 2244-3 | X93334 | Homo sapiens mitochondrial DNA, complete | 0 |
| | | | genome | |
| 9981 | I:1720149:06A01:G09 | AF069604 | Homo sapiens myosin light chain kinase isoform 4 | 0 |
| | | | (MLCK) mRNA, partial cds | |
| 27917 | 035JN002.H04 | XM_015978 | Homo sapiens hypothetical protein FLJ22969 | 1.8E−92 |
| | | | (FLJ22969), mRNA | |
| 8488 | I:1808529:05B01:D01 | AJ293647 | Homo sapiens partial IL4RA gene for interleukin-4 | 1.1E−125 |
| | | | receptor alfa chain, exon 11, ECSSQV allele | |
| 22793 | M00057283C:D06 | AF161410 | Homo sapiens HSPC292 mRNA, partial cds | 0 |
| 26883 | 035JN005.C03 | AF161410 | Homo sapiens HSPC292 mRNA, partial cds | 0 |
| 11540 | I:1909488:10B01:B11 | XM_027739 | Homo sapiens duodenal cytochrome b (FLJ23462), | 0 |
| | | | mRNA | |
| 17707 | I:489882:14A01:F02 | X99474 | H. sapiens mRNA for chloride channel, CIC-6c | 0 |
| 20649 | NIH50_41452 | Z14136 | H. sapiens gene for spermidine/spermine N1- | 0 |
| | | | acetyltransferase | |
| 24004 | M00056163C:H09 | AF107495 | Homo sapiens FWP001 and putative FWP002 | 0 |
| | | | mRNA, complete cds | |
| 11836 | I:1806769:01B02:F11 | X93036 | H. sapiens mRNA for MAT8 protein | 0 |
| 24932 | M00054963C:C09 | M26152 | Homo sapiens serum amyloid A (SAA) mRNA, | 0 |
| | | | complete cds | |
| 19143 | RG:149960:10006:D04 | AK003448 | Mus musculus 18 days embryo cDNA, RIKEN full- | 8.9E−21 |
| | | | length enriched library, clone: 1110004P15, full | |
| | | | insert sequence | |
| 26257 | 035JN013.B08 | J04056 | Human carbonyl reductase mRNA, complete cds | 0 |
| 21239 | M00054679B:B03 | J02619 | Human Z type alpha-1-antitrypsin gene, complete | 0 |
| | | | cds (exons 2-5) | |
| 16959 | I:1426031:14B01:B07 | AY035783 | Homo sapiens laminin 5 beta 3 subunit (LAMB3) | 3.8E−121 |
| | | | mRNA, complete cds | |
| 2568 | M00022158D:C11 | XM_036609 | Homo sapiens laminin, beta 3 (nicein (125 kD), | 0 |
| | | | kalinin (140 kD), BM600 (125 kD)) (LAMB3), mRNA | |
| 25936 | 035JN020.A07 | XM_036608 | Homo sapiens laminin, beta 3 (nicein (125 kD), | 0 |
| | | | kalinin (140 kD), BM600 (125 kD)) (LAMB3), mRNA | |
| 23041 | M00054797C:G10 | XM_046649 | Homo sapiens nuclear factor of kappa light | 0 |
| | | | polypeptide gene enhancer in B-cells inhibitor, | |
| | | | alpha (NFKBIA), mRNA | |
| 9206 | I:1822716:05B01:C08 | BC008059 | Homo sapiens, clone IMAGE: 2967491, mRNA | 0 |
| 25105 | M00054824C:H04 | BC009110 | Homo sapiens, clone MGC: 17355 IMAGE: 3453825, | 0 |
| | | | mRNA, complete cds | |
| 24779 | M00057061D:G07 | | | |
| 22451 | M00043372B:B06 | X00947 | Human alpha 1-antichymotrypsin gene fragment | 0 |
| 22291 | M00054785D:G05 | X00947 | Human alpha 1-antichymotrypsin gene fragment | 0 |
| 21143 | M00055146A:D11 | | | |
| 24751 | M00054676B:D07 | X03083 | Human lactate dehydrogenase-A gene exon 7 and | 0 |
| | | | 3′ flanking region | |
| 24294 | M00056163D:E01 | X03083 | Human lactate dehydrogenase-A gene exon 7 and | 9.4E−110 |
| | | | 3′ flanking region | |
| 24006 | M00056163D:E01 | X03083 | Human lactate dehydrogenase-A gene exon 7 and | 0 |
| | | | 3′ flanking region | |
| 25678 | 035Jn031.H01 | AK001670 | Homo sapiens cDNA FLJ10808 fis, clone | 4.9E−53 |
| | | | NT2RP4000879, weakly similar to UBIQUITIN- | |
| | | | ACTIVATING ENZYME E1 | |
| 22027 | M00056534C:E08 | XM_003512 | Homo sapiens amphiregulin (schwannoma-derived | 0 |
| | | | growth factor) (AREG), mRNA | |
| 29495 | 035JN022.E12 | D83761 | Homo sapiens mRNA for mother against dpp (Mad) | 0 |
| | | | related protein, complete cds | |
| 24577 | M00056654B:G02 | XM_038306 | Homo sapiens dual specificity phosphatase 6 | 0 |
| | | | (DUSP6), mRNA | |
| 23527 | M00055865C:D04 | | | |
| 17090 | I:341491:13B01:A01 | BC004490 | Homo sapiens, v-fos FBJ murine osteosarcoma | 3.8E−98 |
| | | | viral oncogene homolog, clone MGC: 11074 | |
| | | | IMAGE: 3688670, mRNA, complete cds | |
| 25137 | M00057167A:C07 | | | |
| 23772 | M00056360A:E07 | BC004490 | Homo sapiens, v-fos FBJ murine osteosarcoma | 0 |
| | | | viral oncogene homolog, clone MGC: 11074 | |
| | | | IMAGE: 3688670, mRNA, complete cds | |
| 1659 | M00001350B:D10 | BC004490 | Homo sapiens, v-fos FBJ murine osteosarcoma | 0 |
| | | | viral oncogene homolog, clone MGC: 11074 | |
| | | | IMAGE: 3688670, mRNA, complete cds | |
| 8497 | I:2170638:05A01:A07 | BC006169 | Homo sapiens, Similar to SH3-domain binding | 5.2E−125 |
| | | | protein 5 (BTK-associated), clone MGC: 13234 | |
| | | | IMAGE: 4025362, mRNA, complete cds | |
| 25272 | M00054621A:D09 | AF161435 | Homo sapiens HSPC317 mRNA, partial cds | 0 |
| 21216 | M00056194B:G06 | XM_002844 | Homo sapiens procollagen-lysine, 2-oxoglutarate 5- | 0 |
| | | | dioxygenase (lysine hydroxylase) 2 (PLOD2), | |
| | | | mRNA | |
| 11939 | I:2938757:02A02:B05 | D43767 | Human mRNA for chemokine, complete cds | 0 |
| 9191 | I:1421929:05A01:D02 | X63629 | H. sapiens mRNA for p cadherin | 2.4E−90 |
| 3429 | 2024-3 | AF002697 | Homo sapiens E1B 19K/Bcl-2-binding protein Nip3 | 0 |
| | | | mRNA, nuclear gene encoding mitochondrial | |
| | | | protein, complete cds | |
| 2725 | 2024-1 | AF002697 | Homo sapiens E1B 19K/Bcl-2-binding protein Nip3 | 0 |
| | | | mRNA, nuclear gene encoding mitochondrial | |
| | | | protein, complete cds | |
| 19923 | I:1001356:13A01:B11 | BC006318 | Homo sapiens, erythrocyte membrane protein band | 1.7E−103 |
| | | | 4.9 (dematin), clone MGC: 12740 IMAGE: 4125804, | |
| | | | mRNA, complete cds | |
| 20457 | I:1923289:19A01:E06 | XM_035603 | Homo sapiens gap junction protein, beta 5 | 0 |
| | | | (connexin 31.1) (GJB5), mRNA | |
| 24773 | M00057055D:B11 | | | |
| 24119 | M00042886D:H10 | BC006260 | Homo sapiens, Similar to N-myc downstream | 4.4E−114 |
| | | | regulated, clone MGC: 11293 IMAGE: 3946764, | |
| | | | mRNA, complete cds | |
| 3908 | M00027080A:E06 | M60756 | Human histone H2B.1 mRNA, 3′ end | 0 |
| 8560 | I:2346704:06B01:H01 | AJ000334 | Homo sapiens mRNA for cytosolic asparaginyl- | 0 |
| | | | tRNA synthetase | |
| 24588 | M00055411A:C10 | L19779 | Homo sapiens histone H2A.2 mRNA, complete cds | 0 |
| 4047 | M00007997C:B08 | XM_009091 | Homo sapiens glycogen synthase 1 (muscle) | 0 |
| | | | (GYS1), mRNA | |
| 28344 | 035JN011.E11 | XM_050471 | Homo sapiens glycogen synthase 1 (muscle) | 0 |
| | | | (GYS1), mRNA | |
| 27561 | 035JN001.F04 | XM_001472 | Homo sapiens v-jun avian sarcoma virus 17 | 0 |
| | | | oncogene homolog (JUN), mRNA | |
| 3272 | M00022165C:E12 | NM_001024 | Homo sapiens ribosomal protein S21 (RPS21), | 0 |
| | | | mRNA | |
| 26735 | 035JN030.A08 | XM_010408 | Homo sapiens RAB9-like protein (RAB9L), mRNA | 0 |
| 24900 | M00054500D:C08 | BC004427 | Homo sapiens, proteasome (prosome, macropain) | 0 |
| | | | subunit, alpha type, 7, clone MGC: 3755 | |
| | | | IMAGE: 2819923, mRNA, complete cds | |
| 9472 | I:2510171:04B01:H08 | X04503 | Human SLPI mRNA fragment for secretory | 0 |
| | | | leucocyte protease inhibitor | |
| 9979 | I:1623318:06A01:F09 | L31409 | Homo sapiens creatine transporter mRNA, | 2.2E−45 |
| | | | complete cds | |
| 21996 | M00042467B:B04 | L00160 | Human phosphoglycerate kinase (pgk) mRNA, | 0 |
| | | | exons 2 to last | |
| 22312 | M00055035D:F05 | | | |
| 11327 | I:3139773:05A01:H11 | L00160 | Human phosphoglycerate kinase (pgk) mRNA, | 2.6E−21 |
| | | | exons 2 to last | |
| 18240 | RG:1927470:10015:H08 | V00572 | Human mRNA encoding phosphoglycerate kinase | 0 |
| 21922 | M00054848A:D12 | AF139065 | Homo sapiens desmoplakin I mRNA, partial cds | 0 |
| 22290 | M00057002D:H01 | | | |
| 10390 | I:1405391:03B02:C09 | AF056979 | Homo sapiens clone YAN1 interferon-gamma | 0 |
| | | | receptor mRNA, complete cds | |
| 2212 | M00008098B:F06 | U19247 | Homo sapiens interferon-gamma receptor alpha | 0 |
| | | | chain gene, exon 7 and complete cds | |
| 20213 | RG:221172:10007:C11 | S74774 | p59fyn(T) = OKT3-induced calcium influx regulator | 2.9E−103 |
| | | | [human, Jurkat J6 T cell line, mRNA Partial, 1605 | |
| | | | nt] | |
| 24955 | M00055929D:D04 | | | |
| 19574 | I:635178:13B02:C10 | XM_033944 | Homo sapiens superoxide dismutase 2, | 0 |
| | | | mitochondrial (SOD2), mRNA | |
| 19969 | RG:501476:10010:A05 | U14394 | Human tissue inhibitor of metalloproteinases-3 | 0 |
| | | | mRNA, complete cds | |
| 8570 | I:1696224:06B01:E07 | X70684 | C. aethiops mRNA for heat shock protein 70 | 5.6E−25 |
| 18519 | I:1997703:13A01:D09 | X52947 | Human mRNA for cardiac gap junction protein | 0 |
| 9616 | I:3200341:06B02:H02 | Y00106 | Human gene for beta-adrenergic receptor (beta-2 | 0 |
| | | | subtype) | |
| 22334 | M00055067D:H12 | | | |
| 17459 | I:2056395:13A02:B07 | M77349 | Human transforming growth factor-beta induced | 2.5E−121 |
| | | | gene product (BIGH3) mRNA, complete cds | |
| 25193 | M00056763B:A12 | X68277 | H. sapiens CL 100 mRNA for protein tyrosine | 0 |
| | | | phosphatase | |
| 25191 | M00056763B:A12 | X68277 | H. sapiens CL 100 mRNA for protein tyrosine | 0 |
| | | | phosphatase | |
| 9448 | I:2455617:04B01:D02 | XM_051799 | Homo sapiens guanosine monophosphate | 0 |
| | | | reductase (GMPR), mRNA | |
| 25224 | RG:950682:10003:D06 | BC002536 | Homo sapiens, phosphofructokinase, platelet, clone | 0 |
| | | | MGC: 2192 IMAGE: 3140233, mRNA, complete cds | |
| 20218 | RG:2158297:10016:E11 | BC002536 | Homo sapiens, phosphofructokinase, platelet, clone | 0 |
| | | | MGC: 2192 IMAGE: 3140233, mRNA, complete cds | |
| 3089 | NIH50_26184 | D25328 | Human mRNA for platelet-type | 2E−108 |
| | | | phosphofructokinase, complete cds | |
| 23985 | NIH50_26184 | | | |
| 19953 | NIH50_26184 | D25328 | Human mRNA for platelet-type | 2E−108 |
| | | | phosphofructokinase, complete cds | |
| 11506 | NIH50_26184 | | | |
| 22362 | M00056349A:F08 | M10546 | Human mitochondrial DNA, fragment M1, encoding | 1.2E−86 |
| | | | transfer RNAs, cytochrome oxidase I, and 2 URFs | |
| 25516 | 035JN011.G01 | XM_011470 | Homo sapiens myristoylated alanine-rich protein | 0 |
| | | | kinase C substrate (MARCKS, 80K-L) (MACS), | |
| | | | mRNA | |
| 25757 | 037XN005.H07 | AF017116 | Homo sapiens type-2 phosphatidic acid | 0 |
| | | | phosphohydrolase (PAP2) mRNA, complete cds | |
| 24814 | M00042773B:E09 | M17733 | Human thymosin beta-4 mRNA, complete cds | 0 |
| 21994 | M00042465B:E04 | M17733 | Human thymosin beta-4 mRNA, complete cds | 0 |
| 27117 | 037XN001.H03 | BC001631 | Homo sapiens, prothymosin beta 4, clone | 0 |
| | | | MGC: 2219 IMAGE: 3536637, mRNA, complete cds | |
| 24681 | NIH50_41452 | | | |
| 22745 | M00056592A:B08 | NM_003739 | Homo sapiens aldo-keto reductase family 1, | 0 |
| | | | member C3 (3-alpha hydroxysteroid | |
| | | | dehydrogenase, type II) (AKR1C3), mRNA | |
| 24233 | M00055873C:B06 | | | |
| 2001 | M00001381A:F03 | XM_035387 | Homo sapiens ribosomal protein, large, P1 | 0 |
| | | | (RPLP1), mRNA | |
| 21179 | NIH50_43550 | | | |
| 17147 | NIH50_43550 | AK026515 | Homo sapiens cDNA: FLJ22862 fis, clone | 0 |
| | | | KAT01966, highly similar to HSLDHAR Human | |
| | | | mRNA for lactate dehydrogenase-A | |
| 8700 | NIH50_43550 | | | |
| 21214 | M00056193B:D06 | BC006260 | Homo sapiens, Similar to N-myc downstream | 0 |
| | | | regulated, clone MGC: 11293 IMAGE: 3946764, | |
| | | | mRNA, complete cds | |
| 26422 | 037XN003.D08 | BC006260 | Homo sapiens, Similar to N-myc downstream | 0 |
| | | | regulated, clone MGC: 11293 IMAGE: 3946764, | |
| | | | mRNA, complete cds | |
| 22837 | M00055891C:B09 | | | |
| 21965 | M00057029A:G09 | | | |
| 25541 | 035JN013.D01 | AK026310 | Homo sapiens cDNA: FLJ22657 fis, clone | 0 |
| | | | HSI07791, highly similar to HUMCYB5 Human | |
| | | | cytochrome b5 mRNA | |
| 18302 | I:1738248:09B02:G08 | XM_016114 | Homo sapiens hypothetical protein FLJ22501 | 0 |
| | | | (FLJ22501), mRNA | |
| 24049 | M00054706B:G04 | AF107495 | Homo sapiens FWP001 and putative FWP002 | 0 |
| | | | mRNA, complete cds | |
| 26326 | 035JN023.D08 | AK025906 | Homo sapiens cDNA: FLJ22253 fis, clone | 0 |
| | | | HRC02763 | |
| 2254 | M00004085C:C02 | AK025703 | Homo sapiens cDNA: FLJ22050 fis, clone | 0 |
| | | | HEP09454 | |
| 10296 | I:2868216:07B02:D09 | AK025703 | Homo sapiens cDNA: FLJ22050 fis, clone | 0 |
| | | | HEP09454 | |
| 20044 | I:2547084:09B01:F05 | XM_016847 | Homo sapiens hypothetical protein FLJ22002 | 0 |
| | | | (FLJ22002), mRNA | |
| 28806 | 035JN028.D05 | AK025504 | Homo sapiens cDNA: FLJ21851 fis, clone | 0 |
| | | | HEP01962 | |
| 17566 | I:446969:17B02:G07 | AK023217 | Homo sapiens cDNA FLJ13155 fis, clone | 2E−115 |
| | | | NT2RP3003433 | |
| 19005 | I:2674167:09A02:G09 | AK022968 | Homo sapiens cDNA FLJ12906 fis, clone | 0 |
| | | | NT2RP2004373 | |
| 3567 | M00023369D:C05 | | | |
| 21983 | M00057081B:H03 | | | |
| 458 | M00022134B:E08 | XM_037412 | Homo sapiens hypothetical gene supported by | 0 |
| | | | BC008993 (LOC91283), mRNA | |
| 22331 | M00057138A:E11 | | | |
| 21411 | M00055833D:B03 | | | |
| 22972 | M00056956D:B01 | | | |
| 24533 | RG:1643392:10014:C11 | | | |
| 24853 | M00056617D:F07 | AK020869 | Mus musculus adult retina cDNA, RIKEN full-length | 6.5E−59 |
| | | | enriched library, clone: A930017A02, full insert | |
| | | | sequence | |
| 23753 | M00054915A:G02 | | | |
| 21502 | M00056193B:D06 | | | |
| 18180 | RG:39422:10005:B02 | | | |
| 23918 | M00056278C:E03 | | | |
| 24144 | RG:1982961:20001:H05 | | | |
| 19996 | RG:1283072:10012:F11 | BC009107 | Homo sapiens, clone MGC: 17352 IMAGE: 3449913, | 0 |
| | | | mRNA, complete cds | |
| 11528 | I:1899534:10B01:D05 | | | |
| 20506 | I:1969044:18B01:E12 | AB048286 | Homo sapiens GS1999full mRNA, complete cds | 0 |
| 23833 | RG:1656861:10014:E10 | | | |
| 20042 | I:1873176:09B01:E05 | BC001909 | Homo sapiens, clone IMAGE: 3537447, mRNA, | 0 |
| | | | partial cds | |
| 24977 | M00055820D:F01 | | | |
| 11646 | I:1723142:08B02:G11 | AK014612 | Mus musculus 0 day neonate skin cDNA, RIKEN | 4.6E−45 |
| | | | full-length enriched library, clone: 4633401I05, full | |
| | | | insert sequence | |
| 24872 | RG:773612:10011:D06 | | | |
| 10577 | I:2174196:08A01:A10 | | | |
| 21710 | RG:1091554:10003:G01 | | | |
| 18556 | RG:31082:10004:F09 | | | |
| 29433 | 035JN014.F12 | AK001805 | Homo sapiens cDNA FLJ10943 fis, clone | 0 |
| | | | OVARC1001360 | |
| 29273 | 037XN005.F12 | | | |
| 28763 | 035JN018.G11 | AJ310543 | Homo sapiens mRNA for EGLN1 protein | 1.9E−40 |
| 27887 | RG:2364147:8119908:A10 | | | |
| 27450 | 035JN032.F09 | | | |
| 27255 | 035JN006.E09 | XM_027456 | Homo sapiens hypothetical gene supported by | 1.2E−57 |
| | | | AK000584 (LOC89942), mRNA | |
| 27226 | 035JN004.F09 | | | |
| 26550 | 035JN008.D08 | | | |
| 26508 | 035JN004.G02 | | | |
| 26483 | RG:2377371:8119908:C08 | | | |
| 26334 | 035JN023.H08 | AF364547 | Homo sapiens methylmalonyl-CoA epimerase | 0 |
| | | | mRNA, complete cds; nuclear gene for | |
| | | | mitochondrial product | |
| 26027 | 035JN030.G01 | | | |
| 25977 | 035JN022.F07 | | | |
| 25965 | 035JN022.H01 | | | |
| 25844 | 035JN008.C07 | | | |
| 25834 | 035JN008.F01 | AB048289 | Bos taurus lae mRNA for lipoate-activating enzyme, | 3.1E−35 |
| | | | complete cds | |
| 25816 | 035JN004.E07 | | | |
| 25746 | 037XN007.B07 | | | |
| 25742 | 037XN007.H01 | | | |
| 25741 | 037XN005.H01 | | | |
| 25712 | 037XN003.A07 | | | |
| 25642 | 035Jn027.F01 | | | |
| 25621 | 035JN021.D07 | AK027321 | Homo sapiens cDNA FLJ14415 fis, clone | 0 |
| | | | HEMBA1004889, weakly similar to Human C3f | |
| | | | mRNA | |
| 25614 | 035JN023.H01 | | | |
| 25603 | 035JN021.C01 | | | |
| 25556 | 035JN015.C07 | | | |
| 25555 | 035JN013.C07 | | | |
| 25540 | 035JN015.C01 | | | |
| 23576 | RG:1984769:20002:D10 | | | |
| 22566 | RG:1996656:20003:C03 | | | |
| 9036 | DD182 | | | |
| 4164 | M00007932B:E06 | | | |
| 4146 | 2179-5 | | | |
| 4091 | M00026845A:E01 | | | |
| 4072 | M00023398A:G12 | | | |
| 4022 | M00022127D:B06 | | | |
| 3965 | M00005406A:f04 | | | |
| 3954 | M00005400B:E1 | | | |
| 3872 | M00007974D:B04 | | | |
| 3869 | M00003868C:A03 | | | |
| 3838 | M00007052A:C09 | XM_048272 | Homo sapiens similar to Ras-related GTP-binding | 0 |
| | | | protein (H. sapiens) (LOC92951), mRNA | |
| 3806 | 2168-2 | | | |
| 3798 | 2138-4 | | | |
| 3792 | 2171-5 | | | |
| 3788 | 2156-4 | | | |
| 3767 | M00001355D:H12 | | | |
| 3458 | M00007160D:E10 | | | |
| 3251 | M00005471A:a04 | | | |
| 3194 | DF821 | | | |
| 3102 | 2167-1 | | | |
| 3094 | 2138-3 | | | |
| 2671 | M00023431A:D02 | | | |
| 2634 | M00008025D:A04 | | | |
| 2567 | M00008061B:A12 | | | |
| 2317 | M00001502D:E09 | | | |
| 1958 | M00023296B:B09 | | | |
| 1680 | 2169-5 | | | |
| 1625 | M00001542C:G08 | | | |
| 1445 | M00023335C:C09 | | | |
| 1320 | 2207-5 | | | |
| 974 | 2161-1 | | | |
| 726 | DO15 | | | |
| 718 | ER418 | | | |
| 703 | M00004189D:A11 | | | |
| 652 | M00007070A:C08 | | | |
| 630 | 2203-2 | | | |
| 593 | M00001373A:A06 | X93036 | H. sapiens mRNA for MAT8 protein | 0 |
| 532 | M00022005A:H05 | | | |
| 272 | 2168-5 | | | |
| 256 | M00001406C:H12 | | | |
| 57 | M00023371B:H02 | | | |

TABLE 2

| | | 2D T4-2/ | 3D T4-2/ | 3D S1/2D | 3D T4-2/ | 3D T4-2/ | 3D T4-2/B1 | 3D T4-2/ |
|---|---|---|---|---|---|---|---|---|
| SEQ ID NO. | SPOT ID | 2D S1 | 3D S1 | S1 | 2D T4-2 | EGFR Ab | Integrin Ab | Tyr |
| 1 | 10594 | 0.6 | 2.2 | 0.6 | 1.9 | 3.0 | 1.0 | 2.9 |
| 2 | 21851 | 1.0 | 1.0 | 1.0 | 3.5 | 1.3 | 1.0 | 1.0 |
| 3 | 20990 | 1.6 | 4.6 | 1.0 | 1.5 | 1.0 | 1.0 | 1.0 |
| 4 | 18641 | 1.0 | 0.6 | 2.6 | 1.7 | 1.0 | 1.6 | 1.0 |
| 5 | 17229 | 0.3 | 0.8 | 1.0 | 2.1 | 1.0 | 1.0 | 1.0 |
| 6 | 25930 | 1.0 | 1.0 | 1.0 | 1.0 | 1.0 | 1.0 | 1.0 |
| 7 | 20701 | 1.6 | 2.9 | 1.3 | 2.7 | 4.5 | 1.9 | 5.8 |
| 8 | 20346 | 1.7 | 2.7 | 1.4 | 2.6 | 4.3 | 2.0 | 5.2 |
| 9 | 21247 | 1.0 | 4.4 | 1.5 | 3.0 | 3.4 | 2.6 | 4.7 |
| 10 | 23062 | 0.6 | 2.5 | 0.6 | 1.8 | 3.3 | 1.4 | 2.7 |
| 11 | 25666 | 1.0 | 2.9 | 0.6 | 2.0 | 3.6 | 1.0 | 2.3 |
| 12 | 19001 | 8.5 | 14.2 | 1.0 | 1.0 | 4.8 | 1.7 | 8.0 |
| 13 | 10897 | 1.0 | 3.1 | 4.5 | 1000.0 | 13.3 | 4.6 | 18.4 |
| 14 | 1960 | 0.3 | 1.5 | 3.0 | 13.7 | 3.9 | 2.4 | 4.9 |
| 15 | 26381 | 1.0 | 1.0 | 1.0 | 0.9 | 1.0 | 1.0 | 1.0 |
| 16 | 26719 | 0.4 | 1.0 | 0.6 | 2.8 | 1.2 | 1.7 | 1.0 |
| 17 | 27152 | 4.2 | 3.0 | 2.2 | 1.5 | 1.3 | 1.0 | 1.3 |
| 18 | 10926 | 0.7 | 1.9 | 0.9 | 2.1 | 3.7 | 1.5 | 3.3 |
| 19 | 28980 | 0.6 | 1.4 | 1.0 | 2.4 | 1.0 | 1.0 | 1.0 |
| 20 | 1236 | 1.0 | 2.8 | 0.8 | 2.1 | 2.2 | 1.8 | 3.2 |
| 21 | 29350 | 0.5 | 0.6 | 1.2 | 2.1 | 1.4 | 1.0 | 1.0 |
| 22 | 26242 | 1.0 | 1.0 | 0.6 | 2.2 | 1.0 | 1.0 | 2.0 |
| 23 | 4098 | 1.4 | 3.9 | 0.6 | 2.1 | 2.7 | 1.3 | 3.1 |
| 24 | 17432 | 0.4 | 0.3 | 2.4 | 2.1 | 0.3 | 0.9 | 0.3 |
| 25 | 1785 | 0.5 | 0.4 | 2.4 | 2.0 | 0.3 | 1.0 | 0.3 |
| 26 | 28856 | 8.5 | 0.9 | 2.5 | 0.3 | 0.6 | 1.0 | 0.5 |
| 27 | 18791 | 1.0 | 0.2 | 0.3 | 4.1 | 1.0 | 1.0 | 1.3 |
| 28 | 22950 | 3.9 | 4.1 | 1.2 | 1.0 | 2.1 | 1.0 | 2.4 |
| 29 | 1882 | 2.4 | 4.1 | 0.9 | 1.8 | 3.2 | 1.5 | 4.7 |
| 30 | 23886 | 1.0 | 1.0 | 1.2 | 2.1 | 1.0 | 1.0 | 1.0 |
| 31 | 24995 | 2.0 | 1.6 | 2.1 | 1.0 | 1.0 | 1.0 | 1.0 |
| 32 | 24477 | 1.0 | 1.9 | 1.0 | 4.2 | 2.7 | 1.3 | 1.8 |
| 33 | 21681 | 1.7 | 7.1 | 0.6 | 2.0 | 2.8 | 1.0 | 3.6 |
| 34 | 9557 | 1.6 | 7.5 | 0.8 | 1.0 | 3.0 | 1.0 | 2.5 |
| 35 | 22033 | 2.8 | 3.7 | 1.0 | 0.9 | 2.2 | 1.0 | 2.7 |
| 36 | 873 | 1.0 | 4.0 | 1.0 | 2.7 | 1.7 | 1.0 | 1.0 |
| 37 | 17144 | 1.0 | 0.5 | 3.6 | 1.4 | 1.0 | 1.0 | 1.0 |
| 38 | 26970 | 6.0 | 15.3 | 0.2 | 0.6 | 2.9 | 1.0 | 5.4 |
| 39 | 21402 | 0.2 | 1.0 | 2.8 | 6.9 | 2.4 | 1.0 | 3.6 |
| 40 | 27074 | 1.7 | 2.5 | 2.3 | 3.2 | 1.6 | 1.0 | 2.0 |
| 41 | 10963 | 0.5 | 0.3 | 2.1 | 0.5 | 1.0 | 1.0 | 0.7 |
| 42 | 29525 | 0.6 | 1.0 | 0.7 | 2.4 | 1.7 | 1.3 | 1.0 |
| 43 | 25514 | 1000.0 | 1.0 | 1.0 | 1.0 | 0.5 | 1.0 | 1.0 |
| 44 | 26612 | 0.4 | 0.5 | 1.6 | 2.8 | 0.8 | 1.0 | 0.8 |
| 45 | 24600 | 1.6 | 2.7 | 1.0 | 2.0 | 1.0 | 1.2 | 1.4 |
| 46 | 9741 | 2.3 | 5.0 | 1.0 | 2.2 | 1.7 | 1.0 | 1.0 |
| 47 | 23689 | 1.0 | 2.6 | 0.8 | 1.8 | 2.3 | 1.0 | 2.7 |
| 48 | 22352 | 1.0 | 2.9 | 0.7 | 1.6 | 2.4 | 1.0 | 2.4 |
| 49 | 23806 | 1.0 | 0.4 | 1.3 | 2.3 | 1.0 | 1.4 | 1.4 |
| 50 | 12285 | 1.0 | 1.0 | 1.0 | 1.0 | 0.8 | 1.0 | 0.5 |
| 51 | 27638 | 0.6 | 1.0 | 0.8 | 2.2 | 2.1 | 1.0 | 1.0 |
| 52 | 9663 | 1.0 | 1.0 | 1.0 | 1000.0 | 1.0 | 1.0 | 1.0 |
| 53 | 26850 | 1.0 | 0.2 | 9.1 | 2.1 | 1.3 | 1.6 | 2.2 |
| 54 | 10204 | 2.9 | 2.3 | 0.8 | 0.6 | 3.1 | 1.4 | 2.4 |
| 55 | 1318 | 2.0 | 0.9 | 2.3 | 0.5 | 0.6 | 1.1 | 0.7 |
| 56 | 25922 | 1.0 | 0.8 | 1.0 | 1.0 | 1.0 | 1.0 | 1.0 |
| 57 | 26347 | 1.0 | 1.0 | 1.0 | 1.0 | 1.0 | 1.0 | 1.0 |
| 58 | 20361 | 1.0 | 1.0 | 1.0 | 2.0 | 1.0 | 1.0 | 1.0 |
| 59 | 28672 | 0.6 | 2.1 | 0.6 | 2.1 | 1.4 | 1.0 | 1.7 |
| 60 | 25520 | 0.5 | 0.3 | 2.3 | 1.3 | 1.0 | 0.7 | 0.5 |
| 61 | 1723 | 1.0 | 0.5 | 5.1 | 3.5 | 1.0 | 3.1 | 1.0 |
| 62 | 28863 | 0.8 | 1.3 | 1.0 | 2.3 | 1.7 | 1.7 | 1.7 |
| 63 | 25526 | 5.9 | 1.7 | 1.0 | 0.6 | 0.6 | 0.7 | 0.4 |
| 64 | 27936 | 1.0 | 1.0 | 3.2 | 3.1 | 1.9 | 3.1 | 1.5 |
| 65 | 26851 | 1.0 | 0.7 | 3.2 | 2.7 | 1.6 | 2.4 | 1.3 |
| 66 | 25107 | 1.0 | 5.8 | 1.0 | 2.6 | 2.6 | 1.6 | 2.6 |
| 67 | 24912 | 1.0 | 2.9 | 1.0 | 2.4 | 1.6 | 1.3 | 1.8 |
| 68 | 25169 | 1.0 | 0.7 | 2.5 | 1.5 | 1.0 | 1.0 | 1.0 |
| 69 | 25600 | 1.6 | 1.4 | 2.9 | 2.1 | 0.7 | 0.9 | 0.5 |
| 70 | 28706 | 0.2 | 0.5 | 0.6 | 2.1 | 1.3 | 1.2 | 1.0 |
| 71 | 26377 | 0.6 | 0.3 | 2.2 | 1.0 | 1.2 | 1.3 | 1.0 |
| 72 | 19460 | 2.4 | 1.5 | 2.5 | 1.3 | 1.0 | 1.0 | 0.8 |
| 73 | 25243 | 1.0 | 0.7 | 2.2 | 1.0 | 1.0 | 1.0 | 1.0 |
| 74 | 20018 | 1.0 | 1.0 | 1.0 | 2.6 | 1.0 | 1.0 | 1.0 |
| 75 | 918 | 1.0 | 1.7 | 1.3 | 2.1 | 2.0 | 1.6 | 2.4 |
| 76 | 25027 | 1.0 | 1.0 | 1.0 | 1.0 | 1.0 | 1.0 | 1.0 |
| 77 | 29089 | 0.6 | 0.5 | 0.8 | 2.1 | 1.0 | 1.0 | 1.0 |
| 78 | 9141 | 1.0 | 1.0 | 1.0 | 1.0 | 1.0 | 1.0 | 1.0 |
| 79 | 12005 | 1.0 | 1.0 | 2.2 | 1.0 | 1.0 | 1.0 | 1.0 |
| 80 | 12148 | 1.0 | 1.0 | 1.0 | 1.0 | 1.0 | 1.0 | 1.0 |
| 81 | 17394 | 0.4 | 0.6 | 2.1 | 2.0 | 1.0 | 1.0 | 1.0 |
| 82 | 27017 | 2.8 | 3.3 | 0.8 | 1.0 | 2.4 | 1.8 | 2.8 |
| 83 | 25809 | 1.0 | 1.0 | 1000.0 | 1.0 | 1.0 | 1.0 | 1.0 |
| 84 | 8719 | 0.1 | 1.0 | 2.3 | 2.1 | 0.4 | 0.5 | 0.3 |
| 85 | 21030 | 0.4 | 1.0 | 1.3 | 2.1 | 1.4 | 1.6 | 1.4 |
| 86 | 11436 | 0.7 | 0.4 | 2.0 | 1.0 | 0.6 | 0.8 | 0.6 |
| 87 | 10374 | 1.5 | 1.5 | 3.5 | 2.7 | 0.4 | 1.0 | 0.3 |
| 88 | 19037 | 3.0 | 3.3 | 0.9 | 1.5 | 2.7 | 1.4 | 3.7 |
| 89 | 398 | 1.6 | 6.9 | 1.1 | 3.3 | 2.4 | 1.0 | 4.5 |
| 90 | 18773 | 1.9 | 5.1 | 1.0 | 3.9 | 3.8 | 2.0 | 6.1 |
| 91 | 3583 | 0.5 | 0.7 | 1.0 | 2.0 | 2.5 | 1.0 | 1.5 |
| 92 | 3418 | 1.8 | 3.2 | 1.2 | 2.4 | 1.6 | 1.0 | 1.2 |
| 93 | 18985 | 9.2 | 3.1 | 1.0 | 0.6 | 2.3 | 1.1 | 2.5 |
| 94 | 25861 | 3.4 | 1.5 | 2.0 | 0.8 | 0.8 | 0.9 | 0.6 |
| 95 | 3317 | 0.9 | 2.3 | 1.0 | 3.4 | 1.9 | 1.0 | 1.0 |
| 96 | 8743 | 0.2 | 0.7 | 1.0 | 4.3 | 1.8 | 1.0 | 1.7 |
| 97 | 26240 | 0.2 | 1.0 | 1.0 | 5.3 | 1.9 | 1.9 | 1.1 |
| 98 | 28562 | 0.3 | 0.2 | 2.0 | 1.0 | 0.5 | 0.5 | 0.6 |
| 99 | 16877 | 1.0 | 2.6 | 1.1 | 2.6 | 1.7 | 1.5 | 1.3 |
| 100 | 25955 | 1.0 | 1.0 | 1.0 | 1.0 | 1.0 | 1.0 | 1.0 |
| 101 | 26308 | 0.2 | 0.4 | 1.0 | 2.2 | 0.7 | 0.8 | 0.6 |
| 102 | 4140 | 1.9 | 6.7 | 0.7 | 2.1 | 3.0 | 1.0 | 3.5 |
| 103 | 3436 | 1.8 | 6.3 | 0.6 | 2.2 | 3.1 | 1.3 | 3.3 |
| 104 | 25612 | 1.0 | 12.5 | 1.0 | 1.0 | 2.1 | 1.0 | 2.9 |
| 105 | 12257 | 1.0 | 1.0 | 2.0 | 1.0 | 0.8 | 0.9 | 0.8 |
| 106 | 9111 | 0.5 | 0.5 | 2.2 | 1.3 | 1.5 | 1.0 | 0.7 |
| 107 | 17620 | 0.3 | 0.8 | 1.0 | 3.2 | 2.7 | 2.1 | 1.0 |
| 108 | 26025 | 1.0 | 2.9 | 1.1 | 2.2 | 2.3 | 1.0 | 2.6 |
| 109 | 19271 | 0.5 | 1.3 | 0.7 | 2.2 | 1.6 | 1.2 | 1.5 |
| 110 | 4151 | 0.4 | 4.2 | 1.2 | 11.1 | 4.2 | 1.0 | 2.9 |
| 111 | 26569 | 0.7 | 2.2 | 0.8 | 2.9 | 2.3 | 1.7 | 2.6 |
| 112 | 10344 | 1.0 | 1.0 | 1.0 | 1.0 | 1.0 | 1.0 | 1.0 |
| 113 | 832 | 1.0 | 3.3 | 1.0 | 2.4 | 3.7 | 2.2 | 4.0 |
| 114 | 12071 | 1.8 | 1.5 | 2.2 | 1.0 | 1.3 | 0.8 | 1.4 |
| 115 | 12271 | 0.6 | 4.9 | 1.9 | 14.9 | 20.8 | 4.0 | 24.1 |
| 116 | 11433 | 0.5 | 0.4 | 5.7 | 3.0 | 1.7 | 1.8 | 1.0 |
| 117 | 20917 | 1.0 | 2.8 | 0.9 | 2.6 | 1.7 | 1.4 | 1.7 |
| 118 | 25810 | 1.1 | 3.8 | 1.0 | 2.9 | 1.5 | 1.3 | 1.5 |
| 119 | 12039 | 1.0 | 1.0 | 3.6 | 1.0 | 1.0 | 1.0 | 1.0 |
| 120 | 25499 | 1.0 | 1.0 | 1.0 | 1.0 | 1.0 | 1.0 | 1.0 |
| 121 | 25557 | 1.0 | 1.8 | 1.0 | 0.8 | 1.0 | 1.0 | 1.0 |
| 122 | 9917 | 2.5 | 2.7 | 0.7 | 1.6 | 3.8 | 1.2 | 3.6 |
| 123 | 19505 | 0.4 | 1.7 | 0.7 | 3.8 | 1.7 | 1.6 | 1.4 |
| 124 | 17491 | 0.6 | 1.7 | 0.7 | 2.5 | 1.6 | 1.3 | 1.4 |
| 125 | 10683 | 0.4 | 1.9 | 0.6 | 3.6 | 1.7 | 1.4 | 1.1 |
| 126 | 1936 | 0.2 | 0.6 | 0.6 | 3.1 | 1.0 | 1.8 | 1.0 |
| 127 | 828 | 0.1 | 1.0 | 0.5 | 3.0 | 1.0 | 1.7 | 1.2 |
| 128 | 9558 | 1.0 | 1.0 | 1.0 | 1.0 | 1.0 | 1.0 | 1.0 |
| 129 | 20164 | 2.0 | 1.1 | 2.5 | 1.7 | 1.0 | 1.0 | 0.8 |
| 130 | 969 | 1.0 | 1.0 | 2.7 | 1.0 | 1.0 | 1.5 | 0.7 |
| 131 | 9910 | 0.4 | 1.0 | 0.8 | 3.2 | 1.9 | 1.3 | 1.4 |
| 132 | 2427 | 1.3 | 0.7 | 3.0 | 2.8 | 0.8 | 1.9 | 1.0 |
| 133 | 19990 | 1.0 | 7.9 | 2.8 | 34.7 | 1.0 | 1.0 | 1.0 |
| 134 | 20605 | 3.0 | 1.2 | 2.1 | 1.0 | 1.3 | 1.2 | 0.8 |
| 135 | 10650 | 0.5 | 1.7 | 0.5 | 2.9 | 2.8 | 0.6 | 3.4 |
| 136 | 25963 | 2.6 | 3.5 | 0.7 | 1.0 | 3.3 | 1.0 | 2.3 |
| 137 | 25562 | 3.2 | 5.9 | 0.7 | 1.0 | 4.2 | 1.0 | 4.8 |
| 138 | 9377 | 0.6 | 1.0 | 1.0 | 2.1 | 1.9 | 2.0 | 1.6 |
| 139 | 17618 | 1.0 | 0.7 | 2.3 | 3.2 | 0.8 | 0.7 | 0.8 |
| 140 | 12136 | 1.0 | 1.0 | 3.8 | 1.0 | 1.0 | 1.0 | 1.0 |
| 141 | 17373 | 1.0 | 0.4 | 6.1 | 2.4 | 1.0 | 1.0 | 1.0 |
| 142 | 18577 | 1.0 | 0.3 | 0.3 | 4.6 | 1.0 | 1.0 | 1.0 |
| 143 | 3143 | 1.7 | 1.3 | 2.6 | 2.3 | 0.7 | 1.0 | 0.5 |
| 144 | 17737 | 6.1 | 0.7 | 3.4 | 0.3 | 0.5 | 1.3 | 0.4 |
| 145 | 20029 | 1.0 | 0.6 | 2.3 | 1.0 | 1.0 | 1.0 | 0.5 |
| 146 | 18537 | 1.0 | 1.3 | 2.1 | 2.6 | 1.3 | 1.0 | 1.2 |
| 147 | 10090 | 1.0 | 1.7 | 2.1 | 2.8 | 1.5 | 1.0 | 1.2 |
| 148 | 12102 | 1.0 | 1.0 | 3.9 | 1.0 | 1.0 | 1.0 | 1.0 |
| 149 | 8487 | 4.7 | 2.4 | 1.0 | 1.0 | 2.3 | 1.1 | 2.2 |
| 150 | 9252 | 1.3 | 3.8 | 0.3 | 1.0 | 2.1 | 1.6 | 2.5 |
| 151 | 25605 | 1.0 | 1.0 | 1.0 | 1.0 | 0.5 | 0.5 | 1.0 |
| 152 | 29652 | 1.0 | 2.9 | 1.5 | 2.9 | 2.0 | 1.5 | 2.1 |
| 153 | 10858 | 1.0 | 0.8 | 2.0 | 1.0 | 1.0 | 1.0 | 0.7 |
| 154 | 1261 | 0.2 | 0.6 | 1.0 | 2.9 | 0.8 | 0.8 | 0.9 |
| 155 | 4156 | 12.4 | 0.8 | 3.1 | 0.2 | 0.6 | 1.0 | 0.3 |
| 156 | 3452 | 10.6 | 0.8 | 2.8 | 0.3 | 0.6 | 1.0 | 0.4 |
| 157 | 2748 | 10.8 | 0.8 | 3.1 | 0.2 | 0.5 | 1.0 | 0.4 |
| 158 | 2046 | 9.2 | 1.0 | 2.4 | 0.3 | 0.5 | 1.2 | 0.4 |
| 159 | 2044 | 11.7 | 0.8 | 2.8 | 0.2 | 0.6 | 1.4 | 0.4 |
| 160 | 1342 | 10.5 | 0.9 | 2.8 | 0.2 | 0.5 | 1.2 | 0.4 |
| 161 | 1326 | 12.2 | 1.0 | 2.7 | 0.2 | 0.5 | 1.0 | 0.4 |
| 162 | 9981 | 0.2 | 1.5 | 0.3 | 2.5 | 1.2 | 1.6 | 0.5 |
| 163 | 27917 | 1.9 | 2.5 | 0.5 | 1.0 | 2.1 | 1.4 | 2.3 |
| 164 | 8488 | 4.3 | 2.4 | 1.0 | 0.5 | 2.9 | 0.9 | 3.6 |
| 165 | 22793 | 1.9 | 2.6 | 0.5 | 1.0 | 2.2 | 1.8 | 2.1 |
| 166 | 26883 | 2.4 | 3.7 | 0.5 | 1.0 | 2.5 | 2.0 | 2.0 |
| 167 | 11540 | 0.7 | 1.0 | 1.3 | 2.8 | 0.8 | 1.0 | 0.5 |
| 168 | 17707 | 1.0 | 0.6 | 2.6 | 1.0 | 1.0 | 1.0 | 1.0 |
| 169 | 20649 | 2.3 | 2.6 | 0.5 | 0.4 | 3.0 | 1.0 | 3.1 |
| 170 | 24004 | 1.0 | 2.5 | 1.8 | 3.6 | 2.3 | 1.0 | 2.8 |
| 171 | 11836 | 1.2 | 5.0 | 0.9 | 3.7 | 1.3 | 1.0 | 0.8 |
| 172 | 24932 | 1.8 | 0.8 | 6.5 | 2.1 | 0.8 | 1.0 | 0.5 |
| 173 | 19143 | 0.6 | 1.6 | 0.7 | 2.0 | 1.7 | 1.2 | 1.4 |
| 174 | 26257 | 1.9 | 1.3 | 2.2 | 1.7 | 0.7 | 1.0 | 0.6 |
| 175 | 21239 | 9.4 | 9.2 | 0.5 | 0.4 | 2.4 | 1.0 | 2.7 |
| 176 | 16959 | 0.6 | 2.1 | 0.8 | 2.1 | 3.0 | 1.4 | 2.5 |
| 177 | 2568 | 0.7 | 1.9 | 0.7 | 2.2 | 3.0 | 1.3 | 2.4 |
| 178 | 25936 | 1.0 | 2.4 | 0.7 | 2.0 | 3.1 | 1.5 | 2.4 |
| 179 | 23041 | 0.7 | 1.0 | 2.1 | 2.6 | 1.0 | 1.4 | 1.0 |
| 180 | 9206 | 5.7 | 1.8 | 4.6 | 1.0 | 1.0 | 0.7 | 0.9 |
| 181 | 25105 | 1.6 | 1.3 | 2.1 | 1.0 | 1.0 | 0.7 | 0.8 |
| 182 | 24779 | 1.0 | 1.0 | 1.0 | 2.9 | 2.3 | 1.4 | 1.2 |
| 183 | 22451 | 1.0 | 0.2 | 2.1 | 1.0 | 1.4 | 0.6 | 1.0 |
| 184 | 22291 | 0.2 | 0.2 | 2.1 | 1.0 | 0.6 | 0.6 | 0.5 |
| 185 | 21143 | 1.0 | 7.2 | 0.7 | 2.0 | 2.6 | 1.1 | 2.4 |
| 186 | 24751 | 1.7 | 5.0 | 0.7 | 2.1 | 2.4 | 1.3 | 4.0 |
| 187 | 24294 | 1.7 | 3.9 | 0.8 | 2.4 | 2.6 | 1.1 | 3.9 |
| 188 | 24006 | 1.7 | 6.3 | 0.8 | 2.5 | 2.4 | 1.0 | 4.0 |
| 189 | 25678 | 1.0 | 1.0 | 1.0 | 1.0 | 1.0 | 1.0 | 1.0 |
| 190 | 22027 | 8.7 | 7.0 | 0.4 | 0.2 | 5.1 | 2.0 | 5.2 |
| 191 | 29495 | 1.0 | 1.0 | 1.0 | 1.0 | 1.0 | 1.0 | 1.0 |
| 192 | 24577 | 6.8 | 3.2 | 0.8 | 0.4 | 3.8 | 1.3 | 2.1 |
| 193 | 23527 | 0.3 | 2.1 | 1.6 | 6.4 | 2.7 | 2.1 | 3.4 |
| 194 | 17090 | 1.0 | 4.9 | 0.7 | 2.3 | 3.1 | 2.3 | 3.6 |
| 195 | 25137 | 1.0 | 1.0 | 0.4 | 3.8 | 1.0 | 2.5 | 4.1 |
| 196 | 23772 | 0.6 | 6.8 | 0.5 | 3.7 | 12.6 | 3.6 | 9.2 |
| 197 | 1659 | 1.0 | 7.5 | 0.3 | 3.2 | 17.8 | 4.1 | 20.3 |
| 198 | 8497 | 1.3 | 0.4 | 2.2 | 0.5 | 1.0 | 1.0 | 1.0 |
| 199 | 25272 | 8.0 | 6.0 | 1.0 | 0.6 | 2.2 | 1.0 | 2.9 |
| 200 | 21216 | 1.0 | 1.0 | 0.6 | 2.0 | 2.5 | 2.0 | 2.2 |
| 201 | 11939 | 1.0 | 1.0 | 1.0 | 1.0 | 1.0 | 1.0 | 1.0 |
| 202 | 9191 | 1.8 | 2.2 | 1.3 | 1.1 | 2.2 | 1.0 | 2.0 |
| 203 | 3429 | 0.7 | 3.4 | 0.8 | 3.5 | 3.0 | 1.5 | 3.7 |
| 204 | 2725 | 0.8 | 3.4 | 1.0 | 3.4 | 2.6 | 1.6 | 4.1 |
| 205 | 19923 | 1.0 | 1.1 | 2.9 | 1.0 | 1.7 | 1.4 | 1.2 |
| 206 | 20457 | 1.0 | 2.0 | 1.0 | 2.3 | 2.9 | 1.0 | 2.3 |
| 207 | 24773 | 0.2 | 1.0 | 0.8 | 2.0 | 1.6 | 1.0 | 1.0 |
| 208 | 24119 | 0.2 | 4.6 | 1.1 | 15.9 | 2.7 | 1.0 | 3.4 |
| 209 | 3908 | 0.3 | 0.5 | 1.1 | 2.3 | 1.7 | 1.0 | 1.0 |
| 210 | 8560 | 1.9 | 0.7 | 2.2 | 0.5 | 1.0 | 1.0 | 0.7 |
| 211 | 24588 | 0.3 | 0.5 | 1.0 | 2.0 | 1.0 | 1.0 | 1.4 |
| 212 | 4047 | 0.5 | 1.2 | 1.0 | 2.1 | 1.9 | 1.0 | 1.8 |
| 213 | 28344 | 0.8 | 1.0 | 1.0 | 2.0 | 1.7 | 1.5 | 2.7 |
| 214 | 27561 | 1.0 | 1.0 | 1.0 | 2.4 | 1.2 | 1.2 | 1.3 |
| 215 | 3272 | 0.6 | 0.8 | 1.0 | 2.1 | 1.3 | 1.6 | 1.0 |
| 216 | 26735 | 1.0 | 1.0 | 1.0 | 1.0 | 1.0 | 1.0 | 1.0 |
| 217 | 24900 | 0.3 | 0.8 | 2.9 | 5.7 | 2.2 | 1.1 | 3.0 |
| 218 | 9472 | 2.2 | 5.0 | 1.0 | 2.0 | 1.5 | 0.8 | 1.7 |
| 219 | 9979 | 1.3 | 3.3 | 1.5 | 3.9 | 3.4 | 1.4 | 2.5 |
| 220 | 21996 | 1.0 | 4.7 | 1.0 | 3.4 | 2.5 | 1.0 | 2.4 |
| 221 | 22312 | 1.2 | 4.4 | 1.2 | 3.3 | 2.2 | 1.1 | 2.2 |
| 222 | 11327 | 1.4 | 6.2 | 1.4 | 2.7 | 2.7 | 1.0 | 2.2 |
| 223 | 18240 | 2.0 | 4.5 | 1.0 | 2.2 | 2.1 | 1.0 | 2.7 |
| 224 | 21922 | 0.7 | 1.4 | 0.8 | 2.1 | 1.8 | 1.0 | 1.3 |
| 225 | 22290 | 0.7 | 1.6 | 0.9 | 2.1 | 1.5 | 1.2 | 1.3 |
| 226 | 10390 | 1.3 | 1.0 | 2.6 | 1.6 | 0.8 | 1.0 | 0.6 |
| 227 | 2212 | 1.9 | 1.0 | 2.8 | 1.0 | 0.6 | 1.6 | 0.8 |
| 228 | 20213 | 0.4 | 1.0 | 1.0 | 1.0 | 1.0 | 1.0 | 1.0 |
| 229 | 24955 | 0.9 | 2.9 | 1.0 | 3.3 | 0.8 | 0.8 | 1.0 |
| 230 | 19574 | 1.0 | 0.6 | 3.7 | 3.1 | 1.5 | 2.6 | 1.4 |
| 231 | 19969 | 1.0 | 1.0 | 1.0 | 3.1 | 1.0 | 1.0 | 1.0 |
| 232 | 8570 | 0.4 | 1.2 | 1.0 | 2.6 | 1.2 | 0.8 | 0.6 |
| 233 | 18519 | 3.5 | 2.9 | 2.6 | 1.8 | 1.8 | 1.0 | 2.0 |
| 234 | 9616 | 0.6 | 2.0 | 1.0 | 2.3 | 1.2 | 1.2 | 1.0 |
| 235 | 22334 | 0.2 | 0.7 | 2.9 | 8.5 | 1.7 | 1.1 | 3.4 |
| 236 | 17459 | 0.1 | 0.7 | 2.7 | 18.8 | 4.0 | 1.3 | 4.6 |
| 237 | 25193 | 1.0 | 0.8 | 1.0 | 2.3 | 1.0 | 1.3 | 1.0 |
| 238 | 25191 | 0.2 | 0.8 | 0.7 | 2.5 | 1.3 | 1.5 | 1.2 |
| 239 | 9448 | 0.6 | 1.0 | 1.0 | 2.3 | 0.8 | 0.8 | 0.5 |
| 240 | 25224 | 5.6 | 14.4 | 1.0 | 2.3 | 6.0 | 1.5 | 9.6 |
| 241 | 20218 | 6.1 | 12.3 | 0.7 | 1.7 | 5.6 | 1.6 | 9.0 |
| 242 | 3089 | 7.0 | 15.7 | 0.7 | 2.3 | 7.3 | 1.8 | 8.0 |
| 243 | 23985 | 5.8 | 17.2 | 0.9 | 2.1 | 6.8 | 1.8 | 8.1 |
| 244 | 19953 | 6.2 | 13.5 | 0.8 | 1.8 | 6.4 | 1.7 | 10.4 |
| 245 | 11506 | 4.1 | 13.3 | 1.0 | 1.4 | 4.4 | 1.6 | 7.2 |
| 246 | 22362 | 1.0 | 0.7 | 4.1 | 2.1 | 1.2 | 1.8 | 1.0 |
| 247 | 25516 | 0.7 | 10.1 | 0.4 | 4.0 | 14.7 | 4.7 | 8.1 |
| 248 | 25757 | 0.6 | 0.4 | 2.4 | 1.0 | 1.0 | 1.3 | 0.9 |
| 249 | 24814 | 0.5 | 2.8 | 0.3 | 1.0 | 3.5 | 1.4 | 4.4 |
| 250 | 21994 | 0.5 | 3.2 | 0.3 | 1.0 | 3.6 | 1.0 | 4.3 |
| 251 | 27117 | 1.0 | 2.8 | 0.3 | 1.0 | 3.9 | 1.0 | 4.9 |
| 252 | 24681 | 1.8 | 2.6 | 0.6 | 0.5 | 3.2 | 1.5 | 3.0 |
| 253 | 22745 | 0.3 | 2.4 | 1.4 | 8.1 | 2.8 | 2.3 | 3.5 |
| 254 | 24233 | 1.9 | 3.9 | 1.3 | 2.3 | 1.3 | 0.8 | 2.2 |
| 255 | 2001 | 1.0 | 1.0 | 1.5 | 2.1 | 1.0 | 1.0 | 1.0 |
| 256 | 21179 | 2.0 | 7.9 | 0.7 | 1.9 | 2.1 | 1.0 | 4.3 |
| 257 | 17147 | 1.3 | 4.3 | 0.7 | 1.7 | 2.4 | 1.2 | 3.9 |
| 258 | 8700 | 1.5 | 7.3 | 0.7 | 1.6 | 3.1 | 1.0 | 2.7 |
| 259 | 21214 | 0.3 | 5.4 | 1.2 | 15.5 | 3.1 | 1.0 | 3.6 |
| 260 | 26422 | 0.4 | 3.7 | 1.0 | 12.7 | 3.9 | 1.0 | 3.3 |
| 261 | 22837 | 0.7 | 1.0 | 2.1 | 2.4 | 1.2 | 1.5 | 0.9 |
| 262 | 21965 | 1.0 | 1.0 | 1.0 | 2.2 | 2.4 | 1.0 | 1.0 |
| 263 | 25541 | 4.5 | 2.7 | 2.7 | 0.8 | 1.0 | 1.3 | 0.8 |
| 264 | 18302 | 1.1 | 0.9 | 2.1 | 1.0 | 1.0 | 1.0 | 1.0 |
| 265 | 24049 | 1.0 | 2.6 | 1.5 | 2.5 | 2.3 | 1.4 | 2.4 |
| 266 | 26326 | 9.2 | 1.5 | 3.2 | 0.7 | 0.7 | 0.9 | 1.0 |
| 267 | 2254 | 1.6 | 3.3 | 1.0 | 2.8 | 2.0 | 1.1 | 3.1 |
| 268 | 10296 | 0.9 | 1.7 | 2.9 | 5.0 | 2.1 | 1.0 | 1.3 |
| 269 | 20044 | 1.0 | 0.8 | 2.0 | 1.0 | 1.0 | 1.0 | 1.0 |
| 270 | 28806 | 2.8 | 1.1 | 2.1 | 1.0 | 0.9 | 1.2 | 0.8 |
| 271 | 17566 | 7.5 | 4.2 | 0.7 | 0.5 | 2.5 | 1.0 | 2.5 |
| 272 | 19005 | 1.0 | 0.8 | 1.0 | 2.1 | 1.0 | 1.0 | 1.0 |
| 273 | 3567 | 1.0 | 1.0 | 1.0 | 1.0 | 1.0 | 1.0 | 1.0 |
| 274 | 21983 | 0.1 | 1.0 | 3.1 | 25.6 | 3.4 | 1.0 | 4.7 |
| 275 | 458 | 1.0 | 2.1 | 0.6 | 1.0 | 1.6 | 2.1 | 2.3 |
| 276 | 22331 | 0.6 | 2.1 | 0.4 | 1.0 | 2.2 | 1.0 | 2.8 |
| 277 | 21411 | 0.7 | 1.5 | 1.0 | 2.5 | 1.0 | 1.0 | 1.0 |
| 278 | 22972 | 1.0 | 2.2 | 0.5 | 1.0 | 2.2 | 1.4 | 2.4 |
| 279 | 24533 | 1.0 | 2.5 | 1.0 | 2.0 | 2.0 | 2.7 | 3.2 |
| 280 | 24853 | 1.0 | 2.6 | 2.1 | 2.1 | 2.4 | 1.3 | 2.1 |
| 281 | 23753 | 0.7 | 1.5 | 1.3 | 2.1 | 2.0 | 1.7 | 2.3 |
| 282 | 21502 | 0.3 | 4.8 | 1.0 | 10.8 | 2.6 | 1.0 | 2.9 |
| 283 | 18180 | 0.3 | 0.8 | 0.8 | 2.4 | 0.9 | 1.4 | 0.7 |
| 284 | 23918 | 0.7 | 2.3 | 0.4 | 1.0 | 2.4 | 1.2 | 3.5 |
| 285 | 24144 | 1.0 | 1.0 | 1.0 | 1.0 | 1.0 | 1.6 | 1.0 |
| 286 | 19996 | 1.5 | 2.5 | 0.7 | 1.2 | 2.1 | 0.9 | 2.5 |
| 287 | 11528 | 1.0 | 1.0 | 1.0 | 2.1 | 1.0 | 1.0 | 1.0 |
| 288 | 20506 | 2.2 | 0.9 | 3.2 | 0.8 | 1.3 | 1.6 | 1.0 |
| 289 | 23833 | 1.0 | 0.5 | 2.1 | 1.0 | 1.0 | 1.0 | 0.7 |
| 290 | 20042 | 3.8 | 1.6 | 2.3 | 0.8 | 1.0 | 1.0 | 1.0 |
| 291 | 24977 | 1.0 | 1.0 | 2.1 | 1.0 | 2.3 | 1.4 | 1.4 |
| 292 | 11646 | 1.0 | 1.0 | 0.8 | 1000.0 | 1.0 | 1.0 | 1.7 |
| 293 | 24872 | 1.0 | 1.4 | 0.8 | 2.5 | 1.4 | 1.2 | 1.3 |
| 294 | 10577 | 1.0 | 1.0 | 1.0 | 1.0 | 1.0 | 1.0 | 1.0 |
| 295 | 21710 | 1.0 | 0.2 | 2.2 | 0.7 | 1.6 | 1.0 | 1.2 |
| 296 | 18556 | 0.0 | 1.0 | 1.0 | 1.0 | 1.0 | 1.0 | 1.0 |
| 297 | 29433 | 1.0 | 0.5 | 1.0 | 2.1 | 1.0 | 1.0 | 1.0 |
| 298 | 29273 | 1.0 | 2.2 | 1.0 | 2.2 | 1.0 | 1.3 | 1.0 |
| 299 | 28763 | 1.6 | 2.7 | 1.0 | 2.2 | 1.8 | 1.3 | 2.5 |
| 300 | 27887 | 0.1 | 0.2 | 1.1 | 2.7 | 0.8 | 1.0 | 0.6 |
| 301 | 27450 | 2.6 | 11.3 | 0.2 | 1.0 | 4.4 | 3.3 | 7.3 |
| 302 | 27255 | 0.6 | 1.6 | 0.8 | 2.3 | 1.7 | 1.4 | 1.5 |
| 303 | 27226 | 1.0 | 1.3 | 1.0 | 2.6 | 1.8 | 1.0 | 1.0 |
| 304 | 26550 | 4.2 | 17.9 | 0.2 | 1.0 | 6.9 | 2.9 | 9.2 |
| 305 | 26508 | 1.0 | 1.4 | 1.0 | 1.0 | 1.0 | 1.0 | 1.0 |
| 306 | 26483 | 1.2 | 2.2 | 0.6 | 1.0 | 2.1 | 1.4 | 2.7 |
| 307 | 26334 | 1.0 | 0.5 | 3.0 | 1.0 | 0.6 | 0.8 | 0.5 |
| 308 | 26027 | 1.0 | 1.0 | 1.0 | 1.5 | 1.0 | 1.0 | 1.0 |
| 309 | 25977 | 1.0 | 1.0 | 1.0 | 1.0 | 1.0 | 1.0 | 1.0 |
| 310 | 25965 | 1.0 | 1.0 | 1.0 | 1.0 | 1.0 | 1.0 | 1.0 |
| 311 | 25844 | 1.0 | 1.0 | 1.0 | 1.0 | 1.0 | 1.0 | 1.0 |
| 312 | 25834 | 1000.0 | 1.0 | 1.0 | 1.0 | 0.4 | 1.0 | 1.0 |
| 313 | 25816 | 1.0 | 1.0 | 1.0 | 1.0 | 1.0 | 1.0 | 1.0 |
| 314 | 25746 | 1.0 | 1.0 | 1.0 | 1.0 | 1.0 | 1.0 | 1.0 |
| 315 | 25742 | 1.0 | 1.0 | 1.0 | 1.0 | 0.5 | 1.0 | 1.0 |
| 316 | 25741 | 1.0 | 1.0 | 1.0 | 1.0 | 1.0 | 1.0 | 1.0 |
| 317 | 25712 | 1.0 | 1.0 | 1.0 | 1.0 | 1.0 | 1.0 | 1.0 |
| 318 | 25642 | 1.0 | 1.0 | 1.0 | 1.0 | 1.0 | 1.0 | 1.0 |
| 319 | 25621 | 0.6 | 0.8 | 2.1 | 2.0 | 1.3 | 1.2 | 1.0 |
| 320 | 25614 | 1.0 | 1.0 | 1.0 | 1.0 | 1.0 | 1.0 | 1.0 |
| 321 | 25603 | 1.0 | 1.0 | 1.0 | 1.0 | 1.0 | 1.0 | 1.0 |
| 322 | 25556 | 1.8 | 0.7 | 1.0 | 0.8 | 1.0 | 1.0 | 1.0 |
| 323 | 25555 | 1.0 | 2.9 | 1.0 | 1.0 | 1.5 | 1.3 | 1.0 |
| 324 | 25540 | 1.0 | 1.0 | 1.0 | 1.2 | 1.0 | 1.0 | 1.0 |
| 325 | 23576 | 0.0 | 1.0 | 1.0 | 1.0 | 1.0 | 1.5 | 1.4 |
| 326 | 22566 | 1.0 | 1.0 | 1.0 | 1.0 | 1.0 | 1.3 | 1.0 |
| 327 | 9036 | 1.9 | 3.8 | 0.6 | 1.8 | 2.6 | 1.3 | 3.1 |
| 328 | 4164 | 1.0 | 1.0 | 2.2 | 1.0 | 1.5 | 1.0 | 0.9 |
| 329 | 4146 | 0.8 | 3.7 | 0.9 | 4.4 | 3.3 | 1.0 | 4.3 |
| 330 | 4091 | 1.0 | 1.0 | 1.0 | 2.5 | 1.7 | 1.0 | 0.9 |
| 331 | 4072 | 1.0 | 1.0 | 2.1 | 1.0 | 5.9 | 2.0 | 5.1 |
| 332 | 4022 | 3.5 | 4.5 | 0.8 | 1.0 | 3.0 | 1.0 | 3.2 |
| 333 | 3965 | 1.9 | 5.6 | 0.4 | 1.0 | 5.5 | 2.3 | 4.1 |
| 334 | 3954 | 1.0 | 2.7 | 1.3 | 3.6 | 2.8 | 1.9 | 2.6 |
| 335 | 3872 | 1.0 | 3.2 | 1.3 | 2.8 | 4.0 | 1.8 | 3.9 |
| 336 | 3869 | 1.0 | 1.0 | 5.8 | 3.8 | 1.0 | 0.7 | 0.6 |
| 337 | 3838 | 1.0 | 1.6 | 1.2 | 2.0 | 2.6 | 1.7 | 1.9 |
| 338 | 3806 | 0.6 | 2.6 | 0.9 | 3.7 | 3.0 | 1.0 | 3.4 |
| 339 | 3798 | 10.2 | 0.9 | 2.9 | 0.3 | 0.7 | 1.0 | 0.4 |
| 340 | 3792 | 1.0 | 1.0 | 1.0 | 2.7 | 2.9 | 1.0 | 2.5 |
| 341 | 3788 | 1.7 | 5.4 | 1.2 | 3.4 | 2.5 | 1.3 | 2.7 |
| 342 | 3767 | 1.1 | 2.2 | 0.7 | 1.7 | 2.5 | 1.0 | 2.5 |
| 343 | 3458 | 1.2 | 3.3 | 0.7 | 2.0 | 2.6 | 1.0 | 2.2 |
| 344 | 3251 | 0.4 | 0.5 | 1.4 | 2.7 | 1.4 | 1.0 | 1.0 |
| 345 | 3194 | 1.0 | 2.3 | 1.3 | 3.1 | 2.2 | 1.3 | 3.2 |
| 346 | 3102 | 0.5 | 3.2 | 1.0 | 4.8 | 2.9 | 1.0 | 2.3 |
| 347 | 3094 | 11.5 | 0.8 | 2.7 | 0.3 | 0.6 | 1.0 | 0.4 |
| 348 | 2671 | 0.8 | 1.6 | 1.0 | 2.2 | 2.8 | 1.9 | 1.0 |
| 349 | 2634 | 0.9 | 2.8 | 0.4 | 1.0 | 3.8 | 1.7 | 4.0 |
| 350 | 2567 | 4.6 | 3.3 | 0.8 | 0.6 | 2.6 | 1.0 | 3.3 |
| 351 | 2317 | 1.0 | 1.0 | 2.4 | 1.0 | 1.0 | 1.0 | 1.1 |
| 352 | 1958 | 0.3 | 0.6 | 1.0 | 2.6 | 0.9 | 0.8 | 0.9 |
| 353 | 1680 | 0.3 | 4.7 | 1.0 | 17.7 | 2.7 | 1.0 | 4.5 |
| 354 | 1625 | 2.2 | 7.8 | 0.5 | 1.8 | 3.1 | 1.7 | 3.4 |
| 355 | 1445 | 0.2 | 0.6 | 1.0 | 2.7 | 0.8 | 0.9 | 0.9 |
| 356 | 1320 | 4.9 | 1.0 | 2.4 | 0.4 | 0.6 | 1.2 | 0.5 |
| 357 | 974 | 0.6 | 3.1 | 1.1 | 3.2 | 2.4 | 1.4 | 3.7 |
| 358 | 726 | 1.0 | 1.0 | 1.0 | 1.0 | 1.0 | 1.0 | 1.0 |
| 359 | 718 | 0.4 | 2.6 | 0.5 | 2.7 | 1.6 | 1.0 | 1.0 |
| 360 | 703 | 1.0 | 4.1 | 1.0 | 2.4 | 1.6 | 1.0 | 1.7 |
| 361 | 652 | 2.8 | 4.4 | 1.6 | 2.3 | 1.0 | 1.6 | 1.0 |
| 362 | 630 | 6.9 | 1.0 | 2.2 | 0.3 | 0.6 | 1.0 | 0.5 |
| 363 | 593 | 1.0 | 4.3 | 1.0 | 2.3 | 1.0 | 1.0 | 1.0 |
| 364 | 532 | 1.3 | 4.7 | 1.0 | 2.4 | 2.6 | 2.2 | 4.0 |
| 365 | 272 | 0.7 | 2.7 | 0.9 | 3.1 | 2.4 | 1.3 | 4.3 |
| 366 | 256 | 0.6 | 3.2 | 0.5 | 1.9 | 2.8 | 1.0 | 3.4 |
| 367 | 57 | 0.5 | 1.4 | 1.0 | 2.3 | 0.9 | 1.0 | 0.7 |

TABLE 11

| SEQ ID NO | SPOT ID |
|---|---|
| 1 | 10594 |
| 2 | 21851 |
| 3 | 20990 |
| 4 | 18641 |
| 5 | 19037 |
| 6 | 398 |
| 7 | 18773 |
| 8 | 3583 |
| 9 | 3418 |
| 10 | 145306 |
| 11 | 3418 |
| 12 | 3418 |
| 13 | 18985 |
| 14 | 17229 |
| 15 | 25930 |
| 16 | 25930 |
| 17 | 20701 |
| 18 | 20346 |
| 19 | 20346 |
| 20 | 21247 |
| 21 | 21247 |
| 22 | 23062 |
| 23 | 25666 |
| 24 | 25666 |
| 25 | 19001 |
| 26 | 10897 |
| 27 | 10897 |
| 28 | 10897 |
| 29 | 1960 |
| 30 | 146262 |
| 31 | 26381 |
| 32 | 26381 |
| 33 | 26719 |
| 34 | 26719 |
| 35 | 27152 |
| 36 | 10926 |
| 37 | 28980 |
| 38 | 1236 |
| 39 | 29350 |
| 40 | 29350 |
| 41 | 26242 |
| 42 | 4098 |
| 43 | 145253 |
| 44 | 4098 |
| 45 | 17432 |
| 46 | 17432 |
| 47 | 1785 |
| 48 | 1785 |
| 49 | 1785 |
| 50 | 28856 |
| 51 | 28856 |
| 52 | 18791 |
| 53 | 18791 |
| 54 | 22950 |
| 55 | 22950 |
| 56 | 1882 |
| 57 | 23886 |
| 58 | 24995 |
| 59 | 24995 |
| 60 | 24477 |
| 61 | 21681 |
| 62 | 21681 |
| 63 | 9557 |
| 64 | 9557 |
| 65 | 22033 |
| 66 | 873 |
| 67 | 17144 |
| 68 | 26970 |
| 69 | 26970 |
| 70 | 21402 |
| 71 | 27074 |
| 72 | 27074 |
| 73 | 10963 |
| 74 | 10963 |
| 75 | 29525 |
| 76 | 29525 |
| 77 | 25514 |
| 78 | 25514 |
| 79 | 26612 |
| 80 | 26612 |
| 81 | 24600 |
| 82 | 9741 |
| 83 | 9741 |
| 84 | 9741 |
| 85 | 23689 |
| 86 | 23689 |
| 87 | 22352 |
| 88 | 23806 |
| 89 | 12285 |
| 90 | 27638 |
| 91 | 27638 |
| 92 | 9663 |
| 93 | 9663 |
| 94 | 26850 |
| 95 | 10204 |
| 96 | 10204 |
| 97 | 10204 |
| 98 | 25922 |
| 99 | 25922 |
| 100 | 26347 |
| 101 | 26347 |
| 102 | 20361 |
| 103 | 20361 |
| 104 | 28672 |
| 105 | 28672 |
| 106 | 25520 |
| 107 | 25520 |
| 108 | 1723 |
| 109 | 1723 |
| 110 | 28863 |
| 111 | 25526 |
| 112 | 25526 |
| 113 | 27936 |
| 114 | 27936 |
| 115 | 26851 |
| 116 | 25107 |
| 117 | 25107 |
| 118 | 25107 |
| 119 | 24912 |
| 120 | 24912 |
| 121 | 25169 |
| 122 | 25600 |
| 123 | 25600 |
| 124 | 28706 |
| 125 | 28706 |
| 126 | 26377 |
| 127 | 26377 |
| 128 | 19460 |
| 129 | 25243 |
| 130 | 20018 |
| 131 | 20018 |
| 132 | 918 |
| 133 | 25027 |
| 134 | 29089 |
| 135 | 29089 |
| 136 | 9141 |
| 137 | 9141 |
| 138 | 9141 |
| 139 | 12005 |
| 140 | 12148 |
| 141 | 12148 |
| 142 | 17394 |
| 143 | 27017 |
| 144 | 27017 |
| 145 | 25809 |
| 146 | 8719 |
| 147 | 8719 |
| 148 | 21030 |
| 149 | 21030 |
| 150 | 11436 |
| 151 | 11436 |
| 152 | 10374 |
| 153 | 10374 |
| 154 | 25861 |
| 155 | 25861 |
| 156 | 3317 |
| 157 | 3317 |
| 158 | 8743 |
| 159 | 26240 |
| 160 | 26240 |
| 161 | 28562 |
| 162 | 16877 |
| 163 | 25955 |
| 164 | 26308 |
| 165 | 26308 |
| 166 | 4140 |
| 167 | 3436 |
| 168 | 25612 |
| 169 | 25612 |
| 170 | 12257 |
| 171 | 12257 |
| 172 | 9111 |
| 173 | 9111 |
| 174 | 17620 |
| 175 | 26025 |
| 176 | 26025 |
| 177 | 19271 |
| 178 | 4151 |
| 179 | 4151 |
| 180 | 26569 |
| 181 | 26569 |
| 182 | 10344 |
| 183 | 10344 |
| 184 | 10344 |
| 185 | 832 |
| 186 | 832 |
| 187 | 12071 |
| 188 | 12071 |
| 189 | 12271 |
| 190 | 11433 |
| 191 | 20917 |
| 192 | 25810 |
| 193 | 12039 |
| 194 | 12039 |
| 195 | 25499 |
| 196 | 25499 |
| 197 | 25557 |
| 198 | 25557 |
| 199 | 9917 |
| 200 | 19505 |
| 201 | 17491 |
| 202 | 10683 |
| 203 | 10683 |
| 204 | 1936 |
| 205 | 828 |
| 206 | 9558 |
| 207 | 9558 |
| 208 | 20164 |
| 209 | 969 |
| 210 | 969 |
| 211 | 9910 |
| 212 | 2427 |
| 213 | 19990 |
| 214 | 20605 |
| 215 | 20605 |
| 216 | 10650 |
| 217 | 10650 |
| 218 | 25963 |
| 219 | 25963 |
| 220 | 25562 |
| 221 | 25562 |
| 222 | 3429 |
| 223 | 2725 |
| 224 | 19923 |
| 225 | 20457 |
| 226 | 20457 |
| 227 | 24773 |
| 228 | 24119 |
| 229 | 3908 |
| 230 | 3908 |
| 231 | 8560 |
| 232 | 8560 |
| 233 | 9377 |
| 234 | 9377 |
| 235 | 17618 |
| 236 | 12136 |
| 237 | 17373 |
| 238 | 18577 |
| 239 | 18577 |
| 240 | 3143 |
| 241 | 17737 |
| 242 | 17737 |
| 243 | 20029 |
| 244 | 20029 |
| 245 | 18537 |
| 246 | 18537 |
| 247 | 12102 |
| 248 | 12102 |
| 249 | 8487 |
| 250 | 9252 |
| 251 | 9252 |
| 252 | 25605 |
| 253 | 25605 |
| 254 | 29652 |
| 255 | 10858 |
| 256 | 1261 |
| 257 | 4156 |
| 258 | 4156 |
| 259 | 3452 |
| 260 | 3452 |
| 261 | 2748 |
| 262 | 2046 |
| 263 | 2046 |
| 264 | 2044 |
| 265 | 2044 |
| 266 | 1342 |
| 267 | 1342 |
| 268 | 1326 |
| 269 | 1326 |
| 270 | 9981 |
| 271 | 9981 |
| 272 | 27917 |
| 273 | 8488 |
| 274 | 22793 |
| 275 | 22793 |
| 276 | 26883 |
| 277 | 26883 |
| 278 | 11540 |
| 279 | 17707 |
| 280 | 20649 |
| 281 | 20649 |
| 282 | 24004 |
| 283 | 24004 |
| 284 | 11836 |
| 285 | 11836 |
| 286 | 11836 |
| 287 | 24932 |
| 288 | 19143 |
| 289 | 19143 |
| 290 | 26257 |
| 291 | 26257 |
| 292 | 21239 |
| 293 | 21239 |
| 294 | 16959 |
| 295 | 2568 |
| 296 | 25936 |
| 297 | 25936 |
| 298 | 23041 |
| 299 | 9206 |
| 300 | 25105 |
| 301 | 25105 |
| 302 | 24779 |
| 303 | 22451 |
| 304 | 22451 |
| 305 | 22291 |
| 306 | 22291 |
| 307 | 21143 |
| 308 | 24751 |
| 309 | 24751 |
| 310 | 24294 |
| 311 | 24294 |
| 312 | 24006 |
| 313 | 24006 |
| 314 | 25678 |
| 315 | 25678 |
| 316 | 22027 |
| 317 | 29495 |
| 318 | 29495 |
| 319 | 24577 |
| 320 | 24577 |
| 321 | 24577 |
| 322 | 23527 |
| 323 | 17090 |
| 324 | 25137 |
| 325 | 23772 |
| 326 | 1659 |
| 327 | 8497 |
| 328 | 25272 |
| 329 | 21216 |
| 330 | 21216 |
| 331 | 21216 |
| 332 | 11939 |
| 333 | 11939 |
| 334 | 11939 |
| 335 | 9191 |
| 336 | 3429 |
| 337 | 24588 |
| 338 | 4047 |
| 339 | 28344 |
| 340 | 28344 |
| 341 | 27561 |
| 342 | 3272 |
| 343 | 26735 |
| 344 | 26735 |
| 345 | 24900 |
| 346 | 24900 |
| 347 | 9472 |
| 348 | 9472 |
| 349 | 9979 |
| 350 | 21996 |
| 351 | 22312 |
| 352 | 11327 |
| 353 | 18240 |
| 354 | 18240 |
| 355 | 21922 |
| 356 | 21922 |
| 357 | 22290 |
| 358 | 10390 |
| 359 | 10390 |
| 360 | 2212 |
| 361 | 20213 |
| 362 | 20213 |
| 363 | 24955 |
| 364 | 19574 |
| 365 | 19969 |
| 366 | 8570 |
| 367 | 18519 |
| 368 | 9616 |
| 369 | 9616 |
| 370 | 17459 |
| 371 | 17459 |
| 372 | 25193 |
| 373 | 25193 |
| 374 | 25193 |
| 375 | 25191 |
| 376 | 22566 |
| 377 | 4164 |
| 378 | 4146 |
| 379 | 4072 |
| 380 | 4022 |
| 381 | 3954 |
| 382 | 3838 |
| 383 | 3806 |
| 384 | 3798 |
| 385 | 3792 |
| 386 | 3788 |
| 387 | 3458 |
| 388 | 3194 |
| 389 | 3102 |
| 390 | 25191 |
| 391 | 25191 |
| 392 | 9448 |
| 393 | 9448 |
| 394 | 25224 |
| 395 | 20218 |
| 396 | 3089 |
| 397 | 3089 |
| 398 | 19953 |
| 399 | 19953 |
| 400 | 22362 |
| 401 | 25516 |
| 402 | 25516 |
| 403 | 25757 |
| 404 | 24814 |
| 405 | 21994 |
| 406 | 27117 |
| 407 | 22745 |
| 408 | 24233 |
| 409 | 2001 |
| 410 | 2001 |
| 411 | 2001 |
| 412 | 17147 |
| 413 | 21214 |
| 414 | 21214 |
| 415 | 21214 |
| 416 | 26422 |
| 417 | 21965 |
| 418 | 25541 |
| 419 | 25541 |
| 420 | 18302 |
| 421 | 18302 |
| 422 | 24049 |
| 423 | 24049 |
| 424 | 26326 |
| 425 | 26326 |
| 426 | 2254 |
| 427 | 162502 |
| 428 | 10296 |
| 429 | 20044 |
| 430 | 28806 |
| 431 | 17566 |
| 432 | 17566 |
| 433 | 19005 |
| 434 | 3567 |
| 435 | 159223 |
| 436 | 3567 |
| 437 | 3567 |
| 438 | 458 |
| 439 | 21411 |
| 440 | 22972 |
| 441 | 24853 |
| 442 | 21502 |
| 443 | 18180 |
| 444 | 23918 |
| 445 | 24144 |
| 446 | 19996 |
| 447 | 11528 |
| 448 | 20506 |
| 449 | 20506 |
| 450 | 23833 |
| 451 | 20042 |
| 452 | 20042 |
| 453 | 11646 |
| 454 | 10577 |
| 455 | 10577 |
| 456 | 18556 |
| 457 | 29433 |
| 458 | 28763 |
| 459 | 27450 |
| 460 | 27450 |
| 461 | 27255 |
| 462 | 26550 |
| 463 | 26550 |
| 464 | 26508 |
| 465 | 26334 |
| 466 | 26334 |
| 467 | 26027 |
| 468 | 26027 |
| 469 | 25977 |
| 470 | 25977 |
| 471 | 25965 |
| 472 | 25965 |
| 473 | 25844 |
| 474 | 25844 |
| 475 | 25834 |
| 476 | 25816 |
| 477 | 25746 |
| 478 | 25712 |
| 479 | 25621 |
| 480 | 25621 |
| 481 | 25614 |
| 482 | 25614 |
| 483 | 25603 |
| 484 | 25603 |
| 485 | 25556 |
| 486 | 25556 |
| 487 | 25555 |
| 488 | 25555 |
| 489 | 3094 |
| 490 | 2567 |
| 491 | 1958 |
| 492 | 1680 |
| 493 | 1445 |
| 494 | 1320 |
| 495 | 974 |
| 496 | 652 |
| 497 | 630 |
| 498 | 593 |
| 499 | 256 |

That which is claimed is:

1. A method for inhibiting a cancerous phenotype of a cell, said method comprising: contacting a cancerous mammalian cell with an agent for inhibition of DKFZp5661133 activity.

2. The method of claim 1, wherein said test cell is a breast cell.

3. The method of claims 1-2, wherein said cancerous phenotype is aberrant cellular proliferation relative to a normal cell.

4. The method of claims 1-3, wherein said cancerous phenotype is loss of contact inhibition of cell growth.

5. The method of claims 1-4, wherein said agent is selected from the group consisting of a small molecule, an antibody, an antisense polynucleotide, and an RNAi molecule.

6. The method of claims 1-6, wherein said inhibition is associated with a reduction in a level of DKFZp566I133 protein.

7. The method of claims 1-7, wherein said inhibition is associated with a reduction in a level of DKFZp566I133 RNA.

8. The method of claims 1-8, wherein said inhibition is associated with a reduction in a level of activity of DKFZp566I133 protein.

9. A method for detecting a cancerous cell, said method comprising: detecting a level of DKFZp566I133 or fragment thereof in a test sample obtained from a cell of a subject,
comparing the level of DKFZp566I133 to a control level of DKFZp5661133,
wherein the presence of a cancerous cell is indicated by detection of said level and comparison to a control level of DKFZp566I133.

10. The method of claim 9, wherein said cancerous cell is a cancerous breast cell.

11. The method of claims 9-10, wherein said gene product is nucleic acid.

12. The method of claims 9-11, wherein said gene product is a polypeptide.

13. The method of claims 9-12, wherein said detecting step uses a polymerase chain reaction.

14. The method of claims 9-13, wherein said detecting step uses hybridization.

15. The method of claims 9-14, wherein said sample is a sample of breast tissue.

16. The method of claims 9-15, wherein said level of said product is indicative of the cancerous state of the cell of the test sample.

17. A method of treating a subject with cancer, said method comprising: administering to a subject a pharmaceutically effective amount of an agent, wherein said agent modulates the activity of DKFZp566I133.

18. The method of claim 17, wherein said cancer is breast cancer.

19. The method of claims 17-18, wherein said agent is selected from the group consisting of a small molecule, an antibody, an antisense polynucleotide, and an RNAi molecule.

20. A method for assessing the tumor burden of a subject, said method comprising:
detecting a level of DKFZp566I133 in a test sample from a subject,
wherein the level of DKFZp566I133 in the test sample is indicative of the tumor burden in the subject.

21. A method for identifying an agent that modulates a biological activity of a gene product differentially expressed in a cancerous cell as compared to a normal cell, said method comprising: contacting a candidate agent with a DKFZp566I133; and
detecting modulation of a biological activity of DKFZp566I133 relative to a level of biological activity of DKFZp566I133 in the absence of the candidate agent.

22. The method of claim 21, wherein said cancerous cell and said normal cell are breast cells.

23. The method of claims 21-22, wherein said detecting is by assessing expression of said gene product.

24. The method of claim 23, wherein expression is assessed by detecting a polynucleotide gene product.

25. The method of claims 23-24, wherein expression is assessed by detecting a polypeptide gene product.

26. The method of claims 21-25, wherein said candidate agent is selected from the group consisting of a small molecule, an antibody, an antisense polynucleotide, and an RNAi molecule.

27. The method of claims 21-26, wherein said biological activity is modulation of a cancerous phenotype.

28. The method of claim 27, wherein said cancerous phenotype is abnormal cellular proliferation.

29. The method of claim 27-28, wherein said cancerous phenotype is loss of contact inhibition.

30. An isolated polynucleotide comprising at least 15 contiguous nucleotides of a sequence selected from the group consisting of SEQ ID NO:1-499.
